SEQUENCE LISTING (280 sequences)

SEQ ID NO: 1 - protein, 12 aa, synthetic construct
ELLEELLEELLE

SEQ ID NO: 2 - protein, 12 aa, synthetic construct
DLLDDLLDDLLE

SEQ ID NO: 3 - protein, 7 aa, synthetic construct
GFGFVGG

SEQ ID NO: 4 - protein, 12 aa, synthetic construct
QLLQQLLQQLLQ

SEQ ID NO: 5 - skipped

SEQ ID NO: 6 - protein, 31 aa, synthetic construct
ELLEELLEELLEGFGFVGGQLLQQLLQQLLQ

SEQ ID NO: 7 - protein, 33 aa, synthetic construct
ELLEELLEELLEGFGFVGGQLLQQLLQQLLQKK

SEQ ID NO: 8 - protein, 36 aa, synthetic construct
  SITE 35: note = X = naphthylalanine
  MOD_RES 36: note = Glycine comprising a -C(O)-NH2 group
ELLEELLEELLEGFGFVGGQLLQQLLQQLLQKKGXG

SEQ ID NO: 9 - protein, 31 aa, synthetic construct
DLLDDLLDDLLEGFGFVGGQLLQQLLQQLLQ

SEQ ID NO: 10 - protein, 33 aa, synthetic construct
DLLDDLLDDLLEGFGFVGGQLLQQLLQQLLQKK

SEQ ID NO: 11 - protein, 36 aa, synthetic construct
  MOD_RES 36: note = Gycine comprising a -C(O)-NH2 group
  SITE 35: note = X = naphthylalanine
DLLDDLLDDLLEGFGFVGGQLLQQLLQQLLQKKGXG

SEQ ID NO: 12 - protein, 37 aa, synthetic construct
  SITE 35: note = X =naphthylalanine
DLLDDLLDDLLEGFGFVGGQLLQQLLQQLLQKKGXKG

SEQ ID NO: 13 - protein, 34 aa, synthetic construct
  MOD_RES 34: note = Glycine comprising a -C(O)-NH2 group
ELLEELLEELLEGFGFVGGQLLQQLLQQLLQKKG

SEQ ID NO: 14 - protein, 36 aa, synthetic construct; identical to SEQ ID NO: 8
  SITE 35: note = X = naphthylalanine
  MOD_RES 36: note = Glycine comprising -C(O)-NH2 group

SEQ ID NO: 15 - protein, 36 aa, synthetic construct
  MOD_RES 36: note = Glycine comprising a -C(O)-NH2 group
ELLEELLEELLEGFGFVGGQLLQQLLQQLLQKKGWG

SEQ ID NO: 16 - protein, 13 aa, synthetic construct
ELLEELLEELLEL

SEQ ID NO: 17 - protein, 14 aa, synthetic construct
ELLEELLEELLELL

SEQ ID NO: 18 - protein, 35 aa, synthetic construct
  MOD_RES 35: note = Glycine comprising -C(O)NH2
ELLEELLEELLELGFGFVGGQLLQQLLQQLLQKKG

SEQ ID NO: 19 - protein, 36 aa, synthetic construct
  MOD_RES 36: note = Glycine comprising -C(O)NH2
ELLEELLEELLELLGFGFVGGQLLQQLLQQLLQKKG

SEQ ID NO: 20 - protein, 12 aa, synthetic construct
EIIEEIIEEIIE

SEQ ID NO: 21 - protein, 34 aa, synthetic construct
  MOD_RES 34: note = Glycine comprising -C(O)NH2
EIIEEIIEEIIEGFGFVGGQLLQQLLQQLLQKKG

SEQ ID NO: 22 - protein, 22 aa, synthetic construct
  MOD_RES 22: note = Glycine comprising -C(O)NH2
ELLEELLEELLEGFGFVGGKKG

SEQ ID NO: 23 - protein, 24 aa, synthetic construct
  MOD_RES 24: note = Glycine comprising -C(O)NH2
ELLEELLEELLELLGFGFVGGKKG

SEQ ID NO: 24 - protein, 12 aa, synthetic construct
ELLQELLQELLE

SEQ ID NO: 25 - protein, 34 aa, synthetic construct
  MOD_RES 34: note = Glycine comprising -C(O)NH2
ELLQELLQELLEGFGFVGGQLLQQLLQQLLQKKG

SEQ ID NO: 26 - protein, 12 aa, synthetic construct
ELLQELLQELLQ

SEQ ID NO: 27 - protein, 34 aa, synthetic construct
  MOD_RES 34: note = Glycine comprising -C(O)NH2
ELLQELLQELLQGFGFVGGQLLQQLLQQLLQKKG

SEQ ID NO: 28 - protein, 12 aa, synthetic construct
QLLEQLLEELLE

SEQ ID NO: 29 - protein, 34 aa, synthetic construct
  MOD_RES 34: note = Glycine comprising -C(O)NH2
QLLEQLLEELLEGFGFVGGQLLQQLLQQLLQKKG

SEQ ID NO: 30 - protein, 12 aa, synthetic construct
ELLEQLLQELLE

SEQ ID NO: 31 - protein, 34 aa, synthetic construct
  MOD_RES 34: note = Glycine comprising -C(O)NH2
ELLEQLLQELLEGFGFVGGQLLQQLLQQLLQKKG

SEQ ID NO: 32 - protein, 12 aa, synthetic construct
QLLEELLEELLQ

SEQ ID NO: 33 - protein, 34 aa, synthetic construct
  MOD_RES 34: note = Glycine comprising -C(O)NH2
QLLEELLEELLQGFGFVGGQLLQQLLQQLLQKKG

SEQ ID NO: 34 - protein, 12 aa, synthetic construct
ELLQELLEQLLE

SEQ ID NO: 35 - protein, 34 aa, synthetic construct
  MOD_RES 34: note = Glycine comprising -C(O)NH2
ELLQELLEQLLEGFGFVGGQLLQQLLQQLLQKKG

SEQ ID NO: 36 - protein, 12 aa, synthetic construct
  SITE 1: note = D-glutamic acid
  SITE 2..3: note = D-leucine
  SITE 4..5: note = D-glutamic acid
  SITE 6..7: note = D-leucine
  SITE 8..9: note = D-glutamic acid
  SITE 10..11: note = D-leucine
  SITE 12: note = D-glutamic acid
ELLEELLEELLE

SEQ ID NO: 37 - protein, 34 aa, synthetic construct; identical to SEQ ID NO: 13
  SITE 1: note = D-glutamic acid
  SITE 2..3: note = D-leucine
  SITE 4..5: note = D-glutamic acid
  SITE 6..7: note = D-leucine
  SITE 8..9: note = D-glutamic acid
  SITE 10..11: note = D-leucine
  MOD_RES 34: note = Glycine comprising -C(O)NH2

SEQ ID NO: 38 - protein, 16 aa, synthetic construct
ELLEELLEELLEELLE

SEQ ID NO: 39 - protein, 42 aa, synthetic construct
  MOD_RES 42: note = Glycine comprising -C(O)-NH2
ELLEELLEELLEELLEGFGFVGGQLLQQLLQQLLQQLLQKKG

SEQ ID NO: 40 - protein, 14 aa, synthetic construct
LEELLEELEELLEE

SEQ ID NO: 41 - protein, 14 aa, synthetic construct
LQQLLQQLQQLLQQ

SEQ ID NO: 42 - protein, 38 aa, synthetic construct
  MOD_RES 38: note = Glycine comprising -C(O)NH2
LEELLEELEELLEEGFGFVGGLQQLLQQLQQLLQQKKG

SEQ ID NO: 43 - protein, 19 aa, synthetic construct
EEEELLEELLEELLEELLE

SEQ ID NO: 44 - protein, 19 aa, synthetic construct
EEELLEELLEELLEELLEE

SEQ ID NO: 45 - protein, 19 aa, synthetic construct
EELLEELLEELLEELLEEE

SEQ ID NO: 46 - protein, 18 aa, synthetic construct
EEELLEELLEELLEELLE

SEQ ID NO: 47 - protein, 18 aa, synthetic construct
ELLEELLEELLEELLEEE

SEQ ID NO: 48 - protein, 18 aa, synthetic construct
EELLEELLEELLEELLEE

SEQ ID NO: 49 - protein, 17 aa, synthetic construct
EELLEELLEELLEELLE

SEQ ID NO: 50 - protein, 17 aa, synthetic construct
ELLEELLEELLEELLEE

SEQ ID NO: 51 - protein, 16 aa, synthetic construct
QLLQQLLQQLLQQLLQ

SEQ ID NO: 52 - protein, 45 aa, synthetic construct
  MOD_RES 45: note = Glycine comprising -C(O)NH2
EEEELLEELLEELLEELLEGFGFVGGQLLQQLLQQLLQQLLQKKG

SEQ ID NO: 53 - protein, 45 aa, synthetic construct
  MOD_RES 45: note = Glycine comprising -C(O)NH2
EEELLEELLEELLEELLEEGFGFVGGQLLQQLLQQLLQQLLQKKG

SEQ ID NO: 54 - protein, 45 aa, synthetic construct
  MOD_RES 45: note = Glycine comprising -C(O)NH2
EELLEELLEELLEELLEEEGFGFVGGQLLQQLLQQLLQQLLQKKG

SEQ ID NO: 55 - protein, 44 aa, synthetic construct
  MOD_RES 44: note = Glycine comprising -C(O)NH2
EEELLEELLEELLEELLEGFGFVGGQLLQQLLQQLLQQLLQKKG

SEQ ID NO: 56 - protein, 44 aa, synthetic construct
  MOD_RES 44: note = Glycine comprising -C(O)NH2
ELLEELLEELLEELLEEEGFGFVGGQLLQQLLQQLLQQLLQKKG

SEQ ID NO: 57 - protein, 44 aa, synthetic construct
  MOD_RES 44: note = Glycine comprising -C(O)NH2
EELLEELLEELLEELLEEGFGFVGGQLLQQLLQQLLQQLLQKKG

SEQ ID NO: 58 - protein, 43 aa, synthetic construct
  MOD_RES 43: note = Glycine comprising -C(O)NH2
EELLEELLEELLEELLEGFGFVGGQLLQQLLQQLLQQLLQKKG

SEQ ID NO: 59 - protein, 43 aa, synthetic construct
  MOD_RES 43: note = Glycine comprising -C(O)NH2
ELLEELLEELLEELLEEGFGFVGGQLLQQLLQQLLQQLLQKKG

SEQ ID NO: 60 - protein, 15 aa, synthetic construct
EEEQLLEELLEELLQ

SEQ ID NO: 61 - protein, 15 aa, synthetic construct
EEQLLEELLEELLQE

SEQ ID NO: 62 - protein, 15 aa, synthetic construct
EQLLEELLEELLQEE

SEQ ID NO: 63 - protein, 14 aa, synthetic construct
EEQLLEELLEELLQ

SEQ ID NO: 64 - protein, 14 aa, synthetic construct
QLLEELLEELLQEE

SEQ ID NO: 65 - protein, 14 aa, synthetic construct
EQLLEELLEELLQE

SEQ ID NO: 66 - protein, 13 aa, synthetic construct
EQLLEELLEELLQ

SEQ ID NO: 67 - protein, 13 aa, synthetic construct
QLLEELLEELLQE

SEQ ID NO: 68 - protein, 13 aa, synthetic construct
EQLLEELLEELLQ

SEQ ID NO: 69 - protein, 37 aa, synthetic construct
  MOD_RES 37: note = Glycine comprising -C(O)NH2
EEEQLLEELLEELLQGFGFVGGQLLQQLLQQLLQKKG

SEQ ID NO: 70 - protein, 37 aa, synthetic construct
  MOD_RES 37: note = Glycine comprising -C(O)NH2
EEQLLEELLEELLQEGFGFVGGQLLQQLLQQLLQKKG

SEQ ID NO: 71 - protein, 37 aa, synthetic construct
  MOD_RES 37: note = Glycine comprising -C(O)NH2
EQLLEELLEELLQEEGFGFVGGQLLQQLLQQLLQKKG

SEQ ID NO: 72 - protein, 36 aa, synthetic construct
  MOD_RES 36: note = Glycine comprising -C(O)NH2
EEQLLEELLEELLQGFGFVGGQLLQQLLQQLLQKKG

SEQ ID NO: 73 - protein, 36 aa, synthetic construct
  MOD_RES 36: note = Glycine comprising -C(O)NH2
QLLEELLEELLQEEGFGFVGGQLLQQLLQQLLQKKG

SEQ ID NO: 74 - protein, 36 aa, synthetic construct
  MOD_RES 36: note = Glycine comprising -C(O)NH2
EQLLEELLEELLQEGFGFVGGQLLQQLLQQLLQKKG

SEQ ID NO: 75 - protein, 35 aa, synthetic construct
  MOD_RES 35: note = Glycine comprising -C(O)NH2
EQLLEELLEELLQGFGFVGGQLLQQLLQQLLQKKG

SEQ ID NO: 76 - protein, 35 aa, synthetic construct
  MOD_RES 35: note = Glycine comprising -C(O)NH2
QLLEELLEELLQEGFGFVGGQLLQQLLQQLLQKKG

SEQ ID NO: 77 - protein, 35 aa, synthetic construct; identical to SEQ ID NO: 75
  MOD_RES 35: note = Glycine comprising -C(O)NH2

SEQ ID NO: 78 - protein, 12 aa, synthetic construct
  SITE 3: note = X = alpha-methyl leucine
ELXEELLEELLE

SEQ ID NO: 79 - protein, 34 aa, synthetic construct
  MOD_RES 3: note = X = alpha-methyl leucine
  MOD_RES 34: note = Glycine comprising -C(O)NH2
ELXEELLEELLEGFGFVGGQLLQQLLQQLLQKKG

SEQ ID NO: 80 - protein, 12 aa, synthetic construct
  SITE 2: note = X = alpha-methyl leucine
  SITE 6: note = X = alpha-methyl leucine
EXLEEXLEELLE

SEQ ID NO: 81 - protein, 12 aa, synthetic construct
  SITE 2: note = X = alpha-methyl leucine
QXLQQLLQQLLQ

SEQ ID NO: 82 - protein, 34 aa, synthetic construct
  MOD_RES 34: note = Glycine comprising -C(O)NH2
  SITE 2: note = X = alpha-methyl leucine
  SITE 6: note = X = alpha-methyl leucine
EXLEEXLEELLEGFGFVGGQLLQQLLQQLLQKKG

SEQ ID NO: 83 - protein, 34 aa, synthetic construct
  MOD_RES 34: note = Glycine comprising -C(O)NH2
  SITE 21: note = X = alpha-methyl leucine
ELLEELLEELLEGFGFVGGQXLQQLLQQLLQKKG

SEQ ID NO: 84 - protein, 12 aa, synthetic construct
DLLDELLDELLE

SEQ ID NO: 85 - protein, 12 aa, synthetic construct
ELLDELLDELLE

SEQ ID NO: 86 - protein, 12 aa, synthetic construct
ELLEDLLEDLLE

SEQ ID NO: 87 - protein, 36 aa, synthetic construct; identical to SEQ ID NO: 8
  MOD_RES 36: note = Glycine comprising -C(O)NH2
  SITE 35: note = X = naphthylalanine

SEQ ID NO: 88 - protein, 36 aa, synthetic construct; identical to SEQ ID NO: 11
  SITE 35: note = X = naphthylalanine
  MOD_RES 36: note = Glycine comprising -C(O)NH2

SEQ ID NO: 89 - protein, 36 aa, synthetic construct
  MOD_RES 36: note = Glycine comprising -C(O)NH2
  SITE 35: note = X = naphthylalanine
DLLDELLDELLEGFGFVGGQLLQQLLQQLLQKKGXG

SEQ ID NO: 90 - protein, 36 aa, synthetic construct
  MOD_RES 36: note = Glycine comprising -C(O)NH2
  SITE 35: note = X = naphthylalanine
ELLDELLDELLEGFGFVGGQLLQQLLQQLLQKKGXG

SEQ ID NO: 91 - protein, 36 aa, synthetic construct
  MOD_RES 36: note = Glycine comprising -C(O)NH2
  SITE 35: note = X = naphthylalanine
ELLEDLLEDLLEGFGFVGGQLLQQLLQQLLQKKGXG

SEQ ID NO: 92 - protein, 12 aa, synthetic construct
RLLRRLLRRLLR

SEQ ID NO: 93 - protein, 20 aa, synthetic construct
DLLDDLLDDLLDDLLDDLLE

SEQ ID NO: 94 - protein, 16 aa, synthetic construct
DLLDDLLDDLLDDLLE

SEQ ID NO: 95 - protein, 12 aa, synthetic construct
DLLDDLLDDLLE

SEQ ID NO: 96 - protein, 8 aa, synthetic construct
DLLDDLLE

SEQ ID NO: 97 - protein, 4 aa, synthetic construct
DLLE

SEQ ID NO: 98 - protein, 20 aa, synthetic construct
QLLQQLLQQLLQQLLQQLLQ

SEQ ID NO: 99 - protein, 16 aa, synthetic construct
QLLQQLLQQLLQQLLQ

SEQ ID NO: 100 - protein, 8 aa, synthetic construct
QLLQQLLQ

SEQ ID NO: 101 - protein, 4 aa, synthetic construct
QLLQ

SEQ ID NO: 102 - protein, 36 aa, synthetic construct
  MOD_RES 36: note = Glycine comprising -C(O)NH2
  SITE 35: note = X = naphthylalanine
RLLRRLLRRLLRGFGFVGGQLLQQLLQQLLQKKGXG

SEQ ID NO: 103 - protein, 52 aa, synthetic construct
  MOD_RES 52: note = Glycine comprising -C(O)NH2
  SITE 51: note = X = naphthylalanine
DLLDDLLDDLLDDLLDDLLEGFGFVGGQLLQQLLQQLLQQLLQQLLQKKGXG

SEQ ID NO: 104 - protein, 44 aa, synthetic construct
  MOD_RES 44: note = Glycine comprising -C(O)NH2
  SITE 43: note = X = naphthylalanine
DLLDDLLDDLLDDLLEGFGFVGGQLLQQLLQQLLQQLLQKKGXG

SEQ ID NO: 105 - protein, 36 aa, synthetic construct; identical to SEQ ID NO: 11
  MOD_RES 36: note = Glycine comprising -C(O)NH2
  SITE 35: note = X = naphthylalanine

SEQ ID NO: 106 - protein, 28 aa, synthetic construct
  MOD_RES 28: note = Glycine comprising -C(O)NH2
  SITE 27: note = X = naphthylalanine
DLLDDLLEGFGFVGGQLLQQLLQKKGXG

SEQ ID NO: 107 - protein, 20 aa, synthetic construct
  MOD_RES 20: note = Glycine comprising -C(O)NH2
  SITE 19: note = X = naphthylalanine
DLLEGFGFVGGQLLQKKGXG

SEQ ID NO: 108 - protein, 37 aa, synthetic construct
  SITE 35: note = X = naphthylalanine
ELLEELLEELLEGFGFVGGQLLQQLLQQLLQKKGXKG

SEQ ID NO: 109 - protein, 16 aa, synthetic construct
DLDDLDLLDLDDLDLL

SEQ ID NO: 110 - protein, 15 aa, synthetic construct
DLLLDDLLLDDLLLE

SEQ ID NO: 111 - protein, 16 aa, synthetic construct
QLQQLQLLQLQQLQLL

SEQ ID NO: 112 - protein, 15 aa, synthetic construct
QLLLQQLLLQQLLLQ

SEQ ID NO: 113 - protein, 44 aa, synthetic construct
  MOD_RES 44: note = Glycine comprising -C(O)NH2
  SITE 43: note = X = naphthylalanine
DLDDLDLLDLDDLDLLGFGFVGGQLQQLQLLQLQQLQLLKKGXG

SEQ ID NO: 114 - protein, 42 aa, synthetic construct
  MOD_RES 42: note = Glycine comprising -C(O)NH2
  SITE 41: note = X = naphthylalanine
DLLLDDLLLDDLLLEGFGFVGGQLLLQQLLLQQLLLQKKGXG

SEQ ID NO: 115 - protein, 14 aa, synthetic construct
ELLEELLEELLELL

SEQ ID NO: 116 - protein, 8 aa, synthetic construct
ELLEELLE

SEQ ID NO: 117 - protein, 4 aa, synthetic construct
ELLE

SEQ ID NO: 118 - protein, 26 aa, synthetic construct
  MOD_RES 26: note = Glycine comprising -C(O)NH2
  SITE 24: note = X = naphthylalanine
  MOD_RES 25: note = X = lysine with azide side chain modification
ELLEELLEELLELLGFGFVGGKGXXG

SEQ ID NO: 119 - protein, 25 aa, synthetic construct
  MOD_RES 25: note = Glycine comprising -C(O)NH2
  SITE 23: note = X = naphthylalanine
  MOD_RES 24: note = X = lysine with azide side chain modification
ELLEELLEELLEGFGFVGGKKGXXG

SEQ ID NO: 120 - protein, 24 aa, synthetic construct
  MOD_RES 24: note = Glycine comprising -C(O)NH2
  SITE 22: note = X = naphthylalanine
  MOD_RES 23: note = X = lysine with azide side chain modification
ELLEELLEELLEGFGFVGGKGXXG

SEQ ID NO: 121 - protein, 20 aa, synthetic construct
  MOD_RES 20: note = Glycine comprising -C(O)NH2
  SITE 18: note = X = naphthylalanine
  MOD_RES 19: note = X = lysine with azide side chain modification
ELLEELLEGFGFVGGKGXXG

SEQ ID NO: 122 - protein, 16 aa, synthetic construct
  MOD_RES 16: note = Glycine comprising -C(O)NH2
  SITE 15: note = X = naphthylalanine
  MOD_RES 13: note = X = lysine with azide side chain modification
ELLEGFGFVGGKXGXG

SEQ ID NO: 123 - protein, 24 aa, synthetic construct
  MOD_RES 24: note = Glycine comprising -C(O)NH2
  SITE 23: note = X = naphthylalanine
  MOD_RES 21: note = X is lysine with azide side chain modification
DLLDDLLDDLLEGFGFVGGKXGXG

SEQ ID NO: 124 - protein, 33 aa, synthetic construct
  MOD_RES 33: note = Glycine comprising -C(O)NH2
  SITE 32: note = X = naphthylalanine
DLLDDLLDDLLEPKPLQLLQQLLQQLLQKKGXG

SEQ ID NO: 125 - protein, 31 aa, synthetic construct
  MOD_RES 31: note = Glycine comprising -C(O)NH2
  SITE 30: note = X = naphthylalanine
DLLDDLLDDLLEVGQLLQQLLQQLLQKKGXG

SEQ ID NO: 126 - protein, 31 aa, synthetic construct
  MOD_RES 31: note = Glycine comprising -C(O)NH2
  SITE 14: note = X = citrulline
  SITE 30: note = X = naphthylalanine
DLLDDLLDDLLEVXQLLQQLLQQLLQKKGXG

SEQ ID NO: 127 - protein, 29 aa, synthetic construct
  MOD_RES 29: note = Glycine comprising -C(O)NH2
  SITE 28: note = X = naphthylalanine
DLLDDLLDDLLEQLLQQLLQQLLQKKGXG

SEQ ID NO: 128 - protein, 33 aa, synthetic construct
  MOD_RES 33: note = Glycine comprising -C(O)NH2
ELLEELLEELLEGGGGGGGQLLQQLLQQLLQKG

SEQ ID NO: 129 - protein, 33 aa, synthetic construct
  MOD_RES 33: note = Glycine comprising -C(O)NH2
ELLEELLEELLEGFGFVGGQLLQQLLQQLLQKG

SEQ ID NO: 130 - protein, 31 aa, synthetic construct
  MOD_RES 31: note = Glycine comprising -C(O)NH2
  SITE 17: note = X = aminocaproic acid
ELLEELLEELLEGFGFXQLLQQLLQQLLQKG

SEQ ID NO: 131 - protein, 38 aa, synthetic construct
  MOD_RES 38: note = Glycine comprising -C(O)NH2
  MOD_RES 14: note = X is lysine with azide side chain modification
  SITE 37: note = X = naphthylalanine
ELLEELLEELLEGXGFGFVGGQLLQQLLQQLLQKKGXG

SEQ ID NO: 132 - protein, 37 aa, synthetic construct
  MOD_RES 37: note = Glycine comprising -C(O)NH2
  MOD_RES 19: note = X is lysine with azide side chain modification
  SITE 36: note = X = naphthylalanine
ELLEELLEELLEGFGFVGXGQLLQQLLQQLLQKKGXG

SEQ ID NO: 133 - protein, 36 aa, synthetic construct
  MOD_RES 36: note = Glycine comprising -C(O)NH2
  MOD_RES 14: note = X is phenylalanine with azide side chain modification
  SITE 35: note = X = naphthylalanine
ELLEELLEELLEGXGFVGGQLLQQLLQQLLQKKGXG

SEQ ID NO: 134 - protein, 12 aa, synthetic construct
SLLSSLLSSLLS

SEQ ID NO: 135 - protein, 33 aa, synthetic construct
  MOD_RES 33: note = Glycine comprising -C(O)NH2
ELLEELLEELLEGFGFVGGSLLSSLLSSLLSKG

SEQ ID NO: 136 - protein, 12 aa, synthetic construct
ALLAQLLAQLLQ

SEQ ID NO: 137 - protein, 12 aa, synthetic construct
ALLAALLAALLA

SEQ ID NO: 138 - protein, 33 aa, synthetic construct
  MOD_RES 33: note = Glycine comprising -C(O)NH2
ELLEELLEELLEGFGFVGGALLAQLLAQLLQKG

SEQ ID NO: 139 - protein, 33 aa, synthetic construct
  MOD_RES 33: note = Glycine comprising -C(O)NH2
ELLEELLEELLEGFGFVGGALLAALLAALLAKG

SEQ ID NO: 140 - protein, 12 aa, synthetic construct
QLLQQLLQQLQQ

SEQ ID NO: 141 - protein, 12 aa, synthetic construct
QQLQQLLQQLQQ

SEQ ID NO: 142 - protein, 34 aa, synthetic construct
  MOD_RES 34: note = Glycine comprising -C(O)NH2
ELLEELLEELLEGFGFVGGQLLQQLLQQLQQKKG

SEQ ID NO: 143 - protein, 34 aa, synthetic construct
  MOD_RES 34: note = Glycine comprising -C(O)NH2
ELLEELLEELLEGFGFVGGQQLQQLLQQLQQKKG

SEQ ID NO: 144 - protein, 34 aa, synthetic construct
  MOD_RES 34: note = Glycine comprising -C(O)NH2
ELLEELLEELLEGFGFVGGELLEELLEELLEKKG

SEQ ID NO: 145 - protein, 12 aa, synthetic construct
DLLDDLLDDLLD

SEQ ID NO: 146 - protein, 33 aa, synthetic construct
  MOD_RES 33: note = Glycine comprising -C(O)NH2
ELLEELLEELLEGFGFVGGDLLDDLLDDLLDKG

SEQ ID NO: 147 - protein, 21 aa, synthetic construct
  MOD_RES 21: note = Glycine comprising -C(O)NH2
ELLEELLEELLEGFGFVGGKG

SEQ ID NO: 148 - protein, 12 aa, synthetic construct
QLLAQLLAQLLQ

SEQ ID NO: 149 - protein, 33 aa, synthetic construct
  MOD_RES 33: note = Glycine comprising -C(O)NH2
ELLEELLEELLEGFGFVGGQLLAQLLAQLLQKG

SEQ ID NO: 150 - protein, 12 aa, synthetic construct
QLLEELLEELLQ

SEQ ID NO: 151 - protein, 12 aa, synthetic construct
ELLEQLLQELLE

SEQ ID NO: 152 - protein, 12 aa, synthetic construct
ELLQQLLEQLLE

SEQ ID NO: 153 - protein, 12 aa, synthetic construct
QLLEQLLQQLLE

SEQ ID NO: 154 - protein, 12 aa, synthetic construct
QLLQELLEQLLQ

SEQ ID NO: 155 - protein, 12 aa, synthetic construct
ELLQQLLQQLLE

SEQ ID NO: 156 - protein, 12 aa, synthetic construct
QLLEQLLQQLLQ

SEQ ID NO: 157 - protein, 12 aa, synthetic construct
QLLEQLLEQLLE

SEQ ID NO: 158 - protein, 12 aa, synthetic construct
ELLQELLQQLLE

SEQ ID NO: 159 - protein, 33 aa, synthetic construct
  MOD_RES 33: note = Glycine comprising -C(O)NH2
QLLEELLEELLQGFGFVGGQLLQELLEQLLQKG

SEQ ID NO: 160 - protein, 33 aa, synthetic construct
  MOD_RES 33: note = Glycine comprising -C(O)NH2
QLLEELLEELLQGFGFVGGQLLEQLLQQLLEKG

SEQ ID NO: 161 - protein, 33 aa, synthetic construct
  MOD_RES 33: note = Glycine comprising -C(O)NH2
QLLEELLEELLQGFGFVGGQLLQQLLEQLLEKG

SEQ ID NO: 162 - protein, 33 aa, synthetic construct
  MOD_RES 33: note = Glycine comprising -C(O)NH2
QLLEELLEELLQGFGFVGGELLQELLQELLQKG

SEQ ID NO: 163 - protein, 33 aa, synthetic construct
  MOD_RES 33: note = Glycine comprising -C(O)NH2
QLLEELLEELLQGFGFVGGELLQQLLQQLLEKG

SEQ ID NO: 164 - protein, 33 aa, synthetic construct
  MOD_RES 33: note = Glycine comprising -C(O)NH2
ELLEQLLQELLEGFGFVGGQLLEQLLQQLLQKG

SEQ ID NO: 165 - protein, 33 aa, synthetic construct
  MOD_RES 33: note = Glycine comprising -C(O)NH2
ELLEQLLQELLEGFGFVGGQLLEQLLQQLLEKG

SEQ ID NO: 166 - protein, 33 aa, synthetic construct
  MOD_RES 33: note = Glycine comprising -C(O)NH2
ELLEQLLQELLEGFGFVGGQLLQQLLEQLLEKG

SEQ ID NO: 167 - protein, 33 aa, synthetic construct
  MOD_RES 33: note = Glycine comprising -C(O)NH2
ELLEQLLQELLEGFGFVGGELLQELLQELLQKG

SEQ ID NO: 168 - protein, 33 aa, synthetic construct
  MOD_RES 33: note = Glycine comprising -C(O)NH2
ELLQELLQELLQGFGFVGGELLQELLQELLQKG

SEQ ID NO: 169 - protein, 33 aa, synthetic construct
  MOD_RES 33: note = Glycine comprising -C(O)NH2
ELLQELLQELLQGFGFVGGQLLEQLLEQLLEKG

SEQ ID NO: 170 - protein, 33 aa, synthetic construct
  MOD_RES 33: note = Glycine comprising -C(O)NH2
ELLQQLLEQLLEGFGFVGGELLQELLQQLLEKG

SEQ ID NO: 171 - protein, 33 aa, synthetic construct
  MOD_RES 33: note = Glycine comprising -C(O)NH2
QLLEQLLQQLLEGFGFVGGQLLEQLLQQLLEKG

SEQ ID NO: 172 - protein, 16 aa, synthetic construct
QLLQQLLQQLLQQLLQ

SEQ ID NO: 173 - protein, 16 aa, synthetic construct
ELLQQLLQQLLQQLLQ

SEQ ID NO: 174 - protein, 16 aa, synthetic construct
QLLEQLLQQLLQQLLQ

SEQ ID NO: 175 - protein, 16 aa, synthetic construct
QLLQELLQQLLQQLLQ

SEQ ID NO: 176 - protein, 16 aa, synthetic construct
QLLQQLLEQLLQQLLQ

SEQ ID NO: 177 - protein, 16 aa, synthetic construct
QLLQQLLQELLQQLLQ

SEQ ID NO: 178 - protein, 16 aa, synthetic construct
QLLQQLLQQLLEQLLQ

SEQ ID NO: 179 - protein, 16 aa, synthetic construct
QLLQQLLQQLLQELLQ

SEQ ID NO: 180 - protein, 16 aa, synthetic construct
QLLQQLLQQLLQQLLE

SEQ ID NO: 181 - protein, 16 aa, synthetic construct
ELLQQLLQQLLQQLLE

SEQ ID NO: 182 - protein, 43 aa, synthetic construct; identical to SEQ ID NO: 58
  MOD_RES 43: note = Glycine comprising -C(O)NH2

SEQ ID NO: 183 - protein, 43 aa, synthetic construct
  MOD_RES 43: note = Glycine comprising -C(O)NH2
EELLEELLEELLEELLEGFGFVGGELLQQLLQQLLQQLLQKKG

SEQ ID NO: 184 - protein, 43 aa, synthetic construct
  MOD_RES 43: note = Glycine comprising -C(O)NH2
EELLEELLEELLEELLEGFGFVGGQLLEQLLQQLLQQLLQKKG

SEQ ID NO: 185 - protein, 43 aa, synthetic construct
  MOD_RES 43: note = Glycine comprising -C(O)NH2
EELLEELLEELLEELLEGFGFVGGQLLQELLQQLLQQLLQKKG

SEQ ID NO: 186 - protein, 43 aa, synthetic construct
  MOD_RES 43: note = Glycine comprising -C(O)NH2
EELLEELLEELLEELLEGFGFVGGQLLQQLLEQLLQQLLQKKG

SEQ ID NO: 187 - protein, 43 aa, synthetic construct
  MOD_RES 43: note = Glycine comprising -C(O)NH2
EELLEELLEELLEELLEGFGFVGGQLLQQLLQELLQQLLQKKG

SEQ ID NO: 188 - protein, 43 aa, synthetic construct
  MOD_RES 43: note = Glycine comprising -C(O)NH2
EELLEELLEELLEELLEGFGFVGGQLLQQLLQQLLEQLLQKKG

SEQ ID NO: 189 - protein, 43 aa, synthetic construct
  MOD_RES 43: note = Glycine comprising -C(O)NH2
EELLEELLEELLEELLEGFGFVGGQLLQQLLQQLLQELLQKKG

SEQ ID NO: 190 - protein, 43 aa, synthetic construct
  MOD_RES 43: note = Glycine comprising -C(O)NH2
EELLEELLEELLEELLEGFGFVGGQLLQQLLQQLLQQLLEKKG

SEQ ID NO: 191 - protein, 43 aa, synthetic construct
  MOD_RES 43: note = Glycine comprising -C(O)NH2
EELLEELLEELLEELLEGFGFVGGELLQQLLQQLLQQLLEKKG

SEQ ID NO: 192 - protein, 17 aa, synthetic construct
  MOD_RES 17: note = Glycine comprising -C(O)NH2
  SITE 16: note = X = naphthylalanine
QLLQQLLQQLLQKKGXG

SEQ ID NO: 193 - protein, 35 aa, synthetic construct
  MOD_RES 35: note = Glycine comprising -C(O)NH2
ELLEELLEELLEGFGFVGGQLLQQLLQQLLQKKKG

SEQ ID NO: 194 - protein, 33 aa, synthetic construct; identical to SEQ ID NO: 7

SEQ ID NO: 195 - protein, 36 aa, synthetic construct
  MOD_RES 36: note = Glycine comprising -C(O)NH2
ELLEELLEELLEGFGFVGGQLLQQLLQQLLQKKKKG

SEQ ID NO: 196 - protein, 36 aa, synthetic construct
  MOD_RES 36: note = Glycine comprising -C(O)NH2
  SITE 35: note = X = naphthylalanine
DLLDDLLDDLLEGFGFVGGQLLQQLLQQLLQRRGXG

SEQ ID NO: 197 - protein, 35 aa, synthetic construct
  MOD_RES 35: note = Glycine comprising -C(O)NH2
  MOD_RES 1: note = Tryptophan modified with acetyl group
WELLEELLEELLEGFGFVGGQLLQQLLQQLLQKKG

SEQ ID NO: 198 - protein, 37 aa, synthetic construct
  MOD_RES 37: note = Glycine comprising -C(O)NH2
  SITE 35..36: note = X = naphthylalanine
ELLEELLEELLEGFGFVGGQLLQQLLQQLLQKKGXXG

SEQ ID NO: 199 - protein, 35 aa, synthetic construct; identical to SEQ ID NO: 75
  MOD_RES 35: note = Glycine comprising -C(O)NH2

SEQ ID NO: 200 - protein, 36 aa, synthetic construct
  MOD_RES 36: note = Glycine comprising -C(O)NH2
QLLEELLEELLQGFGFVGGQLLQQLLQQLLQEKKEG

SEQ ID NO: 201 - protein, 35 aa, synthetic construct
  MOD_RES 35: note = Glycine comprising -C(O)NH2
QLLEELLEELLQGFGFVGGQLLQQLLQQLLQKKEG

SEQ ID NO: 202 - protein, 35 aa, synthetic construct
  MOD_RES 35: note = Glycine comprising -C(O)NH2
QLLEELLEELLQGFGFVGGQLLQQLLQQLLQEKKG

SEQ ID NO: 203 - protein, 12 aa, synthetic construct
ELLQELLQELLE

SEQ ID NO: 204 - protein, 12 aa, synthetic construct
ELLQELLQELLQ

SEQ ID NO: 205 - protein, 12 aa, synthetic construct
QLLEQLLEELLE

SEQ ID NO: 206 - protein, 12 aa, synthetic construct
QLLEELLEELLQ

SEQ ID NO: 207 - protein, 12 aa, synthetic construct
ELLQELLEQLLE

SEQ ID NO: 208 - protein, 41 aa, synthetic construct
  MOD_RES 41: note = Glycine comprising -C(O)NH2
ELLQELLQELLEGFGFVGGELLQELLQELLEGFGFVGGKKG

SEQ ID NO: 209 - protein, 41 aa, synthetic construct
  MOD_RES 41: note = Glycine comprising -C(O)NH2
ELLQELLQELLQGFGFVGGELLQELLQELLQGFGFVGGKKG

SEQ ID NO: 210 - protein, 41 aa, synthetic construct
  MOD_RES 41: note = Glycine comprising -C(O)NH2
QLLEQLLEELLEGFGFVGGQLLEQLLEELLEGFGFVGGKKG

SEQ ID NO: 211 - protein, 41 aa, synthetic construct
  MOD_RES 41: note = Glycine comprising -C(O)NH2
QLLEQLLEQLLEGFGFVGGQLLEQLLEQLLEGFGFVGGKKG

SEQ ID NO: 212 - protein, 41 aa, synthetic construct; identical to SEQ ID NO: 211
  MOD_RES 41: note = Glycine comprising -C(O)NH2

SEQ ID NO: 213 - protein, 41 aa, synthetic construct
  MOD_RES 41: note = Glycine comprising -C(O)NH2
QLLEELLEELLQGFGFVGGQLLEELLEELLQGFGFVGGKKG

SEQ ID NO: 214 - protein, 41 aa, synthetic construct
  MOD_RES 41: note = Glycine comprising -C(O)NH2
ELLQELLEQLLEGFGFVGGELLQELLEQLLEGFGFVGGKKG

SEQ ID NO: 215 - protein, 33 aa, synthetic construct
  MOD_RES 33: note = Glycine comprising -C(O)NH2
KELLEELLEELLEGFGFVGGQLLQQLLQQLLQG

SEQ ID NO: 216 - protein, 34 aa, synthetic construct
  MOD_RES 34: note = Glycine comprising -C(O)NH2
ELLEELLEELLEGFGFVGGKQLLQQLLQQLLQKG

SEQ ID NO: 217 - protein, 33 aa, synthetic construct
  MOD_RES 33: note = Glycine comprising -C(O)NH2
QLLQQLLQQLLQKGFGFVGGELLEELLEELLEG

SEQ ID NO: 218 - protein, 34 aa, synthetic construct
  MOD_RES 34: note = Glycine comprising -C(O)NH2
QLLQQLLQQLLQKKGFGFVGGELLEELLEELLEG

SEQ ID NO: 219 - protein, 35 aa, synthetic construct
  MOD_RES 35: note = Glycine comprising -C(O)NH2
QLLQQLLQQLLQKKKGFGFVGGELLEELLEELLEG

SEQ ID NO: 220 - protein, 36 aa, synthetic construct
  MOD_RES 36: note = Glycine comprising -C(O)NH2
QLLQQLLQQLLQKKKKGFGFVGGELLEELLEELLEG

SEQ ID NO: 221 - protein, 36 aa, synthetic construct
  MOD_RES 36: note = Glycine comprising -C(O)NH2
ELLEELLEELLEGKKKGFGFVGGQLLQQLLQQLLQG

SEQ ID NO: 222 - protein, 36 aa, synthetic construct
  MOD_RES 36: note = Glycine comprising -C(O)NH2
QLLQQLLQQLLQGKKKGFGFVGGELLEELLEELLEG

SEQ ID NO: 223 - protein, 40 aa, synthetic construct
  MOD_RES 40: note = Glycine comprising -C(O)NH2
ELLEELLEELLEGFGFVGGKGFGFVGGQLLQQLLQQLLQG

SEQ ID NO: 224 - protein, 36 aa, synthetic construct
  MOD_RES 36: note = Glycine comprising -C(O)NH2
  SITE 17: note = X = aminocaproic acid
  SITE 23: note = X = aminocaproic acid
ELLEELLEELLEGFGFXKGFGFXQLLQQLLQQLLQG

SEQ ID NO: 225 - protein, 36 aa, synthetic construct
  MOD_RES 36: note = Glycine comprising -C(O)NH2
  SITE 17: note = X = aminocaproic acid
  SITE 23: note = X = aminocaproic acid
ELLEQLLQELLEGFGFXKGFGFXQLLQELLEQLLQG

SEQ ID NO: 226 - protein, 37 aa, synthetic construct
  MOD_RES 37: note = Glycine comprising -C(O)NH2
  SITE 35: note = X = naphthylalanine
  MOD_RES 36: note = X is lysine modified with a Z group
DLLDDLLDDLLEGFGFVGGQLLQQLLQQLLQKKGXXG

SEQ ID NO: 227 - skipped

SEQ ID NO: 228 - protein, 33 aa, synthetic construct; identical to SEQ ID NO: 7

SEQ ID NO: 229 - DNA, 25 bp, synthetic construct
  source 1..25: mol_type = other DNA
ggccaaacctcggcttacctgaaat

SEQ ID NO: 230 - DNA, 25 bp, synthetic construct
  source 1..25: mol_type = other DNA
gcaatatgaaacctcttacctcagt

SEQ ID NO: 231 - protein, 36 aa, synthetic construct
  MOD_RES 36: note = Glycine comprising -C(O)NH2
  MOD_RES 16: note = X = phenylalanine with azide side chain modification
  SITE 35: note = X = naphthylalanine
ELLEELLEELLEGFGXVGGQLLQQLLQQLLQKKGXG

SEQ ID NO: 232 - protein, 22 aa, synthetic construct
  MOD_RES 22: note = Glycine comprising -C(O)NH2
GELLEELLEELLEGFGFVGGKG

SEQ ID NO: 233 - protein, 7 aa, synthetic construct
  SITE 5: note = X = citrulline
GGGVXGG

SEQ ID NO: 234 - protein, 7 aa, synthetic construct
GGGVKGG

SEQ ID NO: 235 - protein, 36 aa, synthetic construct
ELLEELLEELLEGFGFVGGQLLQQLLQQLLQKKGKG

SEQ ID NO: 236 - protein, 36 aa, synthetic construct
ELLEELLEELLEGFGFVGGQLLAQLLAQLLQKKGKG

SEQ ID NO: 237 - protein, 35 aa, synthetic construct
ELLEELLEELLEGFGFVGGQLLAQLLAQLLQKGKG

SEQ ID NO: 238 - DNA, 24 bp, synthetic construct
  source 1..24: mol_type = other DNA
ttctggatgcagactttgtggcct

SEQ ID NO: 239 - DNA, 22 bp, synthetic construct
  source 1..22: mol_type = other DNA
agggcaggccattcctctttca

SEQ ID NO: 240 - DNA, 28 bp, synthetic construct
  source 1..28: mol_type = other DNA
gatccatggacatttgactggtatttcc

SEQ ID NO: 241 - DNA, 15 bp, synthetic construct
  source 1..15: mol_type = other DNA
cagcgagacaaccag

SEQ ID NO: 242 - DNA, 25 bp, synthetic construct
  source 1..25: mol_type = other DNA
tccactggaagaagttgattatttc

SEQ ID NO: 243 - DNA, 15 bp, synthetic construct
  source 1..15: mol_type = other DNA
agagacagcgagaca

SEQ ID NO: 244 - DNA, 25 bp, synthetic construct
  source 1..25: mol_type = other DNA
gcttcctgaatgcccaaagaaacac

SEQ ID NO: 245 - protein, 13 aa, synthetic construct
GELLEELLEELLE

SEQ ID NO: 246 - protein, 34 aa, synthetic construct; identical to SEQ ID NO: 13
  MOD_RES 34: note = Glycine modified with a t-butyl amine group

SEQ ID NO: 247 - protein, 34 aa, synthetic construct; identical to SEQ ID NO: 82
  MOD_RES 34: note = Glycine comprising -C(O)NH2
  SITE 2: note = X = alpha-methyl leucine
  SITE 6: note = X = alpha-methyl leucine

SEQ ID NO: 248 - protein, 34 aa, synthetic construct; identical to SEQ ID NO: 13
  MOD_RES 34: note = Glycine comprising -C(O)NH

SEQ ID NO: 249 - protein, 36 aa, synthetic construct; identical to SEQ ID NO: 8
  MOD_RES 36: note = Glycine comprising -C(O)NH
  SITE 35: note = X = naphthylalanine

SEQ ID NO: 250 - protein, 34 aa, synthetic construct; identical to SEQ ID NO: 13
  MOD_RES 34: note = Glycine modified with an i-butyl amine group

SEQ ID NO: 251 - protein, 35 aa, synthetic construct; identical to SEQ ID NO: 193
  MOD_RES 34: note = Glycine modified with an i-butyl amine group

SEQ ID NO: 252 - protein, 32 aa, synthetic construct
ELLEELLEELLEGFGFVGGQLLQQLLQQLLQK

SEQ ID NO: 253 - protein, 32 aa, synthetic construct; identical to SEQ ID NO: 252

SEQ ID NO: 254 - protein, 32 aa, synthetic construct; identical to SEQ ID NO: 252

SEQ ID NO: 255 - protein, 33 aa, synthetic construct; identical to SEQ ID NO: 7

SEQ ID NO: 256 - protein, 33 aa, synthetic construct; identical to SEQ ID NO: 7

SEQ ID NO: 257 - protein, 34 aa, synthetic construct; identical to SEQ ID NO: 13
  MOD_RES 34: note = Glycine comprising -C(O)NH2

SEQ ID NO: 258 - protein, 33 aa, synthetic construct; identical to SEQ ID NO: 7

SEQ ID NO: 259 - protein, 33 aa, synthetic construct; identical to SEQ ID NO: 129
  MOD_RES 33: note = Glycine comprising -C(O)NH2

SEQ ID NO: 260 - protein, 34 aa, synthetic construct; identical to SEQ ID NO: 13
  MOD_RES 34: note = Glycine comprising -C(O)NH2

SEQ ID NO: 261 - protein, 34 aa, synthetic construct; identical to SEQ ID NO: 13
  MOD_RES 34: note = Glycine comprising -C(O)NH2

SEQ ID NO: 262 - protein, 35 aa, synthetic construct; identical to SEQ ID NO: 193
  MOD_RES 35: note = Glycine comprising -C(O)NH2

SEQ ID NO: 263 - protein, 34 aa, synthetic construct; identical to SEQ ID NO: 13
  MOD_RES 34: note = Glycine comprising -C(O)NH2

SEQ ID NO: 264 - protein, 36 aa, synthetic construct; identical to SEQ ID NO: 15
  MOD_RES 36: note = Glycine comprising -C(O)NH2
  MOD_RES 1: note = Glutamic acid modified with an acetyl group

SEQ ID NO: 265 - protein, 33 aa, synthetic construct
  MOD_RES 33: note = Glycine comprising -C(O)NH2
KQLLQQLLQQLLQGFGFVGGELLEELLEELLEG

SEQ ID NO: 266 - protein, 13 aa, synthetic construct
GQLLQQLLQQLLQ

SEQ ID NO: 267 - protein, 33 aa, synthetic construct; identical to SEQ ID NO: 129
  MOD_RES 33: note = Glycine comprising -C(O)NH2

SEQ ID NO: 268 - protein, 33 aa, synthetic construct; identical to SEQ ID NO: 129
  MOD_RES 33: note = Glycine comprising -C(O)NH2

SEQ ID NO: 269 - protein, 33 aa, synthetic construct; identical to SEQ ID NO: 129
  MOD_RES 33: note = Glycine comprising -C(O)NH2

SEQ ID NO: 270 - protein, 36 aa, synthetic construct; identical to SEQ ID NO: 235

SEQ ID NO: 271 - protein, 33 aa, synthetic construct; identical to SEQ ID NO: 129
  MOD_RES 33: note = Glycine comprising -C(O)NH2

SEQ ID NO: 272 - protein, 35 aa, synthetic construct
  SITE 34: note = X = naphthylalanine
  MOD_RES 35: note = Glycine comprising -C(O)NH2
QLLEQLLQQLLEGFGFVGGQLLEQLLQQLLEKGXG

SEQ ID NO: 273 - protein, 35 aa, synthetic construct
  MOD_RES 35: note = Glycine comprising -C(O)NH2
  SITE 34: note = X = naphthylalanine
ELLEELLEELLEGFGFVGGQLLAQLLAQLLQKGXG

SEQ ID NO: 274 - protein, 35 aa, synthetic construct
  SITE 34: note = X = naphthylalanine
  MOD_RES 35: note = Glycine comprising -C(O)NH2
ELLEELLEELLEGFGFVGGQLLQQLLQQLLQKGXG

SEQ ID NO: 275 - protein, 34 aa, synthetic construct
  SITE 33: note = X = naphthylalanine
  MOD_RES 34: note = Glycine comprising -C(O)NH2
RLLRRLLRRLLRGFGFVGGQLLQQLLQQLLQGXG

SEQ ID NO: 276 - protein, 17 aa, synthetic construct
  MOD_RES 17: note = Glycine comprising -C(O)NH2
  SITE 16: note = X = naphthylalanine
RLLRRLLRRLLRKKGXG

SEQ ID NO: 277 - protein, 15 aa, synthetic construct
  SITE 14: note = X = naphthylalanine
  MOD_RES 15: note = Glycine comprising -C(O)NH2
RLLRRLLRRLLRGXG

SEQ ID NO: 278 - protein, 15 aa, synthetic construct
  MOD_RES 15: note = Glycine comprising -C(O)NH2
  SITE 14: note = X = naphthylalanine
ELLEELLEELLEGXG

SEQ ID NO: 279 - protein, 16 aa, synthetic construct
  SITE 15: note = X = naphthylalanine
  MOD_RES 16: note = Glycine comprising -C(O)NH2
ELLEELLEELLEKGXG

SEQ ID NO: 280 - protein, 35 aa, synthetic construct
  MOD_RES 35: note = Glycine comprising -C(O)NH2
  SITE 34: note = X = naphthylalanine
ELLEELLEELLEGFGFVGGQLLQQAAQQLLQKGXG

(19) United States
(12) Patent Application Publication
(10) Pub. No.: US 2026/0048134 A1
(43) Pub. Date: Feb. 19, 2026

(54) PEPTIDE-BASED DELIVERY AGENT AND METHOD OF MAKING AND USING THE SAME

(71) Applicant: EndoRel Biosciences LLC, Portland, OR (US)

(72) Inventors: Jean C. Summerton, Portland, OR (US); Jeremy D. Bushman, Portland, OR (US); James E. Summerton, Philomath, OR (US)

(73) Assignee: EndoRel Biosciences LLC, Portland, OR (US)

(21) Appl. No.: 19/103,114

(22) PCT Filed: Aug. 11, 2023
(86) PCT No.: PCT/US2023/072094

§ 371 Date: Feb. 11, 2025

Related U.S. Application Data
(60) Provisional application No. 63/397,601, filed on Aug. 12, 2022.

(51) Int. Cl. A61K 47/68; A61K 47/64; G01N 33/50

(52) U.S. Cl. CPC A61K 47/6803; A61K 47/64; A61K 47/6849; G01N 33/5014; G01N 33/5023; G01N 2500/10

(57) ABSTRACT

Disclosed herein is a delivery agent that facilitates effective delivery of drugs and other compounds across lipid layers. The delivery agent disclosed herein provides lipid solubility under selected conditions and aqueous solubility under other conditions, and can effectively deliver compounds into the cell cytosol. The disclosed delivery agent also avoids deleterious interactions with serum and thus provide efficient in vivo delivery of therapeutic agents.

### FIELD

The present disclosure concerns peptide-based delivery agents that facilitate delivery of compounds into a cell.

### INCORPORATION OF ELECTRONIC SEQUENCE LISTING

The electronic sequence listing, submitted herewith as an XML file named 10413-107991-02.xml (341,757 bytes), created on Aug. 11, 2023, is herein incorporated by reference in its entirety.

### CROSS REFERENCE TO RELATED APPLICATION

This application claims the benefit of and priority to the earlier filing date of U.S. Provisional Application No. 63/397,601, filed on Aug. 12, 2022, the entirety of which is incorporated herein by reference.

### BACKGROUND

Lipid layers, such as those of cell membranes (including plasma membranes and vesicle membranes), can constitute barriers to effective drug delivery. For optimal delivery, a drug should freely dissolve in both the aqueous compartments of the body and the lipid layers that enclose those compartments. Although many low-molecular-weight compounds of low-to-moderate polarity can pass directly through lipid layers, compounds with greater polarity and/or higher molecular weight (such as nucleic acids and proteins) generally enter eukaryotic cells only via endocytosis or related processes. During endocytosis, compounds are taken into the cell via progressive invagination of a region of the membrane, eventually forming a closed vesicle, or endosome, within the cell. The endosome may then merge with a lysosome, resulting in entrapment inside the vesicles and exposure of the internalized compounds to degradative enzymes.

Thus, improved agents are needed that facilitate more effective delivery of drugs and other compounds across lipid layers, such as agents that afford lipid solubility under selected conditions and aqueous solubility under other conditions, and that can effectively deliver compounds into the cell cytosol.

### SUMMARY

Aspects of the present disclosure concern a peptide-based delivery agent comprising: a lytic peptide group having a structure according to a formula [X¹Y¹Y¹X¹]ₘ, wherein each X¹ independently for each occurrence is a basic amino acid, an acidic amino acid, a non-polar amino acid, or a derivative thereof, provided that at least one X¹ is a basic amino acid or an acidic amino acid; each Y¹ independently for each occurrence is a non-polar amino acid or a derivative thereof; and m is an integer selected from 2 to 8; a cleavable linker group; a mask peptide group having a structure according to a formula [X²Y²Y²X²]_{m′}, wherein each X² independently for each occurrence is an acidic amino acid, a non-polar amino acid, or a derivative thereof, provided that at least one X² is an acidic amino acid; each Y² independently for each occurrence is a non-polar amino acid or a derivative thereof; and m′ is an integer selected from 2 to 8; and an anchor group selected from a heteroaliphatic group, a dibenzocyclooctyne compound, an antibody or antibody fragment, a biotin group, an avidin group, a streptavidin group, or a neutravidin group; a targeting group selected from a cell, an antibody or antibody fragment, a peptide, a biomimetic peptide, an aptamer, a sugar, or a small targeting molecule; or a combination thereof.

In some aspects of the disclosure, the delivery agent has a structure according to Formula IA or IB

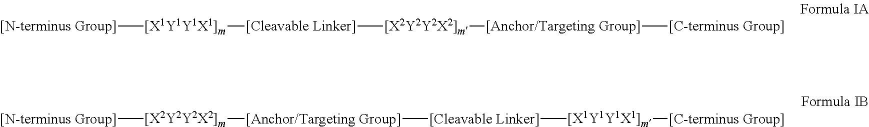

wherein each X¹ independently for each occurrence is a basic amino acid, an acidic amino acid, a non-polar amino acid, or a derivative thereof, provided that at least one X¹ is a basic amino acid or an acidic amino acid; each Y¹ independently for each occurrence is a non-polar amino acid or a derivative thereof; each of m and m′ independently for each occurrence is an integer selected from 2 to 8; each X² independently for each occurrence is an acidic amino acid, a non-polar amino acid, or a derivative thereof, provided that at least one X² is an acidic amino acid; each Y² independently for each occurrence is a non-polar amino acid or a derivative thereof; the cleavable linker is an amino acid sequence that is two to ten amino acids in length; the anchor group, if present, is selected from a heteroaliphatic group, a dibenzocyclooctyne compound, an antibody or antibody fragment, a dibenzocyclooctyne compound, a biotin group, an avidin group, a streptavidin group, a dior a neutravidin group; the targeting group, if present, is selected from a cell, an antibody or antibody fragment, a peptide, a biomimetic peptide, an aptamer, a sugar, or a small targeting molecule; or a combination thereof; the C-terminus group comprises an amine-terminated glycine moiety; and the N-terminus group comprises a capping group or a fluorophore.

Aspects of the present disclosure also concern a composition, comprising: a peptide-based delivery agent according to the present disclosure; and a therapeutic agent.

Aspects of the present disclosure also concern a method, comprising contacting a cell with a delivery agent and/or a composition according to the present disclosure.

Aspects of the present disclosure also concern a method, comprising administering to a subject a therapeutically effective amount of a delivery agent and/or a composition according to the present disclosure.

Aspects of the present disclosure also concern a method of identifying a therapeutic compound, comprising: contacting a cell with a peptide-based delivery agent according to the present disclosure and one or more compounds; determining an effect of the one or more compounds on the contacted cell; and comparing the effect of the one or more compounds on the contacted cell to a control; wherein a differential effect of the one or more compounds on the contacted cell relative to the control indicates that the one or more compounds is a therapeutic compound.

Aspects of the present disclosure also concern a kit, comprising a container, wherein the container comprises a peptide-based delivery agent or a composition according to the present disclosure, and wherein the container is selected from a syringe, vial, tube, ampule, capsule, or bottle.

The foregoing and other objects and features of the present disclosure will become more apparent from the following detailed description, which proceeds with reference to the accompanying figures.

### BRIEF DESCRIPTION OF THE DRAWINGS

FIG. 1 is a schematic illustration depicting entry of a peptide-based delivery agent as disclosed herein and a therapeutic agent into the cytosol of a eukaryotic cell by endocytotic uptake.

FIGS. 2A and 2B show a helical wheel diagram of an exemplary, EXXE motif-based lytic peptide group (FIG. 2A) and how the distribution of acidic residues results in faces of polar and non-polar regions; and FIG. 2B is a schematic illustrating interactions between an exemplary lytic peptide group backbone and an exemplary mask peptide group backbone.

FIGS. 3A and 3B are graphs showing average luciferase activity (LUC) per protein in 1% serum (FIG. 3A) and 80% serum (FIG. 3B) for delivery agents 23-6 and 23-7, discussed herein, in combination with a control Morpholino compound.

FIG. 4 is a graph of average luciferase activity (LUC) per protein in 1% serum for delivery agents 23-10, 23-16, 23-20, and a positive control, as discussed herein.

FIGS. 5A and 5B are graphs showing average luciferase activity (LUC) per protein in 1% serum (FIG. 5A) and 80% serum (FIG. 5B) for delivery agents 30-2, 30-4, 30-6, 30-10, 30-14, 30-16, 30-18, and 30-22, discussed herein.

FIGS. 6A and 6B are graphs showing average luciferase activity (LUC) per protein in 80% serum using 12 μM delivery agent (FIG. 6A) for delivery agents 40-1 and 40-46, and 80% serum using 6 μM delivery agent (FIG. 6B) for delivery agents 42-9 and 42-39, discussed herein.

FIG. 7 is a graph showing average luciferase activity (LUC) per protein in 1% serum for delivery agents 40-1, 40-46, 42-9, and 42-39, discussed herein.

FIGS. 8A-8C show helical wheel diagrams for delivery agents 23-26 (FIG. 8A) and 50-22 (FIG. 8B) and average luciferase activity (LUC) per protein in 80% serum (FIG. 8C).

FIG. 9 is a graph showing average luciferase activity (LUC) per protein in 80% serum using delivery agents 23-26, 51-11, 51-12, 51-13, 51-14, 51-15, 51-16, 51-17, 51-18, discussed herein and a control of morpholino only.

FIG. 10 is a graph showing average luciferase activity (LUC) per protein in 80% serum using delivery agents 23-26, 51-24, 51-25, 51-26, 51-27, 51-28, 51-29, 51-30, 51-31, 50-23, discussed herein and a control of morpholino only.

FIG. 11 is a graph showing average luciferase activity (LUC) per protein in 1% serum using delivery agents 56-3, 56-5, 23-26, and a control, discussed herein, at different concentrations.

FIGS. 12A and 12B are graphs showing average luciferase activity (LUC) per protein in 80% serum delivery agents 60-16, 60-17, 60-4, 62-9, and 23-26, discussed herein.

FIG. 13 is a graph showing average luciferase activity (LUC) per protein in 80% serum using a control and delivery agents 23-26, 88-8, 88-9, 88-10, 88-11, and 88-12, discussed herein.

FIG. 14 is a graph showing average luciferase activity (LUC) per protein in 80% serum using untreated cells and delivery agents 104-3+4, 109-7, 114-1, 114-3, 114-12, 114-30, and 114-33, discussed herein.

FIGS. 15A-15D show helical wheel diagrams for delivery agents 114-12 (FIG. 15A), 114-6 (FIG. 15B), and 114-9 (FIG. 15C), as well as average luciferase activity (LUC) per protein for these delivery agents and compound 104-3+4 in 80% serum (FIG. 15D).

FIG. 16 is a graph of % fraction of compound bound to human serum albumin as a function of number of amino acids in the lytic peptide group.

FIG. 17 is a graph showing average luciferase activity (LUC) per protein in 80% serum using untreated cells or cells treated with delivery agents 104-3+4, 114-12, 114-18, 114-21, 114-24, or 114-27, discussed herein.

FIGS. 18A and 18B are graphs showing average luciferase activity (LUC) per protein in 1% serum (FIG. 18A) and 80% serum (FIG. 18B) for delivery agents 21-11 and 19-1, discussed herein.

FIGS. 19A and 19B are graphs showing average luciferase activity (LUC) per protein in 1% serum (FIG. 19A) and 80% serum (FIG. 19B) for delivery agents 36-47 and 36-48, discussed herein.

FIG. 20 is a graph showing average luciferase activity (LUC) per protein in 80% serum for delivery agents 96-1, 96-4, 96-5, 96-6, and 96-7, discussed herein.

FIG. 21 is a graph showing average luciferase activity (LUC) per protein in 80% serum for delivery agents 26-17, 26-2, discussed herein, and a negative control.

FIGS. 22A and 22B are graphs showing average luciferase activity (LUC) per protein in 1% serum (FIG. 22A) and 80% serum (FIG. 22B) for delivery agents 19-1, 19-3, 19-5, discussed herein, and two controls.

FIGS. 23A and 23B are graphs showing average luciferase activity (LUC) per protein in 1% serum (FIG. 23A) and 80% serum (FIG. 23B) for delivery agents 23-12, 23-13, and 23-14, discussed herein.

FIGS. 24A and 24B are graphs showing average luciferase activity (LUC) per protein in 1% serum (FIG. 24A) and 80% serum (FIG. 24B) for delivery agents 21-12, 21-13, and 19-1 with differing anchor group tail lengths, discussed herein.

FIG. 25 is a graph showing average luciferase activity (LUC) per protein in 1% serum for delivery agents 23-15 and 19-1 discussed herein.

FIGS. 26A-26D are graphs showing average luciferase activity (LUC) per protein in 1% serum at 3 μM (FIG. 26A), 80% serum at 9 μM (FIG. 26B), 1% serum at 2 μM (FIG. 26C), 80% serum at 9 μM (FIG. 26D), for delivery agents 19-1 and 21-8, discussed herein.

FIG. 27 is a graph showing average luciferase activity (LUC) per protein in 80% serum for delivery agents 40-31 to 40-46, discussed herein.

FIG. 28 is a graph showing average luciferase activity (LUC) per protein in 80% serum for delivery agents 23-26 and 53-6 to 53-15, discussed herein, along with a control with no treatment.

FIG. 29A-29C are graphs showing average luciferase activity (LUC) per protein in 1% serum at 1 or 3 μM (FIG. 29A) for delivery agents 96-10 in different DMSO concentrations, 23-26, and a control; 80% serum at 12 μM (FIG. 29B) for delivery agents 96-10, 23-26, and a control; and 80% serum at 9 or 18 μM (FIG. 29C) for delivery agents 26-13A with and without DMSO, discussed herein.

FIGS. 30A and 30B are graphs showing average luciferase activity (LUC) per protein in 80% serum (FIG. 30A) and 1% serum (FIG. 30B) for delivery agents 21-1, 21-4, and 21-6, discussed herein, and a control.

FIG. 31 is a graph showing average luciferase activity (LUC) per protein in 80% serum for differing concentrations of delivery agents 19-1, 21-4, and 21-6, discussed herein, and a control.

FIGS. 32A and 32B are graphs showing average luciferase activity (LUC) per protein in 80% serum for delivery agents 23-27 and 23-4, discussed herein.

FIG. 33 is a graph showing average luciferase activity (LUC) per protein in 80% serum for delivery agents 19-1, 23-4, and 23-5 discussed herein.

FIGS. 34A and 34B are graphs showing average luciferase activity (LUC) per protein in 80% serum at 18 μM (FIG. 34A) for delivery agents 26-7a/a′ and 26-7b/b′, discussed herein; and 80% serum at 9 μM (FIG. 34B) for delivery agents 26-14A and 26-14B with differing anchor group tail lengths, discussed herein.

FIG. 35 is a graph showing average luciferase activity (LUC) per protein in 80% serum for differing concentrations of delivery agents 26-14A-D with differing anchor group tail lengths, discussed herein.

FIG. 36 is a graph showing average luciferase activity (LUC) per protein in 80% serum for differing concentrations of delivery agents 26-15A/B with differing anchor tail lengths, discussed herein, and a control.

FIG. 37 is a graph showing average luciferase activity (LUC) per protein in 80% serum for delivery agents 26-2A and 26-11 (with no anchor group), and delivery agents 26-2B and 26-13, discussed herein, as well as a control.

FIG. 38 is a graph showing average luciferase activity (LUC) per protein in 80% serum for delivery agents 33-24 and 23-26, discussed herein, and a control and without treatment.

FIGS. 39A and 39B are graphs showing average luciferase activity (LUC) per protein in 80% serum for delivery agents 40-23A, 40-46, discussed herein, and a control (FIG. 39A); and 80% serum for delivery agents 40-23A, 40-23B and 40-46, discussed herein (FIG. 39B).

FIG. 40 is a graph showing average luciferase activity (LUC) per protein in 80% serum for delivery agents 21-4a, 21-4b, 21-4b′, 21-6a, 21-6b, and 21-7a, all with differing anchor group tail lengths, discussed herein.

FIG. 41 is a graph showing average luciferase activity (LUC) per protein in 80% serum for delivery agents 60-1b, 60-1a, and 23-26, discussed herein, and a control.

FIG. 42 is a graph showing average luciferase activity (LUC) per protein in 80% serum for delivery agents 104-3+4, 109-1, and 109-8 discussed herein, and a control.

FIG. 43 is a graph showing average luciferase activity (LUC) per protein in 80% serum for delivery agents 23-26, 78-1, and 78-2, discussed herein, and a control.

FIGS. 44A and 44B are graphs showing average luciferase activity (LUC) per protein in 80% serum for delivery agents 23-26, 85-2 C, and 85-2 P, discussed herein, and a control (FIG. 44A); and 80% serum for delivery agents 23-26, 85-2C, 85-2 P, 86-1 C, 86-1 P, 86-4 C, and 86-4 P, discussed herein, and a control (FIG. 44B).

FIG. 45 is a graph showing average luciferase activity (LUC) per protein in 80% serum for delivery agents 23-26, 85-A C, and 86-5A P, discussed herein, and a control.

FIG. 46 is a graph showing average luciferase activity (LUC) per protein in 80% serum for delivery agents 23-26, 50-23, 50-24, 50-25, and 50-26, discussed herein.

FIGS. 47A and 47B are graphs showing average luciferase activity (LUC) per protein in 1% serum (FIG. 47A) and 80% serum (FIG. 47B) for delivery agents 23-26, 30-3, 30-5, 30-7, 30-9, 30-11, 30-15, 30-17, and 30-22, discussed herein.

FIGS. 48A and 48B are graphs showing average luciferase activity (LUC) per protein in 1% serum (FIG. 48A) and 80% serum (FIG. 48B) for delivery agents 19-1, 19-17A, and 19-17C, discussed herein, and two controls.

FIGS. 49A and 49B are graphs showing average luciferase activity (LUC) per protein in 1% serum (FIG. 49A) and 80% serum (FIG. 49B) for delivery agents 21-4 and 21-5, discussed herein, and a control.

FIGS. 50A and 50B are graphs showing average luciferase activity (LUC) per protein in 1% serum (FIG. 50A) and 80% serum (FIG. 50B) for delivery agents 19-1 and 22-11, discussed herein, and a control.

FIGS. 51A and 51B are graphs showing average luciferase activity (LUC) per protein in 80% serum for delivery agents 24-1, 23-27, 23-1, and 23-4, discussed herein.

FIGS. 52A and 52B are graphs showing average luciferase activity (LUC) per protein in 1% serum (FIG. 52A) and 80% serum (FIG. 52B) for delivery agents 23-1 to 23-5, discussed herein.

FIGS. 53A and 53B are graphs showing average luciferase activity (LUC) per protein in 1% serum (FIG. 53A) and 80% serum (FIG. 53B) for delivery agents 21-4, 22-1, and 22-12, discussed herein, and a control.

FIG. 54 is a graph showing average luciferase activity (LUC) per protein in 80% serum for delivery agents 21-1, 33-26a, 33-26b, 33-26c, 34-32, 34-33, and 34-34, discussed herein.

FIG. 55 is a graph showing average luciferase activity (LUC) per protein in 80% serum for delivery agents 104-3+4 and 109-2, discussed herein.

FIGS. 56A and 56B are graphs showing average luciferase activity (LUC) per protein in 80% serum for delivery agents 23-26, MO+23-26, Vivo-MO+23-26, Vivo-MO, and no treatment (FIG. 56A) and 80% serum (FIG. 56B) for delivery agents MO+23-26, Vivo-MO, and Vivo-MO+23-26, discussed herein, at different concentrations.

FIG. 57 is a graph showing average luciferase activity (LUC) per protein in 80% serum for delivery agents 23-26+a and 23-26+b, discussed herein, and no treatment.

FIG. 58 is a graph showing average luciferase activity (LUC) per protein in 80% serum for delivery agents 23-26+a and 23-26+b, and 23-26 without added Morpholino, discussed herein.

FIG. 59 is a graph showing average luciferase activity (LUC) per protein in 80% serum for delivery agents 102-3 and 98-1-MO conjugate, discussed herein, and no treatment.

FIG. 60 is a graph showing average luciferase activity (LUC) per protein in 80% serum for the compound discussed herein in Example 59 and a control.

FIG. 61 is a graph showing average luciferase activity (LUC) per protein in 80% serum for delivery agents 104-3+4P+MO and 107-8-MO conjugate, discussed herein, and a control.

FIGS. 62A and 62B are graphs showing cell viability results for compound 104-3+4 (FIG. 62A) and saporin delivery results when a saporin toxin is combined with compound 104-3+4 (FIG. 62B).

FIG. 63 is an image showing mRNA (isolated from muscle tissue) amplified using RT-PCR, and then analyzed using gel electrophoresis.

FIG. 64 is an image showing mRNA (isolated from muscle tissue) amplified using RT-PCR, and then analyzed using gel electrophoresis.

FIGS. 65A and 65B are graphs showing average luciferase activity (LUC) per protein in 1% serum (FIG. 65A) and 80% serum (FIG. 65B) for compound 16-6 and two controls.

FIGS. 66A and 66B are graphs showing average luciferase activity (LUC) per protein in 1% serum (FIG. 66A) and 80% serum (FIG. 66B) for compound 19-1 and two controls.

FIGS. 67A and 67B are graphs showing average luciferase activity (LUC) per protein in 80% serum (FIG. 67A) and 10% serum (FIG. 67B) for delivery agents 215-2 DBCO, 216-A DBCO, and 216-B DBCO.

FIG. 68 is a graph showing average luciferase activity (LUC) per protein in 10% serum for delivery agents 215-2 FA, 216-A FA, 216-B, FA, 215-2 DBCO, 216-A DBCO, 216-B DBCO, wherein “FA” is free amine.

FIG. 69 is a graph showing average luciferase activity (LUC) per protein in 10% serum for delivery agents 218-1, 218-2, 219-1, and 219-2.

FIGS. 70A-70C are graphs showing results from evaluating different delivery agents disclosed herein, wherein FIG. 70A shows GFP expression in 10% serum for controls and delivery agents 215-2 Ab, 216-2 Ab, 216-1Ab, 215-2 PEG12-BCN, and 216-1 PEG12-BCN; FIG. 70B shows average luciferase activity (LUC) in 10% serum for HeLa Luc/705 cells transfected with transferrin receptor (TFRC); and FIG. 70C shows average luciferase activity (LUC) in 10% serum for HeLa Luc/705 cells that were not transfected.

FIG. 71 shows average luciferase activity (LUC) in 80% serum for delivery agents 224-2, 225-2, and 216-2.

FIG. 72 shows average luciferase activity (LUC) in 80% serum for delivery agents 223-2 and 89-5.

### SEQUENCE LISTING

The nucleic acid sequences and amino acid sequences listed in the accompanying sequence listing are shown using standard letter abbreviations for nucleotide bases and amino acids as defined in 37 C.F.R. § 1.822. Only one strand of each nucleic acid sequence is shown, but the complementary strand is understood as included by any reference to the displayed strand.
SEQ ID NO: 1 is an amino acid sequence of a lytic peptide group.
SEQ ID NO: 2 is an amino acid sequence of a lytic peptide group.
SEQ ID NO: 3 is an amino acid sequence of a cleavable linker.
SEQ ID NO: 4 is an amino acid sequence of a mask peptide group.
SEQ ID NO: 5 is an amino acid sequence of a C-terminal moiety.
SEQ ID NO: 6 is the amino acid sequence of a lytic peptide group, cleavable linker, and mask peptide group of a delivery agent.
SEQ ID NO: 7 is the amino acid sequence of a lytic peptide group, cleavable linker, mask peptide group, and amino acid portions of two anchor groups of a delivery agent.
SEQ ID NO: 8 is the amino acid sequence of a lytic peptide group, cleavable linker, mask peptide group, amino acid portions of two anchor groups, and C-terminal moiety of a delivery agent.
SEQ ID NO: 9 is the amino acid sequence of a lytic peptide group, cleavable linker, and mask peptide group of a delivery agent.
SEQ ID NO: 10 is the amino acid sequence of a lytic peptide group, cleavable linker, mask peptide group, and amino acid portions of two anchor groups of a delivery agent.

SEQ ID NO: 11 is the amino acid sequence of a lytic peptide group, cleavable linker, mask peptide group, amino acid portions of two anchor groups, and C-terminal moiety of a delivery agent.
SEQ ID NO: 12 is the amino acid sequence of a lytic peptide group, cleavable linker, mask peptide group, amino acid portions of two anchor groups, and C-terminal moiety of a delivery agent.
SEQ ID NO: 13 is the amino acid sequence of a lytic peptide group, cleavable linker, mask peptide group, amino acid portions of two anchor groups, and C-terminal moiety of a delivery agent, such as that included in delivery agents 21-4, 40-46, 42-39, 23-4, and 23-26.
SEQ ID NO: 14 is the amino acid sequence of a lytic peptide group, cleavable linker, mask peptide group, amino acid portions of two anchor groups, and C-terminal moiety of a delivery agent.
SEQ ID NO: 15 is the amino acid sequence of a lytic peptide group, cleavable linker, mask peptide group, amino acid portions of two anchor groups, and C-terminal moiety of a delivery agent.
SEQ ID NO: 16 is an amino acid sequence of a lytic peptide group.
SEQ ID NO: 17 is an amino acid sequence of a lytic peptide group.
SEQ ID NO: 18 is the amino acid sequence of delivery agent 23-6.
SEQ ID NO: 19 is the amino acid sequence of delivery agent 23-7.
SEQ ID NO: 20 is an amino acid sequence of a lytic peptide group.
SEQ ID NO: 21 is the amino acid sequence of delivery agent 23-10.
SEQ ID NO: 22 is the amino acid sequence of delivery agent 23-16.
SEQ ID NO: 23 is the amino acid sequence of delivery agent 23-20.
SEQ ID NO: 24 is an amino acid sequence of a lytic peptide group.
SEQ ID NO: 25 is the amino acid sequence of delivery agent 30-2.
SEQ ID NO: 26 is an amino acid sequence of a lytic peptide group.
SEQ ID NO: 27 is the amino acid sequence of delivery agent 30-4.
SEQ ID NO: 28 is an amino acid sequence of a lytic peptide group.
SEQ ID NO: 29 is the amino acid sequence of delivery agent 30-6.
SEQ ID NO: 30 is an amino acid sequence of a lytic peptide group.
SEQ ID NO: 31 is the amino acid sequence of delivery agent 30-10.
SEQ ID NO: 32 is an amino acid sequence of a lytic peptide group.
SEQ ID NO: 33 is the amino acid sequence of delivery agent 30-14.
SEQ ID NO: 34 is an amino acid sequence of a lytic peptide group.
SEQ ID NO: 35 is the amino acid sequence of delivery agent 30-16.
SEQ ID NO: 36 is an amino acid sequence of a lytic peptide group.
SEQ ID NO: 37 is the amino acid sequence of delivery agent 30-18.
SEQ ID NO: 38 is an amino acid sequence of a lytic peptide group.
SEQ ID NO: 39 is the amino acid sequence of delivery agent 40-1.
SEQ ID NO: 40 is an amino acid sequence of a lytic peptide group.
SEQ ID NO: 41 is an amino acid sequence of a mask peptide group.
SEQ ID NO: 42 is the amino acid sequence of delivery agent 50-22.
SEQ ID NO: 43 is an amino acid sequence of a lytic peptide group.
SEQ ID NO: 44 is an amino acid sequence of a lytic peptide group.
SEQ ID NO: 45 is an amino acid sequence of a lytic peptide group.

SEQ ID NO: 46 is an amino acid sequence of a lytic peptide group.
SEQ ID NO: 47 is an amino acid sequence of a lytic peptide group.
SEQ ID NO: 48 is an amino acid sequence of a lytic peptide group.
SEQ ID NO: 49 is an amino acid sequence of a lytic peptide group.
SEQ ID NO: 50 is an amino acid sequence of a lytic peptide group.
SEQ ID NO: 51 is an amino acid sequence of a mask peptide group.
SEQ ID NO: 52 is the amino acid sequence of delivery agent 51-11.
SEQ ID NO: 53 is the amino acid sequence of delivery agent 51-12.
SEQ ID NO: 54 is the amino acid sequence of delivery agent 51-13.
SEQ ID NO: 55 is the amino acid sequence of delivery agent 51-14.
SEQ ID NO: 56 is the amino acid sequence of delivery agent 51-15.
SEQ ID NO: 57 is the amino acid sequence of delivery agent 51-16.
SEQ ID NO: 58 is the amino acid sequence of delivery agent 51-17.
SEQ ID NO: 59 is the amino acid sequence of delivery agent 51-18.
SEQ ID NO: 60 is an amino acid sequence of a lytic peptide group.
SEQ ID NO: 61 is an amino acid sequence of a lytic peptide group.
SEQ ID NO: 62 is an amino acid sequence of a lytic peptide group.
SEQ ID NO: 63 is an amino acid sequence of a lytic peptide group.
SEQ ID NO: 64 is an amino acid sequence of a lytic peptide group.
SEQ ID NO: 65 is an amino acid sequence of a lytic peptide group.
SEQ ID NO: 66 is an amino acid sequence of a lytic peptide group.
SEQ ID NO: 67 is an amino acid sequence of a lytic peptide group.
SEQ ID NO: 68 is an amino acid sequence of a lytic peptide group.
SEQ ID NO: 69 is the amino acid sequence of delivery agent 51-24.
SEQ ID NO: 70 is the amino acid sequence of delivery agent 51-25.
SEQ ID NO: 71 is the amino acid sequence of delivery agent 51-26.
SEQ ID NO: 72 is the amino acid sequence of delivery agent 51-27.
SEQ ID NO: 73 is the amino acid sequence of delivery agent 51-28.
SEQ ID NO: 74 is the amino acid sequence of delivery agent 51-29.
SEQ ID NO: 75 is the amino acid sequence of delivery agent 51-30.
SEQ ID NO: 76 is the amino acid sequence of delivery agent 51-31.
SEQ ID NO: 77 is the amino acid sequence of delivery agent 50-23.
SEQ ID NO: 78 is an amino acid sequence of a lytic peptide group.
SEQ ID NO: 79 is the amino acid sequence of delivery agent 56-5.
SEQ ID NO: 80 is the amino acid sequence of a lytic peptide group.
SEQ ID NO: 81 is the amino acid sequence of a mask peptide group.
SEQ ID NO: 82 the amino acid sequence of delivery agent 60-16.
SEQ ID NO: 83 the amino acid sequence of delivery agent 60-17.
SEQ ID NO: 84 is the amino acid sequence of a lytic peptide group.
SEQ ID NO: 85 is the amino acid sequence of a lytic peptide group.
SEQ ID NO: 86 is the amino acid sequence of a lytic peptide group.

SEQ ID NO: 87 the amino acid sequence of delivery agent 88-8.
SEQ ID NO: 88 the amino acid sequence of delivery agent 88-9.
SEQ ID NO: 89 the amino acid sequence of delivery agent 88-10.
SEQ ID NO: 90 the amino acid sequence of delivery agent 88-11.
SEQ ID NO: 91 the amino acid sequence of delivery agent 88-12.
SEQ ID NO: 92 is the amino acid sequence of a lytic peptide group.
SEQ ID NO: 93 is the amino acid sequence of a lytic peptide group.
SEQ ID NO: 94 is the amino acid sequence of a lytic peptide group.
SEQ ID NO: 95 is the amino acid sequence of a lytic peptide group.
SEQ ID NO: 96 is the amino acid sequence of a lytic peptide group.
SEQ ID NO: 97 is the amino acid sequence of a lytic peptide group.
SEQ ID NO: 98 is the amino acid sequence of a mask peptide group.
SEQ ID NO: 99 is the amino acid sequence of a mask peptide group.
SEQ ID NO: 100 is the amino acid sequence of a mask peptide group.
SEQ ID NO: 101 is the amino acid sequence of a mask peptide group.
SEQ ID NO: 102 is the amino acid sequence of delivery agent 109-7.
SEQ ID NO: 103 is the amino acid sequence of delivery agent 114-1.
SEQ ID NO: 104 is the amino acid sequence of delivery agent 114-3.
SEQ ID NO: 105 is the amino acid sequence of delivery agent 114-12.
SEQ ID NO: 106 is the amino acid sequence of delivery agent 114-30.
SEQ ID NO: 107 is the amino acid sequence of delivery agent 114-33.
SEQ ID NO: 108 is the amino acid sequence of a lytic peptide group, cleavable linker, mask peptide group, amino acid portions of two anchor groups, and C-terminal moiety of a delivery agent.
SEQ ID NO: 109 is the amino acid sequence of a lytic peptide group.
SEQ ID NO: 110 is the amino acid sequence of a lytic peptide group.
SEQ ID NO: 111 is the amino acid sequence of a mask peptide group.
SEQ ID NO: 112 is the amino acid sequence of a mask peptide group.
SEQ ID NO: 113 is the amino acid sequence of delivery agent 114-6.
SEQ ID NO: 114 is the amino acid sequence of delivery agent 114-9.
SEQ ID NO: 115 is the amino acid sequence of a lytic peptide group.
SEQ ID NO: 116 is the amino acid sequence of a lytic peptide group.
SEQ ID NO: 117 is the amino acid sequence of a lytic peptide group.
SEQ ID NO: 118 is the amino acid sequence of delivery agent 97-1.
SEQ ID NO: 119 is the amino acid sequence of delivery agent 97-2.
SEQ ID NO: 120 is the amino acid sequence of delivery agent 97-3.
SEQ ID NO: 121 is the amino acid sequence of delivery agent 97-5.
SEQ ID NO: 122 is the amino acid sequence of delivery agent 97-7.
SEQ ID NO: 123 is the amino acid sequence of delivery agent 91-6.
SEQ ID NO: 124 is the amino acid sequence of delivery agent 114-18.
SEQ ID NO: 125 is the amino acid sequence of delivery agent 114-21.
SEQ ID NO: 126 is the amino acid sequence of delivery agent 114-24.

SEQ ID NO: 127 is the amino acid sequence of delivery agent 114-27.
SEQ ID NO: 128 is the amino acid sequence of delivery agent 21-11.
SEQ ID NO: 129 is the amino acid sequence of delivery agent 19-1, 36-47, 26-2, 21-1, 23-27, and 26-2B.
SEQ ID NO: 130 is the amino acid sequence of delivery agent 36-48.
SEQ ID NO: 131 is the amino acid sequence of delivery agent 96-1.
SEQ ID NO: 132 is the amino acid sequence of delivery agent 96-4.
SEQ ID NO: 133 is the amino acid sequence of delivery agent 96-5.
SEQ ID NO: 134 is the amino acid sequence of a mask peptide group.
SEQ ID NO: 135 is the amino acid sequence of delivery agent 26-17.
SEQ ID NO: 136 is the amino acid sequence of a mask peptide group.
SEQ ID NO: 137 is the amino acid sequence of a mask peptide group.
SEQ ID NO: 138 is the amino acid sequence of delivery agent 19-3.
SEQ ID NO: 139 is the amino acid sequence of delivery agent 19-5.
SEQ ID NO: 140 is the amino acid sequence of a mask peptide group.
SEQ ID NO: 141 is the amino acid sequence of a mask peptide group.
SEQ ID NO: 142 is the amino acid sequence of delivery agent 23-12.
SEQ ID NO: 143 is the amino acid sequence of delivery agent 23-13.
SEQ ID NO: 144 is the amino acid sequence of delivery agent 23-14.
SEQ ID NO: 145 is the amino acid sequence of a mask peptide group.
SEQ ID NO: 146 is the amino acid sequence of delivery agent 21-13.
SEQ ID NO: 147 is the amino acid sequence of delivery agent 23-15 and 21-12.
SEQ ID NO: 148 is the amino acid sequence of a mask peptide group.
SEQ ID NO: 149 is the amino acid sequence of delivery agent 21-8.
SEQ ID NO: 150 is the amino acid sequence of a lytic peptide group.
SEQ ID NO: 151 is the amino acid sequence of a lytic peptide group.
SEQ ID NO: 152 is the amino acid sequence of a lytic peptide group.
SEQ ID NO: 153 is the amino acid sequence of a lytic peptide group and/or mask group.
SEQ ID NO: 154 is the amino acid sequence of a mask peptide group.
SEQ ID NO: 155 is the amino acid sequence of a mask peptide group.
SEQ ID NO: 156 is the amino acid sequence of a mask peptide group.
SEQ ID NO: 157 is the amino acid sequence of a mask peptide group.
SEQ ID NO: 158 is the amino acid sequence of a mask peptide group.
SEQ ID NO: 159 is the amino acid sequence of delivery agent 40-31.
SEQ ID NO: 160 is the amino acid sequence of delivery agent 40-32.
SEQ ID NO: 161 is the amino acid sequence of delivery agent 40-33.
SEQ ID NO: 162 is the amino acid sequence of delivery agent 40-34.
SEQ ID NO: 163 is the amino acid sequence of delivery agent 40-35.
SEQ ID NO: 164 is the amino acid sequence of delivery agent 40-36.
SEQ ID NO: 165 is the amino acid sequence of delivery agent 40-37.
SEQ ID NO: 166 is the amino acid sequence of delivery agent 40-38.

SEQ ID NO: 167 is the amino acid sequence of delivery agent 40-39.
SEQ ID NO: 168 is the amino acid sequence of delivery agent 40-40.
SEQ ID NO: 169 is the amino acid sequence of delivery agent 40-41.
SEQ ID NO: 170 is the amino acid sequence of delivery agent 40-42.
SEQ ID NO: 171 is the amino acid sequence of delivery agent 40-43.
SEQ ID NO: 172 is the amino acid sequence of a mask peptide group.
SEQ ID NO: 173 is the amino acid sequence of a mask peptide group.
SEQ ID NO: 174 is the amino acid sequence of a mask peptide group.
SEQ ID NO: 175 the amino acid sequence of is a mask peptide group.
SEQ ID NO: 176 the amino acid sequence of is a mask peptide group.
SEQ ID NO: 177 the amino acid sequence of is a mask peptide group.
SEQ ID NO: 178 the amino acid sequence of is a mask peptide group.
SEQ ID NO: 179 the amino acid sequence of is a mask peptide group.
SEQ ID NO: 180 the amino acid sequence of is a mask peptide group.
SEQ ID NO: 181 the amino acid sequence of is a mask peptide group.
SEQ ID NO: 182 is the amino acid sequence of delivery agent 53-6.
SEQ ID NO: 183 is the amino acid sequence of delivery agent 53-7.
SEQ ID NO: 184 is the amino acid sequence of delivery agent 53-8.
SEQ ID NO: 185 is the amino acid sequence of delivery agent 53-9.
SEQ ID NO: 186 is the amino acid sequence of delivery agent 53-10.
SEQ ID NO: 187 is the amino acid sequence of delivery agent 53-11.
SEQ ID NO: 188 is the amino acid sequence of delivery agent 53-12.
SEQ ID NO: 189 is the amino acid sequence of delivery agent 53-13.
SEQ ID NO: 190 is the amino acid sequence of delivery agent 53-14.
SEQ ID NO: 191 is the amino acid sequence of delivery agent 53-15.
SEQ ID NO: 192 is the amino acid sequence of delivery agent 96-10.
SEQ ID NO: 193 is the amino acid sequence of delivery agent 21-6 and/or 23-5.
SEQ ID NO: 194 is the amino acid sequence of delivery agent 26-13.
SEQ ID NO: 195 is the amino acid sequence of delivery agent 21-7b.
SEQ ID NO: 196 is the amino acid sequence of delivery agent 109-8.
SEQ ID NO: 197 is the amino acid sequence of delivery agent 78-2.
SEQ ID NO: 198 is the amino acid sequence of delivery agent 86-5.
SEQ ID NO: 199 is the amino acid sequence of delivery agent 50-23.
SEQ ID NO: 200 is the amino acid sequence of delivery agent 50-24.
SEQ ID NO: 201 is the amino acid sequence of delivery agent 50-25.
SEQ ID NO: 202 is the amino acid sequence of delivery agent 50-26.
SEQ ID NO: 203 is the amino acid sequence of a mask peptide group.
SEQ ID NO: 204 is the amino acid sequence of a mask peptide group.
SEQ ID NO: 205 is the amino acid sequence of a mask peptide group.
SEQ ID NO: 206 is the amino acid sequence of a mask peptide group.
SEQ ID NO: 207 is the amino acid sequence of a mask peptide group.

SEQ ID NO: 208 is the amino acid sequence of delivery agent 30-3.
SEQ ID NO: 209 is the amino acid sequence of delivery agent 30-5.
SEQ ID NO: 210 is the amino acid sequence of delivery agent 30-7.
SEQ ID NO: 211 is the amino acid sequence of delivery agent 30-9.
SEQ ID NO: 212 is the amino acid sequence of delivery agent 30-11.
SEQ ID NO: 213 is the amino acid sequence of delivery agent 30-15.
SEQ ID NO: 214 is the amino acid sequence of delivery agent 30-17.
SEQ ID NO: 215 is the amino acid sequence of delivery agent 19-17C.
SEQ ID NO: 216 is the amino acid sequence of delivery agent 21-5.
SEQ ID NO: 217 is the amino acid sequence of delivery agent 22-11.
SEQ ID NO: 218 is the amino acid sequence of delivery agent 23-1.
SEQ ID NO: 219 is the amino acid sequence of delivery agent 23-2.
SEQ ID NO: 220 is the amino acid sequence of delivery agent 23-3.
SEQ ID NO: 221 is the amino acid sequence of delivery agent 22-1.
SEQ ID NO: 222 is the amino acid sequence of delivery agent 22-12.
SEQ ID NO: 223 is the amino acid sequence of delivery agent 34-32.
SEQ ID NO: 224 is the amino acid sequence of delivery agent 34-33.
SEQ ID NO: 225 is the amino acid sequence of delivery agent 34-34.
SEQ ID NO: 226 is the amino acid sequence of delivery agent 109-2.
SEQ ID NO: 227 is the amino acid sequence of a cleavable linker.
SEQ ID NO: 228 is the amino acid sequence of delivery agent 33-3.
SEQ ID NO: 229 is the nucleic acid sequence of a Morpholino 1.
SEQ ID NO: 230 is the nucleic acid sequence of a Morpholino 2.
SEQ ID NO: 231 is the amino acid sequence of delivery agent 96-6.
SEQ ID NO: 232 is the amino acid sequence of delivery agent 16-6.
SEQ ID NO: 233 is the amino acid sequence of a cleavable linker.
SEQ ID NO: 234 is the amino acid sequence of a cleavable linker.
SEQ ID NO: 235 is the amino acid sequence of delivery agent 5.
SEQ ID NO: 236 is the amino acid sequence of delivery agent 7.
SEQ ID NO: 237 is the amino acid sequence of delivery agent 8.
SEQ ID NO: 238 the nucleic acid sequence of a forward DNA primer spanning dystrophin exon 21 and exon 24.
SEQ ID NO: 239 is the nucleic acid sequence of a reverse DNA primer spanning dystrophin exon 21 and exon 24.
SEQ ID NO: 240 is the nucleic acid sequence of a morpholino.
SEQ ID NO: 241 is the nucleic acid sequence of a morpholino.
SEQ ID NO: 242 is the nucleic acid sequence of a morpholino.
SEQ ID NO: 243 is the nucleic acid sequence of a morpholino.
SEQ ID NO: 244 is the nucleic acid sequence of a morpholino.
SEQ ID NO: 245 is the amino acid sequence of the branch of delivery agent 22-1.

SEQ ID NO: 246 is the amino acid sequence of delivery agent 30-22, 21-4, 40-46, 42-39, 23-4, and 23-26.
SEQ ID NO: 247 is the amino acid sequence of delivery agent 62-9.
SEQ ID NO: 248 is the amino acid sequence of delivery agent 60-4.
SEQ ID NO: 249 is the amino acid sequence of delivery agent 96-7, 86-4, and 102-3.
SEQ ID NO: 250 is the amino acid sequence of delivery agent 26-13A.
SEQ ID NO: 251 is the amino acid sequence of delivery agent 26-15A.
SEQ ID NO: 252 is the amino acid sequence of delivery agent 26-7a′.
SEQ ID NO: 253 is the amino acid sequence of delivery agent 26-7b.
SEQ ID NO: 254 is the amino acid sequence of delivery agent 26-7b′.
SEQ ID NO: 255 is the amino acid sequence of delivery agent 26-14B.
SEQ ID NO: 256 is the amino acid sequence of delivery agent 26-14D.
SEQ ID NO: 257 is the amino acid sequence of delivery agent 26-2A.
SEQ ID NO: 258 is the amino acid sequence of delivery agent 26-11.
SEQ ID NO: 259 is the amino acid sequence of delivery agent 40-23B.
SEQ ID NO: 260 is the amino acid sequence of delivery agent 21-4b.
SEQ ID NO: 261 is the amino acid sequence of delivery agent 21-4b′.
SEQ ID NO: 262 is the amino acid sequence of delivery agent 21-6b.
SEQ ID NO: 263 is the amino acid sequence of delivery agent 60-1b.
SEQ ID NO: 264 is the amino acid sequence of delivery agent 78-1.
SEQ ID NO: 265 is the amino acid sequence of delivery agent 19-17A.
SEQ ID NO: 266 is the amino acid sequence of the branch of delivery agent 22-12.
SEQ ID NO: 267 is the amino acid sequence of delivery agent 33-26a.
SEQ ID NO: 268 is the amino acid sequence of delivery agent 33-26b.
SEQ ID NO: 269 is the amino acid sequence of delivery agent 33-26c.
SEQ ID NO: 270 is the amino acid sequence of delivery agent 6.
SEQ ID NO: 271 is the amino acid sequence of delivery agent 40-23A.
SEQ ID NO: 272 is the amino acid sequence of delivery agent 215-2 DBCO, 215-2 FA, 215-2 PEG12-BCN, and 215-2 Ab.
SEQ ID NO: 273 is the amino acid sequence of delivery agent 216-A DBCO, 216-A FA, 216-1 PEG12-BCN, and 216-1 Ab.
SEQ ID NO: 274 is the amino acid sequence of delivery agent 216-B DBCO, 216-B FA, and 217-2.
SEQ ID NO: 275 is the amino acid sequence of delivery agent 218-2.
SEQ ID NO: 276 is the amino acid sequence of delivery agent 219-1.
SEQ ID NO: 277 is the amino acid sequence of delivery agent 219-2.
SEQ ID NO: 278 is the amino acid sequence of delivery agent 224-2, which does not comprise a mask peptide group.
SEQ ID NO: 279 is the amino acid sequence of delivery agent 225-2, which does not comprise a mask peptide group.
SEQ ID NO: 280 is the amino acid sequence of a delivery agent 223-2.

### DETAILED DESCRIPTION

### I. Explanation and Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Where a range of values is provided, it is understood that each intervening value, to the tenth of the unit of the lower limit unless the context clearly dictates otherwise, between the upper and lower limit of that range and any other stated or intervening value in that stated range is encompassed within the present disclosure. The upper and lower limits of these smaller ranges which may independently be included in the smaller ranges is also encompassed within the present disclosure, subject to any specifically excluded limit in the stated range. Where the stated range includes one or both of the limits, ranges excluding either or both of those included limits are also included in the present disclosure.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from any discussed prior art, the embodiment numbers are not approximates unless the word “about” is recited. Furthermore, not all alternatives recited herein are equivalents. And, in any numerical ranges, the specifically recited endpoints are contemplated as part of the range.

Compounds disclosed herein may contain one or more asymmetric elements such as stereogenic centers, chiral axes and the like, e.g., asymmetric carbon atoms, so that the chemical conjugates can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory

All forms (for example solvates, optical isomers, enantiomeric forms, polymorphs, free compound and salts) of a probe may be employed either alone or in combination.

To facilitate review of the various aspects of the disclosure, the following explanations of specific terms are provided. Certain functional group terms include a “—” symbol at the beginning of the functional group formula; this symbol is not a part of the functional group, but instead denotes how the functional group connects to the formulas described herein. For example, a functional group with a formula “—OC(O)R^{b}” is attached to an atom of the functionalized compound by the oxygen atom of the functional group that is next to the “—” symbol.

Administer, Administering, Administration: As used herein, administering a delivery agent and/or a therapeutic agent (e.g., a delivery agent as described herein and a therapeutic agent, such as a morpholino or other therapeutic described herein) to a subject means to apply, give, or bring the agent into contact with the subject, by any effective route. Administration can be accomplished by a variety of routes, such as, for example, intravenous, intratumoral, topical, oral, subcutaneous, transdermal, intrathecal, intramuscular, intraperitoneal, intranasal, and similar routes, or combinations thereof. Exemplary routes of administration are described herein.

Aliphatic: A substantially hydrocarbon-based compound, or a radical thereof (e.g., C₆H₁₃, for a hexane radical), including alkanes, alkenes, alkynes, including cyclic versions thereof, and further including straight- and branched-chain arrangements, and all stereo and position isomers as well. Unless expressly stated otherwise, an aliphatic group contains from one to twenty-five carbon atoms; for example, from one to fifteen, from one to ten, from one to six, or from one to four carbon atoms. The term “lower aliphatic” refers to an aliphatic group containing from one to ten carbon atoms. An aliphatic chain may be substituted or unsubstituted. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group). Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality. In some examples, a substituted aliphatic group includes at least one sp³-hybridized carbon or two sp²-hybridized carbons bonded with a double bond or at least two sp-hybridized carbons bonded with a triple bond.

Alkyl: A hydrocarbon group having a saturated carbon chain. The chain may be cyclic (e.g., cycloalkyl), branched or unbranched. Examples, without limitation, of alkyl groups include methyl, ethyl, propyl, butyl, pentyl, hexyl, heptyl, octyl, nonyl, and decyl. The term lower alkyl means the chain includes 1-10 carbon atoms. The terms alkenyl and alkynyl refer to hydrocarbon groups having carbon chains containing one or more double or triple bonds, respectively. In some examples, a substituted alkyl group includes at least one sp³-hybridized carbon.

Aliphatic-aryl: An aryl group that is or can be coupled to a compound disclosed herein, wherein the aryl group is or becomes coupled through an aliphatic group.

Aliphatic-heteroaryl: A heteroaryl group that is or can be coupled to a compound disclosed herein, wherein the heteroaryl group is or becomes coupled through an aliphatic group.

Amine: —NR^{b}R^{c}, wherein each of Rt and Re independently is selected from hydrogen, aliphatic, aryl, heteroaliphatic, aliphatic-aryl, heteroaryl, aliphatic-heteroaryl, heteroaliphatic-aryl, heteroaliphatic-heteroaryl, and any combination thereof.

Amino Acid: An organic acid containing both a basic amino group (e.g., —NH₂) and an acidic carboxyl group (e.g., —COOH). The amino acids that are protein constituents are α-amino acids wherein the —NH₂ group is attached to the carbon atom adjacent the —COOH group.

Antibody: A polypeptide ligand (such as an immunoglobulin, antigen-binding fragment, or derivative thereof) comprising at least one variable region that recognizes and binds (such as specifically recognizes and specifically binds) an epitope of an antigen. The term “antibody” is used herein in the broadest sense and encompasses various antibody structures, including but not limited to monoclonal antibodies, polyclonal antibodies, multispecific antibodies (e.g., bispecific antibodies), targeting antibodies, and antigen binding fragments, so long as they exhibit the desired antigen-binding activity. Antibodies are characterized by reacting specifically with the antigen in some demonstrable way. A therapeutic antibody recognizes and binds to the antigen receptor to activate or inhibit a series of biological processes, such as for blocking cancer cell growth and/or triggering an immune system response.

Non-limiting examples of antibodies include, for example, intact immunoglobulins and variants and antigen binding fragments thereof that retain binding affinity for the antigen. Examples of antigen binding fragments include but are not limited to Fv, Fab, dsFv. Fab′, Fab′-SH, F(ab′)₂; diabodies; linear antibodies; single-chain antibody molecules (e.g., scFv and ds-scFv); and multispecific antibodies formed from antibody fragments. Antibody fragments include antigen binding fragments either produced by the modification of whole antibodies or those synthesized de novo using recombinant DNA methodologies (see, e.g., Kontermann and Dübel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies). Antibodies also include defucosylated forms of disclosed antibodies.

An antibody may have one or more binding sites. If there is more than one binding site, the binding sites may be identical to one another or may be different. For instance, a naturally occurring immunoglobulin has two identical binding sites, a single-chain antibody or Fab fragment has one binding site, while a bispecific or bifunctional antibody has two different binding sites.

Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and has some functionally similar to mammalian IgG and IgE. IgW and IgNAR antibodies are produced by cartilaginous fish, while IgX antibodies are found in amphibians

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A “monoclonal antibody” is an antibody produced by a single clone of lymphocytes or by a cell into which the coding sequence of a single antibody has been transfected. Monoclonal antibodies include humanized monoclonal antibodies.

Cancer: A cancer is characterized by abnormal or uncontrolled cell growth (malignant cells). Other features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original cancer site and migrate to other parts of the body for example via the bloodstream or lymph system.

The “cancer burden” in a subject can be measured as the number, volume, and/or weight of one or more tumors. A tumor that does not metastasize is referred to as “benign.” A tumor that invades the surrounding tissue and/or can metastasize is a cancer (and is referred to as “malignant”).

In some examples, the cancer is a hematological cancer, such as leukemias (including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia) or chronic leukemias (such as chronic myelocytic (granulocytic) leukemia, chronic myelogenous leukemia, and chronic lymphocytic leukemia)), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. In specific non-limiting examples, the lymphoid malignancy can be adult T cell leukemia, cutaneous T cell lymphoma, anaplastic large cell lymphoma, Hodgkin's lymphoma, or a diffuse large B cell lymphoma.

Examples of solid cancers, such as sarcomas and carcinomas, include fibrosarcoma, myxosarcoma, liposarcoma, chondrosarcoma, cholangiocarcinoma (particularly Intrahepatic cholangiocarcinoma), osteogenic sarcoma, and other sarcomas, synovioma, mesothelioma, Ewing's tumor, leiomyosarcoma, rhabdomyosarcoma, colorectal cancer (e.g., colorectal carcinoma), lymphoid malignancy, pancreatic cancer, breast cancer (including basal breast carcinoma, ocular melanoma, ductal carcinoma and lobular breast carcinoma, triple-negative breast cancer), uterine/endometrial cancers, neuroendrocrine cancer, lung cancers (including non-small lung cancer), ovarian cancer, prostate cancer, hepatocellular carcinoma, angiosarcoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, gallbladder cancer, esophageal cancer, kidney cancer (e.g., renal cell carcinoma), melanoma, hepatoma, bile duct carcinoma, choriocarcinoma, Wilms' tumor, cervical cancer, brain cancer, pleural cancer, bronchial cancer, testicular cancer, seminoma, bladder carcinoma, and CNS cancers (such as a glioma, astrocytoma, medulloblastoma, craniopharyrgioma, ependymoma, pinealoma, hemangioblastoma, acoustic neuroma, oligodendroglioma, meningioma, melanoma, neuroblastoma, and retinoblastoma). Lymphoma can be a solid cancer in some presentations. In particular, non-limiting examples, a cancer is colorectal cancer, kidney cancer, or melanoma.

Cathepsin B: A lysosomal cysteine protease with endopeptidase activity (cleaving internal bonds) favored at neutral/alkaline pH values and exopeptidase (carboxydipeptidase) activity favored at acidic pH values, and may play a role in protein turnover. Cathepsin B can hydrolyze proteins with broad specificity. However, its cleavage specificity prefers, but is not restricted to, basic amino acids at the P1 position and hydrophobic or arginine residues at the P2 position. Mature cathepsin B is composed of a 25-26 kDa heavy chain and a 5 kDa light chain, which are linked by a dimer of disulfide.

Chemical Linker: A molecule or group of atoms positioned between two moieties. For example, a delivery agent described herein may include a chemical linker between a component of a disclosed delivery agent (e.g., an anchor group, a targeting group, a C-terminus group, or the like) and a therapeutic agent. Typically, chemical linkers are bifunctional and thus the chemical linker comprises a functional group at each end, wherein the functional groups are used to couple the linker to the delivery agent and the therapeutic agent. The two functional groups may be the same, (referred to as a homobifunctional linker), or different (referred to as a heterobifunctional linker).

Chemotherapeutic Agent: Any chemical or biological agent with therapeutic usefulness in treating diseases characterized by abnormal cell growth. For example, chemotherapeutic agents can be useful for the treatment of a solid cancer, such as a sarcoma, carcinoma, lymphoma, colorectal or skin cancer. Particular examples of chemotherapeutic agents that can be used include microtubule binding agents, DNA intercalators or cross-linkers, DNA synthesis inhibitors, DNA and RNA transcription inhibitors, antibodies, enzymes, enzyme inhibitors, gene regulators, and angiogenesis inhibitors. In one embodiment, a chemotherapeutic agent is a radioactive compound. Other chemotherapeutic agents that can be used are provided in Sausville and Longo, Principles of Cancer Treatment, Chapter 69 in Harrison's Principles of Internal Medicine (20^{th} ed.), McGraw-Hill. 2018; Niederhuber et al., Cancer Pharmacology, Ch. 25 in Abeloff's Clinical Oncology (6^{th} ed.), Elsevier, 2019; Gullatte et al., Clinical Guide to Antineoplastic Therapy: A Chemotherapy Handbook (4^{th} ed.). Oncology Nursing Society, 2020; Chabner and Longo, Cancer Chemotherapy, Immunotherapy and Biotherapy: Principles and Practice (6^{th} ed.), Lippincott Williams & Wilkins, 2018; Skeel, Handbook of Cancer Chemotherapy (9^{th} ed.), Lippincott Williams & Wilkins, 2016. Combination chemotherapy is the administration of more than one chemotherapeutic agent to treat cancer.

Cleavable Linker: A chemical group and/or peptide sequence positioned between two moieties. For example, a delivery agent described herein may include a cleavable linker between a lytic peptide group and a mask peptide group. In some aspects of the disclosure, the cleavable linker comprises a peptide sequence that can be cleaved so as to separate a lytic peptide group from a mask peptide group. In some aspects of the disclosure, such cleavable linkers are cleaved by an enzyme. Suitable cleavable linkers are described herein.

Combination, Combination therapy: A treatment modality that combines two or more therapeutic components, such as a delivery agent disclosed herein and one or more therapeutic agents, for the treatment of a condition or disease, such as an inherited condition, a rare disease, a cancer, an immune condition, or an infectious disease. As used herein, therapeutic components (such as a disclosed delivery agent, one or more therapeutic agents, or a conjugate formed from these two therapeutic components) provided in combination may be contacted with a cell and/or administered to a subject substantially simultaneously or sequentially in any order, at two or more different times, or a combination thereof. The one or more therapeutic agents of a combination may target several biological pathways in a characteristically synergistic or an additive manner to treat the condition or disease. A combination therapy disclosed herein may be useful for treating one type of condition or disease, such as a cancer (such as melanoma), or two or more different conditions or diseases, such as two or more types of cancers (such as kidney cancer and colorectal cancer).

Conservative variant: “Conservative” amino acid substitutions are those substitutions that do not substantially affect or decrease the affinity or activity of a protein, such as an antibody or peptide, such as a lytic peptide group, a masking peptide group, or a cleavable linker as disclosed herein. The term “conservative variant” also includes the use of a substituted amino acid in place of an unsubstituted parent amino acid, provided that the variant retains activity. Non-conservative substitutions are those that reduce an activity of a protein.

Conservative amino acid substitution tables providing functionally similar amino acids exist. The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Contacting: Placement in direct physical association, including both a solid and liquid form. In one example, contacting includes association between a therapeutic component (such as a delivery agent and/or a therapeutic agent as disclosed herein) in a liquid medium and one or more cells (such as cells in a subject or cells in a culture). Contacting can occur in vitro with isolated cells or tissue, or in vivo by administering to a subject.

Derivative: A compound that is derived from a similar compound, or a compound that can be imagined to arise from another compound, for example, if one atom is replaced with another atom or group of atoms.

Directly or Indirectly Coupled: The phrase “directly coupled” as used herein means that the referenced groups or compounds are chemically coupled to one another with nothing in between. The phrase “indirectly coupled” as used herein means that the referenced groups are chemically coupled to one another through another moiety (e.g., a functional group, a linker, or a combination thereof).

Effective Amount, Therapeutically Effective Amount: The term “effective amount” or “therapeutically effective amount” refers to the amount of an agent (such as one or more delivery agents provided herein alone, in combination, or potentially in combination with other therapeutic agent(s)) that is sufficient to induce a desired biological result. That result may be introduction of a therapeutic agent into the cytosol of a cell, and/or amelioration or alleviation of the signs, symptoms, or causes of a disease (such as a reduced cancer burden in a subject), or any other desired alteration of a biological system. The effective amount can vary with the condition being treated, the stage of advancement of the condition, and the type and concentration of formulation applied. In some aspects of the disclosure, an effective amount of a combination of therapeutic agents disclosed herein is an amount which, when administered to a subject, is sufficient to engender a detectable therapeutic response. Such a response may comprise, for instance, a reduced burden of an inherited condition, a rare disease, a cancer, an immune condition, or an infectious disease. Appropriate amounts in any given instance will be readily apparent or capable of determination by routine experimentation, such as administration of the therapeutic agent combination (such as in a combination therapy for treatment or an inherited condition, a rare disease, a cancer, an immune condition, or an infectious disease) and observation of a response in the subject.

In one embodiment, a therapeutically effective amount is the amount necessary to eliminate, reduce the size, or prevent metastasis of a tumor, such as reduce a tumor size and/or volume by at least 10%, at least 20%, at least 50%, at least 75%, at least 80%, at least 90%, at least 95%, or even 100%, and/or reduce the number and/or size/volume of metastases by at least 10%, at least 20%, at least 50%, at least 75%, at least 80%, at least 90%, at least 95%, or even 100%, for example as compared to a size/volume/number prior to treatment.

Fluorescent dye, Fluorochrome, or Fluorophore: A component of a molecule that causes the molecule to be fluorescent. The component can be a functional group of a molecule that absorbs energy of a specific wavelength and re-emits energy at a different (but equally specific) wavelength. In some aspects of the disclosure, the fluorophore can fluoresce when the compound (or a sample or composition comprising the compound) is exposed to an excitation source or after being cleaved from a compound embodiment. In some aspects of the disclosure, the component of a molecule that causes the molecule to be fluorescent is a dye. In some such aspects, the amount and wavelength of the emitted energy depend on both the dye and the chemical environment of the dye. Many dyes can include, but are not limited to, Fmoc-Lys(Mca)-OH, Fmoc-Asp(EDANS)-OH, Fmoc-Glu(EDANS)-OH, Fmoc-Lys(Dabcyl)-OH, Fmoc-Lys(Dnp)-OH, p-cyanophenylalanine, 5-cyanotryptophan, 4-cyanotryptophan, 2-cyanophenylalanine, 7-azatryptophan, 7-cyanotryptophan, β-(1-Azulenyl)-L-alanine, Acridon-2-ylalanine, L-Leucine 7-amido-4-methylcoumarin, trans-4-Hydroxy-L-proline 7-amido-4-methylcoumarin, Alexa 488, Alexa 532, Alexa 546, Alexa 568, Alexa 594, Alexa 633, Alexa 647, Atto 465, Atto 488, Atto 532, Atto 550, Atto 565, Atto 647N, Atto 655, BODIPY-TMR, Cy3, Sulfo-Cy3, Cy3B, Sulfo-Cy5, Dyomics 654, Oregon Green 488, Oregon Green 514, Sulforhodamine B, Texas Red, Tetramethylrhodamine, fluorescein isothiocyanate (FITC), R-phycoerythrin (PE), PE-Texas Red Tandem, PE-Cy5 Tandem, propidium iodem, EGFP, EYGP, ECF, DsRed, allophycocyanin (APC). PerCp, SYTOX Green, courmarin, Alexa Fluors (350, 430, 488, 532, 546, 555, 568, 594, 633, 647, 660, 680, 700, 750), Cy2, Cy3, Cy3.5, Cy5, Cy5.5, Cy7, Hoechst 33342, DAPI, Hoechst 33258, SYTOX Blue, chromomycin A3, mithramycin, YOYO-1, SYTOX Orange, ethidium bromide, 7-AAD, acridine orange, TOTO-1, TO-PRO-1, thiazole orange, TOTO-3, TO-PRO-3, thiazole orange, propidium iodide (PI), LDS 751, Indo-1, Fluo-3, DCFH, DHR, SNARF, Y66F, Y66H, EBFP, GFPuv, ECFP, GFP, AmCyanl, Y77W, S65A, S65C. S65L, S65T, ZsGreenl, ZsYellowl, DsRed2, DsRed monomer, AsRed2, mRFP1, HcRedl, monochlorobimane, calcein, the DyLight Fluors, cyanine, hydroxycoumarin, aminocoumarin, methoxycoumarin, Cascade Blue, Lucifer Yellow, NBD, PE-Cy5 conjugates, PE-Cy7 conjugates, APC-Cy7 conjugates, Red 613, fluorescein, FluorX, BODIDY-FL, TRITC, X-rhodamine, Lissamine Rhodamine B, TruRed, and derivatives thereof.

Haloallphatic: An aliphatic group wherein one or more hydrogen atoms, such as one to 10 hydrogen atoms, independently is replaced with a halogen atom, such as fluoro, bromo, chloro, or iodo.

Haloaliphatic-aryl: An aryl group that is or can be coupled to a compound disclosed herein, wherein the aryl group is or becomes coupled through a haloaliphatic group.

Haloaliphatic-heteroaryl: A heteroaryl group that is or can be coupled to a compound disclosed herein, wherein the heteroaryl group is or becomes coupled through a haloaliphatic group.

Haloheteroaliphatic: A heteroaliphatic group wherein one or more hydrogen atoms, such as one to 10 hydrogen atoms, independently is replaced with a halogen atom, such as fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group. Exemplary heteroaliphatic groups include, but are not limited to, aliphatic groups comprising an ether, a thioether, an ester, an amine, a carboxy, a carbonyl, or an amide.

Heteroallphatic-aryl: An aryl group that is or can be coupled to a compound disclosed herein, wherein the aryl group is or becomes coupled through a heteroaliphatic group.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group.

Heteroatom: An atom other than carbon or hydrogen, such as (but not limited to) oxygen, nitrogen, sulfur, silicon, boron, selenium, or phosphorous. In particular disclosed aspects, such as when valency constraints do not permit, a heteroatom does not include a halogen atom.

Immune disease or condition: A disorder or disease, such as an autoimmune disorder or disease, in which the immune system produces an immune response (e.g., a B cell or a T cell response) against an endogenous antigen, with consequent injury to tissues. The injury may be localized to certain organs, such as thyroiditis, or may involve a particular tissue at different locations, such as Goodpasture's disease, or may be systemic, such as lupus erythematosus.

In some examples, autoimmune diseases include systemic lupus erythematosus, Sjogren's syndrome, rheumatoid arthritis, type I diabetes mellitus, Wegener's granulomatosis, inflammatory bowel disease, polymyositis, dermatomyositis, multiple endocrine failure, Schmidt's syndrome, autoimmune uveitis, Achalasia, Autoimmune Encephalitis, Addison's disease, adrenalitis, Graves' disease, Eosinophilic granulomatosis with polyangiitis, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, multiple sclerosis (including Balo disease), subacute cutaneous lupus erythematosus, hypoparathyroidism, Dressler's syndrome, myasthenia gravis, Autoimmune vasculitis, autoimmune thrombocytopenia, idiopathic thrombocytopenia purpura, hemolytic anemia, pemphigus vulgaris, pemphigus, dermatitis herpetiformis, alopecia arcata, pemphigoid, autoimmune hemolytic anemia, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), male and female autoimmune infertility, ankylosing spondylitis, ulcerative colitis, Hurst's disease, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, Inclusion body myositis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, ocular cicatricial pemphigoid, eczema, lymphomatoid granulomatosis, Behcet's disease, Caplan's syndrome, Kawasaki's disease, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, paroxysmal nocturnal hemoglobinuria, glomerulonephritis, graft versus host disease, transplantation rejection, human immunodeficiency virus infection, echovirus infection, cardiomyopathy, post vaccination syndromes, congenital rubella infection, Eaton-Lambert syndrome, relapsing polychondritis, cryoglobulinemia, juvenile idiopathic arthritis, Waldenstrom's macroglobulemia, Berger's disease, rubulavirus, and Evan's syndrome. Diseases that may cause or contribute to an autoimmune disease can include Alzheimer's disease, parvovirus infection, rubella virus infection, dengue virus infection, Epstein-Barr virus infection Hodgkin's and non-Hodgkin's lymphoma, renal cell carcinoma, multiple myeloma, and malignant melanoma.

Infectious disease: Also known as transmissible disease or communicable disease, infectious diseases are illnesses resulting from an infection. Infections are caused by infectious agents, including viruses, viroids, prions, bacteria; nematodes, such as parasitic roundworms and pinworms; arthropods, such as ticks, mites, fleas, and lice; fungi, such as ringworm; and other macroparasites, such as tapeworms and other helminths. Hosts fight infections using the immune system, such as the innate response (e.g., in mammals), which involves inflammation, followed by an adaptive response. Medications used to treat infections include antibiotics, antivirals, antifungals, antiprotozoals, and antihelminthics. Specific, non-limiting examples of infectious diseases include human immunodeficiency syndrome (HIV), human papillomavirus (HPV), hepatitis B virus (HBV), hepatitis C virus (HVC), tuberculosis (TB), and malaria.

Inherited condition: A condition that results from one or more germline mutations inherited from one or both parents. Such conditions may be simply inherited and thus classified as Mendelian or single gene conditions, or can be complexly inherited (e.g., multifactorial) and classified as non-Mendelian. Mutations associated with an inherited condition (also known as an inherited disease or disorder, or a congenital condition, disease, or disorder) can include substitutions, insertions, inversions, point mutations, deletions, mismatches, copy number variations, and/or translocations. Exemplary inherited conditions that can be treated or inhibited using a disclosed delivery agent include those described herein and in The Online Metabolic and Molecular Bases of Inherited Disease (Valle et al. (Eds.), 2019, McGraw Hill, https://ommbid.mhmedical.com/content.aspx?bookid=2709&sectionid=2-3 69235).

Inhibiting or Treating a Condition or Disease: Inhibiting the full development of a disease or condition, for example, in a subject who has or is at risk of having an inherited condition, a rare disease, a cancer, an immune condition, or an infectious disease. “Treatment” refers to a therapeutic intervention that ameliorates a sign or symptom of a disease or pathological condition, such as a cancer (such as a reduced cancer burden in a subject), after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters that are specific to the particular disease, such as improved survival of a subject having an inherited condition, a rare disease, a cancer, an immune condition, or an infectious disease. Treatment may be assessed by objective or subjective parameters; including, but not limited to, the results of a physical examination, imaging, or a blood test. A “prophylactic” treatment is a treatment administered to a subject who does not exhibit signs of a disease or exhibits only early signs for the purpose of decreasing the risk of developing pathology, such as to prevent the occurrence or recurrence of a cancer, immune condition, or infectious disease.

Moiety: A moiety is a fragment of a molecule, or a portion of a conjugate.

Morpholino: A type of oligomer molecule used to modify, such as reduce or prevent, gene expression. The molecular structure of a morpholino can include DNA bases attached to a backbone of methylenemorpholine rings linked through phosphorodiamidate groups. Morpholinos block access of other molecules to small (generally approximately 25 base), specific sequences of the base-pairing surfaces of RNA, such as by binding to complementary sequences of RNA (or single-stranded DNA) by standard nucleic acid base-pairing.

Muscular dystrophy: A term used to refer to a group of genetic disorders that lead to progressive muscle weakness. Muscular dystrophy can result in skeletal muscle weakness and defects in skeletal muscle proteins, leading to a variety of impaired physiological functions. Existing treatments typically focus on ameliorating the effects of the disease and improving the patient's quality of life, such as through physical therapy or through the provision of orthopedic devices.

Mutated genes associated with muscular dystrophy are responsible for encoding a number of proteins associated with the costameric protein network. Such proteins include laminin-2, collagen, dystroglycan, integrins, caveolin-3, ankyrin, dystrophin, α-dystrobrevin, vinculin, plectin, BPAG1b, muscle LIM protein, desmin, actinin-associated LIM protein, α-actin, titin, telethonin, cypher, myotilin, and the sarcoglycan/sarcospan complex.

The most common form of muscular dystrophy is Duchenne's muscular dystrophy (DMD), affecting 1 in 3,500 live male births. DMD is an X-linked recessive disorder characterized by a mutation in the gene that codes for dystrophin. Dystrophin is a cytoskeletal protein close to 430 kDa in size. This protein works to connect the cell's cytoskeleton and extracellular matrix. The loss of dystrophin in DMD patients leads to a loss of muscle fiber attachment at the extracellular matrix during contraction, which ultimately leads to progressive fiber damage, membrane leakage and a loss of muscle function. Most patients die before they reach the age of 30 due to respiratory or cardiac failure.

Beckers muscular dystrophy (also known as Benign pseudohypertrophic muscular dystrophy) is related to DMD in that both result from a mutation in the dystrophin gene, but in DMD no functional dystrophin is produced making DMD much more severe than BMD. BMD is an X-linked recessive inherited disorder characterized by slowly progressive muscle weakness of the legs and pelvis. BMD is a type of dystrophinopathy, which includes a spectrum of muscle diseases in which there is insufficient dystrophin produced in the muscle cells, results in instability in the structure of muscle cell membrane. This is caused by mutations in the dystrophin gene, which encodes the protein dystrophin. The pattern of symptom development of BMD is similar to DMD, but with a later, and much slower rate of progression.

Congenital muscular dystrophies are caused by gene mutations. Fukuyama congenital muscular dystrophy (FCMD) and congenital muscular dystrophy type 1A (MDC1A) are examples of congenital muscular dystrophies. MDC1A is a congenital muscular dystrophy due to a genetic mutation in the LAMA2 gene which results in lack of or complete loss of laminin-α2 protein. This loss of laminin-α2 leads to an absence of laminins-211/221. Laminins-211/221 are major components of the extracellular matrix and play a key role in muscle cell development. During muscle cell differentiation laminin binds to the α7β1 integrin. Without laminin-α2, muscle fibers are unable to adhere to the basement membrane and myotubes undergo apoptosis. Muscle regeneration also fails, leading to a loss of muscle repair and an increase in muscle fibrosis and inflammation. This chronic tissue injury is a major cause of morbidity and mortality in MDC1A.

Congenital muscular dystrophies (CMD) and limb-girdle muscular dystrophy (LGMD) are common forms of highly heterogeneous muscular dystrophies which can be distinguished by their age at onset. In CMD, onset of symptoms is at birth or within the first 6 months of life; in LGMD onset of symptoms is in late childhood, adolescence or even adult life. Inheritance in LGMD can be autosomal dominant (LGMD type 1) or autosomal recessive (LGMD type 2), CMD is recessively inherited. CMD and LGMD can overlap both clinically and genetically.

MDC1A is a progressive muscle wasting disease that results in children being confined to a wheelchair, requiring ventilator assistance to breathe and premature death. Symptoms are detected at birth with poor muscle tone and “floppy” baby syndrome. DMD, BMD and LGMD are progressive muscle degenerative diseases usually diagnosed at 3-5 years of age when children show developmental delay including ability to walk and climb stairs. The disease is progressive and children are usually confined to a wheelchair in their teens and require ventilator assistance.

FCMD is an inherited condition that predominantly affects the muscles, brain, and eyes. Congenital muscular dystrophies are a group of genetic conditions that cause muscle weakness and wasting (atrophy) beginning very early in life. Fukuyama congenital muscular dystrophy affects the skeletal muscles, which are muscles the body uses for movement. The first signs of the disorder appear in early infancy and include a weak cry, poor feeding, and weak muscle tone (hypotonia). Weakness of the facial muscles often leads to a distinctive facial appearance including droopy eyelids (ptosis) and an open mouth. In childhood, muscle weakness and joint deformities (contractures) restrict movement and interfere with the development of motor skills such as sitting, standing, and walking. Fukuyama congenital muscular dystrophy also impairs brain development. People with this condition have a brain abnormality called cobblestone lissencephaly, in which the surface of the brain develops a bumpy, irregular appearance (like that of cobblestones). These changes in the structure of the brain lead to significantly delayed development of speech and motor skills and moderate to severe intellectual disability. Social skills are less severely impaired. Most children with Fukuyama congenital muscular dystrophy are never able to stand or walk, although some can sit without support and slide across the floor in a seated position. More than half of all affected children also experience seizures. Other signs and symptoms of Fukuyama congenital muscular dystrophy include impaired vision, other eye abnormalities, and slowly progressive heart problems after age 10. As the disease progresses, affected people may develop swallowing difficulties that can lead to a bacterial lung infection called aspiration pneumonia. Because of the serious medical problems associated with Fukuyama congenital muscular dystrophy, most people with the disorder live only into late childhood or adolescence.

FCMD is seen almost exclusively in Japan, where it is the second most common form of childhood muscular dystrophy (after Duchenne muscular dystrophy). Fukuyama congenital muscular dystrophy has an estimated incidence of 2 to 4 per 100,000 Japanese infants.

FCMD is caused by mutations in the FKTN gene which encodes fukutin. The most common mutation in the FKTN gene reduces the amount of fukutin produced within cells. A shortage of fukutin likely prevents the normal modification of α-dystroglycan, which disrupts that protein's normal function. Without functional α-dystroglycan to stabilize muscle cells, muscle fibers become damaged as they repeatedly contract and relax with use. The damaged fibers weaken and die over time, leading to progressive weakness and atrophy of the skeletal muscles.

Defective α-dystroglycan also affects the migration of neurons during the early development of the brain. Instead of stopping when they reach their intended destinations, some neurons migrate past the surface of the brain into the fluid-filled space that surrounds it. Because FCMD involves a malfunction of α-dystroglycan, this condition is described as a dystroglycanopathy.

Facioscapulohumeral muscular dystrophy (FHMD) is a form of muscular dystrophy associated with progressive muscle weakness and loss of muscle tissue. Unlike DMD and BMD which mainly affect the lower body. FSHD affects the upper body mainly the face, shoulder and upper arm muscles. However, it can affect muscles around the pelvis, hips, and lower leg. Symptoms for FSHD often do not appear until age 10-26, but it is not uncommon for symptoms to appear much later. In some cases, symptoms never develop. Symptoms are usually mild and very slowly become worse. Facial muscle weakness is common, and may include eyelid drooping, inability to whistle, decreased facial expression, depressed or angry facial expression, difficulty pronouncing words, shoulder muscle weakness (leading to deformities such as pronounced shoulder blades (scapular winging) and sloping shoulders), weakness of the lower, hearing loss and possible heart conditions.

Peptide: Any chain of amino acids, regardless of length or post-translational modification (e.g., glycosylation or phosphorylation). “Peptide” is used interchangeably with protein or polypeptide, and is used herein to refer to a polymer of amino acid residues. “Peptide” applies to amino acid polymers including naturally occurring amino acid polymers and non-naturally occurring amino acid polymers as well as amino acid polymers in which one or more amino acid residues is a non-natural amino acid, for example an artificial chemical mimetic of a corresponding naturally occurring amino acid. A “residue” refers to an amino acid or amino acid mimetic incorporated into a polypeptide by an amide bond or amide bond mimetic. A peptide has an amino terminal (N-terminal) end and a carboxy terminal (C-terminal) end.

Pharmaceutically acceptable carriers: The pharmaceutically acceptable carriers useful in the aspects of this disclosure are conventional. Remington: The Science and Practice of Pharmacy, (23^{rd} ed.) by Adeboye Adejare, Academic Press (2020), describes compositions and formulations suitable for pharmaceutical delivery of therapeutic agents and delivery agents herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polar: A polar compound is one in which electrons are not equally shared between the atoms such that areas of positive and negative charges are permanently separated. Polar compounds typically are soluble in water (hydrophilic). Exemplary polar molecules are polar amino acids, such as arginine, asparagine, aspartate, cysteine, glutamine, glutamate, histidine, lysine, serine, threonine, and tyrosine.

Psychiatric Disorder: Also known as mental health disorders, psychiatric disorder refers to a wide range of mental health conditions that can affect mood, thinking and behavior. Examples of mental illness include mood disorders, psychotic disorders, anxiety disorders, personality disorders, eating disorders, dementia-related disorders, and addictive behaviors. A particular example of a psychiatric disorder disclosed herein is depression. Depression is a mood disorder characterized by symptoms such as reduced mood, loss of interest and enjoyment, and/or reduced energy.

Nonpolar: A nonpolar compound is one in which electrons are equally, or nearly equally, shared between the atoms. Nonpolar compounds typically are insoluble in water (hydrophobic). Exemplary nonpolar molecules are nonpolar amino acids, such as alanine, glycine, isoleucine, leucine, methionine, phenylalanine, proline, tryptophan, and valine.

Rare Disease: A disease or condition that affects less than 200,000 people in the United States.

Specifically Bind: A binding reaction wherein, under designated conditions, an antibody binds preferentially to a particular target protein, peptide or polysaccharide (such as a tumor associated antigen) and does not bind in a significant amount to other proteins or polysaccharides present in the sample or subject (such as on a cell surface). Specific binding also occurs between streptavidin and biotin, between avidin and biotin, or between neutravidin and biotin. Specific binding can be determined by suitable methods.

With reference to an antibody-antigen complex, specific binding of the antigen and antibody can have a K_{D} of less than 10⁻⁷ Molar, such as less than 10⁻⁸ Molar, 10⁻⁹, or even less than 10⁻¹⁰ Molar. As an example, the affinity of streptavidin for biotin is discussed below.

K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

The antibodies used in the methods disclosed herein specifically bind to a defined target, such as a tumor associated antigen, such as on the surface of a cell. Thus, an antibody that specifically binds to an epitope on a tumor associated antigen is an antibody that binds substantially to the tumor associated antigen, including cells or tissue expressing the tumor associated antigen. Streptavidin, avidin, and neutravidin bind substantially to biotin. It is, of course, recognized that a certain degree of non-specific interaction may occur between an antibody or conjugate including an antibody (such as an antibody that specifically binds an antigen of interest or conjugate including such antibody) and a non-target (such as a cell that does not express the antigen). Typically, specific binding results in a much stronger association between the antibody and protein or cells bearing the antigen than between the antibody and protein or cells lacking the antigen. Similarly, specific binding for streptavidin, avidin, or neutravidin is between the streptavidin, avidin, or neutravidin, and biotin-labeled molecules (such as antibodies or aptamers), and not to molecules (such as antibodies or aptamers) labeled with a different marker. Specific binding typically results in greater than 2-fold, such as greater than 5-fold, greater than 10-fold, or greater than 100-fold increase in amount of bound antibody (per unit time) to a protein including the epitope (or labeled with biotin) or cell or tissue expressing the target epitope as compared to a protein lacking this epitope (or lacking biotin).

Subject: An organism, such as a vertebrate, such as a mammal, for example a human. Mammals include, but are not limited to, murines, simians, humans, farm animals, sport animals, and pets. In one example, the subject is a non-human mammalian subject, such as a monkey or other non-human primate, mouse, rat, rabbit, pig, goat, sheep, dog, cat, horse, or cow. In some examples the subject is a reptile, amphibian, fish or bird. Subjects can serve as a source of samples analyzed using the disclosed methods and devices.

Sugar: A compound that can be used within the context of this disclosure for targeting a specific tissue. For example, a sugar can be used in combination with a delivery agent according to the present disclosure (such as by coupling it with the delivery agent) in order to facilitate delivering the delivery agent to a particular tissue. In some aspects of the disclosure, a sugar can be used as a targeting group of the delivery agent. Solely by way of example, the sugar GalNAc can be used to target hepatic tissues thereby allowing the delivery agent's use in treating hepatic diseases. Other sugars can include, but are not limited to, glucose (such as for targeting the GLUT1 receptor at the blood-brain barrier), mannose (such as for targeting mannose-6-phosphate receptor in lung, brain, and immune cells), and the like.

Therapeutic Agent: A therapeutic agent includes treating agents, prophylactic agents, and replacement agents. A therapeutic agent may thus be any substance or any combination of substances that is useful for achieving an end or result, such as ameliorating a specific set of conditions in a subject with a disease or a disorder, for example, a substance or combination of substances (such as in a combination therapy for treating an inherited condition, a rare disease, a cancer, an immune condition, or an infectious disease), such as a substance or combination of substances useful for inhibiting cancer growth or metastasis in a subject. Therapeutic agents include proteins, nucleic acid molecules, compounds, small molecules, organic compounds, inorganic compounds, or other molecules of interest. Exemplary therapeutic agents include nucleic acid-based therapeutic agents (such as oligonucleotides, such as nucleic acid analogs, such as morpholinos), protein-based therapeutic agents, and derivatives thereof. In examples, a therapeutic agent of use herein may be a chemotherapeutic, a therapeutic antibody, an immune therapeutic, an antidepressant, an antiviral, or an antibiotic. Exemplary therapeutic agents further include saporin, cisplatin, methotrexate, fluorouracil, doxorubicin, cyclophosphamide, chlorambucil, vinblastine, vincristine, docetaxel, or paclitaxel, chlorhexidine, triclosan, xylitol, or octadecene-1-amine hydrofluoride, 1-hexadecylamine hydrofluoride, or a combination thereof.

### II. Introduction

A variety of therapeutic molecules must achieve intracellular delivery into the cell cytosol for activity. The functional delivery of larger, more polar therapeutic molecules to the cell cytosol remains a challenge, as such molecules typically are unable to directly cross cell membranes and instead are internalized via endocytosis or a similar mechanism. Endocytosis is a process by which extracellular material is taken into a cell via an invagination of the cell membrane, which closes to form a vesicle within the cell known as an endosome. Endocytosis may be receptor-mediated, where the extracellular compound binds to a specific receptor on the cell surface, or extracellular compounds may be imported nonspecifically, by virtue of their presence near the cell membrane. The latter process is also known as fluid-phase endocytosis or pinocytosis. A related process, potocytosis, takes compounds into the cell via vesicles near the cell surface known as caveolae. In each of these processes, the vesicles enclosing the extracellular compound become increasingly acidic after vesicle formation.

While peptide compositions for endosomal delivery exist in the art, they exhibit significant drawbacks that deleteriously affect in vivo use/activity. For example, such peptide compositions experience interaction with biological components, such as serum, resulting in excretion from the body such as through the kidneys. These interactions can reduce the free, active concentration of the drug and diminish their fitness for use in the clinic and other in vivo applications. Delivery agents of the present disclosure, however, can avoid such issues with binding to biological components. Without being limited to a single theory, it currently is believed that the disclosed delivery agents exhibit better in vivo activity due to using a specially designed mask peptide group that protects the active lytic peptide group of the delivery agent. This masking protection reduces or prevents interaction of the lytic peptide group with biological components (such as serum) prior to activation of the lytic peptide group within organelles of the endolysosomal pathway. The disclosed delivery agents further comprise additional structural components that lend to their use and efficacy in in vivo settings. Solely by way of example, and without intending to be limiting, a cleavable linker group can be positioned between the lytic peptide group and the mask peptide group to facilitate detaching the lytic peptide group from the mask peptide group once in the endosome, such as by enzymatic cleavage by an enzyme (e.g., cathepsin B). Solely by way of example, a cleavable linker that can be cleaved by cathepsin B can be used to reduce the amount of time the delivery agent and therapeutic agent are exposed to degradative enzymes. In yet some additional aspects, an anchor group is used as a structural component of a delivery agent to thereby enhance cell surface binding of the delivery agent, thus increasing uptake of the delivery agent (and in some aspects, one or more therapeutic agents) into the cell, such as through endocytosis as described above. In yet some additional aspects, the anchor group can be replaced or supplemented with a targeting group that facilitates directing the delivery agent to a particular location.

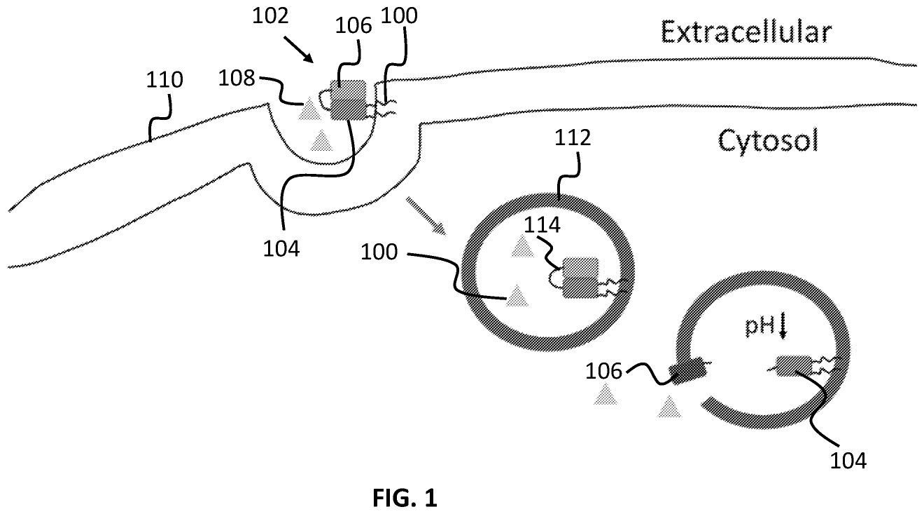

A schematic showing the process by which a delivery agent according to the present disclosure acts to facilitate delivery of a therapeutic agent into cytosol of a cell is illustrated in FIG. 1. As can be seen by FIG. 1, anchor group 100 of delivery agent 102 helps to direct and anchor the delivery agent to the extracellular membrane. Mask peptide group 104 helps disguise or protect lytic peptide group 106 from surrounding serum. Therapeutic agents 108 that are located in proximity to, or that are coupled (covalently or non-covalently) with delivery agent 102, are also present. After endocytosis into cell 110, delivery agent 102 and therapeutic agents 108 are enclosed within endosome 112. As the pH within the endosome decreases, cleavable linker 114 is cleaved, releasing lytic peptide group 106 from mask peptide group 104. This further allows lytic peptide group 106 to enter the cell membrane and, subsequently, therapeutic agents 108 to pass through the endosomal membrane and into the cytosol of the cell.

### III. Peptide-Based Delivery Agent Embodiments

The present disclosure provides a peptide-based delivery agent (also referred to herein as a “delivery agent”) that delivers a therapeutic molecule into the cytosol of a cell, such that the therapeutic molecule retains a therapeutic activity upon delivery into the cytosol. Such therapeutic molecules can be covalently or non-covalently (such as electrostatically) bound to the delivery agent, or they can be unbound, such as merely associated with the delivery agent in that they are located within close proximity of one another. The disclosed delivery agent is water-soluble, amphiphilic, membrane-destabilizing constructs. Without being bound to a particular theory, following internalization into a cell, the disclosed delivery agents may destabilize (e.g., lyse) a membrane of a membrane-bound compartment, such as an endosomal, endolysosomal, or lysosomal membrane in the cell, and/or may form one or more pores or otherwise disrupt the integrity of a membrane (such as a membrane of an endosome, endolysosome, or lysosome) of a cell, by transitioning from a polar to a nonpolar form as the pH decreases in a membrane-bound compartment relative to physiological pH. A disclosed delivery agent can thus deliver a therapeutic molecule into the cytosol of a cell, such as by lysing a membrane of a membrane-bound compartment, or by forming one or more pores in a compartment membrane or by locally disrupting the integrity of the compartment membrane, following internalization of the delivery agent and the therapeutic molecule into the same membrane-bound compartment of the cell. In some aspects of the disclosure wherein a cleavable linker is used that is rapidly cleaved, lysis or pore formation or local disruption by the delivery agent releases the therapeutic molecule from the membrane-bound compartment (such as the endosome, endolysosome, or lysosome) prior to exposure of the therapeutic molecule to many degradative enzymes.

The delivery agents described herein can have a structure comprising an N-terminus group, a lytic peptide group, a cleavable linker, a mask peptide group, an anchor group (and/or targeting group), a C-terminus group, and an optional fluorophore (which can be bound to different regions of the delivery agent, such as at the C or N-termini). In some aspects of the disclosure, the lytic peptide group provides an N-terminus group of the delivery agent in that an N-terminus of an amino acid of the lytic peptide group can be functionalized to provide the N-terminus group. In yet some additional aspects, the anchor group (and/or targeting group) provides a C-terminus group of the delivery agent in that a C-terminus of an amino acid of the anchor group (and/or targeting group) can be functionalized to provide the C-terminus group. To reduce interactions between the delivery agent and biological components, such as serum, that may otherwise reduce or eliminate delivery agent activity (such as in a subject or in a cell culture), the lytic peptide group is protected by a specially designed mask peptide group. In particular aspects, the mask peptide group is configured to be symmetrical to the lytic peptide group from the N- to C-terminus. When a delivery agent is internalized into a cell within an endosome, endolysosome, or lysosome, the mask peptide group can separate from the lytic peptide group via cleavage of the cleavable linker, exposing the lytic peptide group within the endosome, endolysosome, or lysosome. In some aspects of the disclosure, the anchor group improves association of (e.g., binding of) the delivery agent and a cell membrane, such as a plasma membrane. In aspects comprising a targeting group in addition to (or in place of) an anchor group, the targeting group can facilitate targeted delivery of the delivery agent to a particular location.

According to some aspects of the disclosure, the delivery agent can have a structure comprising (1) a lytic peptide group having a structure according to a formula [X¹Y¹Y¹X¹]ₘ, wherein m is an integer selected from 2 to 8 and each X¹ and each Y¹ independently for each occurrence are amino acids; (2) a cleavable linker; (3) a mask peptide group having a structure according to a formula [X²Y²Y²X²]_{m′}, wherein m′ is an integer selected from 2 to 8 and each X² and each Y² independently for each occurrence are amino acids; and (4) an anchor group (and/or a targeting group). In some aspects of the disclosure, the delivery agent can further comprise a fluorophore group. In yet additional aspects, the delivery agent can further comprise a bound therapeutic agent. Typically, an amino acid of the lytic peptide group can be functionalized so as to provide an N-terminus group and an amino acid of the anchor group (and/or targeting group) can be functionalized so as to provide a C-terminus group.

In particular aspects, the delivery agent can have a peptide backbone structure satisfying general Formula IA or IB, illustrated below:

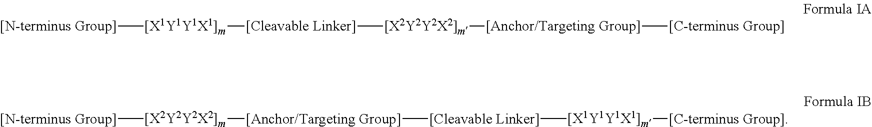

As shown in Formulas IA and IB, the lytic peptide group is indirectly coupled to the mask peptide group via a cleavable linker. In some aspects of the disclosure, the bolded X¹ group of the lytic peptide group can be functionalized to provide an N-terminus group, which can comprise a capping group or a fluorophore, or can itself provide the N-terminus of the peptide backbone of the delivery agent. The anchor group (or any targeting group) is bound to a C-terminus group, which can further comprise or be bound to an optional fluorophore group. Additional components, compounds, and/or structural features can be attached to the peptide backbone of the delivery agent to provide, for example, conjugates comprising covalently bound therapeutic agents, targeting groups, and the like. Representative aspects of lytic peptide groups, cleavable linker groups, mask peptide groups, anchor groups (and/or targeting groups), optional fluorophore groups, as well as suitable N- and C-terminus groups, are described below.

### A. N-Terminus Group

In some aspects of the disclosure, the N-terminus group of the delivery agent includes a capping group (e.g., an acetyl group) or a fluorophore. In one embodiment, the N-terminus group comprises an acetyl group that is bound to an amino acid of the lytic peptide group (e.g., an X¹ group of the lytic peptide group). In some aspects of the disclosure, the acetyl group is MeC(O)R, wherein R is the remainder of the delivery agent's peptide backbone. In another embodiment, the N-terminus group comprises a 2-(methylamino)benzamide moiety (or a derivative thereof) that is bound to an amino acid of the lytic peptide group. In particular aspects, the N-terminus comprises

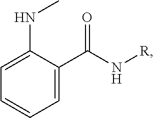

wherein R represents the remainder of the delivery agent's peptide backbone.

In some aspects of the disclosure, one or more fluorophores are attached to one or more amino acids at or near the N-terminus group of a disclosed delivery agent. In exemplary aspects, the fluorophore(s) can be 3-hydroxyisonicotinealdehyde and/or azulene and the amino acid(s) to which the fluorophore(s) is attached can be a naturally or non-naturally occurring amino acid present in the peptide backbone of the delivery agent and/or any branched peptide sequence stemming therefrom. In some such examples, the one or more fluorophores can be attached to an amine-containing amino acid, such as a lysine or other amine-containing amino acid derivative on the N-terminus group of a disclosed delivery agent.

### B. Lytic Peptide Group

According to some aspects of the disclosure, the lytic peptide group of a disclosed delivery agent can be a group capable of lysing a cell membrane. In some aspects of the disclosure, the lytic peptide group lyses membranes by transitioning from a polar to a nonpolar form as the pH decreases in a membrane-bound compartment of the cell (such as an endosome, endolysosome, or lysosome) relative to physiological pH. Without being bound to a particular theory, insertion of the delivery agent comprising the nonpolar form of the lytic peptide group into the membrane can induce lysis of the membrane, thereby delivering the therapeutic agent into the cytosol of the cell.

A lytic peptide group of the present disclosure can be a group capable of undergoing a transition between a hydrophobic, α-helical form and a hydrophilic form. As detailed below, some aspects of the disclosure of the lytic peptide group contain one or more pairs, and preferably two or more pairs, of carbonyl-containing groups (such as carboxyl groups and/or amides), where the two carbonyl-containing groups of a pair are separated by zero, one, two, or three amino acids. The composition and positioning of the amino acids of the lytic peptide group are such that, in the presence of both an aqueous and a lipid-like phase, the lytic peptide group undergoes, typically between a low pH and a neutral or high pH, such as at a pH between 4.0 and 7.0, a reversible transition between a high-pH form, comprising a multiply-ionized hydrophilic structure in which the side-chain or terminal carbonyl-containing groups are wholly or predominantly in a non-hydrogen-bonded, ionic state, and a low-pH form, comprising a substantially non-ionic α-helical structure rendered lipophilic by hydrogen bonding between paired side-chain or terminal carbonyl-containing groups. The hydrophobic, low-pH form partitions into a lipid environment, while the hydrophilic, high-pH form partitions preferentially into aqueous solution. Thus, a “reversible transition” between the lipophilic form and the hydrophilic form of a polypeptide is a transition between a nonionic, α-helical conformation, wherein side chain carbonyl-containing groups are engaged in intramolecular hydrogen bonding, favored by low pH, and a form wherein side chain carbonyl-containing groups are in ionic, non-hydrogen-bonded states, favored by neutral or high pH. Such a transition may encompass an entire lytic peptide group, or it may occur at a localized region of a lytic peptide group, particularly when the region is in the vicinity of an aqueous/lipid interface, or has a composition that is especially lipophilic and/or especially favors the forming an α-helix. Such a localized region of a lytic peptide group is often effective to initiate entry of the delivery agent into a lipid phase, such as a membrane, even though regions of the delivery agent more remote from the lipid phase may be in a hydrophilic conformation. In yet additional aspects, the lytic peptide group comprises one or more basic amino acids, such as arginine, lysine, histidine, or combinations thereof. Without being limited to a particular theory, such examples do not undergo a transition as described above but can still be used in certain types of delivery agents of the present disclosure.

In some aspects of the disclosure, the lytic peptide group has a structure according to a formula [X¹Y¹Y¹X¹]ₘ. In such aspects, m can be an integer selected from 2 to 8 or more; each X¹ independently for each occurrence can be a basic amino acid (or ionized form thereof), an acidic amino acid (or an ionized form thereof), a non-polar amino acid, or a derivative thereof; and each Y¹ independently for each occurrence can be a non-polar amino acid or a derivative thereof. In some aspects of the disclosure, m is 2, 3, 4, 5, 6, 7, 8 or more than 8. In particular, non-limiting aspects, m is 3. In some aspects of the disclosure, each X¹ independently for each occurrence is an acidic amino acid selected from glutamic acid (and/or glutamate), or aspartic acid (and/or aspartate), glutamine, or a derivative thereof; a basic amino acid selected from arginine, lysine, or a derivative thereof; or alanine (provided that at least one X¹ is other than alanine). In a specific, non-limiting embodiment, each X¹ is glutamic acid (or glutamate), aspartic acid (or aspartate) or a derivative thereof. In another specific, non-limiting embodiment, each X¹ is arginine or lysine, or an ionized form thereof, or a derivative thereof. In another specific, non-limiting embodiment, at least one X¹ is alanine or a derivative thereof, provided that at least one X¹ is other than alanine, such as a basic or acidic amino acid. In some aspects of the disclosure, each Y¹ independently for each occurrence is leucine, α-methyl leucine, methionine, alanine, or 2-amino butyric acid, or a derivative thereof. In a specific, non-limiting embodiment, each Y¹ is leucine, α-methyl leucine, or a derivative thereof. In another specific, non-limiting embodiment, each Y¹ is α-methyl leucine. In another specific, non-limiting embodiment, each Y¹ is leucine.

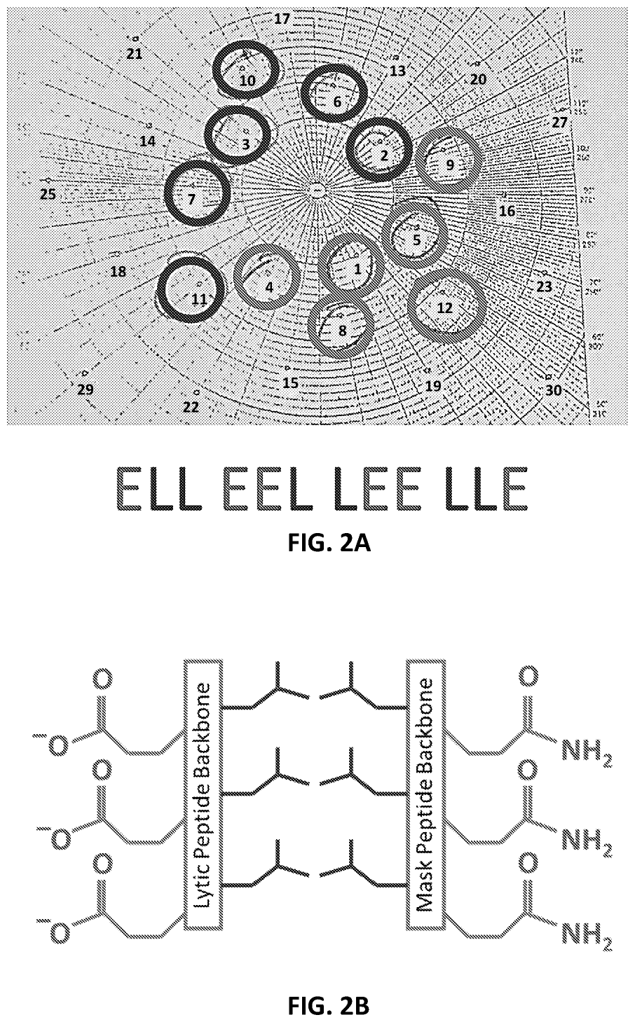

In some aspects of the disclosure, the lytic peptide group has an amino acid sequence at least 85% identical to SEQ ID NO: 1 (ELLEELLEELLE, where E is glutamic acid and L is leucine, see FIG. 2), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 1. In specific, non-limiting aspects, the lytic peptide group has an amino acid sequence comprising or consisting of SEQ ID NO: 1.

In some aspects of the disclosure, the lytic peptide group has an amino acid sequence at least 85% identical to SEQ ID NO: 2 (DLLDDLLDDLLE, where D is aspartic acid, E is glutamic acid, and L is leucine), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 2. In specific, non-limiting aspects, the lytic peptide group has an amino acid sequence comprising or consisting of SEQ ID NO: 2.

In some aspects of the disclosure, the lytic peptide group has an amino acid sequence at least 85% identical to SEQ ID NO: 38 (ELLEELLEELLEELLE, where E is glutamic acid and L is leucine), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 38. In specific, non-limiting aspects, the lytic peptide group has an amino acid sequence comprising or consisting of SEQ ID NO: 38.

In some aspects of the disclosure, the lytic peptide group has an amino acid sequence at least 85% identical to SEQ ID NO: 30 (ELLEQLLQELLE, where E is glutamic acid, Q is glutamine, and L is leucine), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 30. In specific, non-limiting aspects, the lytic peptide group has an amino acid sequence comprising or consisting of SEQ ID NO: 30.

In some aspects of the disclosure, the lytic peptide group has an amino acid sequence at least 85% identical to SEQ ID NO: 153 (QLLEQLLQQLLE, where E is glutamic acid, Q is glutamine, and L is leucine), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 153. In specific, non-limiting aspects, the lytic peptide group has an amino acid sequence comprising or consisting of SEQ ID NO: 153.

In some aspects of the disclosure, the lytic peptide group has an amino acid sequence at least 85% identical to SEQ ID NO: 92 (RLLRRLLRRLLR, where R is arginine and L is leucine), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 92. In specific, non-limiting aspects, the lytic peptide group has an amino acid sequence comprising or consisting of SEQ ID NO: 92.

### C. Cleavable Linker

According to certain aspects of the disclosure, the linker of a disclosed delivery agent can be a cleavable linker capable of separating the lytic peptide group from the remainder of the delivery agent (including the mask peptide group) within a membrane-bound compartment (such as an endosomal, endolysosomal or lysosomal compartment) following internalization of the delivery agent into an endosome of a cell. Cleavage of the cleavable linker can enable dissociating the lytic peptide group from the mask peptide group, thus allowing for the lytic activity of the lytic peptide group to act within the membrane-bound compartment.

In some aspects of the disclosure, the cleavable linker group is cleavable by an enzyme present in the endosome, endolysosome, or lysosome. In particular aspects of the disclosure, the cleavable linker group can be cleaved by cathepsin B.

In some aspects of the disclosure, the cleavable linker includes an amino acid sequence comprising 2-10 (such as 4-10 or 6-10) amino acids in length, such as 2, 3, 4, 5, 6, 7, 8, 9, or 10 amino acids in length. In a specific, non-limiting embodiment, the cleavable linker is seven amino acids in length. In another specific, non-limiting embodiment, the cleavable linker has an amino acid sequence at least 70% identical to SEQ ID NO: 3 (GFGFVGG, where G is glycine, F is phenylalanine, and V is valine), such as at least 75%, at least 80%, at least 85%, at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 3. In specific, non-limiting aspects of the disclosure, the cleavable linker has an amino acid sequence comprising or consisting of SEQ ID NO: 3. In another specific, non-limiting embodiment, the cleavable linker has an amino acid sequence at least 70% identical to SEQ ID NO: 233 (GGGVXGG, where G is glycine, X is citrulline (or Cit), and V is valine), such as at least 75%, at least 80%, at least 85%, at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 233. In specific, non-limiting aspects of the disclosure, the cleavable linker has an amino acid sequence comprising or consisting of SEQ ID NO: 233. In another specific, non-limiting embodiment, the cleavable linker has an amino acid sequence at least 70% identical to SEQ ID NO: 234 (GGGVKGG, where G is glycine, K is lysine, and V is valine), such as at least 75%, at least 80%, at least 85%, at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 234. In specific, non-limiting aspects of the disclosure, the cleavable linker has an amino acid sequence comprising or consisting of SEQ ID NO: 234. In another specific, non-limiting embodiment, the cleavable linker has an amino acid sequence at least 70% identical to SEQ ID NO: 227 (XGGXV, where G is glycine, each X independently is aminohexanoic acid or homoarginine, and V is valine), such as at least 75%, at least 80%, at least 85%, at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 227.

### D. Mask Peptide Group

According to some aspects of the disclosure, the mask peptide group of a disclosed delivery agent can be a group that can reduce or prevent exposure of the lytic peptide group of the delivery agent to biological components (such as of a cell culture system or a subject) prior to internalization of the delivery agent into a cell. In particular aspects of the disclosure, the mask peptide group hides amino acid components of the lytic peptide group from serum such that interactions between such amino acid components and serum are reduced or avoided. In some aspects of the disclosure, hydrophobic residues (e.g., leucines) of the lytic peptide group sequence can deleteriously interact with serum (e.g., serum albumin) and decrease activity of the delivery agent. The mask peptide group is able to decrease and/or prevent these interactions by hiding the hydrophobic residues until the delivery agent reaches a target (e.g., an membrane-bound compartment or other biological target region).

In some instances, a mask peptide group can contain one or more pairs, and preferably two or more pairs, of carbonyl-containing groups (such as amides), where the two carbonyl-containing groups of a pair are separated by zero, one, two, or three amino acids. Without being limited to a single theory, it currently is believed that the composition and positioning of the amino acids of the mask peptide group are such that, in the presence of an aqueous phase, the mask peptide group forms a substantially α-helical structure rendered lipophilic by hydrogen bonding between paired amide side-chains. Further, it is believed the α-helical character of the mask peptide facilitates masking of the lytic peptide. As detailed below, some aspects of the disclosure of the mask peptide group contain one or more amino acids that can be negatively charged under physiological conditions (such as glutamates) in addition to the one or more pairs, and preferably two or more pairs, of carbonyl-containing groups (such as amides). In some instances, the mask may be more soluble in an aqueous environment but still retain sufficient α-helical character in the mask peptide to facilitate masking of the lytic peptide

In some aspects of the disclosure, the mask peptide group of a disclosed delivery agent is symmetrical to the lytic peptide group of the delivery agent. In some independent aspects of the disclosure, however, the mask peptide group of a disclosed delivery agent can have one, two, or three fewer amino acids than the lytic peptide group of the delivery agent. In particular aspects of the disclosure, the mask peptide group has a structure according to a formula [X²Y²Y²X²]_{m′}. In such aspects of the disclosure, m′ can be an integer selected from 2 to 8 or more; each X² independently for each occurrence can be an acidic amino acid (e.g., glutamine, glutamic acid (and/or glutamate), aspartic acid (and/or aspartate), a non-polar amino acid (e.g., alanine), or a derivative thereof; and each Y² independently for each occurrence can be a non-polar amino acid or a derivative thereof. In particular aspects of the disclosure, at least one X² is an acidic amino acid. In some aspects of the disclosure, m′ is 2, 3, 4, 5, 6, 7, 8 or more than 8. In particular, non-limiting aspects of the disclosure, m′ is 3. In a specific, non-limiting embodiment, each X² independently for each occurrence is glutamic acid (and/or glutamate), aspartic acid (and/or aspartate), glutamine, alanine, or a derivative thereof, provided that at least one X² is other than alanine. In a specific, non-limiting embodiment, each X² independently for each occurrence is glutamic acid or a derivative thereof. In another specific, non-limiting embodiment, each X² independently for each occurrence is aspartic acid or a derivative thereof. In another specific, non-limiting embodiment, each X² is glutamine or a derivative thereof. In another specific, non-limiting embodiment, at least one X² is alanine, provided that at least one further X² is other than alanine. In some aspects of the disclosure, each Y² independently for each occurrence is leucine, α-methyl leucine, methionine, alanine, or 2-amino butyric acid, or a derivative thereof. In a specific, non-limiting embodiment, each Y² is leucine or a derivative thereof. In another specific, non-limiting embodiment, each Y² is α-methyl leucine or a derivative thereof. In another specific, non-limiting embodiment, each Y² independently for each occurrence is alanine or a derivative thereof.

In some aspects of the disclosure, the mask peptide group has an amino acid sequence at least 85% identical to SEQ ID NO: 4 (QLLQQLLQQLLQ, where Q is glutamine and L is leucine), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 4. In specific, non-limiting aspects of the disclosure, the mask peptide group has an amino acid sequence comprising or consisting of SEQ ID NO: 4.

In some aspects of the disclosure, the mask peptide group has an amino acid sequence at least 85% identical to SEQ ID NO: 51 (QLLQQLLQQLLQQLLQ, where Q is glutamine and L is leucine), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 51. In specific, non-limiting aspects of the disclosure, the mask peptide group has an amino acid sequence comprising or consisting of SEQ ID NO: 51.

In some aspects of the disclosure, the mask peptide group has an amino acid sequence at least 85% identical to SEQ ID NO: 154 (QLLQELLEQLLQ, where Q is glutamine, L is leucine, and E is glutamic acid), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 154. In specific, non-limiting aspects of the disclosure, the mask peptide group has an amino acid sequence comprising or consisting of SEQ ID NO: 154.

In some aspects of the disclosure, the mask peptide group has an amino acid sequence at least 85% identical to SEQ ID NO: 153 (QLLEQLLQQLLE, where Q is glutamine, L is leucine, and E is glutamic acid), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 153. In specific, non-limiting aspects of the disclosure, the mask peptide group has an amino acid sequence comprising or consisting of SEQ ID NO: 153.

In some aspects of the disclosure, the mask peptide group has an amino acid sequence at least 85% identical to SEQ ID NO: 148 (QLLAQLLAQLLQ, where Q is glutamine, L is leucine, and A is alanine), such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 154. In specific, non-limiting aspects of the disclosure, the mask peptide group has an amino acid sequence comprising or consisting of SEQ ID NO: 148.

E. Anchor and/or Targeting Group

In some aspects of the disclosure, endocytosis of a disclosed delivery agent can be promoted by inclusion of one or more anchor groups (e.g., 1, 2, 3, or 4 anchor groups) in the structure of the delivery agent. In some aspects of the disclosure, the anchor group comprises a heteroaliphatic group, an antibody, a biotin group, an avidin group, a streptavidin group, or a neutravidin group. In particular aspects of the disclosure, the anchor group comprises a lipid anchor group comprising a tail group that facilitates localization to a cell surface.

The anchor groups of the delivery agent disclosed herein typically comprise an amino acid portion that is bound to an amino acid group of the peptide backbone of the delivery agent. In some aspects of the disclosure, the amino acid portion of the anchor group is bound to an amino acid of the mask peptide group, such as an amino acid of the mask peptide group that is positioned towards the C-terminus group of the delivery agent. The anchor group further comprises a tail group that facilitates directing and accumulating the delivery agent at a desired location, such as at a cell surface (e.g., at a cell membrane). In particular aspects of the disclosure, the amino acid portion of the anchor group further provides a C-terminus group that can be functionalized with different components described herein, such as C-terminus group shown in Formula IA or IB, which, as described herein can comprise an amine-terminated glycine moiety, fluorophores, and the like. In particular aspects of the disclosure, the amino acid portion of the anchor group is lysine (K) and the tail group comprises an aliphatic tail. In some aspects of the disclosure, the amino acid portion of the anchor group can be bound to two separate tail groups, such as when an anchor group terminates the delivery agent peptide backbone or terminates a branching group the stems from the delivery agent peptide backbone. In some aspects of the disclosure, the tail group can further comprise a functional group that facilitates binding the aliphatic tail to the amino acid portion. In some aspects of the disclosure, the functional group can be provided by a 2-aminoethyl hydrogen carbonate group

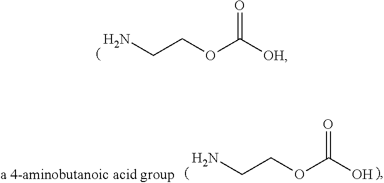 [SMILES: NCCOC(=O)O ; NCCOC(=O)O]

a —CH(NH₂)C(O)— group, or a —CH(N-Me₃)C(O)— group. In some aspects of the disclosure, the aliphatic tail comprises an alkyl chain comprising 6 to 12 carbon atoms in length (e.g., 6, 7, 8, 9, 10, 11, or 12). In yet some additional aspects of the disclosure, the amino group of the 2-aminoethyl hydrogen carbonate group and/or the 4-aminobutanoic acid group can be functionalized so as to provide a quaternary amine. In particular aspects of the disclosure, the amino group is functionalized with an aliphatic group, such as a lower alkyl group comprising 10 or fewer carbon atoms. In some exemplary aspects of the disclosure, the amino group is functionalized with at least one methyl group, with some particular examples comprising two methyl groups. In particular aspects of the disclosure, the tail portion can have a structure selected from any of the structures illustrated below (and wherein the wavy line indicates the connection point to the amino acid portion of the anchor group.

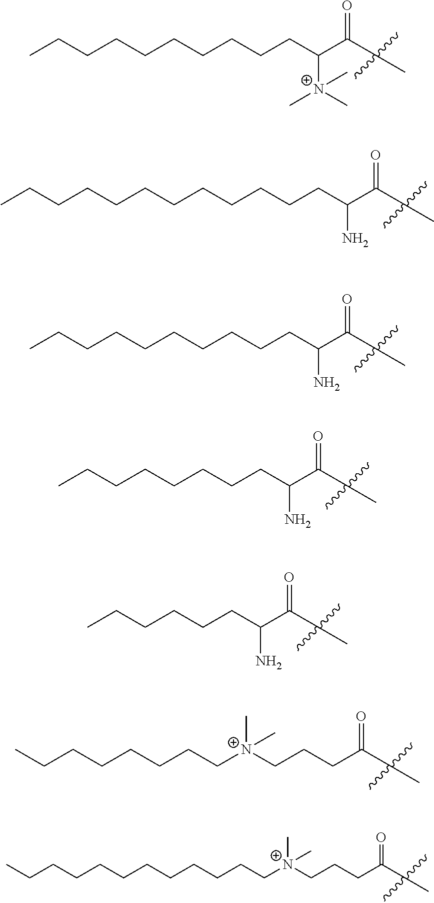

In some aspects of the disclosure, the anchor group can have a structure according to any of Formulas IIA-IID below.

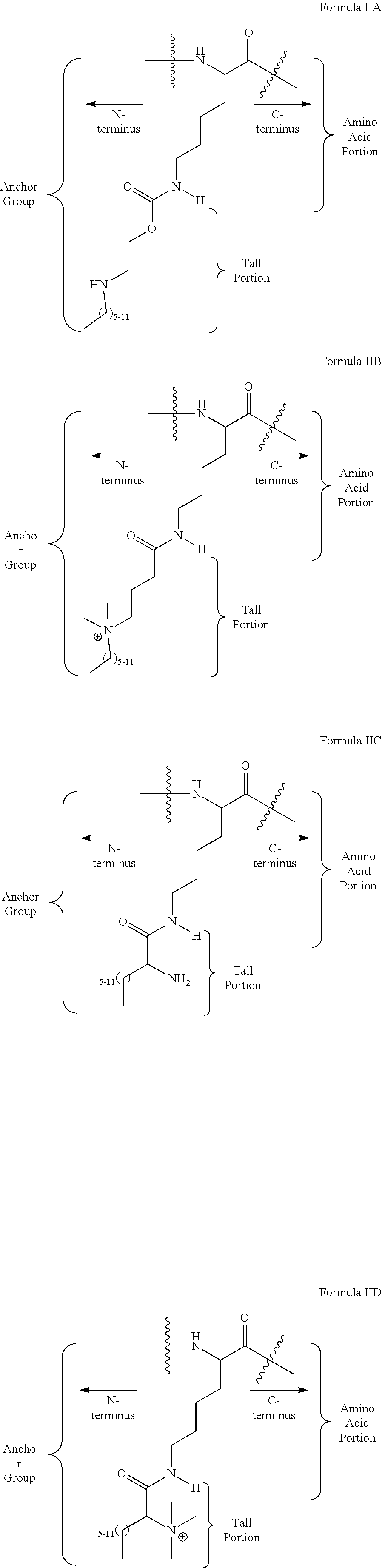

In some aspects of the disclosure, one or more anchor groups can be included in the delivery agent. In some aspects of the disclosure, two anchor groups may be bound directly to one another or may be bound indirectly via one or more amino acids. In some such aspects of the disclosure, the two anchor groups can be identical or different in terms of chemical structure. In some aspects of the disclosure wherein two (or more) anchor groups are directly bound to one another, the amino acid portion of one of the anchor groups may be bound to an amino acid portion of the other anchor group via a peptide bond. Solely by way of example, wherein the amino acid portion of each of two anchor groups is lysine, the amino group of one lysine can be bound to the mask peptide group and its corresponding carboxylic acid group can be bound to the amino group of the second lysine. The carboxylic acid group of the second lysine can in turn be bound to the C-terminus group of the delivery agent. In some aspects of the disclosure, two or more different anchor groups can be included in the delivery agent such as one or more groups that can have a structure according to Formulas IIA, IIB, IIC, or IID and one or more ligands. In receptor-mediated endocytosis, the anchor group attached or complexed to the composition targeted for uptake, is capable of binding (such as specifically binding) to a receptor (such as a receptor specific for the ligand) on the cell surface. Such anchor groups may be used to enhance general endocytotic uptake, or to target specific cell types.

Delivery agents disclosed herein can further comprise a targeting group, either in place of or in addition to any anchor group(s) included in the delivery agent. A targeting group can be utilized to facilitate specific delivery of the delivery agent (and any therapeutic agent coupled thereto) to a specific target (e.g., biological region, cell, or biological structure). Targeting moieties can include, cells, an antibody (or fragment thereof), a peptide, a biomimetic peptide, an aptamer, a sugar, a small targeting molecule, or a combination thereof. Exemplary antibodies can include anti-CD33, anti-CD30, anti-HER2, anti-CD22, anti-nectin4, anti-nectin1, anti-AXL, anti-CD74, anti-ALK, anti-PTK7, anti-PSMA, anti-TM4SF1, anti-CD276, anti-CD20, anti-CD19, anti-CD3, anti-CD71. Exemplary antibody/target combinations can include the interferon-α/β cell surface receptor complex used with targeting Type I interferons (IFNα, IFNβ), death receptors 4 and 5 (DR4/5) targeted with tumor necrosis factor-related apoptosis inducing ligand, EGFRvIII targeted with EGFR targeting antibodies or nanobodies (ENb), and CD36 targeted with thrombospondin. Exemplary peptides and/or biomimetic peptides can include, but are not limited to, iRGD (which binds to integrin receptors in tumors), PEN-221, octreotide, rabies virus glycoprotein-29, miniAp-4, angiopep-1, RGD-4C (cyclic), BT1718, PL1, and the like. Exemplary sugars include, but are not limited to, glucose, mannose, and galactose. Exemplary cells that can be used as targeting groups include, but are not limited to, red blood cells, macrophages, neutrophils, monocytes, T-cells, and the like. Exemplary small targeting molecules can include, but are not limited to, folic acid (for targeting cancers), bisphosphonates (for targeting bone), a dibenzocyclooctyne compound (or “DBCO”) (for targeting a cell surface or other cellular features), and the like. In some embodiments, the dibenzocyclooctyne compound also can be used as an anchor group as it may facilitate binding the delivery agent to a cell surface.

A targeting group can be attached to the delivery agent by way of an attachment point, which can comprise an amino acid (e.g., lysine or cysteine group) of the delivery agent peptide backbone (or any such groups present in a branch stemming from the delivery agent peptide backbone). In some aspects of the disclosure, the targeting group can be coupled to the delivery agent by functionalizing one or more of these amino acids at an available amine, carboxyl, or side chain group of the amino acid. Attachment points can be included in a targeting group of the peptide backbone, a branch stemming from the peptide backbone, and/or a C-terminus group of the peptide backbone. Solely by way of example, a targeting group can be attached to the delivery agent by an amine terminus of a lysine group or by an amine of the side chain of the lysine.

In some aspects of the disclosure, the anchor group and/or targeting group can be attached directly to the delivery agent through any of the above-mentioned attachments, or it can be attached indirectly through a linking group. In some aspects of the disclosure, the linking group can comprise a carbonyl group, an amide group, an alkylene oxide group, or a combination thereof. In some aspects of the disclosure, the linking group has a structure according to Formula A, Formula B, or Formula C illustrated below, wherein the circled wavy bond(s) of Formula A, Formula B, and Formula C denote the point of attachment of the anchor and/or targeting group (or multiple independent anchor and/or targeting groups, such as with Formulas A and C) and the other wavy line of Formula A, Formula B, and Formula C denote attachment to the rest of the delivery agent.

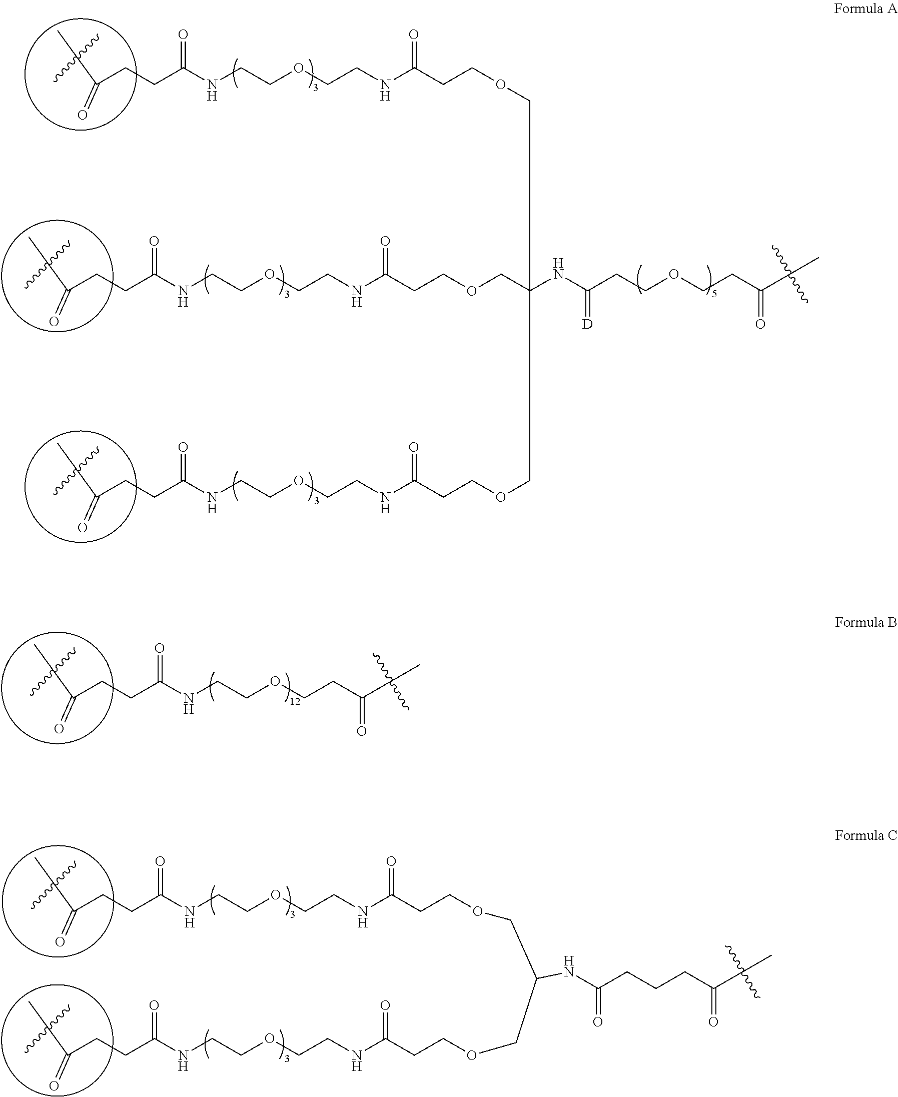

In particular aspects of the disclosure, the delivery agent has an amino acid sequence at least 85% identical to SEQ ID NO: 272, such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 272, wherein SEQ ID NO: 272 is QLLEQLLQQLLEGFGFVGGQLLEQLLQQLLEKGXG, wherein K at position 32 provides one or more DBCO groups bound to the Lys sidechain with a linking group according to any one of Formulas A, B, or C, X is naphthylalanine (Nal), the C-terminal G at position 35 is a modified glycine comprising a —C(O)—NH₂ group, and in some aspects, the N-terminal Q comprises an N-terminus modified with an acetyl group. In specific, non-limiting aspects, the delivery agent has an amino acid sequence comprising or consisting of SEQ ID NO: 272. In any of these aspects, K at position 32 provides (i) three DBCO groups bound to the Lys sidechain with a linking group according to Formula A; (ii) one DBCO group bound to the Lys sidechain with a linking group according to Formula B; or (iii) two DBCO groups bound to the Lys sidechain with a linking group according to Formula C.

In particular aspects of the disclosure, the delivery agent has an amino acid sequence at least 85% identical to SEQ ID NO: 273, such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 273, wherein SEQ ID NO: 273 is ELLEELLEELLEGFGFVGGOLLAQLLAQLLOKGXG, wherein K at position 32 provides one or more DBCO groups bound to the Lys sidechain with a linking group according to any one of Formulas A, B, or C, X is naphthylalanine (Nal), the C-terminal G at position 35 is a modified glycine comprising a —C(O)—NH₂ group, and in some aspects, the N-terminal Q comprises an N-terminus modified with an acetyl group. In specific, non-limiting aspects, the delivery agent has an amino acid sequence comprising or consisting of SEQ ID NO: 273. In any of these aspects, K at position 32 provides (i) three DBCO groups bound to the Lys sidechain with a linking group according to Formula A; (ii) one DBCO group bound to the Lys sidechain with a linking group according to Formula B; or (iii) two DBCO groups bound to the Lys sidechain with a linking group according to Formula C.

In particular aspects of the disclosure, the delivery agent has an amino acid sequence at least 85% identical to SEQ ID NO: 274, such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to SEQ ID NO: 273, wherein SEQ ID NO: 273 is ELLEELLEELLEGFGFVGGQLLQQLLQQLLQKGXG, wherein K at position 32 provides one or more DBCO groups bound to the Lys sidechain with a linking group according to any one of Formulas A, B, or C, X is naphthylalanine (Nal), the C-terminal G at position 35 is a modified glycine comprising a —C(O)—NH₂ group, and in some aspects, the N-terminal Q comprises an N-terminus modified with an acetyl group. In specific, non-limiting aspects, the delivery agent has an amino acid sequence comprising or consisting of SEQ ID NO: 273. In any of these aspects, K at position 32 provides (i) three DBCO groups bound to the Lys sidechain with a linking group according to Formula A; (ii) one DBCO group bound to the Lys sidechain with a linking group according to Formula B; or (iii) two DBCO groups bound to the Lys sidechain with a linking group according to Formula C.

### F. C-Terminus Group

In some aspects of the disclosure, the C-terminus group of the delivery agent includes an amine-terminated glycine moiety, a fluorophore, or both. In particular aspects of the disclosure wherein the C-terminus group includes a fluorophore, the fluorophore can be naphthylalanine or tryptophan, and may be flanked by a glycine moiety on one or both sides of the fluorophore. In particular aspects of the disclosure, the amine-terminated glycine moiety has a structure selected from -Nal-K-G′, -G-K-G′, -Nal-C-G′, -G-Nal-C-G′, -G-C-G′, -Nal-G′, -G-Nal-G′, -G-Nal-K-G′ (SEQ ID NO: 5), —W-G′, -G-W-G′, or -G-W—K-G′, where Nal is naphthylalanine, G is glycine, G′ is a modified glycine comprising a —C(O)-amine group, K is lysine, C is cysteine, and W is tryptophan. In some aspects of the disclosure, any lysine or cysteine of the C-terminus group can serve as attachment points and thus can be used for attaching targeting groups, such as those described herein. In a specific, non-limiting embodiment, the amine-terminated glycine moiety is G′ or -G-Nal-G′, where G′ is a modified glycine including a —C(O)—N(R^{a})₂ group, where each R^{a} independently is hydrogen or aliphatic (e.g., methyl, ethyl, propyl, i-propyl, t-butyl, i-butyl, n-butyl, and the like). In yet additional aspects of the disclosure, the amine-terminated glycine moiety is -G-Nal-K-G′, where G′ is a modified glycine including a —C(O)—N(R^{a})₂ group, where each R^{a} independently is hydrogen or aliphatic (e.g., methyl, ethyl, propyl, i-propyl, t-butyl, i-butyl, n-butyl, and the like).

In some aspects of the disclosure, one or more other types of fluorophores can be attached to one or more amino acids, such as a non-naturally occurring amino acid, present in the C-terminus group of a disclosed delivery agent. In such examples, the one or more fluorophores can be attached to a cysteine or an amine-containing amino acid, such as a lysine or other amine-containing amino acid derivative, present in the C-terminus group of a disclosed delivery agent. In some such aspects of the disclosure, the fluorophore is selected from 3-hydroxyisonicotinealdehyde, azulene, Fmoc-Lys(Mca)-OH, Fmoc-Asp(EDANS)-OH, Fmoc-Glu(EDANS)-OH, Fmoc-Lys(Dabcyl)-OH, Fmoc-Lys(Dnp)-OH, p-cyanophenylalanine, 5-cyanotryptophan, 4-cyanotryptophan, 2-cyanophenylalanine, 7-azatryptophan, 7-cyanotryptophan, β-(1-Azulenyl)-L-alanine, Acridon-2-ylalanine, L-Leucine 7-amido-4-methylcoumarin, trans-4-Hydroxy-L-proline 7-amido-4-methylcoumarin, Alexa 488, Alexa 532, Alexa 546, Alexa 568, Alexa 594, Alexa 633, Alexa 647, Atto 465, Atto 488, Atto 532, Atto 550, Atto 565, Atto 647N, Atto 655, BODIPY-TMR, Cy3, Sulfo-Cy3, Cy3B, Sulfo-Cy5, Dyomics 654, Oregon Green 488, Oregon Green 514, Sulforhodamine B, Texas Red, or Tetramethylrhodamine. In exemplary aspects of the disclosure, the fluorophore is selected from naphthylalanine, tryptophan, 3-hydroxyisonicotinealdehyde, and/or azulene.

In some aspects of the disclosure, a therapeutic agent or a targeting group, can be coupled to the delivery agent, covalently or non-covalently, via the C-terminus group. In some aspects of the disclosure, the therapeutic agent can be coupled to the delivery agent via a disulfide that can be cleaved to separate the delivery agent from the therapeutic agent. In some aspects of the disclosure, the therapeutic agent can be coupled to the delivery agent via a second cleavable linker, which has a similar or identical sequence, or a different sequence, compared to that as described for other cleavable linkers described herein that are present in the delivery agent peptide backbone. In such aspects of the disclosure, the cleavable linker is attached to the amine-terminated glycine moiety. For example, the cleavable linker can be covalently bound to a glycine of the amine-terminated glycine moiety, or it can be bound to a different amino acid of the amine-terminated glycine moiety, such as a lysine or a cysteine in aspects of the disclosure comprising an amine-terminated glycine moiety having a structure as described above. In some aspects of the disclosure wherein the therapeutic agent is coupled to the delivery agent, it can be bound indirectly to the cleavable linker group via a chemical linker group. In some such aspects of the disclosure, the chemical linker group can have a structure according to any of Formulas IIIA-IIID. In yet additional aspects of the disclosure, the chemical linker group can be selected from pyrophosphate diester linker (such as a pyrophosphate diester linker that can be cleaved by phosphatase and pyrophosphates in lysosomes and as described by Kern et al., J. Am. Chem. Soc. 2016, 138(4):1430-1445; and Zheng et al., Acta Pharmaceutica Sinica B. 2021, 11(12):3889-3907), wherein the structures of such pyrophosphate diester linkers disclosed by these documents are incorporated herein by reference); a β-Glucuronide linker (such as a β-Glucuronide linker that can be cleaved by β-glucuronidase, an enzyme present in lysosomes and over expressed in some tumors, and as described by Lu et al., Int J Mol Sci. 2016, 17(4):561, wherein the structures of such β-Glucuronide linkers are incorporated herein by reference); a β-galactose linker (such as a β-galactose linker cleaved by β-galactosidase, an enzyme located in lysosomes and overexpressed in some tumors, and as described by Komatsu et al., J. Am. Chem. Soc. 2006, 128(50):15946-15947; and Zheng et al., Acta Pharmaceutica Sinica B. 2021, 11(12):3889-3907, wherein the structures of such β-galactose linkers disclosed by these documents are incorporated herein by reference); an arylsulfate linker (such as an arylsulfate linker that can be cleaved by sulfatase, an enzyme located in lysosomes and overexpressed in some cancers) and/or a photo-responsive cleavable linker, both of which are described by Table 1 of Zheng et al., Acta Pharmaceutica Sinica B. 2021, 11(12):3889-3907 (the structures of Table 1 hereby being incorporated herein by reference); or another linker that can be cleaved in low pH environments, such as, but not limited to, orthoesters (Srinivasachar et al., Biochemistry, 1989, 28(6):2501-2509, wherein orthoesters described therein are incorporated herein by reference), silyl ether (Parrott et al., J. Am. Chem. Soc. 2010, 132(50):17928-17932, wherein silyl ethers described therein are incorporated herein by reference), an acetal linker (Liu et al., J. Am. Chem. Soc. 2010, 132(5):1500-1501, wherein acetal linkers described therein are incorporated herein by reference), a β-thiopropionate Linker (Oishi et al., Am. Chem. Soc. 2005, 127(6):1624-1625, wherein β-thiopropionate linkers described therein are incorporated herein by reference), a phosphoramidate linkage (Jeong et al., Bioconjugate Chemistry, 2003, 14(2):473-479, wherein phosphoramidate linkages described therein are incorporated herein by reference), an imine linker (Zhao et al., J. Am. Chem. Soc. 2010, 132(37):13016-13025, wherein imine linkages described therein are incorporated herein by reference), a vinyl ether (Shin et al., J. Controlled Release, 2003, 91:187-200, wherein vinyl ether linkages described therein are incorporated herein by reference), or a hydrazone (Kale et al., Bioconjugate Chem. 2007, 18(2):363-370, wherein hydrazone linkages described therein are incorporated herein by reference). With reference to Formulas IIIA-IIID, each p independently is an integer selected from 0 to 20, such as 1 to 15, or 1 to 12, or 1 to 10, or 1 to 8, or 1 to 5, or 1 to 4; and q is an integer selected from 0 or 1 and when q is 0, then the 2^{nd} cleavable linker is not present and the chemical linker group is directly attached to the delivery agent.

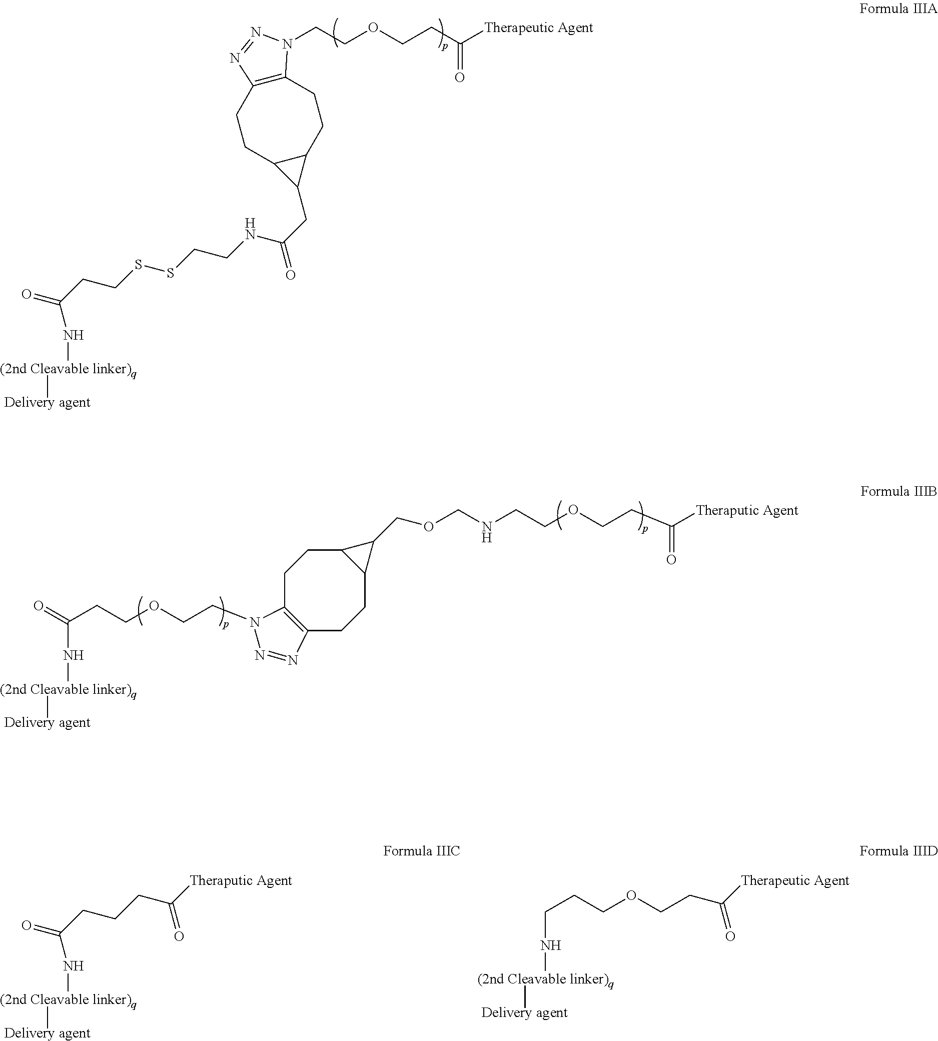

### G. Exemplary Delivery Agent Embodiments

In a specific, non-limiting embodiment, the delivery agent includes (1) an N-terminus group (e.g., an acetyl group); (2) a lytic peptide group having a structure according to a formula [X¹Y¹Y¹X¹]ₘ, wherein m is 3, each X¹ is glutamic acid, and each Y¹ is leucine; (3) a cleavable linker having the amino acid sequence of SEQ ID NO: 3; (4) a mask peptide group having a structure according to a formula [X²Y²Y²X²]_{m′}, wherein m′ is 3, each X² is glutamine, and each Y² is leucine; (5) an anchor group having a structure according to any of Formulas IIA-IID; and (6) a C-terminus group, such as amine-terminated glycine moiety having a structure-G-Nal-G′, wherein G is glycine, Nal is naphthylalanine, and G′ is a modified glycine comprising a —C(O)—N(R^{a})₂ group, wherein each R^{a} independently is hydrogen or aliphatic.

In another specific, non-limiting embodiment, the delivery agent includes (1) an N-terminus group (e.g., an acetyl group); (2) a lytic peptide group having a structure according to a formula [X¹Y¹Y¹X¹]ₘ, wherein m is 3, each X¹ is glutamic acid, and each Y¹ is leucine; (3) a cleavable linker having the amino acid sequence of SEQ ID NO: 3; (4) a mask peptide group having a structure according to a formula [X²Y²Y²X²]_{m′}, wherein m′ is 3, each X² is glutamine, and each Y² is leucine; (5) an anchor group having a structure according to any of Formulas IIA-IID; and (6) a C-terminus group, such as amine-terminated glycine moiety having a structure -G-Nal-G′, wherein G is glycine, Nal is naphthylalanine, and G′ is a modified glycine comprising a —C(O)—N(R^{a})₂ group, wherein each R^{a} independently is hydrogen or aliphatic.

In some aspects of the disclosure, an amino acid sequence of the lytic peptide group, cleavable linker, and mask peptide group comprises any one of SEQ ID NOs: 6 or 9. In some aspects of the disclosure, the amino acid sequence of the lytic peptide group, cleavable linker, and mask peptide group has an amino acid sequence at least 85% identical to any one of SEQ ID NOs: 6 or 9, such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 6 or 9. In specific, non-limiting aspects of the disclosure, the amino acid sequence of the lytic peptide group, cleavable linker, and mask peptide group has an amino acid sequence comprising or consisting of any one of SEQ ID NOs: 6 or 9.

In some aspects of the disclosure, an amino acid sequence of the lytic peptide group, cleavable linker, mask peptide group, and amino acid portions of the anchor groups comprises any one of SEQ ID NOs: 7 or 10. In some aspects of the disclosure, the amino acid sequence of the amino acid sequence of the lytic peptide group, cleavable linker, mask peptide group, and amino acid portions of the anchor groups has an amino acid sequence at least 85% identical to any one of SEQ ID NOs: 7 or 10, such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 7 or 10. In specific, non-limiting aspects of the disclosure, the amino acid sequence of the amino acid sequence of the lytic peptide group, cleavable linker, mask peptide group, and amino acid portions of the anchor groups has an amino acid sequence comprising or consisting of any one of SEQ ID NOs: 7 or 10.

In some aspects of the disclosure, an amino acid sequence of the lytic peptide group, cleavable linker, mask peptide group, amino acid portions of the anchor groups, and C-terminal moiety comprises any one of SEQ ID NOs: 8, 11, 13-15, 39, 149, or 171. In some aspects of the disclosure, the amino acid sequence of the lytic peptide group, cleavable linker, mask peptide group, amino acid portions of the anchor groups, and C-terminal moiety has an amino acid sequence at least 85% identical to any one of SEQ ID NOs: 8, 11, 13-15, 39, 149, or 171, such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 8, 11, 13-15, 39, 149, or 171. In specific, non-limiting aspects of the disclosure, the amino acid sequence of the lytic peptide group, cleavable linker, mask peptide group, amino acid portions of the anchor groups, and C-terminal moiety has an amino acid sequence comprising or consisting of any one of SEQ ID NOs: 8, 11, 13-15, 39, 149, or 171.

SEQ ID NO: 6: ELLEELLEELLEGFGFVGGQLLQQLLQQLLQ, wherein in some aspects of the disclosure, the N-terminal E comprises an N-terminus modified with an acetyl group.

SEQ ID NO: 7: ELLEELLEELLEGFGFVGGQLLQQLLQQLLQKK, wherein each K provides an anchor group and in some aspects of the disclosure, the N-terminal E comprises an N-terminus modified with an acetyl group.

SEQ ID NO: 8: ELLEELLEELLEGFGFVGGOLLQQLLQQLLOKKGXG, wherein each K provides an anchor group, X is naphthylalanine (Nal), the C-terminal G at position 36 is a modified glycine comprising a —C(O)—NH₂ group, and in some aspects of the disclosure, the N-terminal E comprises an N-terminus modified with an acetyl group.

SEQ ID NO: 9: DLLDDLLDDLLEGFGFVGGQLLQQLLOOLLQ, wherein in some aspects of the disclosure, the N-terminal D comprises an N-terminus modified with an acetyl group.

SEQ ID NO: 10: DLLDDLLDDLLEGFGFVGGQLLQQLLQQLLOKK, wherein each K provides an anchor group, wherein in some aspects of the disclosure, the N-terminal D comprises an N-terminus modified with an acetyl group.

SEQ ID NO: 11: DLLDDLLDDLLEGFGFVGGQLLQQLLQQLLOKKGXG, wherein each K provides an anchor group, X is Nal, the C-terminal G at position 36 is a modified glycine comprising a —C(O)—NH₂ group, and in some aspects of the disclosure, the N-terminal D comprises an N-terminus modified with an acetyl group.

SEQ ID NO: 13: ELLEELLEELLEGFGFVGGQLLQQLLQQLLOKKG, wherein each K provides an anchor group and the C-terminal G at position 34 is a modified glycine comprising a —C(O)—NH₂ group, and in some aspects of the disclosure, the N-terminal E comprises an N-terminus modified with an acetyl group or a fluorophore.

SEQ ID NO: 14: ELLEELLEELLEGFGFVGGQLLQQLLQQLLOKKGXG, wherein each K provides an anchor group, X is Nal and the C-terminal G at position 36 is a modified glycine comprising a —C(O)—NH₂ group, and in some aspects of the disclosure, the N-terminal E comprises an N-terminus modified with an acetyl group.

SEQ ID NO: 15: ELLEELLEELLEGFGFVGGQLLQQLLQQLLOKKGWG, wherein each K provides an anchor group and the C-terminal G at position 36 is a modified glycine comprising a —C(O)—NH₂ group, and in some aspects of the disclosure, the N-terminal E comprises an N-terminus modified with an acetyl group.

SEQ ID NO: 39: ELLEELLEELLEELLEGFGFVGGQLLQQLLQQLLQQLLOKKG, wherein each K provides an anchor group, the C-terminal G at position 42 is a modified glycine comprising a —C(O)—NH₂ group, and in some aspects of the disclosure, the N-terminal E comprises an N-terminus modified with an acetyl group.

SEQ ID NO: 149: ELLEELLEELLEGFGFVGGQLLAQLLAQLLOKG, wherein K provides an anchor group and the C-terminal G at position 33 is a modified glycine comprising a —C(O)—NH₂ group, and in some aspects of the disclosure, the N-terminal E comprises an N-terminus modified with an acetyl group.

SEQ ID NO: 171: QLLEQLLQQLLEGFGFVGGQLLEQLLQQLLEKG, wherein K provides an anchor group and the C-terminal G at position 33 is a modified glycine comprising a —C(O)—NH₂ group, and in some aspects of the disclosure, the N-terminal Q comprises an N-terminus modified with an acetyl group.

With reference to all of SEQ ID NOs: 6-11, 13-15, 39, 149, and 171, the N-terminal E, D, or Q amino acids of these sequences comprises an N-terminal amine functionalized with an acetyl group.

Further exemplary, non-limiting examples of the delivery agent are illustrated below, which further comprise conjugated therapeutic agents attached via chemical linker groups and/or second cleavable linker groups.

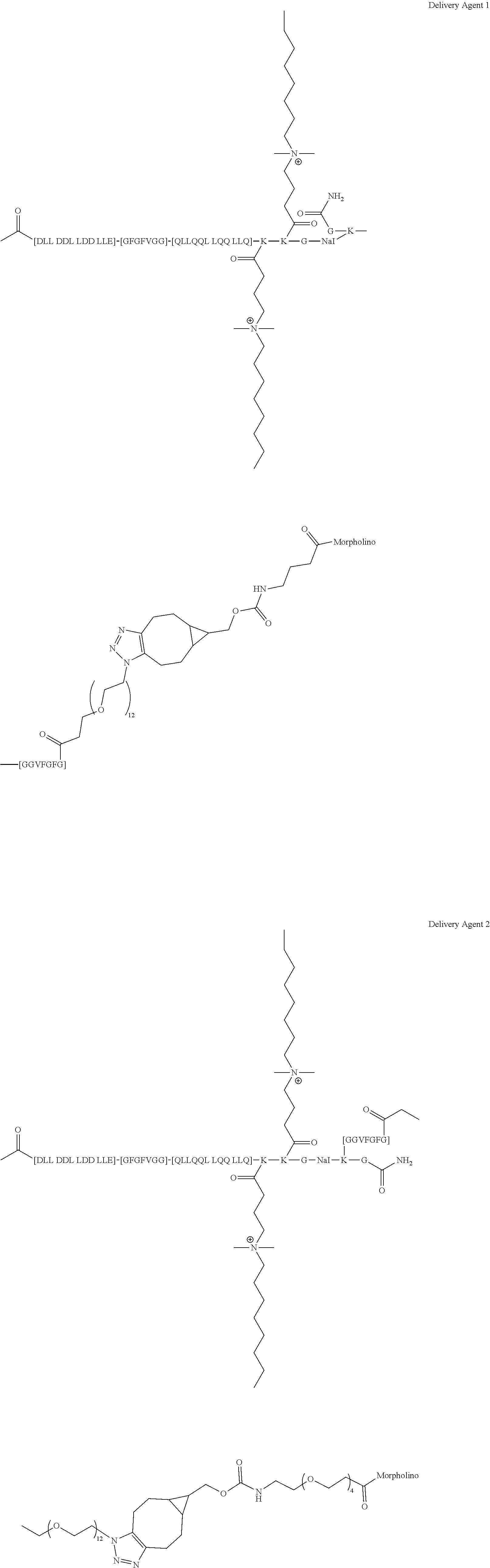

With reference to Delivery Agents 1 and 2, SEQ ID NO: 12 (DLLDDLLDDLLEGFGFVGGQLLQQLLQQLLQKKGXKG, where X is Nal, K at positions 32 and 33 each provides an anchor group. K at position 36 is functionalized with a second cleavable linker that is conjugated to a therapeutic agent via a chemical linker group, and the C-terminal G at position 37 is functionalized with an NH₂ group) is the combined amino acid sequence of the lytic peptide group, cleavable linker, mask peptide group, amino acid portions of two anchor groups, and C-terminal moiety of Delivery Agents 1 and 2, shown above.

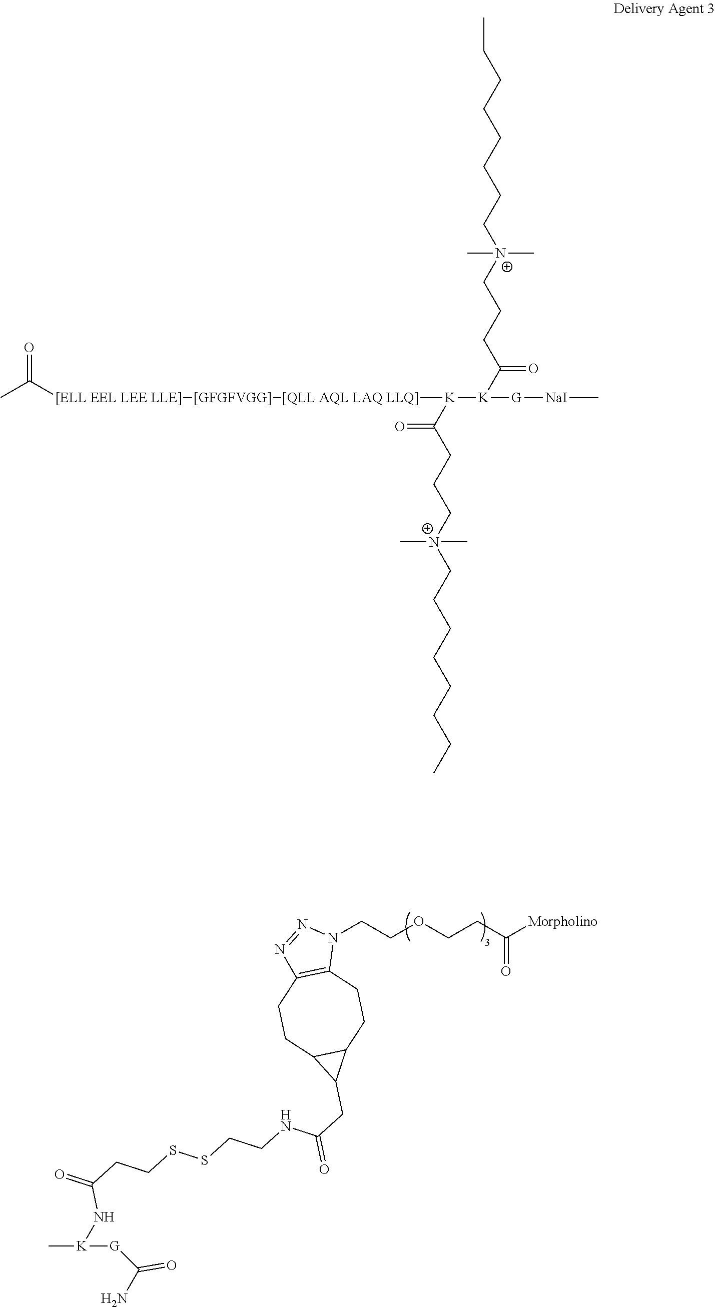

With reference to Delivery Agent 3, SEQ ID NO: 108 (ELLEELLEELLEGFGFVGGOLLQQLLQQLLOKKGXKG, where X is Nal, K at positions 32 and 33 each provides an anchor group. K at position 36 is functionalized with a chemical linker group attached to a therapeutic agent, and the C-terminal G at position 37 is functionalized with an NH₂ group) is the combined amino acid sequence of the lytic peptide group, cleavable linker, mask peptide group, amino acid portions of two anchor groups, and C-terminal moiety of Delivery Agent 3, shown above.

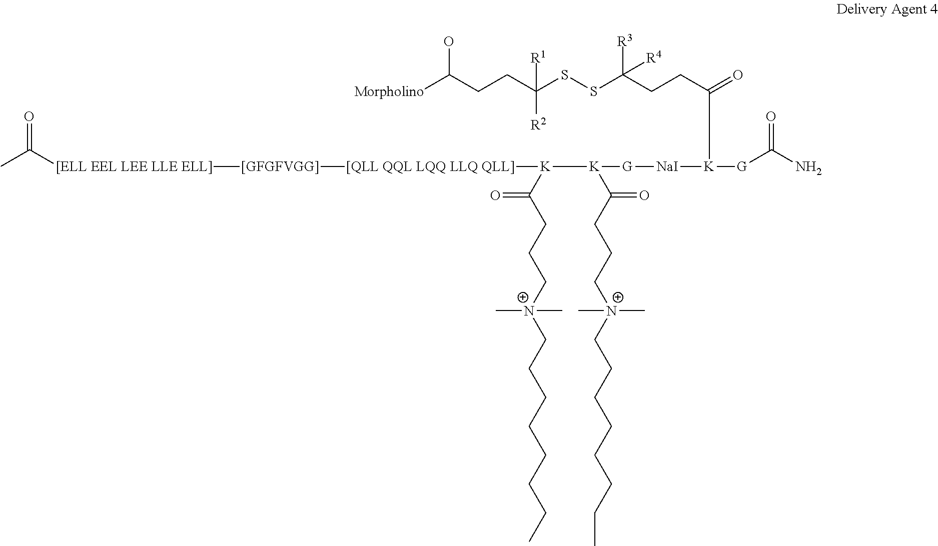

With reference to Delivery Agent 4, each of R¹, R², R³, and R⁴ independently is H, CH₃, or cyclobutyl. SEQ ID NO: 108 is the combined amino acid sequence of the lytic peptide group, cleavable linker, mask peptide group, amino acid portions of two anchor groups, and C-terminal moiety of Delivery Agent 4, shown above.

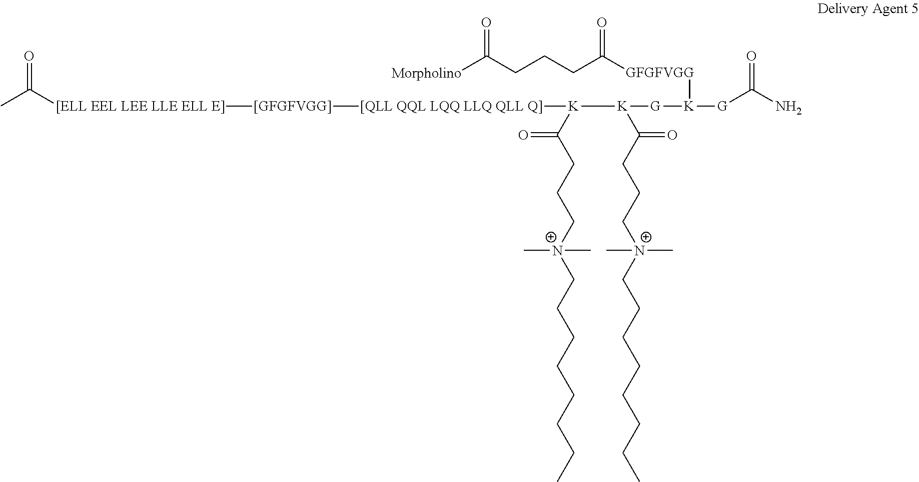

With reference to Delivery Agent 5, SEQ ID NO: 235 (ELLEELLEELLEGFGFVGGOLLQQLLQQLLOKKGKG, where K at positions 32 and 33 each provides an anchor group, K at position 35 is functionalized with a second cleavable linker that is conjugated to a therapeutic agent via a chemical linker group, and the C-terminal G at position 36 is functionalized with an NH₂ group) is the combined amino acid sequence of the lytic peptide group, cleavable linker, mask peptide group, amino acid portions of two anchor groups, and C-terminal moiety of Delivery Agent 5, shown above.

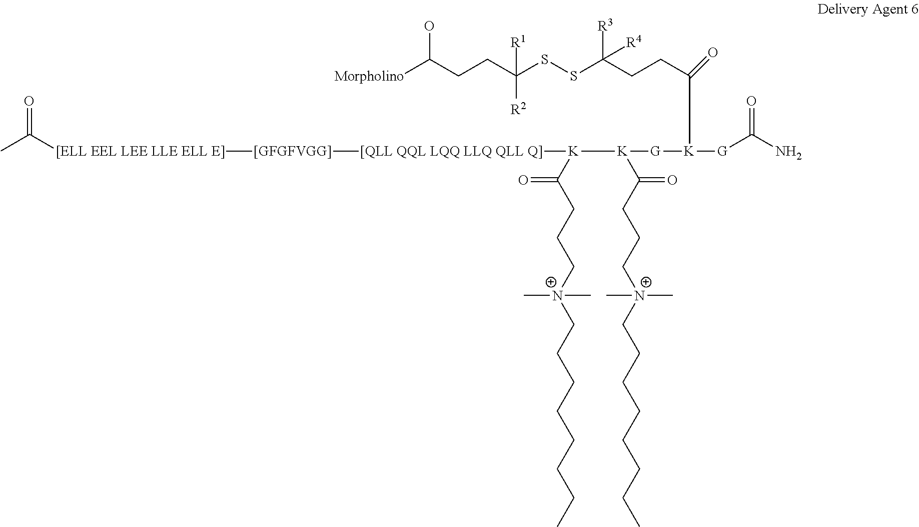

With reference to Delivery Agent 6, each of R¹, R², R³, and R⁴ independently is H, CH₃, or cyclobutyl. SEQ ID NO: 270 (ELLEELLEELLEGFGFVGGQLLQQLLQQLLOKKGKG, where K at positions 32 and 33 each provides an anchor group. K at position 35 is functionalized with a chemical linker group attached to a therapeutic agent, and the C-terminal G at position 36 is functionalized with an NH₂ group) is the combined amino acid sequence of the lytic peptide group, cleavable linker, mask peptide group, amino acid portions of two anchor groups, and C-terminal moiety of Delivery Agent 6, shown above.

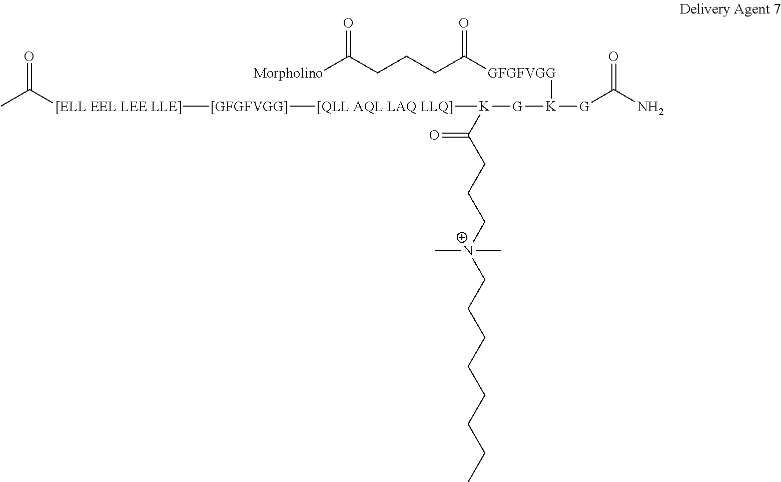

With reference to Delivery Agent 7, SEQ ID NO: 236 (ELLEELLEELLEGFGFVGGQLLAQLLAQLLQKKGKG, where K at positions 32 and 33 each provides an anchor group, K at position 35 is functionalized with a second cleavable linker that is conjugated to a therapeutic agent via a chemical linker group, and the C-terminal G at position 36 is functionalized with an NH₂ group) is the combined amino acid sequence of the lytic peptide group, cleavable linker, mask peptide group, amino acid portions of two anchor groups, and C-terminal moiety of Delivery Agent 7, shown above.

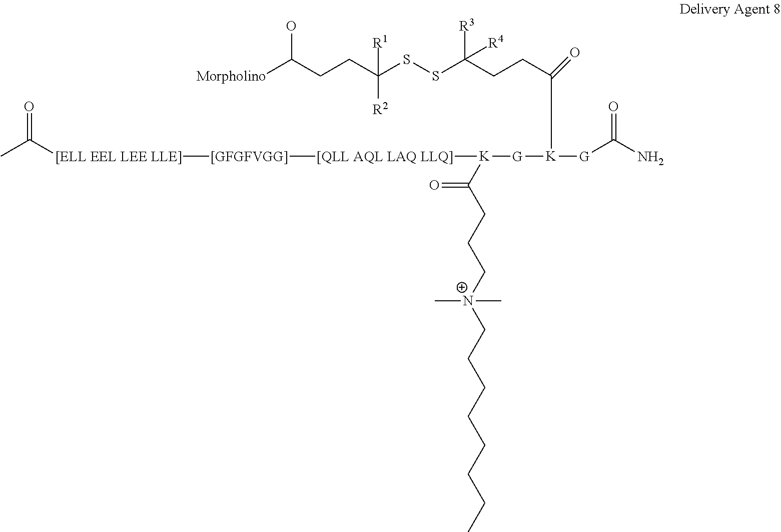

With reference to delivery agent 8, each of R¹, R², R³, and R⁴ independently is H, CH₃, or cyclobutyl and SEQ ID NO: 237 (ELLEELLEELLEGFGFVGGQLLAQLLAQLLOKGKG, where K at position 32 provides an anchor group, K at position 34 is functionalized with a chemical linker group that is attached to a therapeutic agent, and the C-terminal G at position 35 is functionalized with an NH₂ group) is the combined amino acid sequence of the lytic peptide group, cleavable linker, mask peptide group, amino acid portions of two anchor groups, and C-terminal moiety of Delivery Agent 8, shown above.

In some aspects of the disclosure, an amino acid sequence of the lytic peptide group, cleavable linker, mask peptide group, amino acid portions of the anchor groups, and C-terminal moiety (together, all or a portion of an amino acid sequence of a disclosed delivery agent) comprises any one of SEQ ID NOs: 12, 13, 14, 15, 18, 19, 21, 22, 23, 25, 27, 29, 31, 33, 35, 37, 39, 42, 52, 53, 54, 55, 56, 57, 58, 59, 69, 70, 71, 72, 73, 74, 75, 76, 77, 79, 82, 83, 87, 88, 89, 90, 91, 102, 103, 104, 105, 106, 107, 108, 113, 114, 118, 119, 120, 121, 122, 123, 124, 125, 126, 127, 128, 129, 130, 131, 132, 133, 135, 138, 139, 142, 143, 144, 146, 147, 149, 159, 160, 161, 162, 163, 164, 165, 166, 167, 168, 169, 170, 171, 182, 183, 184, 185, 186, 187, 188, 189, 190, 191, 192, 193, 194, 195, 196, 197, 198, 199, 200, 201, 202, 208, 209, 210, 211, 212, 213, 214, 215, 216, 217, 218, 219, 220, 221, 222, 223, 224, 225, 226, 228, 231, 232, 235, 236, 237, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, or 280. In some aspects of the disclosure, all or a portion of the amino acid sequence of a disclosed delivery agent has an amino acid sequence at least 85% identical to any one of SEQ ID NOs: 12, 13, 14, 15, 18, 19, 21, 22, 23, 25, 27, 29, 31, 33, 35, 37, 39, 42, 52, 53, 54, 55, 56, 57, 58, 59, 69, 70, 71, 72, 73, 74, 75, 76, 77, 79, 82, 83, 87, 88, 89, 90, 91, 102, 103, 104, 105, 106, 107, 108, 113, 114, 118, 119, 120, 121, 122, 123, 124, 125, 126, 127, 128, 129, 130, 131, 132, 133, 135, 138, 139, 142, 143, 144, 146, 147, 149, 159, 160, 161, 162, 163, 164, 165, 166, 167, 168, 169, 170, 171, 182, 183, 184, 185, 186, 187, 188, 189, 190, 191, 192, 193, 194, 195, 196, 197, 198, 199, 200, 201, 202, 208, 209, 210, 211, 212, 213, 214, 215, 216, 217, 218, 219, 220, 221, 222, 223, 224, 225, 226, 228, 231, 232, 235, 236, 237, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, or 280 such as at least 86%, at least 87%, and least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 12, 13, 14, 15, 18, 19, 21, 22, 23, 25, 27, 29, 31, 33, 35, 37, 39, 42, 52, 53, 54, 55, 56, 57, 58, 59, 69, 70, 71, 72, 73, 74, 75, 76, 77, 79, 82, 83, 87, 88, 89, 90, 91, 102, 103, 104, 105, 106, 107, 108, 113, 114, 118, 119, 120, 121, 122, 123, 124, 125, 126, 127, 128, 129, 130, 131, 132, 133, 135, 138, 139, 142, 143, 144, 146, 147, 149, 159, 160, 161, 162, 163, 164, 165, 166, 167, 168, 169, 170, 171, 182, 183, 184, 185, 186, 187, 188, 189, 190, 191, 192, 193, 194, 195, 196, 197, 198, 199, 200, 201, 202, 208, 209, 210, 211, 212, 213, 214, 215, 216, 217, 218, 219, 220, 221, 222, 223, 224, 225, 226, 228, 231, 232, 235, 236, 237, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, or 280. In specific, non-limiting aspects of the disclosure, all or a portion of the amino acid sequence of a disclosed delivery agent has an amino acid sequence comprising any one of SEQ ID NOs: 12, 13, 14, 15, 18, 19, 21, 22, 23, 25, 27, 29, 31, 33, 35, 37, 39, 42, 52, 53, 54, 55, 56, 57, 58, 59, 69, 70, 71, 72, 73, 74, 75, 76, 77, 79, 82, 83, 87, 88, 89, 90, 91, 102, 103, 104, 105, 106, 107, 108, 113, 114, 118, 119, 120, 121, 122, 123, 124, 125, 126, 127, 128, 129, 130, 131, 132, 133, 135, 138, 139, 142, 143, 144, 146, 147, 149, 159, 160, 161, 162, 163, 164, 165, 166, 167, 168, 169, 170, 171, 182, 183, 184, 185, 186, 187, 188, 189, 190, 191, 192, 193, 194, 195, 196, 197, 198, 199, 200, 201, 202, 208, 209, 210, 211, 212, 213, 214, 215, 216, 217, 218, 219, 220, 221, 222, 223, 224, 225, 226, 228, 231, 232, 235, 236, 237, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, or 280. In other specific, non-limiting aspects of the disclosure, the amino acid sequence of a disclosed delivery agent has an amino acid sequence consisting of any one of SEQ ID NOs: 12, 13, 14, 15, 18, 19, 21, 22, 23, 25, 27, 29, 31, 33, 35, 37, 39, 42, 52, 53, 54, 55, 56, 57, 58, 59, 69, 70, 71, 72, 73, 74, 75, 76, 77, 79, 82, 83, 87, 88, 89, 90, 91, 102, 103, 104, 105, 106, 107, 108, 113, 114, 118, 119, 120, 121, 122, 123, 124, 125, 126, 127, 128, 129, 130, 131, 132, 133, 135, 138, 139, 142, 143, 144, 146, 147, 149, 159, 160, 161, 162, 163, 164, 165, 166, 167, 168, 169, 170, 171, 182, 183, 184, 185, 186, 187, 188, 189, 190, 191, 192, 193, 194, 195, 196, 197, 198, 199, 200, 201, 202, 208, 209, 210, 211, 212, 213, 214, 215, 216, 217, 218, 219, 220, 221, 222, 223, 224, 225, 226, 228, 231, 232, 235, 236, 237, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, or 280. In particular aspects of the disclosure, all or a portion of the amino acid sequence of a disclosed delivery agent has an amino acid sequence comprising or consisting of any one of SEQ ID NOs: 8, 11, 39, 149, and 171.

### IV. Compositions and Methods of Administration

Provided herein are compositions that comprise a delivery agent disclosed herein. In some examples, a composition further comprises one or more additional therapeutic agents, adjuvants, carriers, buffers, detergents (such as deoxycholic acid), salts, lipids, stabilizers, emulsifiers, solubilizers, or any combination thereof. In some aspects of the disclosure, the composition comprises a therapeutic agent that is covalently or non-covalently bound to the peptide-based delivery agent, or that is merely associated with the delivery agent. In yet additional aspects of the disclosure, two separate compositions can be administered such that one composition comprises the therapeutic agent and the other composition comprises the delivery agent

Methods of administering the disclosed delivery agents are routine, and can be determined by clinicians. A variety of administration regimens are possible for the disclosed delivery agent, such as in combination with one or more therapeutic agents. The disclosed delivery agents are in general administered topically, nasally, intravenously, orally, intracranially, intramuscularly, parenterally or as implants, but even rectal or vaginal use is possible in principle. In some aspects of the disclosure, the disclosed delivery agents are administered intraperitoneally, intratumorally, intravitreally, intracerebrally, transcorneally or intraocularly, intracerebrally, epicutaneously, intradermally, subcutaneously, transdermally, intramuscularly, or via intracerebroventricular injection. The disclosed delivery agents, and one or more therapeutic agents, also may be administered to a subject using a combination of these techniques.

Administration with a therapeutically effective amount can be a single administration or multiple administrations. Administration can involve daily or multi-daily or less than daily (such as weekly, monthly, etc.) doses over a period of a few days to weeks or months, or even years. In particular non-limiting examples, administration involves a once monthly dose, a once every three weeks dose, a once every two weeks dose, a weekly dose, a twice weekly dose, or a daily dose, or a combination thereof. The particular mode/manner of administration and the dosage regimen will be selected by the laboratory technician, attending clinician, or veterinarian (in the case of administration to a non-human animal subject), taking into account the particulars of the case (such as the subject, the disease, the disease state/severity involved, the particular administration, and whether the treatment is prophylactic).

More than one route, such as intratumoral, intravenous, intraperitoneal, intramuscular, subcutaneous, oral, or topical may be used for administration of the delivery agent (such as in combination with one or more therapeutic agents), and particular routes may provide more immediate and more effective responses than other routes. A disclosed delivery agent and one or more therapeutic agents can be administered by the same or different routes. In some aspects of the disclosure, the delivery agent and the one or more therapeutic agents are administered using any suitable route of administration, such as, for example, intravenous or intratumoral administration. In exemplary aspects of the disclosure, the delivery agent and one or more therapeutic agents may be administered by intratumoral injection. Alternatively or additionally, the delivery agent and one or more therapeutic agents may be administered intravenously.

In some aspects of the disclosure, a subject can be administered varying concentrations of the delivery agent and each of the one or more therapeutic agents, one or more times, at one or more different time intervals. In some aspects of the disclosure, the disclosed delivery agent and one or more therapeutic agents are administered such that the effective time period of the delivery agent overlaps with administration of the one or more therapeutic agents. In some aspects of the disclosure, administration of the delivery agent and the one or more therapeutic agents is performed such that the delivery agent and the one or more therapeutic agents can be internalized into a cell, such as a target cell, within the same endosome of the cell.

Pharmaceutical compositions are of use in the disclosed method that include a therapeutically effective amount of a disclosed delivery agent and/or one or more therapeutic agents. These pharmaceutical compositions can include any suitable carrier. For example, formulations suitable for intratumoral, intravenous, intramuscular, subcutaneous, intraperitoneal, or topical administration may comprise sterile aqueous solutions of the active components. Such formulations may be prepared by dissolving a therapeutic agent and/or additional active and/or inactive component(s) in water containing physiologically compatible substances such as sodium chloride (e.g., 0.1-2.0 M), glycine, and the like, and having a buffered pH compatible with physiological conditions to produce an aqueous solution, and rendering said solution sterile. A suitable carrier may be the same for some (such as two or more) or the delivery agent and all of the one or more therapeutic agents administered, or may be different for the delivery agent and all of the one or more therapeutic agents.

Suitable solid or liquid pharmaceutical preparation forms are, for example, aerosols, (micro) capsules, creams, drops, drops or injectable solution in ampoule form, emulsions, granules, powders, suppositories, suspensions, syrups, tablets, coated tablets, and also preparations with protracted release of active compounds, in whose preparation excipients and additives and/or auxiliaries such as binders, coating agents, disintegrants, flavorings, lubricants, solubilizers, sweeteners, or swelling agents are customarily used as described above. The pharmaceutical agents are suitable for use in a variety of drug delivery systems. For a brief review of various methods for drug delivery, see Langer, “New Methods of Drug Delivery,” Science 249:1527-1533 (1990), incorporated by reference herein to the extent not inconsistent with the present disclosure.

The disclosed delivery agents of the present disclosure can be formulated into therapeutically active pharmaceutical agents that can be administered to a subject parenterally or orally. Parenteral administration routes include, but are not limited to epidermal, intraarterial, intramuscular (IM and depot IM), intraperitoneal (IP), intravenous (IV), intrasternal injection or infusion techniques, intranasal (inhalation), intrathecal, injection into the stomach, subcutaneous injections (subcutaneous (SQ and depot SQ), transdermal, topical, and ophthalmic.

The disclosed delivery agents, such as in combination with one or more therapeutic agents, can be mixed or combined with a suitable pharmaceutically acceptable excipients to prepare pharmaceutical agents. Pharmaceutically acceptable excipients include, but are not limited to, alumina, aluminum stearate, buffers (such as phosphates), glycine, ion exchangers (such as to help control release of charged substances), lecithin, partial glyceride mixtures of saturated vegetable fatty acids, potassium sorbate, serum proteins (such as human serum albumin), sorbic acid, water, salts or electrolytes such as cellulose-based substances, colloidal silica, disodium hydrogen phosphate, magnesium trisilicate, polyacrylates, polyalkylene glycols, such as polyethylene glycol, polyethylene-polyoxypropylene-block polymers, polyvinyl pyrrolidone, potassium hydrogen phosphate, protamine sulfate, group 1 halide salts such as sodium chloride, sodium carboxymethylcellulose, waxes, wool fat, and zinc salts, for example. Liposomal suspensions may also be suitable as pharmaceutically acceptable carriers.

Upon mixing or addition of one or more disclosed delivery agents, the resulting mixture may be a solid, solution, suspension, emulsion, or the like. These may be prepared according to suitable methods. The form of the resulting mixture depends upon a number of factors, including the intended mode of administration and the solubility of the agent in the selected carrier. Pharmaceutical carriers suitable for administration of the disclosed delivery agents include any such carriers known to be suitable for the particular mode of administration. In addition, the disclosed delivery agents can also be mixed with other inactive or active materials (such as one or more therapeutic agents) that do not impair the desired action, or with materials that supplement the desired action, or have another action.

Methods for solubilizing may be used where the delivery agents exhibit insufficient solubility in a carrier. Such methods exist and include, but are not limited to, dissolution in aqueous sodium bicarbonate, using cosolvents such as dimethylsulfoxide (DMSO), and using surfactants such as TWEEN® (ICI Americas, Inc., Wilmington, DE).

The disclosed delivery agents can be prepared with carriers that protect them against rapid elimination from the body, such as coatings or time-release formulations. Such carriers include controlled release formulations, such as, but not limited to, microencapsulated delivery systems. A disclosed delivery agent is included in the pharmaceutically acceptable carrier in an amount sufficient to exert a therapeutically useful effect, typically in an amount to avoid undesired side effects, on the treated subject. The therapeutically effective concentration may be determined empirically by testing the compounds in known in vitro and in vivo model systems for the treated condition or disease. For example, mouse models of a condition or disease of interest, such as an inherited condition, a rare disease, a cancer, an immune condition, or an infectious disease, may be used to determine effective amounts or concentrations that can then be translated to other subjects, such as humans.

Injectable solutions or suspensions can be formulated, using suitable non-toxic, parenterally-acceptable diluents or solvents, such as 1,3-butanediol, isotonic sodium chloride solution, mannitol, Ringer's solution, saline solution, or water; or suitable dispersing or wetting and suspending agents, such as sterile, bland, fixed oils, including synthetic mono- or diglycerides, and fatty acids, including oleic acid; a naturally occurring vegetable oil such as coconut oil, cottonseed oil, peanut oil, sesame oil, and the like; glycerine; polyethylene glycol; propylene glycol; or other synthetic solvent; antimicrobial agents such as benzyl alcohol and methyl parabens; antioxidants such as ascorbic acid and sodium bisulfite; buffers such as acetates, citrates, and phosphates; chelating agents such as ethylenediaminetetraacetic acid (EDTA); agents for the adjustment of tonicity such as sodium chloride and dextrose; and combinations thereof. Parenteral preparations can be enclosed in ampoules, disposable syringes, or multiple dose vials made of glass, plastic, or other suitable material. Buffers, preservatives, antioxidants, and the like can be incorporated as required. Where administered intravenously, suitable carriers include physiological saline, phosphate-buffered saline (PBS), and solutions containing thickening and solubilizing agents such as glucose, polyethylene glycol, polypropyleneglycol, and mixtures thereof. Liposomal suspensions, including tissue-targeted liposomes, may also be suitable as pharmaceutically acceptable carriers.

For topical application, a disclosed delivery agent, such as in combination with one or more therapeutic agents, may be made up into a cream, lotion, ointment, solution, or suspension in a suitable aqueous or non-aqueous carrier. Topical application can also be accomplished by transdermal patches or bandages which include the therapeutic substance. Additives can also be included, e.g., buffers such as sodium metabisulphite or disodium edetate; preservatives such as bactericidal and fungicidal agents, including phenyl mercuric acetate or nitrate, benzalkonium chloride, or chlorhexidine; and thickening agents, such as hypromellose.

If the disclosed delivery agent, such as in combination with one or more therapeutic agents, is administered orally as a suspension, the pharmaceutical agents can be prepared according to suitable techniques of pharmaceutical formulation and may contain a suspending agent, such as alginic acid or sodium alginate, bulking agent, such as microcrystalline cellulose, a viscosity enhancer, such as methylcellulose, and sweeteners/flavoring agents. Oral liquid preparations can contain conventional additives such as suspending agents, e.g., gelatin, glucose syrup, hydrogenated edible fats, methyl cellulose, sorbitol, and syrup; emulsifying agents, e.g., acacia, lecithin, or sorbitan monooleate; non-aqueous carriers (including edible oils), e.g., almond oil, fractionated coconut oil, oily esters such as glycerine, propylene glycol, or ethyl alcohol; preservatives such as methyl or propyl p-hydroxybenzoate or sorbic acid; and, if desired, conventional flavoring or coloring agents. When formulated as immediate release tablets, these agents can contain dicalcium phosphate, lactose, magnesium stearate, microcrystalline cellulose, and starch and/or other binders, diluents, disintegrants, excipients, extenders, and lubricants.

If oral administration is desired, a disclosed delivery agent, such as in combination with one or more therapeutic agents, can be provided in a composition that protects it from the acidic environment of the stomach. For example, a disclosed delivery agent can be formulated with an enteric coating that maintains its integrity in the stomach and releases the active compound in the intestine. The disclosed delivery agent can also be formulated in combination with an antacid or other such ingredient.

Oral compositions generally include an inert diluent or an edible carrier and can be compressed into tablets or enclosed in gelatin capsules. For the purpose of oral therapeutic administration, a disclosed delivery agent can be incorporated with excipients and used in the form of capsules, tablets, or troches. Pharmaceutically compatible adjuvant materials or binding agents can be included as part of the composition.

The capsules, pills, tablets, troches, and the like can contain any of the following ingredients or compounds of a similar nature: a binder such as, but not limited to, acacia, corn starch, gelatin, gum tragacanth, polyvinylpyrrolidone, or sorbitol; a filler such as calcium phosphate, glycine, lactose, microcrystalline cellulose, or starch; a disintegrating agent such as, but not limited to, alginic acid and corn starch; a lubricant such as, but not limited to, magnesium stearate, polyethylene glycol, silica, or talc; a glidant, such as, but not limited to, colloidal silicon dioxide; a sweetening agent such as sucrose or saccharin; disintegrants such as potato starch; dispersing or wetting agents such as sodium lauryl sulfate; and a flavoring agent such as peppermint, methyl salicylate, or fruit flavoring.

When the dosage unit form is a capsule, it can contain, in addition to material of the above type, a liquid carrier, such as a fatty oil. In addition, dosage unit forms can contain various other materials that modify the physical form of the dosage unit, for example, coatings of sugar and other enteric agents. A disclosed delivery agent can also be administered as a component of an elixir, suspension, syrup, water, tea, chewing gum, or the like. A syrup may contain, in addition to the active compounds, sucrose or glycerin as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, they can be of the sustained release type so that the compounds need to be administered less frequently.

In some examples, a disclosed delivery agent, such as in combination with one or more therapeutic agents, is injected into the stomach of a subject, is incorporated systemically in the subject, such as in diverse muscle groups. Examples of methods and compositions for administering therapeutic substances which include proteins include those discussed in Banga, Therapeutic Peptides and Proteins: Formulation, Processing, and Delivery Systems 2ed. (2005); Mahato, Biomaterials for Delivery and Targeting of Proteins and Nucleic Acids (2004); McNally, Protein Formulation and Delivery, 2ed. (2007); and Kumar et al., “Novel Delivery Technologies for Protein and Peptide Therapeutics,” Current Pharm. Biotech., 7:261-276 (2006); each of which is incorporated by reference herein to the extent not inconsistent with the present disclosure.

In some aspects of the disclosure, a delivery agent and/or a therapeutic agent can be administered by means of a nanoparticle vehicle. Nanoparticles (NPs) are synthetic particles with dimensions ranging from one to hundreds of nanometers comprising an inorganic core surrounded by an organic layer. Nanoparticles featuring inorganic cores such as gold, silica, superparamagnetic iron oxide (SPIO) are available. In cancer tissue, for example, NPs extravasate from the leaky cancer vasculature to a higher degree than healthy tissue, and remain in the area by the enhanced permeability and retention (EPR) effect. In some aspects of the disclosure, the delivery agent and/or therapeutic agent can be administered by means of a lipid nanoparticle, peptide-based nanoparticle (Bioactive Materials Volume 11, May 2022, Pages 268-282), a nanotube (such as a NanoPortal, NanoPrecision Medical, Emeryville, California, USA), a subcutaneously implantable reservoir (such as NANOPOR®, Delpore, Brisbane, California, USA), a long-acting injectable (such as SABER® or CLOUD™, Durect, Cupertino, California, USA), and/or a transdermal microneedle system (Zosano Pharma, Fremont, California, USA). In particular aspects of the disclosure, a nanoparticle can be combined with a delivery agent and/or therapeutic agent as follows: 1) enclose the delivery agent and/or therapeutic agent within the nanoparticle vehicle; and/or 2) covalently attach, bind, link, or associate the delivery agent and/or therapeutic agent to the outside of the nanoparticle vehicle. To evaluate delivery performance of embodiments using a nanoparticle in combination with a delivery agent and/or therapeutic agent, the HeLa Luc/705 cell luciferase assay discussed herein can be used. Alternatively, or in addition, a biological cell assay that measures (i) a change in a cellular or biomolecular phenotype or (ii) a change in expression of a molecular marker due to delivery of a therapeutic agent or from delivering an exogenous biological molecule can be used, such as measuring fluorescence from expression of green fluorescent protein (GFP) after delivery of GFP RNA into cells. Solely by way of example, if GFP RNA with and without delivery agent was enclosed within a nanoparticle vehicle, and if biological cells are treated with the nanoparticle vehicles in growth media for 24 hours or more, a significantly greater average increase in fluorescence from cells treated with the nanoparticle containing delivery agent would result, compared to cells treated with the nanoparticle excluding the delivery agent.

The dose of each delivery agent and/or each one or more therapeutic agent administered to a subject should be sufficient to induce a beneficial therapeutic response in the subject over time, such as reducing a burden from an inherited condition, a rare disease, a cancer, an immune condition, or an infectious disease in the subject, increasing survival of the subject, reducing the incidence of relapse of an inherited condition, a rare disease, a cancer, an immune condition, or an infectious disease in the subject, or a combination thereof. The beneficial therapeutic response may require one or more doses of one or more of the disclosed delivery agent and/or the one or more therapeutic agents, such as 1, 2, 3, 4, 5, 6, 7, 8, 9, 10, 20, 30, 40, 50, 100, 200, 300, 400, or 500, or more doses, administered at the same or different times. In some implementations, the effective amount of a disclosed delivery agent, such as in combination with a therapeutic agent, is administered as a single dose per time period, such as every three or four months, month, week, or day, or it can be divided into at least two unit dosages for administration over a period. Treatment may be continued as long as necessary to achieve the desired results. For instance, treatment may continue for 3 or 4 weeks up to 12-24 months or longer, including ongoing treatment. The compound can also be administered in several doses intermittently, such as every few days (for example, at least every two, three, four, five, or ten days) or every few weeks (for example at least every two, three, four, five, or ten weeks).

The dose may vary from subject to subject or may be the same. An appropriate dose can be determined, for example, using routine experimentation. Generally, the administration of the disclosed delivery agent and/or one or more therapeutic agents provides a robust, synergistic therapeutic effect for treating the inherited condition, rare disease, cancer, immune condition, or infectious disease in the subject, for example as compared to the use of the therapeutic agents without the delivery agent. Thus, particular dosage regimens can be tailored to a particular subject, condition to be treated, or desired result. For example, when the methods of the present disclosure are used to treat a target disease or condition (such as an inherited condition (such as muscular dystrophy), a rare disease, a cancer, an immune condition, or an infectious disease), an initial treatment regimen can be applied to arrest the condition. Such initial treatment regimen may include administering a higher dosage of a disclosed delivery agent, or administering such material more frequently, such as daily. After a desired therapeutic result has been obtained, a second treatment regimen may be applied, such as administering a lower dosage of the delivery agent or administering such material less frequently, such as monthly, bi-monthly, quarterly, or semi-annually. In such cases, the second regimen may serve as a “booster” to restore or maintain a desired level of muscle regeneration. Similar treatment regimens may be used for other subjects with reduced or impaired muscle regeneration capabilities, such as geriatric subjects.

Amounts effective for various therapeutic treatments of the present disclosure may depend on the severity of the condition or disease and the weight and general state of the subject, as well as the absorption, inactivation, and excretion rates of the delivery agent (and any one or more therapeutic agent administered to the subject along with the delivery agent), the dosage schedule, and amount administered, as well as other factors that are recognizable in the art, particularly with the benefit of the present disclosure. It also should be apparent that the exact dosage and frequency of administration will depend on the particular delivery agent, or other therapeutic agent being administered, the particular condition being treated, the severity of the condition being treated, the age, weight, general physical condition of the particular subject, and other medication the subject may be taking. Typically, dosages used in vitro may provide useful guidance in the amounts useful for in vivo administration of the pharmaceutical composition, and animal models may be used to determine effective dosages for treatment of particular disorders. For example, mouse models of a particular condition or disease (such as an inherited condition, a rare disease, a cancer, an immune condition, or an infectious disease) may be used to determine effective dosages that can then be translated to dosage amount for other subjects, such as humans. Various considerations in dosage determination are described, e.g., in Gilman et al., eds., Goodman And Gilman's: The Pharmacological Bases of Therapeutics, 8^{th} ed., Pergamon Press (1990); and Remington's Pharmaceutical Sciences, 17^{th} ed., Mack Publishing Co., Easton, Pa. (1990), each of which is herein incorporated by reference to the extent not inconsistent with the present disclosure.

In specific examples, the disclosed delivery agent is administered to a subject in an amount sufficient to provide a dose of the delivery agent ranging from 10 nmol/g and 500 nmol/g, such as between 2 nmol/g and 20 nmol/g or between 2 nmol/g and 10 nmol/g. In additional examples, the delivery agent is administered to a subject in an amount sufficient to provide a dose of between 0.01 μg/kg and 1000 mg/kg or between 0.1 mg/kg and 1000 mg/kg, in particular examples this amount is provided per day or per week. In another example, the disclosed delivery agent is administered to a subject in an amount sufficient to provide a dose of agent of between 0.2 mg/kg and 2 mg/kg. In another example, the disclosed delivery agent is administered to a subject in an amount sufficient to provide a dose of agent of between 1.0 mg/kg and 30 mg/kg, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mg/kg. In further examples, the delivery agent is administered to a subject in an amount sufficient to provide a concentration of delivery agent in the administrated material of between 5 nM and 500 nM, such as between 50 nM and 200 nM, or 100 nM. In further examples, the delivery agent is administered to a subject in an amount sufficient to provide a concentration of delivery agent in the administrated material of between 0.1 μM and 500 μM, such as between 5 UM and 30 μM, or 10 μM. In other examples, the delivery agent is administered to a subject between 25 mg/ml and 1 mg/ml, such as 5 mg/ml and 1 mg/ml, 1000 μg/ml and 500 μg/ml. 500 μg/ml and 50 μg/ml, including 500 μg/ml, 400 μg/ml, 300 μg/ml, 250 μg/ml, 200 μg/ml, 150 g/ml, 100 μg/ml, 50 μg/ml, 25 μg/ml, 12.5 μg/ml, 6.25 μg/ml, 3.125 μg/ml, 2.5 μg/ml and 1.25 μg/ml.

Exemplary therapeutic agents that can be administered in combination with a disclosed delivery agents are provided herein. In some aspects of the disclosure, any therapeutic agent that has activity within the cytosol of a cell can be used with the disclosed delivery agents and methods. In particular aspects of the disclosure, a therapeutic agent of use herein is an agent that, in combination with a disclosed delivery agent, can be internalized into a cell (such as a target cell in a subject or in a cell culture) at a higher rate than without the delivery agent, and/or can have a greater therapeutic efficacy in a subject than without the delivery agent. In some aspects of the disclosure, the one or more therapeutic agent is a chemotherapeutic, a therapeutic antibody, an immune therapeutic, an antibiotic, a therapeutic for a psychiatric disorder, a substance abuse disorder, or a combination thereof. In specific, non-limiting aspects of the disclosure, the one or more therapeutic agent is saporin, cisplatin, methotrexate, fluorouracil, doxorubicin, cyclophosphamide, chlorambucil, vinblastine, vincristine, docetaxel, or paclitaxel, chlorhexidine, triclosan, xylitol, or octadecene-1-amine hydrofluoride, 1-hexadecylamine hydrofluoride, Gefitinib, Lapatinib, Olaparib, mitomycin C, Sunitinib, Geftinib, Nintedanib, PD173074, Erdaftinib, Sorafenib, or a combination thereof.

In specific examples, a therapeutic agent is administered to a subject in an amount sufficient to provide a dose of the therapeutic agent ranging from 10 nmol/g and 500 nmol/g, such as between 2 nmol/g and 20 nmol/g or between 2 nmol/g and 10 nmol/g. In additional examples, the therapeutic agent is administered to a subject in an amount sufficient to provide a dose of between 0.01 μg/kg and 1000 mg/kg or between 0.1 mg/kg and 100 mg/kg, or between 100 mg/kg and 1000 mg/kg, in particular examples this amount is provided per day or per week. In another example, the disclosed therapeutic agent is administered to a subject in an amount sufficient to provide a dose of agent of between 0.2 mg/kg and 2 mg/kg. In further examples, the therapeutic agent is administered to a subject in an amount sufficient to provide a concentration of therapeutic agent in the administrated material of between 5 nM and 500 nM, such as between 50 nM and 200 nM, or 100 nM. In further examples, the therapeutic agent is administered to a subject in an amount sufficient to provide a concentration of therapeutic agent in the administrated material of between 0.1 μM and 500 μM, such as between 0.1 μM and 50 μM, or 20 μM. In other examples, the therapeutic agent is administered to a subject between 500 μg/ml and 1 μg/ml, such as 300 μg/ml and 3 μg/ml, 200 μg/ml and 20 μg/ml, including 500 μg/ml, 400 μg/ml, 300 μg/ml, 250 μg/ml, 200 μg/ml, 150 g/ml, 100 μg/ml, 50 μg/ml, 25 μg/ml, 12.5 μg/ml, 6.25 μg/ml, 3.125 μg/ml, 2.5 μg/ml and 1.25 μg/ml.

Therapeutic antisense and ribozyme molecules are also of use in combination with the delivery agents disclosed herein and in the methods disclosed herein. Use of a disclosed delivery agent in combination with an antisense or ribozyme molecule can enhance internalization and therapeutic activity of the antisense or ribozyme molecule, thus increasing the efficacy of the antisense or ribozyme molecule, such as in a subject that has a condition or disease of interest (such as an inherited condition, a rare disease, a cancer, an immune condition, or an infectious disease). Antisense nucleic acids are DNA or RNA molecules that are complementary to at least a portion of a specific mRNA molecule (Weintraub, Scientific American 262:40, 1990). In the cell, the antisense nucleic acids hybridize to the corresponding mRNA, forming a double-stranded molecule. The antisense nucleic acids interfere with the translation of the mRNA, since the cell will not translate an mRNA that is double-stranded. In some aspects of the disclosure, an antisense molecule of use herein is an mRNA, a miRNA, a siRNA, or a CRISPR-related molecule. In one embodiment, therapeutic antisense oligomers of 10-25 nucleotides are administered to a subject in combination with a disclosed delivery agent. The use of antisense methods to inhibit translation of genes is described, for example in Marcus-Sakura, Anal. Biochem. 172:289, 1988.

Generally, the principle behind antisense technology is that an antisense compound hybridizes to a target nucleic acid and affects the modulation of gene expression activity, or function, such as transcription, translation or splicing. The modulation of gene expression can be achieved by, for example, target RNA degradation or occupancy-based inhibition. An example of modulation of target RNA function by degradation is RNase H-based degradation of the target RNA upon hybridization with a DNA-like antisense compound, such as an antisense oligonucleotide. Antisense oligonucleotides can also be used to modulate gene expression, such as splicing, by occupancy-based inhibition, such as by blocking access to splice sites. Antisense compounds provide sequence-specific target gene regulation. This sequence-specificity makes antisense compounds effective tools for the selective modulation of a target nucleic acid of interest. In some aspects of the disclosure, expression of one or more genes of interest is inhibited at least 10%, at least 25%, at least 50%, at least 75%, at least 80%, at least 85%, at least 90%, or at least 95%, such as relative to a control.

Any type of antisense compound that specifically targets and regulates expression of the one or more genes of interest is contemplated for use with the disclosed delivery agents and methods. Such antisense compounds include single-stranded compounds, such as antisense oligonucleotides, and double-stranded compounds, including compounds with at least partial double-stranded structure, including siRNAs, miRNAs, shRNAs, and ribozymes. An antisense molecule also can have flanking sequences (e.g., regulatory sequences). A ribozyme can have any general structure including, without limitation, hairpin, hammerhead, or axehead structures, provided the molecule cleaves RNA. In some aspects of the disclosure, the subject or a cell of a cell culture is administered a therapeutically effective amount of a disclosed delivery agent and a viral vector comprising the nucleic acid inhibitor.

An antisense oligonucleotide of use with the disclosed delivery agent and methods can be, for example, 5, 10, 15, 20, 25, 30, 35, 40, 45 or 50 nucleotides in length. An antisense nucleic acid can be constructed using chemical synthesis and enzymatic ligation reactions. For example, an antisense nucleic acid molecule can be chemically synthesized using naturally occurring nucleotides or variously modified nucleotides designed to increase the biological stability of the molecules or to increase the physical stability of the duplex formed between the antisense and sense nucleic acids, such as phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridin-e, 5-carboxymethylaminomethyluracil, dihydrouracil, beta-D-galactosylqueosine, inosine, amongst others. Methods of screening antisense compounds for specificity exist (see, for example, U.S. Patent Application Publication No. 2003/0228689).

Ribozymes, which are RNA molecules possessing the ability to specifically cleave other single-stranded RNA in a manner analogous to DNA restriction endonucleases, are also of use. Through the modification of nucleotide sequences, which encode these RNAs, it is possible to engineer molecules that recognize specific nucleotide sequences in an RNA molecule and cleave it (Cech, J. Amer. Med. Assn. 260:3030, 1988). An advantage of this approach is that, because they are sequence-specific, only mRNAs with particular sequences are inactivated.

There are two basic types of ribozymes namely, tetrahymena-type (Hasselhoff, Nature 334:585, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-base recognition sequences are preferable to shorter recognition sequences.

Various delivery systems are available and can be used to administer the siRNAs and other inhibitory nucleic acid molecules as therapeutics. Such systems include, for example, encapsulation in liposomes, microparticles, microcapsules, nanoparticles, recombinant cells capable of expressing the therapeutic molecule(s) (see, e.g., Wu et al., J. Biol. Chem. 262, 4429, 1987), construction of a therapeutic nucleic acid as part of a retroviral or other vector, and the like.

In other, non-limiting aspects of the disclosure, the therapeutic agent is a peptide nucleic acid (PNA) (such as those described by Nielsen and Egholm, Current Issues Molec. Biol. (1999) 1(2): 89-104, relevant PNAs of which are incorporated herein by reference). Like DNA, synthetic PNAs bind complementary nucleic acid strands and can specifically target and regulate expression of one or more genes of interest.

In a particular, non-limiting embodiment, the therapeutic agent is a morpholino. Representative morpholinos are discussed in Moulton et al., J Drug Discov Develop and Deliv, 3(2): 1023, 2016, the relevant morpholinos of which are incorporated herein by reference. Morpholinos, such as phosphorodiamidate morpholino oligomers, are oligomer molecules useful for blocking sites on nucleic acids, such as target RNA molecules, and have potential for broad therapeutic uses, such as in targeting pathogens and genetic disorders. Morpholinos are specific, soluble, non-toxic, stable, and effective antisense therapeutic agents. They can target a wide range of RNAs to, for example, block translation of an RNA molecule (such as an mRNA), modify splicing of a pre-mRNA, inhibit miRNA maturation and/or activity, and similar. While morpholinos can be delivered into a range of cultured cells, embryos, and multicellular subjects (such as mammals, such as humans and non-human animals), administering a disclosed delivery agent in combination with one or more therapeutic morpholinos can enhance internalization and therapeutic efficacy of the one or more morpholinos. In another embodiment, the therapeutic agent is a peptide-modified morpholino oligomer (PPMO). In particular aspects of the disclosure, a morpholino having a sequence according to any of the following can be used: GATCCATGGACATTTGACTGGTATTTCC (SEQ ID NO: 240); CAGCGAGACAACCAG (SEQ ID NO: 241); TCCACTGGAAGAAGTTGATTATTTC (SEQ ID NO: 242); AGAGACAGCGAGACA (SEQ ID NO: 243); and GCTTCCTGAATGCCCAAAGAAACAC (SEQ ID NO: 244).

### V. Methods

### A. Methods of Contacting a Cell

Provided herein are methods of contacting a cell with a disclosed composition including a delivery agent as described herein (such as contacting the cell with an effective amount of the disclosed delivery agent). In aspects of the disclosure, the delivery agent of the composition delivers one or more therapeutic agents to the cell's cytosol. A disclosed delivery agent can be contacted with the cell in the same composition or in different compositions as the one or more therapeutic agents, and at the same time or at different times as the one or more therapeutic agents. In some aspects of the disclosure, the cell is contacted with a disclosed delivery agent (such as in combination with one or more therapeutic agents) for at least 1-60 minutes, such as at least 1-10, 10-20, 20-30, 30-40, 40-50, or 50-60 minutes, such as at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes. In some aspects of the disclosure, the cell is contacted with a disclosed delivery agent (such as in combination with one or more therapeutic agents) for at least 1-40 days, such as at least 1, at least 3, at least 5, at least 7, at least 10, at least 14, at least 21, at least 28, at least 35, 10-30, 10-20, 20-30, 15-25, such as at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. In some aspects of the disclosure, the exosomes are added to a culture medium of a cell one time. In other aspects of the disclosure, the exosomes are added to a culture medium of a cell more than once, such as 2-5 times, such as 2, 3, 4, or 5 times over the course of the culture.

In specific examples, a cell is contacted with a disclosed delivery agent at a dose of the delivery agent of between 1 nM and 500 nM, such as between 1 nM and 50 nM, between 50 nM and 100 nM, between 100 nM and 150 nM, between 150 nM and 200 nM, between 200 nM and 250 nM, between 250 nM and 300 nM, between 300 nM and 350 nM, between 350 nM and 400 nM, between 400 nM and 450 nM, or between 450 nM and 500 nM. In additional examples, the cell is contacted with a disclosed delivery agent at a dose of the delivery agent of between 0.5 UM and 100 μM, such as between 0.50 UM and 1 μM, between 1 μM and 5 μM, between 5 M and 10 μM, between 10 UM and 25 μM, between 25 UM and 50 UM, between 50 UM and 75 μM, between 75 UM and 100 μM, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 μM. In other examples, the cell is contacted with a disclosed delivery agent at a dose of the delivery agent of between 500 μg/ml and 1 μg/ml, such as 300 μg/ml and 3 μg/ml, 200 μg/ml and 20 μg/ml, including 500 μg/ml, 400 μg/ml, 300 μg/ml, 250 μg/ml, 200 μg/ml, 150 μg/ml, 100 μg/ml, 50 μg/ml, 25 μg/ml, 12.5 μg/ml, 6.25 μg/ml, 3.125 μg/ml, 2.5 μg/ml and 1.25 μg/ml. In another example, a cell is contacted with a disclosed delivery agent at a dose of the delivery agent of between 0.5 ng/kg and 500 ng/kg, such as 0.5 μg/kg and 500 μg/kg, such as 0.5 mg/kg and 500 mg/kg.

In specific examples, a cell is contacted with one or more therapeutic agents at a dose of the one or more therapeutic agents of between 1 nM and 500 nM, such as between 1 nM and 50 nM, between 50 nM and 100 nM, between 100 nM and 150 nM, between 150 nM and 200 nM, between 200 nM and 250 nM, between 250 nM and 300 nM, between 300 nM and 350 nM, between 350 nM and 400 nM, between 400 nM and 450 nM, or between 450 nM and 500 nM. In additional examples, the cell is contacted with one or more therapeutic agents at a dose of the one or more therapeutic agents of between 0.5 UM and 100 UM, such as between 0.50 M and 1 μM, between 1 μM and 5 μM, between 5 μM and 10 μM, between 10 μM and 25 μM, between 25 μM and 50 μM, between 50 μM and 75 μM, between 75 μM and 100 μM, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 μM. In other examples the cell is contacted with one or more therapeutic agents at a dose of the one or more therapeutic agents of between 500 μg/ml and 1 μg/ml, such as 300 μg/ml and 3 μg/ml, 200 μg/ml and 20 μg/ml, including 500 μg/ml, 400 μg/ml, 300 μg/ml, 250 μg/ml, 200 μg/ml, 150 μg/ml, 100 μg/ml, 50 μg/ml, 25 μg/ml. 12.5 μg/ml, 6.25 μg/ml, 3.125 μg/ml, 2.5 μg/ml and 1.25 μg/ml. In another example, a cell is contacted with one or more therapeutic agents at a dose of the one or more therapeutic agents of between 0.5 ng/kg and 500 ng/kg, such as 0.5 μg/kg and 500 μg/kg, such as 0.5 mg/kg and 500 mg/kg.

In some examples, contacting a cell with a disclosed delivery agent and one or more therapeutic agents can enhance activity (such as measured using methods, such as IC50, Emax, and/or area under the dose-response curve (AUC)), of the one or more therapeutic agents by 2-fold to at least 2,000-fold, such as at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, at least 150-fold, at least 200-fold, at least 250-fold, at least 300-fold, at least 350-fold, at least 400-fold, at least 450-fold, at least 500-fold, at least 550-fold, at least 600-fold, at least 650-fold, at least 700-fold, at least 750-fold, at least 800-fold, at least 850-fold, at least 900-fold, at least 950-fold, at least 1,000-fold, at least 1, 100-fold, at least 1, 150-fold, at least 1,200-fold, at least 1,250-fold, at least 1,300-fold, at least 1,350-fold, at least 1,400-fold, at least 1,450-fold, at least 1,500-fold, at least 1,550-fold, at least 1,600-fold, at least 1,650-fold, at least 1,700-fold, at least 1,750-fold, at least 1,800-fold, at least 1,850-fold, at least 1,900-fold, at least 1,950-fold, or at least 2,000-fold (for example, as compared to methods where the cells are contacted with the one or more therapeutic agents and no delivery agent).

In some examples, contacting a cell with a disclosed delivery agent and one or more therapeutic agents can reduce cell viability (such as measured using methods, such as IC50, Emax, and/or area under the dose-response curve (AUC)) by 2-fold to at least 2,000-fold, such as at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, at least 150-fold, at least 200-fold, at least 250-fold, at least 300-fold, at least 350-fold, at least 400-fold, at least 450-fold, at least 500-fold, at least 550-fold, at least 600-fold, at least 650-fold, at least 700-fold, at least 750-fold, at least 800-fold, at least 850-fold, at least 900-fold, at least 950-fold, at least 1,000-fold, at least 1,100-fold, at least 1, 150-fold, at least 1,200-fold, at least 1,250-fold, at least 1,300-fold, at least 1,350-fold, at least 1,400-fold, at least 1,450-fold, at least 1,500-fold, at least 1,550-fold, at least 1,600-fold, at least 1,650-fold, at least 1,700-fold, at least 1,750-fold, at least 1,800-fold, at least 1,850-fold, at least 1,900-fold, at least 1,950-fold, or at least 2,000-fold (for example, as compared to methods where the cell is contacted with the one or more therapeutic agents and no delivery agent).

In particular aspects of the disclosure, contacting the cell with the composition induces lysis of an endosomal membrane following internalization of the therapeutic agent and the delivery agent into the cell, thereby delivering the therapeutic agent into the cytosol of the cell. In other particular aspects of the disclosure, contacting the cell with the composition induces formation of a pore in an endosomal membrane following internalization of one or more therapeutic agents and a disclosed delivery agent into the cell, thereby delivering the one or more therapeutic agents into the cytosol of the cell. In other particular aspects of the disclosure, contacting the cell with the composition causes local membrane destabilization and/or permeability following internalization of the therapeutic agent and the delivery agent into the cell, thereby delivering the therapeutic agent into the cytosol of the cell (Pei and Buyanova, Bioconjug Chem. 30 (2):273-283, 2019).

In aspects of the present disclosure, the cell can be contacted in vitro or in vivo. In some aspects of the disclosure, the cell is a non-human mammalian cell (such as a cell from a laboratory animal, including, but not limited to, zebrafish or xenopus, or a cell from a veterinary animal, including, but not limited to, dogs and cats, as well as mice, rats, rabbits, sheep, horses, cows, and non-human primates) or a human cell.

Also disclosed herein are methods of identifying therapeutic agents using cells, such as cells in a cell culture system. In some aspects of the disclosure, the method includes contacting the cell with a disclosed delivery agent and one or more test compounds (such as one or more potential therapeutic agents), and determining an effect of the one or more compounds on the contacted cell, such as compared to a control. In some aspects of the disclosure, reduced cell survival indicates that the one or more compounds (or combination of compounds) is a therapeutic agent (or combination of therapeutic agents) that may be of use in treating a particular condition or disease in a subject. In some examples, the method includes determining an IC₅₀ value for one or more test compounds.

In some examples, the method is a high-throughput screening method. Using automation, data processing/control software, liquid handling devices, and sensitive detectors, high-throughput screening allows for rapid testing of high numbers (such as thousands or millions) of molecules (such as test compounds) to determine the efficacy of each molecule for a desired purpose, such as the efficacy of each molecule for use in treating a condition or disease of interest (such as an inherited condition, a rare disease, a cancer, an immune condition, or an infectious disease). In some examples, identification of therapeutic agents using the cells is carried out in microplates, such as 24-well, 48-well, 96-well or 384-well microtiter plates. In some aspects of the disclosure, an outcome indicator, such as an indicator of cell survival or of a particular cellular phenotype (such as an increase or decrease in expression of a molecule of interest, such as a protein of interest), such as a fluorescent or bioluminescent signal, is detected using a microplate reader. A microplate reader detects biological, chemical, or physical events in microtiter plates. A high-intensity lamp passes light to the microtiter well and the light emitted by the reaction in the well is quantified by a detector. Detection modes for microplate assays include absorbance, fluorescence intensity, luminescence, time-resolved fluorescence, and fluorescence polarization. In some aspects of the disclosure, fluorescence intensity is measured using a microplate reader, such as SPECTRAMAX® M5 (Molecular Devices), ELX800™ Absorbance Microplate Reader (BioTek), SpectraFluor (Tecan), or VICTOR3™ (Perkin Elmer). Suitable wavelengths for excitation and corresponding emission wavelengths can be used. Such wavelengths may range from 425 nm to 800 nm, such as 450 nm to 500 nm, or 450 nm to 495 nm, or 485 nm to 505 nm, such as 495 nm to 500 nm, or 620 nm to 800 nm, such as 620 nm to 750 nm. In a particular example, fluorescence intensity is measured using a Tecan SpectraFluor microplate reader using excitation at 485 nm and measuring emission at 535 nm.

In some aspects of the disclosure, the method includes contacting cells with a disclosed delivery agent and one or more test compounds, such as adding the delivery agent and one or more test compounds to intact cells representative of a particular condition or disease of interest (such as an inherited condition, a rare disease, a cancer, an immune condition, or an infectious disease). The delivery agent and the one or more test compounds are incubated with the intact cells for an amount of time to permit the delivery agent and the compound (or compounds) to enter the cells. The delivery agent and the one or more test compound may be incubated with the cells from 1 to 120 minutes, such as from 10 minutes to 100 minutes, 20 minutes to 90 minutes, 30 minutes to 80 minutes, 40 minutes to 70 minutes. 50 minutes to 60 minutes, such as at least five minutes, for example 5 minutes, 10 minutes, 15 minutes, 20 minutes. 30 minutes, 60 minutes, 90 minutes, or 120 minutes. In some aspects of the disclosure, the delivery agent and the one or more test compound may be incubated with cells for 2 to 24 hours, such as 2, 3, 4, 5, 6, 7, 8, 9, 10, 11, 12, 13, 14, 15, 16, 17, 18, 19, 20, 21, 22, 23, or 24 hours. The incubation is carried out at a temperature which permits the delivery agent to enable the one or more test compounds to be internalized into a cell and to be released from the endolysosome into the cytosol of the cell. For example, the incubation of the delivery agent and the one or more test compounds with the intact cells may be at about room temperature, such as at a temperature of about 20° C. to about 25° C. In additional aspects of the disclosure, the cells are incubated at a temperature of about 4° C. to about 56° C., such as about 15° C. to about 50° C., about 22° C. to about 45° C., about 25° C. to about 40° C., or about 30° C. to about 37° C. In a particular example, the delivery agent and the one or more test compounds is incubated with intact cells for 60 minutes at 37° C. In another particular example, the delivery agent and the one or more test compounds is incubated with intact cells for 18 hours at 37° C.

In some aspects of the disclosure, the cells are washed following incubation with the delivery agent and the one or more test compounds to remove any delivery agent and test compound material that has not been internalized into the cells. Washing may be by standard methods, for example by centrifugation of the cells, removal of the resulting supernatant, and resuspension of the cells in a solution. The cells may be resuspended in a physiological buffer, such as phosphate-buffered saline, Hank's balanced salt solution, lactated Ringer's solution, or cell culture media (for example RPMI-1640 or DMEM). The buffers may contain small amounts of solvent (such as 0.5% to 2% ethanol or methanol) or carrier molecules (such as 1% to 4% glucose or fructose). The wash step may be repeated one to six times, such as one time, two times, three times, four times, five times, or six times. In a particular example, the cells are washed three times by centrifugation at 400×g for 2 to 10 minutes, removal of the resulting supernatant, and resuspension in phosphate buffered saline.

In some examples, the screening methods further include selecting test compounds identified as having therapeutic activity (e.g., those that reduce expression of a protein of interest, induce a phenotype of interest in the cells, and/or reduce or enhance survival of the cells). In some examples, the screening methods further include administering such selected compounds individually or in combination, along with a disclosed delivery agent, into a research animal, such as a research mammal, such as a rabbit, non-human primate, cat, dog, mouse, or rat, for further analysis of the efficacy of the delivery agent and the one or more test compounds to treat or inhibit a condition or disease of interest (such as an inherited condition, a rare disease, a cancer, an immune condition, or an infectious disease).

### B. Methods of Treating a Subject

Also provided herein are methods of administering to a subject a therapeutically effective amount of the disclosed compositions. In particular aspects of the disclosure, the therapeutically effective amount of the composition treats, ameliorates, or prevents the onset of a condition or disease in the subject, such as an inherited condition, a rare disease, a cancer, an immune condition, an infectious disease, a psychiatric disorder, or any combination thereof. Thus, disclosed are methods of treating a subject with a condition or disease by administering a delivery agent disclosed herein, such as a therapeutically effective amount of a disclosed delivery agent. In some aspects of the disclosure, the delivery agent is administered in combination with a therapeutically effective amount of one or more therapeutic agents. In some aspects of the disclosure, the condition or disease treated using the disclosed methods is an inherited condition, a rare disease, a cancer, an immune condition, an infectious disease, a psychiatric disorder. In some aspects of the disclosure, the condition or disease is a cancer (e.g., solid cancer (such as sarcomas (e.g., rhabdomyosarcoma, osteogenic sarcoma, Ewing's sarcoma, chondrosarcoma, and alveolar soft part sarcoma); carcinomas (e.g., colorectal carcinoma); and lymphomas, such as Hodgkin's or non-Hodgkin's lymphoma, for example, diffuse large B-cell, follicular, chronic lymphocytic, small lymphocytic, mantle cell, Burkitt's, cutaneous T-cell, AIDS-related, or central nervous system lymphoma); neuroblastoma; gynecological cancer (such as uterine or ovarian cancer); breast cancer; liver cancer; lung cancer; prostate cancer; skin cancer; bone cancer; pancreatic cancer; brain cancer (neuroblastoma or glioma); head or neck cancer; kidney cancer (such as Wilms' tumor); retinoblastoma; adrenocortical tumor; desmoid tumors; desmoplastic small round cell tumor; endocrine tumors; and/or blood cancer (such as myeloma, such as multiple myeloma; lymphoma, such as Hodgkin's or non-Hodgkin's lymphoma, for example, diffuse large B-cell, follicular, chronic lymphocytic, small lymphocytic, mantle cell, Burkitt's, cutaneous T-cell, AIDS-related, or central nervous system lymphoma; or leukemia, such as acute lymphocytic leukemia (ALL) or acute myeloid leukemia (AML))), an immune disorder (e.g., an autoimmune disorder or transplant rejection), or an infectious disease (for example, cytomegalovirus, adenovirus, respiratory syncytial virus, Epstein-Barr virus, HIV infection, or hepatitis virus infection (such as hepatitis C virus or hepatitis B virus), Coronaviruses (such as COVID-19), Ebola Hemorrhagic Fever, Rabies, West Nile virus, yellow fever, herpes types I and II, hantavirus pulmonary syndrome, Marburg virus infection, Lassa virus infection, and Dengue fever).

In some examples, the methods include treating or inhibiting an inherited condition, by administering to a subject a delivery agent disclosed herein, such as in combination with one or more therapeutic agents, using the methods disclosed herein. In some examples, the inherited condition is selected from among Abetalipoproteinemia; Acid Maltase Deficiency/Pompe's disease, ADCY5-induced Dyskinesia; Adenosine Aminohydrolase Deficiency (ADA): Agammaglobulinemia, X-linked, Type 1; Alagille Syndrome; All Hypertrophic and Dilated Cardiomyopathy; Alopecia Universalis Congenita (ALUNC); Alpers Syndrome; Alpha mannosidosis; Alpha-1-Antitrypsin Deficiency; Alpha-Thalassemia-Southeast Asia; Amyotrophic Lateral Sclerosis-Lou Gehrig's Disease; Androgen Insensitivity Syndrome; Aniridia; Ankylosing spondylitis; APC-Associated Polyposis Conditions; Argininosuccinate Lyase Deficiency; Arrhythmogenic Right Ventricular Dysplasia/Cardiomyopathy; Aspartylglucosaminuria; Ataxia with Oculomotor Apraxia Type 2; Ataxia with Vitamin E Deficiency; Ataxia-Telangiectasia; Autoimmune Polyendocrine Syndrome; Beta-Hydroxyisobutyryl CoA Deacylase deficiency (HIBCH deficiency); Beta mannosidosis; Biotinidase Deficiency; Blepharophimosis-ptosis-epicanthus inversus; Bloom Syndrome; Brachydactyly; Brachydactyly-Hypertension Syndrome; Brachydactyly Type B1; Branchiootorenal Spectrum Disorders; BRCA1; Campomelic Dysplasia; Canavan; Cerebrotendinous Xanthomatosis; Ceroid-lipofuscinoses-Batton; Charcot-Marie-Tooth Disease Type 2B; Charcot-Marie-Tooth Neuropathy Type 1B; Charcot-Marie-Tooth Neuropathy Type 2A2; Charge Syndrome; Cherubism; Choroideremia; Citrin Deficiency; Citrullinemia Type I; Coffin-Lowry Syndrome; Cohen Syndrome; Collagen 4A5; Common Variable Immune Deficiency; Congenital Adrenal Hyperplasia; Congenital Cataracts, Facial Dysmorphism, and Neuropathy; Congenital Disorder of Glycosylation Type 1a; Congenital Myasthenic Syndromes; Cornelia de Lange Syndrome; Crohn's disease; Cystic fibrosis; Cystinosis; Darier Disease; Desmin Storage Myopathy; DFNA2 Nonsyndromic Hearing Loss; Diamond-Blackfan Anemia; DNMT1-complex disorder; Double Cortex Syndrome; Duane Syndrome; Duchenne/Becker muscular dystrophy; Dysferlinopathy; Dyskeratosis Congenita; Early-Onset Familial Alzheimer Disease; Early-Onset Primary Dystonia (DYT1); Ehlers Danlos; Ehlers-Danlos Syndrome, Classic Type; Ehlers-Danlos Syndrome, Hypermobility Type; Ehlers-Danlos Syndrome, Kyphoscoliotic Form; Emery-Dreifuss Muscular Dystrophy X linked; Epidermolysis Bullosa Simplex; Fabry Disease; Facioscapulohumeral Muscular Dystrophy; Familial Dysautonomia (HSAN III); Familial Hyperinsulinism (FHI); Familial Hypertrophic Cardiomyopathy: Familial Transthyretin Amyloidosis; Fanconi Anemia; Fatal familial insomnia; Fibrodysplasia Ossificans Progressiva; Fragile X; Friedreich Ataxia; FRMD7-Related Infantile Nystagmus; Fryns Syndrome; Fucosidosis; Galactosemia; Galactosialidosis; Gaucher Disease; Gerstmann-Straussler-Scheinker syndrome; Glut1 Deficiency; Glycine Encephalopathy: Glycogen Storage Disease Type VI; Hemophagocytic Lymphohistiocytosis; Hemophilia A; Hemophilia B; Hepatic Veno-Occlusive Disease with Immunodeficiency; Hereditary Hemorrhagic Telangiectasia; Hereditary Neuropathy with Liability to Pressure Palsies; Hereditary Nonpolyposis Colon Cancer; Hexosaminidase A Deficiency; HFE-Associated Hereditary Hemochromatosis; Holt-Oram Syndrome; Huntington Disease; Hydroxymethylbilane Synthase (HMBS) Deficiency; Hypophosphatasia; Inclusion Body Myopathy 2; Incontinentia Pigmenti; Juvenile Polyposis Syndrome; Kallmann Syndrome; Krabbe Disease; Leber Congenital Amaurosis; Leber congenital amaurosis 10; Leukoencephalopathy with brain stem and spinal cord involvement and lactate elevation (LBSL); Li-Fraumeni Syndrome; Limb-Girdle Muscular Dystrophy Type 2A Calpainopathy; LIS1-Associated Lissencephaly; Long QT Syndrome; Lowe Syndrome; a lysosomal storage disease (such as Gaucher disease) Malignant Hyperthermia Susceptibility; Maple Syrup Urine Disease; MAPT-Related Disorders; Mast Cell Disease; McKusick-Kaufman Syndrome; MECP2-Rett Syndrome; Menkes; Metachromatic Leukodystrophy; Methylmalonic Acidemia; Mucolipidosis II; Multiple Endocrine Neoplasia Type 1; Mucolipidosis I; Mucolipidosis II; Multiple Endocrine Neoplasia Type 2: Myotonia Congenita; Myotonic Dystrophy Type 1: Myotonic Dystrophy Type 2; Nail-Patella Syndrome; Nemaline Myopathy; Neurofibromatosis 1; Neurofibromatosis 2; Noonan Syndrome; Ocular Albinism, X-Linked: Oculocutaneous Albinism Type 1; Oculocutaneous Albinism Type 2; Oculopharyngeal Muscular Dystrophy; Optic Atrophy Type 1; Ornithine Transcarbamylase Deficiency; Osteogenesis Imperfecta; Parkinson Disease; Pendred Syndrome; Peroxisome Biogenesis, Zellweger; Polyneuropathy, hearing loss, ataxia, retinitis pigmentosa, and cataract (PHARC) disease, Phenylketonuria; Polycystic Kidney Disease; Pompe Disease-GSD II: Porphyria; Primary Ciliary Dyskinesia; Progeria/HGPS, Retinitis Pigmentosa; Retinoblastoma; Saethre-Chotzen Syndrome; Schindler disease; SCN9A-Related Inherited Erythromelalgia; SHOX-Related Haploinsufficiency; Sickle Cell Disease; Smith-Lemli-Opitz Syndrome; Smith-Magenis Syndrome; Sotos Syndrome; Spastic Paraplegia 3A; Spastic Paraplegia 7; Spastic Paraplegia 8; Spastic Paraplegia Type 1; Spastic Paraplegia Type 4; Spinal Muscular Atrophy; Spinocerebellar Ataxia 2; Spinocerebellar Ataxia 3; Spinocerebellar Ataxia 7; Spinocerebellar Ataxia Type 1; Stickler Syndrome; Thanatophoric Dysplasia; Thoracic Aortic Aneurysms and Aortic Dissections; Treacher Collins Syndrome; Trimethylaminuria; Tuberous Sclerosis Complex; Udd Distal Myopathy; Usher Syndrome type 1; LBSL disease; Very Long Chain Acyl-Coenzyme A Dehydrogenase Deficiency; von Hippel-Lindau; Waardenburg Syndrome, Type 1; Werner Syndrome; Wilms Tumor; Wilson Disease; Wiskott-Aldrich; X-Linked Adrenal Hypoplasia Congenita; X-Linked Adrenoleukodystrophy; X-Linked Dystonia-Parkinsonism; X-linked Juvenile Retinoschisis; X-linked myotubular Myopathy; X-Linked SCIDS; and Zellweger Syndrome.

In some examples, the subject that has the inherited condition is administered a delivery agent disclosed herein and one or more therapeutic agents targeting the inherited condition. Exemplary therapeutics for treating or inhibiting an inherited condition can be found, for example, in Bick et al., Am J Med Genet C Semin Med Genet. 2021,187 (1):48-54 and Gahl et al., Orphanet J Rare Dis. 2021, 16:308. Particular examples of therapeutic agents that can be used include ivacaftor, tezacaftor, lumacaftor, inhaled antibiotics, allopurinol, statin, granulocyte colony stimulating factor, Clonazepam, EDTA chelation therapy, ezetimibe, cholestryramine, sodium benzoate, ravicti, metronidazole, cysteamine, chlorophyllin, growth hormone, insulin, testosterone, etidronate, anti-hypertensive, metreleptin, lumasiran, N-carbamylglutamate, nitisinone, bezafibrate, phenylbutyrate, sirolimus, diazoxide, somatostatin analogs, nifedipine, glucagon, insulin-like growth factor-1, mTOR inhibitor, GLP-1 receptor antagonists, oral antidiabetic drugs, glibenclamide, leflunomide, rapamycin, emapalumab, TNF inhibitor, corticosteroids, platelet transfusion, red blood cell transfusion, antibacterial prophylaxis, antifungal prophylaxis, Interferon gamma, fludrocortisone, mineralocorticoid receptor antagonists, amiloride, triamterene, eplerenone, calcium channel blocker, clonidine, spironolactone, beta-blocker, antiepileptic, phenytoin, lacosamide, eculizumab, meningococcal vaccine, Haemophilus influenzae vaccine, pneumococcal vaccine, purified C1 inhibitor concentrate, ecallantide, icatibant, lanadelumab, ezogabine, memantine, dextromethorphan, carbamazepine, stiripentol, perampanel, acetylcholine-esterase inhibitors, albuterol, salbutamol, fluoxetine, quinidine, 3,4-diaminopyridine, ephedrine, acetazolamide, mexiletine, lamotrigine, quinine, dantrolene, vigabatrin, decarboxylase inhibitor, levodopa, dopamine agonists, MAO B inhibitors, pramipexole, L-threo-3,4-dihydroxyphenylserine, givosiran, hemin, afamelanotide, methyxanthine, valproic acid, cyclic pyranopterin monophosphate, epalrestat, burosumab, isotretinoin, patisiran, yegsedi, tafamidis, miglustat, ambroxol, factor 8, factor 9, fibrinogen concentrate, factor XIII A-subunit, fresh frozen plasma, cryoprecipitate, factor XIII concentrate, eteplirsen, vyondys 53, deferoxamine, mitapivat, growth hormone receptor antagonist, estrogen, thiazide diuretics, plerixafor, ruxolitinib, abatacept, leniolisib, rituximab, adalimumab, tacrolimus, nonsteroidal anti-inflammatory drugs, ofatumumab, angiotensin-converting enzyme inhibitor, febuxostat, volanesorsen, indomethacin, alfacalcidol, oxcarbazepine, DDAVP, anakinra, rilonacept, canakinumab, colchicine, inflixiimab, tocilizumab, etanercept, ustekinumab, secukinumab, mesalazine, cyclosporine, methotrexate, azathioprine, hydroxychloroquine, selumetinib, imatinib, sunitinib, tolvaptan, levothyroxine, tetrabenazine, omeprazole, ranirestat, chloroquine, inclisiran, evolocumab, “lomitapide”, desferrioxamine, baricitinib, deoxycytidine, deoxythymidine, Benzoate and phenylacetate, Sodium phenylbutyrate, Carglumic acid, Betaine. Pegvaliase, Eliglustat, Velaglucerase alfa, Imiglucerase, Taliglucerase, Agalsidase beta, Agalsidase alfa, Migalastat, Sebelipase alfa, Alglucosidase alfa, Velmanase alfa, Laronidase, Idursulfase, Elosulfase alfa, Galsulfase, Vestronidase alfa, Cerliponase alfa, Cysteamine (enteric coated), Cysteamine hydrochloride eyedrops, Rosuvastatin calcium, Lomitapide, Cholic acid, Chenodeoxycholic acid, Asfotase alfa, Burosumab-twza, Calcium acetate, Alendronate, Ascorbic acid, Thiamine, Trisodium citrate, Levocarnitine, Triheptanoin, Riboflavin, Uridine triacetate, Potassium citrate, Tiopronin, Penicillamine, Trientine HCl, Zinc acetate, Hydroxocobalamin, Inotersen, Patisiran sodium, Teriflunomide, Fingolimod HCl, Siponimod, Rasagiline, Selegiline, Carbidopa/Levodopa, Pitolisant, Sodium oxybate, Deutetrabenazine, Baclofen, Everolimus, Folic acid, Biotin, Rufinamide, Cannabidiol, Midazolam, Levetiracetam, Clobazam, Topiramate, Gabapentin, Riluzole, Radicava, Pyridostigmine Bromide, Amifampridine, Mexiletine hcl, Nusinersen sodium, Octocog alpha, Rurioctocog alfa pegol, Lonoctocog alfa, Emicizumab, Damoctocog alfa pegol, Turoctocog alpha, Simoctocog alfa, Moroctocog alpha, Desmopressin acetate. Recombinant Factor VIII, Efmoroctocog alfa, Factor VIII/von Willebrand factor, Vonicog alfa, Eftrenonacog alfa, Albutrepenonacog alfa, Nonacog alpha, Human coagulation factor IX, Nonacog beta pegol, Nonacog gamma, Recombinant Factor IX. Eptacog alpha (activated), Recombinant Factor Vila, Human coagulation factor X, Catridecacog, Human protein c, Hydroxyurea, Epoetin alfa, Eltrombopag, Deferasirox, Methylene blue injection, Siltuximab, Anagrelide hydrochloride, Ravulizumab, Macapegfilgrastim, Busulfan, Thiotepa, Deferiprone, Caplacizumab, Romiplostim, Ropeginterferon alfa-2b, Immunoglobulin infusion, Methylprednisolone, Golimumab, Mesalamine, 5-aminosalicylic acid, Obeticholic acid, Tocofersolan, C1 inhibitor (human), Icatibant acetate, Danazol, Tranexamic acid, C1-esterase-inhibitor, human, Conestat alfa, IL-1 Receptor antagonist anakinra. Cenegermin, Ciclosporin, Dexamethasone, Somatropin for injection, Octreotide, Lanreotide, Pegvisomant, Pasireotide, Osilodrostat, Ketoconazole, Hydrocortisone, Human chorionic gonadotropin, Gonadotropin-releasing hormone, Mecasermin, Calcitonin-human for injection, Parathyroid hormone, Tasimelteon, Macitentan, Tadalafil, Ambrisentan, Nitric oxide, Sildenafil, Bosentan monohydrate, Selexipag, Iloprost, Parenteral treprostinil, Riociguat, Mannitol, Tezacaftor/ivacaftor, Tobramycin, Aztreonam, Colistimethate sodium, Lumacaftor/ivacaftor, Levofloxacin, Pirfenidone, Nintedanib, Caffeine citrate, Pegademase bovine, CD34+ cells transduced with ADA cDNA, Interferon gamma 1-b, Cromolyn sodium, Amiodarone, Autologous human corneal stem cells, Voretigene neparvovec, Teduglutide, Defibrotide, Ibuprofen, or Dexrazoxane.

In some specific aspects of the disclosure, the inherited condition is muscular dystrophy. In particular examples, the muscular dystrophy is Duchenne muscular dystrophy, Becker muscular dystrophy, limb-girdle muscular dystrophy, congenital muscular dystrophy, facioscapulohumeral muscular dystrophy, myotonic muscular dystrophy, oculopharyngeal muscular dystrophy, distal muscular dystrophy, or Emery-Dreifuss muscular dystrophy. In some aspects of the disclosure, the muscular dystrophy is Duchenne muscular dystrophy. In some aspects of the disclosure, the disease is a demyelinating disease. In some further aspects of the disclosure, the demyelinating disease is multiple sclerosis, Charcot-Marie-Tooth disease, Pelizaeus-Merzbacher disease, encephalomyelitis, neuromyelitis optica, adrenoleukodystrophy, and/or Guillian-Barre syndrome. In some examples, the methods are utilized to treat a subject with one or more signs or symptoms of muscular disease such as a muscular dystrophy, such as, but not limited to DMD.

In particular examples, the subject is administered an effective amount of a disclosed delivery agent in combination with one or more therapeutic agents that reduce or inhibit one or more signs or symptoms associated with muscular dystrophy. Exemplary therapeutic agents can include an α7β1 modulatory agent or laminin-111 protein therapy, which works to stabilize the sarcolemma and reduce muscle degeneration. Therapeutic agents of use herein can further include a component of the extracellular matrix, such as an integrin, dystrophin, dystroglycan, utrophin, or a growth factor. In some examples, the additional therapeutic agent reduces or enhances expression of a substance that enhances the formation or maintenance of the extracellular matrix. In some examples, the additional substance can include aggrecan, angiostatin, cadherins, collagens (including collagen I, collagen III, or collagen IV), decorin, elastin, enactin, endostatin, fibrin, fibronectin, osteopontin, tenascin, thrombospondin, vitronectin, and combinations thereof. Biglycans, glycosaminoglycans (such as heparin), glycoproteins (such as dystroglycan), proteoglycans (such as heparan sulfate), and combinations thereof can also be administered. In specific, non-limiting examples, a subject that has DMD is administered a morpholino in combination with a disclosed delivery agent. Exemplary morpholinos for use in treating DMD include ETEPLIRSEN® (Sarepta Therapeutics).

In some examples, growth stimulants such as cytokines, polypeptides, and growth factors such as brain-derived neurotrophic factor (BDNF), CNF (ciliary neurotrophic factor), EGF (epidermal growth factor), FGF (fibroblast growth factor), glial growth factor (GGF), glial maturation factor (GMF) glial-derived neurotrophic factor (GDNF), hepatocyte growth factor (HGF), insulin, insulin-like growth factors, kerotinocyte growth factor (KGF), nerve growth factor (NGF), neurotropin-3 and -4, PDGF (platelet-derived growth factor), vascular endothelial growth factor (VEGF), and combinations thereof may be administered with a delivery agent using a disclosed method.

In some examples, the methods include treating or inhibiting cancer, such as a hematological malignancy or a solid tumor. In certain aspects of the disclosure, administration of a therapeutically effective amount of a disclosed combination therapy to a subject reduces cancer burden in the subject, increases survival of the subject, reduces the incidence of relapse of a cancer in the subject, induces maturation of dendritic cells in the subject, or a combination thereof. The method can include selecting the subject with the cancer. Examples of hematological malignancies include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), T-cell large granular lymphocyte leukemia, polycythemia vera, lymphoma, Hodgkin's lymphoma, non-Hodgkin's lymphoma (indolent and high grade forms; includes diffuse large B-cell, follicular, chronic lymphocytic, small lymphocytic, mantle cell, Burkitt's, cutaneous T-cell, AIDS-related, or central nervous system lymphoma), multiple myeloma, Waldenstrom's macroglobulinemia, heavy chain disease, myelodysplastic syndrome, hairy cell leukemia, and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoma (includes indolent and high grade forms; Hodgkin's lymphoma; and non-Hodgkin's lymphoma, such as diffuse large B-cell, follicular, chronic lymphocytic, small lymphocytic, mantle cell, Burkitt's, cutaneous T-cell, AIDS-related, or central nervous system lymphoma), pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma, and retinoblastoma).

In particular examples, hematological malignancies that can be inhibited or treated by the methods disclosed herein include but are not limited to multiple myeloma, chronic lymphocytic leukemia, acute lymphocytic leukemia, acute myeloid leukemia, chronic myelogenous leukemia, pro-lymphocytic/myelocytic leukemia, plasma cell leukemia, NK cell leukemia, Waldenstrom macroglobulinemia, Hodgkin's lymphoma, and non-Hodgkin's lymphoma (indolent and high grade forms; includes diffuse large B-cell, follicular, chronic lymphocytic, small lymphocytic, mantle cell, Burkitt's, cutaneous T-cell, AIDS-related, or central nervous system lymphoma). In additional particular examples, solid tumors that can be treated or inhibited by the methods disclosed herein include lung carcinoma, prostate cancer, pancreatic cancer (for example, insulinoma), breast cancer, colorectal adenocarcinoma or squamous cell carcinoma, neuroblastoma, testicular cancer (such as seminoma), and ovarian cancer. In specific, non-limiting examples, the subject has chronic myelogenous leukemia, acute monocytic leukemia, or non-Hodgkin's lymphoma (indolent and high grade forms; includes diffuse large B-cell, follicular, chronic lymphocytic, small lymphocytic, mantle cell, Burkitt's, cutaneous T-cell, AIDS-related, or central nervous system lymphoma).

In some examples, the subject (such as a subject with a cancer) is administered a delivery agent disclosed herein and one or more chemotherapeutic agents and/or radiation therapy. Chemotherapeutic agents useful in the disclosed methods include chemical or biological agent with therapeutic usefulness in the treatment of diseases characterized by abnormal cell growth. Particular examples of chemotherapeutic agents that can be used include microtubule binding agents, DNA intercalators or cross-linkers, DNA synthesis inhibitors, DNA and RNA transcription inhibitors, antibodies, enzymes, enzyme inhibitors, gene regulators, and angiogenesis inhibitors. In one embodiment, a chemotherapeutic agent is a radioactive compound. Other chemotherapeutic agents that can be used are provided in Sausville and Longo, Principles of Cancer Treatment, Chapter 69 in Harrison's Principles of Internal Medicine (20^{th} ed.), McGraw-Hill, 2018; Niederhuber et al., Cancer Pharmacology, Ch. 25 in Abeloff's Clinical Oncology (6^{th} ed.), Elsevier, 2019; Gullatte et al., Clinical Guide to Antineoplastic Therapy: A Chemotherapy Handbook (4^{th} ed.), Oncology Nursing Society, 2020; Chabner and Longo, Cancer Chemotherapy, Immunotherapy and Biotherapy: Principles and Practice (6^{th} ed.), Lippincott Williams & Wilkins. 2018: Skeel, Handbook of Cancer Chemotherapy (9^{th} ed.), Lippincott Williams & Wilkins, 2016. The disclosed methods can include administration of more than one chemotherapeutic agent, such as in combination with a delivery agent disclosed herein. Such chemotherapeutic agents include alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine); antimetabolites such as folic acid analogs (such as methotrexate), pyrimidine analogs (such as 5-FU or cytarabine), and purine analogs, such as mercaptopurine or thioguanine; or natural products, for example vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Additional agents include platinum coordination complexes (such as cis-diamine-dichloroplatinum II, also known as cisplatin), substituted ureas (such as hydroxyurea), methyl hydrazine derivatives (such as procarbazine), and adrenocrotical suppressants (such as mitotane and aminoglutethimide); hormones and antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include adriamycin, melphalan (Alkeran®) Ara-C (cytarabine), carmustine, busulfan, lomustine, carboplatinum, cisplatinum, cyclophosphamide (Cytoxan®), daunorubicin, dacarbazine, 5-fluorouracil, fludarabine, hydroxyurea, idarubicin, ifosfamide, methotrexate, mithramycin, mitomycin, mitoxantrone, nitrogen mustard, paclitaxel (or other taxanes, such as docetaxel), vinblastine, vincristine, VP-16, while newer drugs include gemcitabine (Gemzar®), trastuzumab (Herceptin®), irinotecan (CPT-11), leustatin, navelbine, rituximab (Rituxan®) imatinib (STI-571), Topotecan (Hycamtin®), capecitabine, ibritumomab (Zevalin®), and calcitriol.

Treatment of the conditions and diseases described herein are generally initiated after the development of a condition described herein, or after the initiation of a precursor condition (such as dysplasia or development of a benign tumor). Treatment can be initiated at the early stages of cancer. For instance, treatment can be initiated before a subject manifests symptoms of a condition, such as during a stage I diagnosis or at the time dysplasia is diagnosed. However, treatment can be initiated during any stage of the disease, such as but not limited to stage I, stage II, stage III and stage IV cancers. Treatment prior to the development of the condition, such as treatment upon detecting dysplasia or an early (benign) precursor condition, is referred to herein as treatment of a subject that is “at risk” of developing the condition. In some aspects of the disclosure, administration of a combination therapy can be performed during or after the occurrence of the conditions described herein.

Thus, a subject can be selected for treatment that has, or is at risk for developing a cancer, such as a cancer disclosed herein, such as, for example, a colorectal cancer, a kidney cancer, or a melanoma. Typical subjects intended for administration of the combination therapies disclosed herein include humans, as well as non-human primates and other animals. To identify relevant subjects, accepted screening methods are employed to determine risk factors associated with, and/or to diagnose, a targeted or suspected cancer (such as a colorectal cancer, a kidney cancer, or a melanoma) in a subject, or to determine the status of an existing cancer in the subject. These screening methods include, for example, conventional work-ups to determine environmental, familial, occupational, and other such risk factors that may be associated with the targeted or suspected cancer, as well as diagnostic methods, such as, but not limited to, various histopathological, morphological, and/or cytological analyses to identify or diagnose the targeted cancer. These and other routine methods allow the clinician to select patients in need of therapy. In accordance with these methods and principles, the combination therapies disclosed herein can be administered according to the teachings herein, or other conventional methods, as an independent prophylaxis or treatment program, or as a follow-up, adjunct, or coordinate treatment regimen to other treatments.

In some examples, the methods include treating or inhibiting an immune disease or disorder by administering a delivery agent disclosed herein, such as in combination with one or more therapeutic agents, using the methods disclosed herein to a subject. The immune disease or disorder can be any type of immune system condition, such as a cytokine storm, an immune system disorder (e.g., an inflammatory or autoimmune disorder) or can be immune system conditions associated with another condition and/or disease (e.g., HIV infection or exposure to microgravity). In some non-limiting examples, the immune system disease or disorder is an inflammatory disorder. In specific aspects of the disclosure, the inflammatory disorder can be rheumatoid arthritis, chronic obstructive pulmonary lung disease, inflammatory bowel disease, or systemic lupus erythematosus. In other examples, the immune system disease or disorder is an autoimmune disorder. In certain aspects of the disclosure, the autoimmune disorder is type I diabetes, multiple sclerosis, lupus erythematosus, myasthenia gravis, ankylosing spondylitis, celiac disease, Crohn's disease, Graves' disease, Hashimoto's thyroiditis, transplant rejection, or autoimmune uveitis.

In some examples, the subject (e.g., a subject with an immune disease or disorder, such as an autoimmune disease, transplant rejection, or inflammatory disease) is also administered one or more immunomodulatory therapies (e.g., immunomodulatory biologics, such as muromonab, ipilimumab, abatacept, belatacept, tremelimumab, BMS-936558, CT-011, MK-3475, AMP224, BMS-936559, MPDL3280A, MEDI4736, MGA271, IMP321, BMS-663513, PF-05082566, CDX-1127, anti-OX40, huMAb, OX40L, and TRX518, e.g., Yao et al., Nat Rev Drug Discov, 12 (2):130-146, 2013, and Kamphorst et al., Vaccine, 33 (0 2): B21-B28, 2015, both of which are incorporated herein by reference in their entireties; modulatory cytokines, such as IL-7; mTOR modulatory agents, such as rapamycin; antimicrobial therapy, such as vaccination, antifungals, and/or antibiotics), anti-inflammatory agents (NSAIDS; antileukotrines; immune selective anti-inflammatory derivatives, ImSAIDs; bioactive compounds with anti-inflammatory activities, such as plumbagin and plumericin; and/or steroids), disease-modifying antirheumatic drugs (DMARDs, such as methotrexate, sulfasalazine, leflunomide, hydroxychloroquine, tofacitinib, infliximab, etanercept, adalimumab, certolizumab, golimumab, tocilizumab, anakinra, abatacept, and/or rituximab), antimalarial drugs (e.g., chloroquine and hydroxychloroquine), medical procedures (including surgery and stem cell transplantation); immunosuppressive agents (e.g., for preventing rejection of transplanted organs or tissues, treating autoimmune diseases, and/or inflammatory diseases; e.g., glucocorticoids, such as prednisone, dexamethasone, and hydrocortisone; cytostatics, such as alkylating agents and antimetabolites; antibodies, such as Atgam, thymoglobuline, and T-cell receptor- and IL-2 receptor-directed antibodies; immunophilin-targeting agents, such as cyclosporin, tacrolimus, sirolimus, and everolimus; interferons (IFNs), such as IFNλ and IFNß; opioids; TNF binding proteins, such as infliximab, etanercept, and adalimumab; mycophenolate; and small biological agents, such as fingolimod and myriocin), immune tolerance therapy (e.g., for treating subjects at risk for tissue or organ transplantation rejection, subjects with allergies, and/or subjects with autoimmune disease; e.g., T or B cell-targeting or T or B cell-suppressing drugs, such as CAMPATH-1H, calcineurin inhibitors, rituximab, epratuzumab, belimumab, and atacicept; anti-cluster of differentiation (CD)3 antibodies; abatacept; induction of hematopoietic chimerism, such as mixed hematopoietic chimerism, in which the bone marrow of an organ or a tissue recipient is replaced with the donor's bone marrow or a mixture of the donor and recipient bone marrow to reduce organ or tissue transplant rejection; antigen desensitization; see Nepom et al., Immunol Rev; 241(1): 49-62, 2011, incorporated herein by reference), antihistamines, helminthic therapies (e.g., deliberate infestation of the subject with a helminth or with the ova of a helminth for treating immune disorders).

In some examples, the methods include treating or inhibiting an infectious disease by administering to a subject a delivery agent disclosed herein, such as in combination with one or more therapeutic agents, using the methods disclosed herein. In some examples, the infectious disease is selected from among arboviral infections, botulism, brucellosis, candidiasis, campylobacteriosis, chickenpox, chlamydia, cholera, coronovirus infections, staphylococcus infections, coxsackie virus infections, Creutzfeldt-Jakob disease, cryptosporidiosis, cyclospora infection, cytomegalovirus infections, Epstein-Barr virus infection, dengue fever, diphtheria, ear infections, encephalitis, influenza virus infections, parainfluenza virus infections giardiasis, gonorrhea, Haemophilus influenzae infections, hantavirus infections, viral hepatitis, herpes simplex virus infections, HIV/AIDS, helicobacter infection, human papillomavirus (HPV) infections, infectious mononucleosis, legionellosis, leprosy, leptospirosis, listeriosis, lyme disease, lymphocytic choriomeningitis, malaria, measles, marburg hemorrhagic fever, meningitis, monkeypox, mumps, mycobacteria infection, mycoplasma infection, norwalk virus infection, pertussis, pinworm infection, pneumococcal disease, Streptococcus pneumonia infection, Mycoplasma pneumoniae infection, Moraxella catarrhalis infection, Pseudomonas aeruginosa infection, rotavirus infection, psittacosis, rabies, respiratory syncytial virus infection (RSV), ringworm, rocky mountain spotted fever, rubella, salmonellosis, SARS, scabies, sexually transmitted diseases, shigellosis, shingles, sporotrichosis, streptococcal infections, syphilis, tetanus, trichinosis, tuberculosis, tularemia, typhoid fever, viral meningitis, bacterial meningitis, west Nile virus infection, yellow fever, adenovirus-mediated infections and diseases, retrovirus-mediated infectious diseases and yersiniosis zoonoses. For example, the infectious disease can be influenza, parainfluenza, respiratory syncytial virus. In particular examples, the infectious disease is human immunodeficiency virus (HIV), hepatitis B virus (HBV), hepatitis C virus (HCV), tuberculosis (TB), malaria, tooth decay, or Helicobacter pylori infection.

In some examples, the subject (e.g., a subject with an infectious disease, such as HIV) is also administered one or more anti-infection agents (e.g., antibodies, antifungals, antivirals, and/or antiparasitics). In some examples, the anti-infection agent is an antibiotic, such as a penicillin, amoxicillin, doxycycline, cephalexin, ciprofloxacin, clindamycin, metronidazole, azithromycin, sulfamethoxazole, trimethoprim, amoxicillin, clavulanate, or levofloxacin. In some examples, the anti-infection agent is an antiviral, such as tilorone, vicriviroc, arbidol, or nelfinavir. In specific examples, the infectious disease is HIV, and the subject is also administered antiretroviral agents, such as nucleoside and nucleotide reverse transcriptase inhibitors (nRTI), non-nucleoside reverse transcriptase inhibitors (NNRTI), protease inhibitors, entry inhibitors (or fusion inhibitors), maturation inhibitors, or a broad-spectrum inhibitors, such as natural antivirals. Other exemplary agents include lopinavir, ritonavir, zidovudine, lamivudine, tenofovir, emtricitabine, and efavirenz.

In some examples, the methods include treating or inhibiting a psychiatric disorder by administering a delivery agent disclosed herein, such as in combination with one or more therapeutic agents, using the methods disclosed herein to a subject. The psychiatric disorder can be any type of psychiatric disorder, such as an anxiety disorder, attention-deficit hyperactivity disorder (ADHD), a behavioral and/or emotional disorder in children, bipolar affective disorder, depression, dissociation and dissociative disorders, eating disorders, obsessive compulsive disorder, a panic disorder, paranoia, post-traumatic stress disorder, a psychosis, and/or schizophrenia. In some non-limiting examples, the psychiatric disorder is depression.

In some examples, the subject (e.g., a subject with a psychiatric disorder, such as a subject that has depression) is also administered one or more antidepressants, such as, but not limited to, a selective serotonin uptake inhibitor (SSRI), a tricyclic antidepressant, and/or a monoamine oxidase inhibitor (MAOI). Tricyclic antidepressants include imipramine, amitriptyline, nortriptyline, and desipramine. MAOIs approved for the treatment of depression include phenelzine (NARDIL®), tranylcypromine (PARNATE®), and isocarboxazid (MARPLAN®). SSRIs primarily affect the neurotransmitter serotonin, and can include escitalopram HBr (LEXAPRO®), fluoxetine (PROZAC®), sertraline (ZOLOFT®), fluvoxamine (LUVOX®), paroxetine (PAXIL®), and citalopram (CELEXA®). Additional medication of use affect both norepinephrine and serotonin, for example venlafaxine (EFFEXOR®) and nefazadone (SERZONE®), or agents such as phenelzine (NARDIL®), tranylcypromine (PARNATE®), mirtazepine (REMERON®), nefazodone (SERZONE®), triazolopyridine (TRAZODONE®), and bupropion (WELLBUTRIN®).

### C. Methods of Making Delivery Agent Embodiments

Also disclosed herein is a method for making a delivery agent according to the present disclosure. The delivery agent can be made by coupling components of the delivery agent as discussed herein, such as by performing one or more chemical couplings. In some aspects of the disclosure, the chemical couplings can comprise suitable peptide bond forming reactions, conditions and reagents. The chemical couplings can take place in any suitable order. For example, the chemical coupling can take place sequentially such that a C-terminus group is used as a building block for synthesizing the remaining peptide backbone of the delivery agent. In some aspects of the disclosure, peptide linkages are formed off of the C-terminus group to thereby provide anchor groups attached to the C-terminus group, followed by a mask peptide sequence attached to the anchor groups, followed by a cleavable linker group attached to the mask peptide sequence, followed by a lytic peptide group attached to the cleavable linker group. The lytic peptide can be functionalized so as to provide an N-terminus group as disclosed herein. Other components that can be coupled with the delivery agent can be added during or after the delivery agent synthesis. For example, targeting groups and/or therapeutic agents can be attached to different attachment points within the delivery agent. Solely by way of example, a lysine or cysteine attachment point that is part of the delivery agent peptide backbone (or a branch thereof) can be functionalized with the targeting group and/or the therapeutic agent using a chemical linker group that can be covalently bound to a side chain or C- or N-terminus of the lysine or cysteine. Suitable coupling conditions and reagents can be used to attach such components. In yet some additional aspects of the disclosure, click chemistry can be used to attach therapeutic agents and/or targeting groups to the chemical linker group. For example, click chemistry using azide-alkyne cycloaddition coupling conditions, which are recognized by those in the art, can be used to couple the chemical linker and the therapeutic agent or targeting group. In yet additional aspects of the disclosure, therapeutic agents can be coupled covalently to the delivery agent using reaction conditions suitable for coupling an amine, a cysteine, a carboxylic acid group, or other reactive functional group of an amino acid to one or more functional groups of the therapeutic agent (e.g., activated carbonates, thiocarbonyl groups, activated esters, α,β-unsaturated ketones, olefins, and the like), particularly with the benefit of the present disclosure.

In some aspects of the disclosure, suitable peptide bond forming conditions can include using a coupling reagent including, but are not limited to, 2-(7-aza-1H-benzotriazol-1-yl)-N, N,N′,N′-tetramethylaminium hexafluorophosphate, 2-(1H-benzotriazol-1-yl)-N,N,N′,N′-hexafluorophosphate, 2-(6-chloro-1H-benzotriazol-1-yl)-N,N,N′,N′-tetramethylaminium hexafluorophosphate, 1-hydroxybenzotriazole, dicyclohexylcarbodiimide, diisopropylcarbodiimide, N-(3-dimethylaminopropyl)-N′-ethylcarbodiimide. HCl, benzotriazol-1-yloxy-tris(dimethylamino)-phosphonium hexafluorophosphate, benzotriazol-1-yloxy-tripyrrolidino-phosphonium hexafluorophosphate, bromo-tripyrrolidino-phosphonium hexafluorophosphate, and the like, or a combination thereof; and a base, such as an amine base (e.g., di-isopropylethyl amine, isopropyl amine, n-methylmorpholine, and the like).

### VI. Kits

Provided herein are kits useful for the various aspects of the disclosure described herein. Kits may contain various materials and reagents (e.g., for practicing the methods described herein). For example, a kit may contain reagents including, without limitation, a disclosed delivery agent, one or more therapeutic agents, cells (such as cells representative of a particular condition or disease, and suitable for use in a method of screening one or more test compounds as described herein), cell culture media, serum, as well as other solutions or buffers useful in carrying out the assays and other methods provided herein. Kits may also include control samples, materials useful in the methods described herein, and containers, syringes, vials, tubes, ampules, capsules, or bottles, microtiter plates, and the like in which assay reactions may be conducted. Kits may be packaged in containers, which may include compartments for receiving the contents of the kits, and can include instructions for conducting methods described herein.

The kit can include a label or package insert on or associated with any container of the kit. The label or package insert typically can further include instructions for use of the delivery agent, one or more therapeutic agent, and/or cells provided with the kit, for example for use in the methods disclosed herein. The instructional materials may be written, in an electronic form, or may be visual (such as video files).

For example, a kit can include (1) a peptide-based delivery agent described herein (such as a composition including a delivery agent as described herein); (2) one or more therapeutic agents (in the same composition and/or container as the delivery agent or in different compositions and/or containers); (3) instructions for administering or using the peptide-based delivery agent of (1) and/or the one or more therapeutic agents of (2); (4) one or more administration devices; or (5) the delivery agent of (1) and any combination of (2)-(4).

### VII. Overview of Several Examples

Disclosed herein are examples of a peptide-based delivery agent comprising: a lytic peptide group having a structure according to a formula [X¹Y¹Y¹X¹]ₘ, wherein each X¹ independently for each occurrence is a basic amino acid, an acidic amino acid, a non-polar amino acid, or a derivative thereof, provided that at least one X¹ is a basic amino acid or an acidic amino acid; each Y¹ independently for each occurrence is a non-polar amino acid or a derivative thereof; and m is an integer selected from 2 to 8; a cleavable linker group; a mask peptide group having a structure according to a formula [X²Y²Y²X²]_{m′}, wherein each X² independently for each occurrence is an acidic amino acid, a non-polar amino acid, or a derivative thereof, provided that at least one X² is an acidic amino acid; each Y² independently for each occurrence is a non-polar amino acid or a derivative thereof; and m′ is an integer selected from 2 to 8; and an anchor group selected from a heteroaliphatic group, a dibenzocyclooctyne compound, an antibody or antibody fragment, a biotin group, an avidin group, a streptavidin group, or a neutravidin group; a targeting group selected from a cell, an antibody or antibody fragment, a peptide, a biomimetic peptide, an aptamer, a sugar, or a small targeting molecule; or a combination thereof.

In any or all of the above examples, the delivery agent has a structure according to Formula IA or IB:

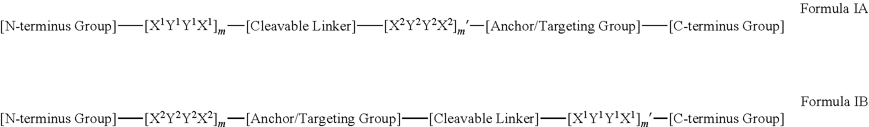

wherein each X¹ independently for each occurrence is a basic amino acid, an acidic amino acid, a non-polar amino acid, or a derivative thereof, provided that at least one X¹ is a basic amino acid or an acidic amino acid; each Y¹ independently for each occurrence is a non-polar amino acid or a derivative thereof; each of m and m′ independently for each occurrence is an integer selected from 2 to 8; each X² independently for each occurrence is an acidic amino acid, a non-polar amino acid, or a derivative thereof, provided that at least one X² is an acidic amino acid; each Y² independently for each occurrence is a non-polar amino acid or a derivative thereof; the cleavable linker is an amino acid sequence that is two to ten amino acids in length; the anchor group, if present, is selected from a heteroaliphatic group, a dibenzocyclooctyne compound, an antibody or antibody fragment, a biotin group, an avidin group, a streptavidin group, or a neutravidin group; the targeting group, if present, is selected from a cell, an antibody or antibody fragment, a peptide, a biomimetic peptide, an aptamer, a sugar, or a small targeting molecule; or a combination thereof; the C-terminus group comprises an amine-terminated glycine moiety; and the N-terminus group comprises a capping group or a fluorophore.

In any or all of the above examples, each X¹ independently for each occurrence is glutamic acid, glutamine, arginine, alanine, aspartic acid, or a derivative thereof, provided that if an X¹ is alanine, then at least one further X¹ is glutamine, glutamic acid, aspartic acid, arginine, or a derivative thereof.

In any or all of the above examples, each X¹ independently for each occurrence is glutamic acid, aspartic acid, or a derivative thereof.

In any or all of the above examples, each X¹ independently for each occurrence is (i) glutamic acid or a derivative thereof; or (ii) aspartic acid or a derivative thereof; or (iii) a combination of glutamic acid and aspartic acid so as to provide a lytic peptide sequence of DLLE or ELLD; or (iv) any combination of (i), (ii), and (iii).

In any or all of the above examples, each Y¹ independently for each occurrence and each Y² independently for each occurrence is leucine, α-methyl leucine, alanine, or a derivative thereof.

In any or all of the above examples, each Y¹ independently for each occurrence and each Y² independently for each occurrence is leucine or a derivative thereof.

In any or all of the above examples, each Y¹ independently for each occurrence and each Y² independently for each occurrence is α-methyl leucine or a derivative thereof.

In any or all of the above examples, each X² independently for each occurrence is glutamine, glutamic acid, aspartic acid, alanine, or a derivative thereof, provided that if an X² is alanine, then at least one further X² is glutamine, glutamic acid, aspartic acid, or a derivative thereof.

In any or all of the above examples, m is 3 and m′ is 3; or m is 4 and m′ is 4.

In any or all of the above examples, each X¹ is glutamic acid; each Y¹ and each Y² is leucine; each X² is glutamine or alanine; m is 3; and m′ is 3; provided that if an X² is alanine, then at least one further X² is glutamine.

In any or all of the above examples, the cleavable linker is cleavable by cathepsin B.

In any or all of the above examples, the cleavable linker comprises an amino acid sequence two to ten amino acids in length.

In any or all of the above examples, the cleavable linker is seven amino acids in length.

In any or all of the above examples, the cleavable linker comprises an amino acid sequence at least 70% identical to SEQ ID NO: 3.

In any or all of the above examples, the cleavable linker comprises an amino acid sequence at least 85% identical to SEQ ID NO: 3.

In any or all of the above examples, the cleavable linker comprises the amino acid sequence of SEQ ID NO: 3.

In any or all of the above examples, the cleavable linker consists of the amino acid sequence of SEQ ID NO: 3.

In any or all of the above examples, the anchor group comprises a lysine moiety wherein a side-chain of the lysine moiety is functionalized with a heteroaliphatic group having a formula —C(O)—X—[CH₂]ₚZ, wherein X is oxygen or CH₂; Z is —N(R)₂, or —N⁺(R)₃, wherein each R independently is hydrogen or aliphatic; and p is an integer ranging from 1 to 3.

In any or all of the above examples, X is oxygen or CH₂; Z is —N(R)₂, wherein one R group is hydrogen and the other R group is C₆-C₁₂ alkyl; and p is 2.

In any or all of the above examples, X is oxygen or CH₂; Z is —N⁺(R)₃, wherein two R groups are methyl and the other R group is C₆-C₁₂ alkyl; and p is 2.

In any or all of the above examples, the anchor group is selected from

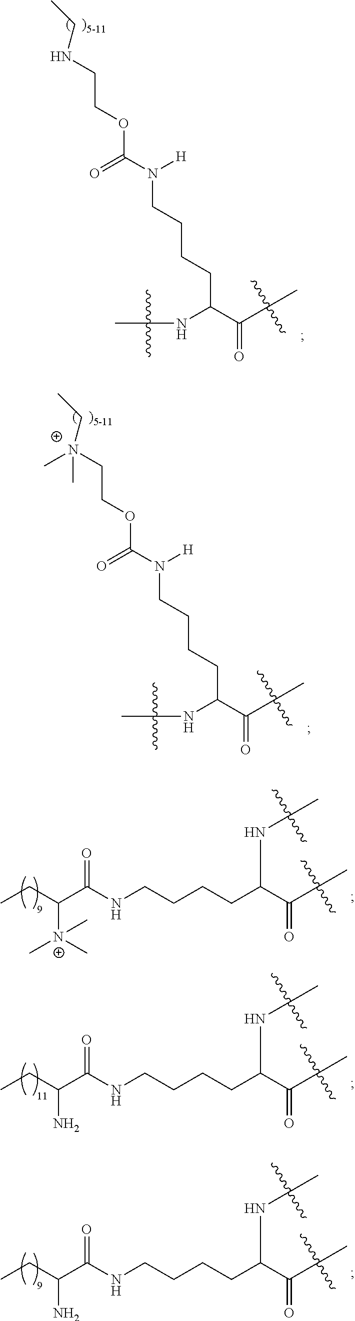

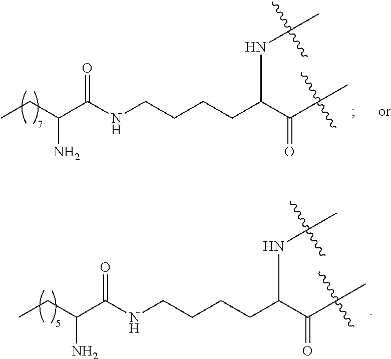

In any or all of the above examples, a plurality of anchor groups is present.

In any or all of the above examples, one, two, or three anchor groups are present.

In any or all of the above examples, the lytic peptide group provides an N-terminus group of the peptide-based delivery agent and the anchor group provides a C-terminus group of the peptide-based delivery agent.

In any or all of the above examples, (i) the N-terminus group is bound to a carbonyl-containing group; and (ii) the C-terminus group is bound to an amine-terminated glycine moiety, a fluorophore, or a combination thereof.

In any or all of the above examples, the carbonyl-containing group is an acetyl group or a fluorophore.

In any or all of the above examples, the fluorophore comprises 2-(methylamino)benzamide.

In any or all of the above examples, the amine-terminated glycine moiety has a structure selected from -Nal-K-G′, -G-Nal-K-G′, -G-K-G′, -Nal-C-G′, -G-Nal-C-G′, -G-C-G′, -Nal-G′, -G-Nal-G′, -W-G′, -G-W-G′, or -G-W—K-G′, wherein Nal is naphthylalanine, G is glycine, G′ is a modified glycine comprising a —C(O)-amine group, W is tryptophan, K is lysine, and C is cysteine.

In any or all of the above examples, the amine-terminated glycine moiety is-G-Nal-G′, wherein G′ is a modified glycine comprising a —C(O)—N(R^{a})₂ group, wherein each R^{a} independently is hydrogen or aliphatic.

In any or all of the above examples, the lytic peptide group has a structure [ELLE]₃; the cleavable linker has an amino acid sequence of SEQ ID NO: 3; the mask peptide group has a structure [QLLQ]₃; and the anchor group has a structure according to that described in any or all of the above examples; and wherein (i) the lytic peptide group provides an N-terminus that is bound to an acetyl group, and (ii) the anchor group is bound to an amine-terminated glycine moiety having a structure-G-Nal-G′, wherein G′ is a modified glycine comprising a —C(O)—N(R^{a})₂ group, wherein each R^{a} independently is hydrogen or aliphatic.

In any or all of the above examples, the lytic peptide group has a structure [ELLE]₄; the cleavable linker has an amino acid sequence of SEQ ID NO: 3; the mask peptide group has a structure [QLLQ]₄; and two anchor groups are present and are bound directly to one another and each anchor group has a structure according to that described in any or all of the above examples; and wherein (i) the lytic peptide group provides an N-terminus that is bound to an acetyl group, and (ii) the anchor group is bound to a modified glycine comprising a —C(O)—N(R^{a})₂ group, wherein each R^{a} independently is hydrogen or aliphatic.

In any or all of the above examples, the lytic peptide group has a structure [QLLE]-[QLLQ]-[QLLE]; the cleavable linker has an amino acid sequence of SEQ ID NO: 3; the mask peptide group has a structure [QLLE]-[QLLQ]-[QLLE]; and the anchor group has a structure according to that described in any or all of the above examples; and wherein (i) the lytic peptide group provides an N-terminus that is bound to an acetyl group, and (ii) the anchor group is bound to an amine-terminated glycine moiety having a structure -G-K-G′, wherein G is glycine, K is lysine, and G′ is a modified glycine comprising a —C(O)—N(R^{a})₂ group, wherein each R^{a} independently is hydrogen or aliphatic.

In any or all of the above examples, the lytic peptide group has a structure [ELLE]₃; the cleavable linker has an amino acid sequence of SEQ ID NO: 1; the mask peptide group has a structure [QLLA]-[QLLA]-[QLLQ]; and an anchor group has a structure according to that described in any or all of the above examples; and wherein (i) the lytic peptide group provides an N-terminus that is bound to an acetyl group, and (ii) the anchor group is bound to an amine-terminated glycine moiety having a structure-G-K-G′, wherein G is glycine, K is lysine, and G′ is a modified glycine comprising a —C(O)—N(R^{a})₂ group, wherein each R^{a} independently is hydrogen or aliphatic.

In any or all of the above embodiments, the peptide-based delivery agent has an amino acid sequence at least 80%, at least 85%, at least 90%, at least 95%, at least 96%, at least 97%, at least 98%, at least 99%, or 100% identical to any one of SEQ ID NOs: 272, 273, or 274 and wherein K at position 32 comprises a linking group selected from Formula A, B, or C, and wherein the linking group is attached to one or more DBCO groups.

Also disclosed herein are examples of a composition, comprising: the peptide-based delivery agent according to any or all of the above examples; and a therapeutic agent.

In any or all of the above examples, the therapeutic agent is covalently or non-covalently bound to the peptide-based delivery agent.

In any or all of the above examples, the therapeutic agent is a chemotherapeutic, a morpholino, a therapeutic antibody, an immune therapeutic, an antibiotic, an antidepressant, or a combination thereof.

In any or all of the above examples, the therapeutic agent is saporin, cisplatin, methotrexate, fluorouracil, doxorubicin, cyclophosphamide, chlorambucil, vinblastine, vincristine, docetaxel, or paclitaxel, chlorhexidine, triclosan, xylitol, or octadecene-1-amine hydrofluoride, 1-hexadecylamine hydrofluoride, Gefitinib, Lapatinib, Olaparib, mitomycin C, Sunitinib, Geftinib, Nintedanib, PD173074, Erdaftinib, Sorafenib, or a combination thereof.

In any or all of the above examples, the therapeutic agent is a morpholino.

In any or all of the above examples, the morpholino comprises a nucleic acid sequence at least 80%, at least 85%, at least 90%, at least 95%, at least 96%, at least 97%, at least 98%, at least 99%, or 100% identical to any one of SEQ ID NOs: 229, 240, 241, 242, 243, and 244.

In any or all of the above examples, (i) the peptide-based delivery agent comprises an antibody as the anchor group or the targeting group (ii) the therapeutic agent is a morpholino and wherein the morpholino is indirectly covalently bound to the peptide-based delivery agent through a linking group.

In any or all of the above examples, the composition further comprises an adjuvant, a carrier, a buffer, a detergent, or a combination thereof.

In any or all of the above examples, the composition is formulated for administration by injection, aerosol delivery, intranasal administration, oral administration, topical administration, or a combination thereof.

Also disclosed herein are examples of a method, comprising contacting a cell with the delivery agent and/or the composition according to any or all of the above examples.

In any or all of the above examples, the peptide-based delivery agent of the composition delivers the therapeutic agent to the cell's cytosol.

In any or all of the above examples, the cell is a non-human mammalian cell or a human cell.

In any or all of the above examples, the cell is contacted in vitro or in vivo.

In any or all of the above examples, contacting the cell with the composition induces lysis of an endosomal membrane following internalization of the therapeutic agent and the peptide-based delivery agent into the cell; or induces pore formation in an endosomal membrane following internalization of the therapeutic agent and the peptide-based delivery agent into the cell; or induces local disruption/destabilization of an endosomal membrane following internalization of the therapeutic agent and the peptide-based delivery agent into the cell; thereby delivering the therapeutic agent into the cytosol of the cell.

In any or all of the above examples, contacting the cell with the composition comprises contacting the cell with the therapeutic agent and the peptide-based delivery agent at the same time or at different times.

Also disclosed here are examples of a method, comprising administering to a subject a therapeutically effective amount of the delivery agent and/or the composition according to any or all of the above examples.

In any or all of the above examples, the subject is a human or a non-human mammal.

In any or all of the above examples, the therapeutically effective amount of the composition treats, ameliorates, or prevents the onset of a condition or disease selected from an inherited condition, a rare disease, a cancer, an immune condition, an infectious disease, a psychiatric disorder, a substance abuse disorder, or any combination thereof.

In any or all of the above examples, administering comprises injection, aerosol delivery, intranasal administration, oral administration, topical administration, or a combination thereof.

In any or all of the above examples, administering comprises providing the therapeutic agent and the peptide-based delivery agent to the subject at the same time or at different times.

In any or all of the above examples, administering comprises providing the therapeutic agent and the peptide-based delivery agent to the subject using the same route of administration or different routes of administration.

Also disclosed herein are examples of a method of identifying a therapeutic compound, comprising: contacting a cell with the peptide-based delivery agent of any or all of the above examples and one or more compounds; determining an effect of the one or more compounds on the contacted cell; and comparing the effect of the one or more compounds on the contacted cell to a control; wherein a differential effect of the one or more compounds on the contacted cell relative to the control indicates that the one or more compounds is a therapeutic compound.

In any or all of the above examples, the method further comprises determining an IC₅₀ value for the one or more compounds.

In any or all of the above examples, the method is a quantitative high-throughput screening method.

In any or all of the above examples, the method further comprises selecting the one or more compounds that have a differential effect on the contacted cell relative to the control.

In any or all of the above examples, the effect of the one or more compounds on the contacted cell comprises reduced survival of the contacted cell compared to the control; increased survival of the contacted cell compared to the control; induction of a phenotype of interest in the contacted cell compared to the control; increased expression of one or more genes in the contacted cell compared to the control; and/or decreased expression of one or more genes in the contacted cell compared to the control.

Disclosed herein are examples of a kit, comprising a container, wherein the container comprises the peptide-based delivery agent of any or all of the above examples or the composition of any one of any or all of the above examples, and wherein the container is selected from a syringe, vial, tube, ampule, capsule, or bottle.

In any or all of the above examples, the kit further comprises (i) instructions for administering or using the peptide-based delivery agent of any or all of the above examples or the composition of any one of any or all of the above examples; (ii) a container comprising a therapeutic agent; (iii) one or more administration devices; or (iv) any combination of (i)-(iii).

### VIII. Examples

The following examples are put forth so as to provide a complete disclosure and description of how to make and use examples of the peptide-based delivery agents and methods described herein and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Unless specified otherwise, the examples below include a delivery agent skeleton having the following structure:
[N-terminus group]-[Lytic]-[CL]-[Mask]-[K(LA)ₙ]-[C-terminus group],
   wherein:
The N-terminus group comprises either a fluorophore or a capping group as follows:
   Fluorophore (“Fluor”):

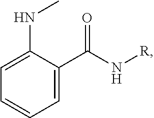

wherein R indicates the attachment to the delivery agent;
Capping Group (“Cap”):

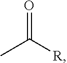

wherein R indicates the attachment to the delivery agent.
The lytic peptide group (“Lytic”) is ELLEELLEELLE (SEQ ID NO: 1);
The cleavable linker (“CL”) is GFGFVGG (SEQ ID NO: 3);
The mask peptide group (“Mask”) is QLLQQLLQQLLQ (SEQ ID NO: 4);
n is the number of anchor groups;
The anchor group (“LA”) structure comprises the specified lysine and a tail group having a structure of

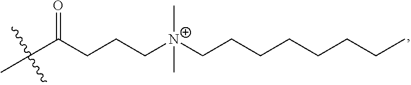 [SMILES: *C(=O)CCC[N+](C)(C)CCCCCCCC]

unless otherwise specified;
   Each tail group of the anchor group is independently covalently bound to the side chain of the specified lysine(s) in the peptide chain, forming a neutral amide linkage, such that “K(LA)” has a structure of

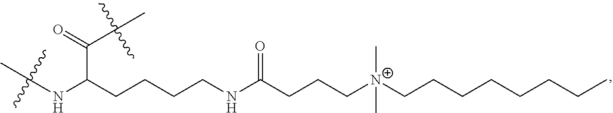 [SMILES: *NC(CCCCNC(=O)CCC[N+](C)(C)CCCCCCCC)C(*)=O]

unless otherwise specified.
The C-terminus group is G′, G-Nal-G′, or G-W-G′, where Nal is naphthylalanine and G′ is a modified glycine comprising a C-terminus of —C(O)NH₂ instead of —C(O)OH, unless otherwise specified.

Unless otherwise specified, the morpholino (also referred to in the Examples as the standard morpholino) used is:

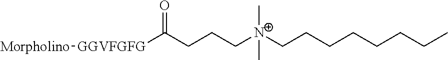

And, for examples that included a control, the control sample can consist of a morpholino (on its own), or it can include a delivery agent absent any morpholino, unless otherwise specified.

For in vivo studies in mice, the Morpholino sequence (“Morpholino” in the above structure, wherein it is “Morpholino 1” having a sequence 5′-GGCCAAACCTCGGCTTACCTGAAAT-3′; SEQ ID NO: 229) is complementary to mouse Dystrophin pre-mRNA for targeted deletion of exon 23. For cell culture in vitro studies, the Morpholino sequence (“Morpholino” in the above structure, wherein it is “Morpholino 2” having a sequence 5′-GCAATATGAAACCTCTTACCTCAGT-3′; SEQ ID NO: 230) targets an aberrant splice site interrupting the firefly luciferase gene stably transfected in Hela cells (Kang et al. Biochemistry 1998, 37, 18, 6235-6239).

Endo-Porter (Gene Tools, Philomath, Oregon) was used as a positive control in some examples. Endo-Porter is a peptide that was developed to facilitate the delivery of Morpholinos into cultured cells. Endo-Porter is an endosomal delivery agent; it is uncharged at neutral pH but becomes cationic when protonated in the low pH of the late endosome. Endo-Porter releases the contents of the acidic endosome into the cytosol; this has been shown for morpholinos as well as oxygen-responsive fluorescent probes. Endo-Porter is poorly soluble in water but is soluble in DMSO. Cultured cells are bathed with fresh medium containing the cargo to be delivered (e.g., morpholinos) and Endo-Porter solution, usually as a formulation in DMSO, is added to the culture medium. When the Endo-Porter enters the aqueous medium it aggregates into particles; the culture should be swirled immediately to disperse the EndoPorter and limit the size of the aggregates. Endo-Porter particles will settle slowly to the bottom of the culture container, bringing them into contact with adherent cells. If suspension cultures are to be delivered with Endo-Porter, the cultures should be gently and continuously swirled (as on a slow orbital shaker) to keep the particles in suspension (Moulton et al., J Drug Discov Develop and Deliv, 3 (2):1023, 2016).

Unless described otherwise, activity of the compounds administered to cell cultures in the examples below was measured using a luciferase reporter assay. Briefly, the HeLa LUC/705 cell line, a positive test system for antisense compounds, are obtained from University of North Carolina. Hela cells were stably transfected with a plasmid containing the Firefly luciferase gene interrupted with a mutated human beta-globin intron. The mutation (IVS2-705) causes aberrant splicing of Luciferase pre-mRNA and prevents translation. Morpholino oligo treatment, targeted to the splice site, corrects splicing and restores luciferase activity.

For the assay, cells were cultured in DMEM with glucose, glutamine, sodium pyruvate (ex. Corning 10-013-CV) with 10% Fetal Bovine Serum (ex. Corning 35-010-CV), and Penicillin/Streptomycin. In some cases, media was supplemented with glutamine (MP Biomedicals Cat #1680149). Cells were trypsinized with 0.25% trypsin-EDTA (ex. Corning 25-053-CI) for cell passage. Morpholinos (sequences provided herein, with certain examples being commercially available from Gene Tools LLC, Philomath OR, USA) with or without delivery agents were mixed with media and/or were added at various stock concentrations to 60-90% confluent Luc/705 HeLa cell plate wells containing media with serum; final solution concentrations of test/control compounds and different percentages of serum used are described in each Example. After an incubation time (16-22 hrs), cells were washed with Phosphate Buffered Saline (PBS 1×, Invitrogen/ThermoFisher Scientific) and lysed with reporter lysis buffer (Promega, Madison WI, USA). After centrifugation, luciferase activity was assessed from lysate supernatant using the Luciferase Assay System (E1501, Promega, Madison WI) in a luminometer (Turner Biosystems). In some cases, luciferase activity was assessed using Bright-Glo™ Luciferase Assay System (E2610, Promega, Madison WI) following the manufacturer's protocol and luminescence was measured in a Spectramax M3 microplate reader (Molecular Devices). In some examples, protein levels were quantitated for normalization using a protein colorimetric assay reagent (ex. Bio-Rad Laboratories Inc. kit #5000001, USA).

To evaluate cell toxicity and Saporin delivery, HeLa cells in 96-well clear bottom microplates (ex. Corning, Corning NY, USA) were treated with either Delivery Agent, Saporin protein (Millipore Sigma, S9896) dissolved in water, or both combined by dilution in DMEM media only and added to plate wells in amounts shown in the Examples for 1 hour. Treatment was removed and replaced with DMEM supplemented media with 10% FBS (culture media described above). After 24 hours, media was removed and cells washed with 1×PBS. Cells were then treated with 2 μM Calcein AM (Biotium, San Francisco CA, USA) for 30 minutes and fluorescence was measured according to the manufacturer's protocol in a SpectraMax fluorescence microplate reader (Molecular Devices).

A method for assessment of Morpholino in vivo delivery was previously published and adapted (Morcos et al. Biotechniques 2008 45:613-623). Successful Morpholino delivery results in deletion of dystrophin exon 23 sequence from mRNA. Studies were performed at Gene Tools LLC with wild-type C57Bl mice obtained from The Jackson Laboratory. Morpholino 1 (sequence 5′-GGCCAAACCTCGGCTTACCTGAAAT-3′; SEQ ID NO: 229; Gene Tools LLC) was dissolved in water and combined with delivery agent in water, and the mixture was i.v. injected once daily for 2 or 3 days before sacrificing the animal 1 day or more after the final injection. RNA from excised muscle tissue was isolated using the MELT Total Nucleic Acid Isolation System (Ambion, Inc., Austin, TX, USA). RT-PCR from total RNA was performed using the SuperScript One-Step RT-PCR System (Invitrogen). Forward (5′-TTCTGGATGCAGACTTTGTGGCCT-3′; SEQ ID NO: 238) and reverse (5′-AGGGCAGGCCATTCCTCTTTCA-3′; SEQ ID NO: 239) DNA primers (Integrated DNA Technologies, USA) spanned dystrophin exon 21 and exon 24. For RT-PCR reactions, conditions were: 55° C. for 30 min; 94° C. for 2 min; 30 cycles of 94° C. for 15 s, 57° C. for 30 s, 68° C. for 1 min; and final extension at 68° C. for 5 min. Predicted sizes for amplified cDNA product are: exon 23 present, 517 bp; exon 23 deletion, 304 bp. Additional details and explanations are described in the Examples.

Exemplary methods for synthesizing exemplary delivery agent examples according to the present disclosure and/or components thereof (e.g., exemplary cleavable linkers, exemplary lytic and/or mask peptide groups, exemplary anchor groups, and/or exemplary morpholino groups) are described below. The below methods can be modified (with the benefit of the present disclosure and/or general knowledge in the art) to make other delivery agent examples and/or components thereof contemplated by the disclosure. Suitable reagent, reaction condition, and/or delivery agent component modifications are recognizable to those in the art, particularly in view of the present disclosure and other information available pertaining to chemical synthesis and/or peptide synthesis.

### Exemplary Delivery Agent Synthesis (Peptide 88-8 from Example 11)

Resin activation: A resin is activated with an amino acid selected as the C-terminal amino acid in the backbone of the delivery agent (e.g., Gly). A Rink Amide ChemMatrix Resin (Gyros Protein Technologies Cat. #RCM-10-HL) was used in this example, but there are other resins that can be used, including but not limited to, Rink Amide MBHA resin (Millipore Sigma cat #85003), NovaSyn TGR Resin (Millipore Sigma Cat #855009), HMBA Resin (AAPPTec Cat #ROZ005, peptide is cleaved from resin with strong base).

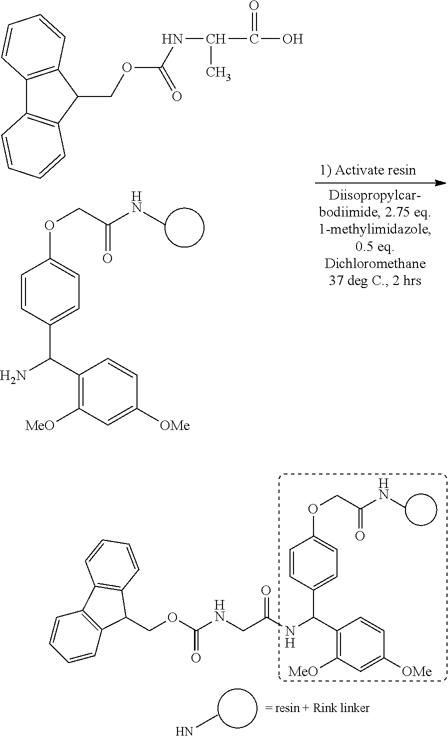

Delivery Agent Component Synthesis: The backbone of the delivery agent (including the lytic peptide group, the cleavable linker, the mask peptide group, and the amine-terminated glycine moiety) is built onto the Fmoc-protected glycine-functionalized resin from C-terminus to N-terminus, starting with a naphthylalanine group (Nal) and ending with glutamic acid.

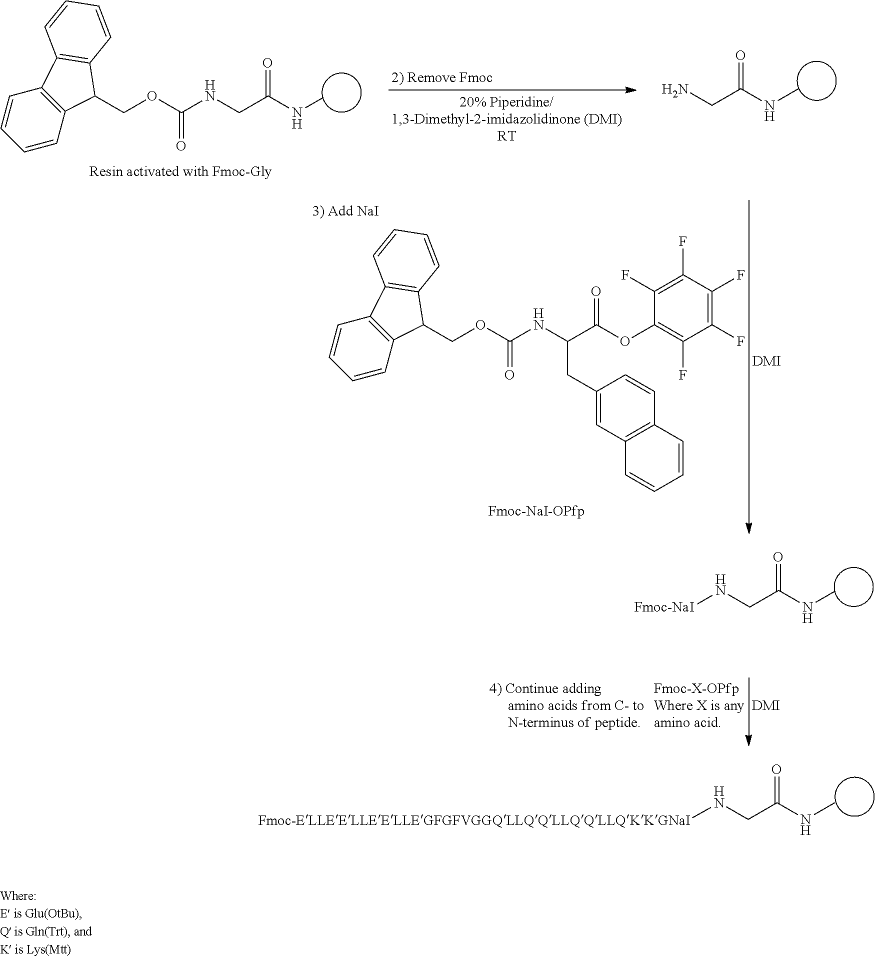

Deprotection and N-terminus Addition: The N-terminal Fmoc is then removed with 20% Piperidine/DMI, and the N-terminus cap/group is added.

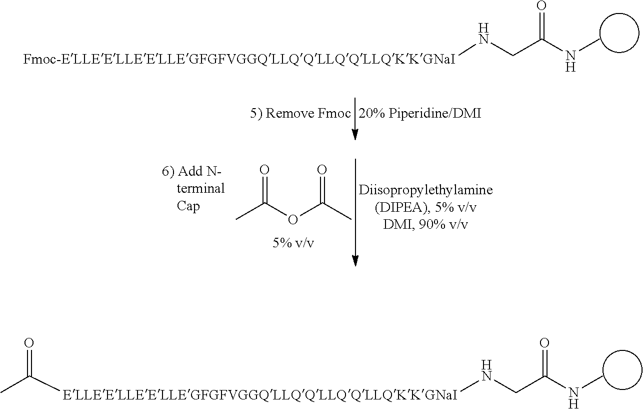

Anchor Group Addition: The protected lysine group(s) (K′=Lys(Mtt)) are then deprotected and lipid anchor group(s) are added to functionalize the side chain of the lysine moieties.

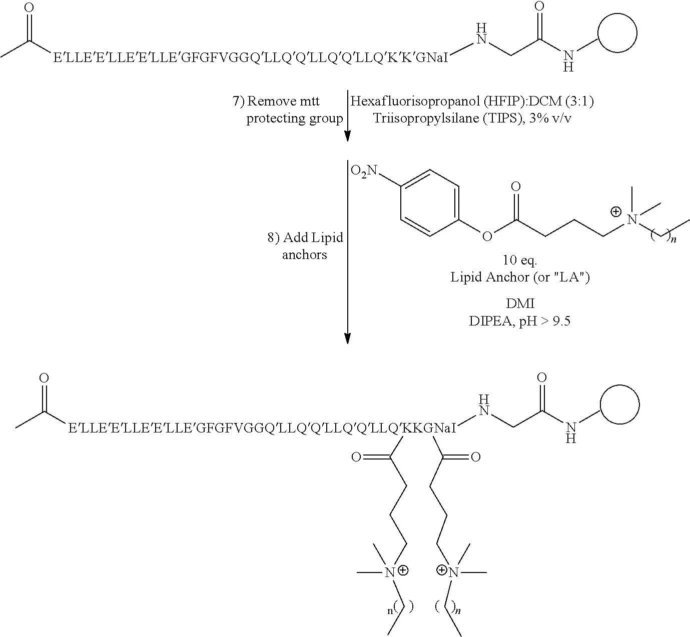

Anchor Group Synthesis: An activated anchor group is made using the method illustrated below. The anchor group is activated for coupling with the delivery agent skeleton using the bis(4-nitropheny) carbonate reagent.

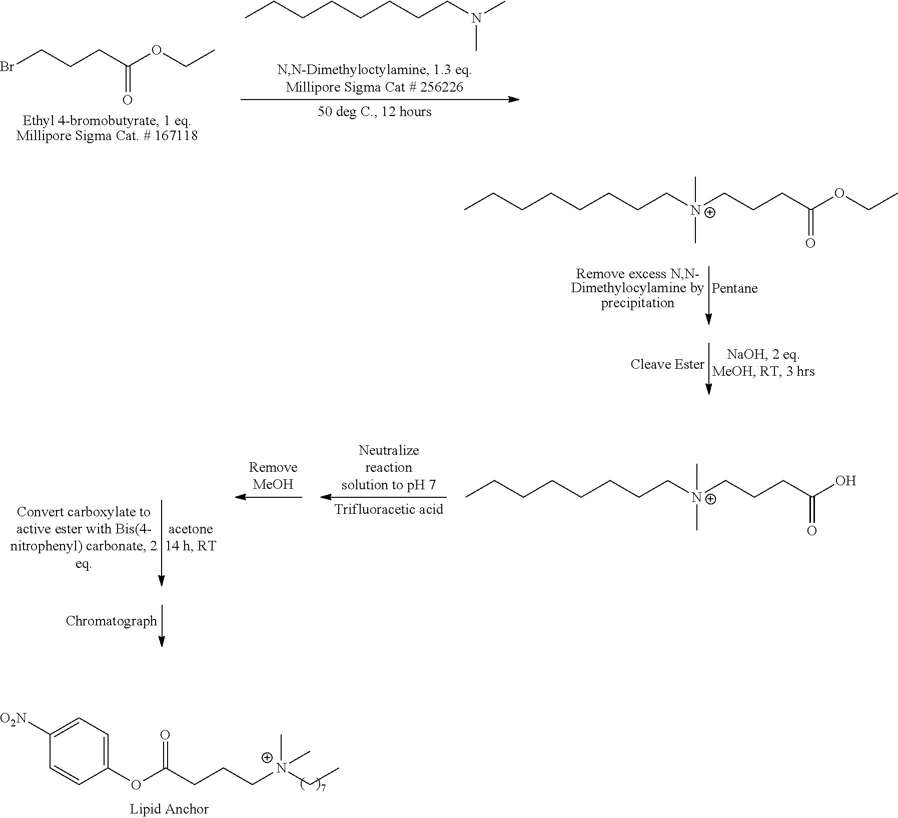

Resin Cleavage: The protective groups on the glutamic acid (E′) and glutamine (Q′) groups are then cleaved to reveal the free amino acids.

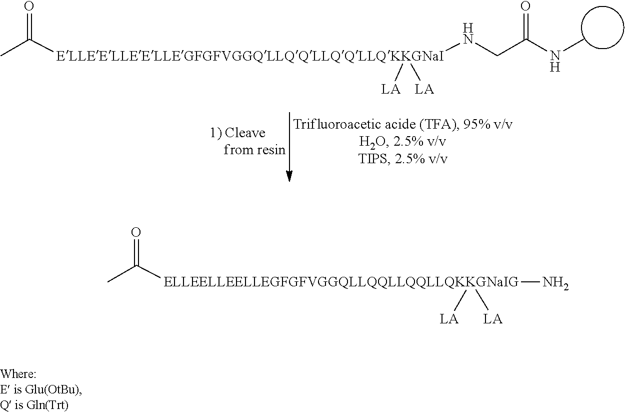

Exemplary Synthesis of Morpholino Conjugated Delivery Agent (98-1-MO Conjugate from Example 58)

Resin Cleavage: The resin is cleaved from a delivery agent comprising a Boc-protected lysine group (K″) attached to the resin, leaving an amine-terminated glycine moiety.

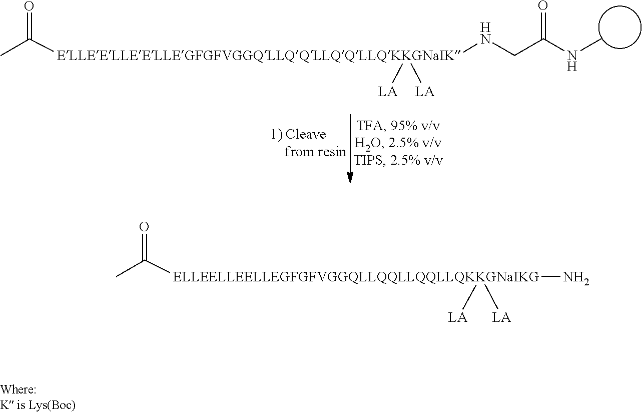

Chemical Linker Addition: A chemical linker group is added to the delivery agent at the now-unprotected lysine moiety via the lysine side chain. In this example, the chemical linker comprises a disulfide moiety and a constrained bicyclooctyne moiety to facilitate conjugation to a Morpholino using click chemistry.

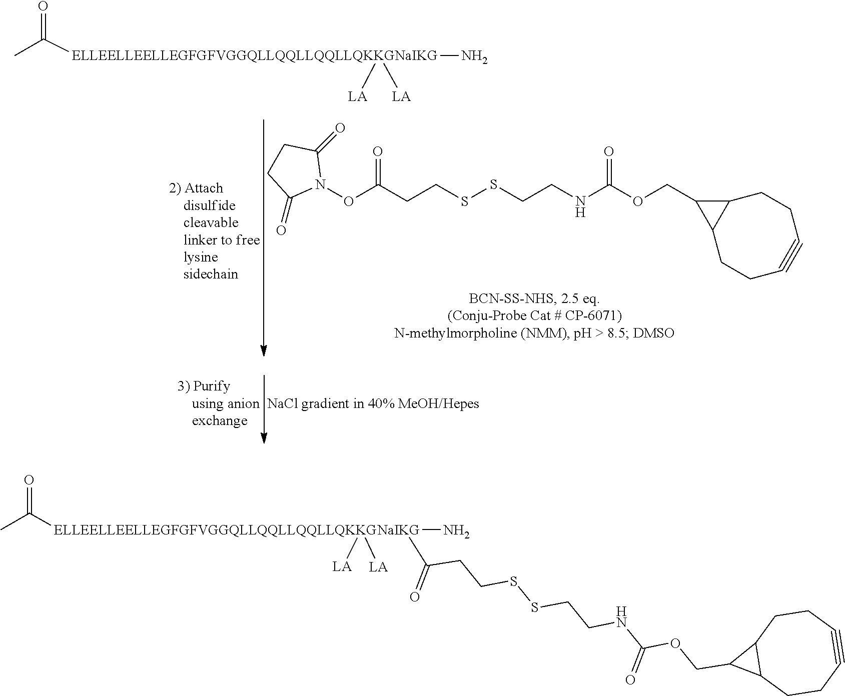

Morpholino Functionalization: A Morpholino group is functionalized with an azide-terminated PEG linker group to provide a Morpholino compound capable of undergoing click chemistry with the chemical linker group of the delivery agent shown above.

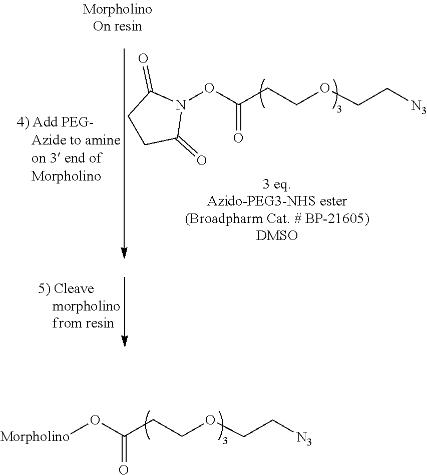

Click Chemistry Coupling: The functionalized Morpholino is coupled to the delivery agent using click chemistry conditions, thereby providing the Morpholino-bound delivery agent.

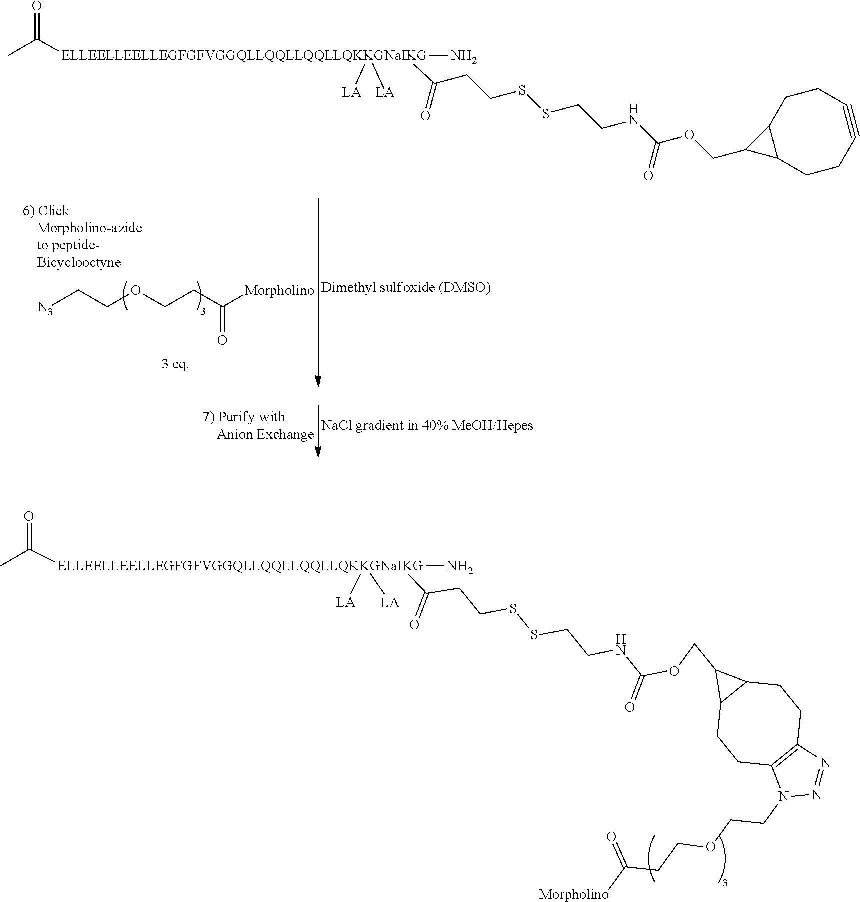

Exemplary Synthesis of Delivery Agent-Morpholino Conjugate with a 2nd GFGFVGG cleavable Linker

A delivery agent embodiment that is coupled to a Morpholino using chemical linker and a second cleavable linker is described in this example. The second cleavable linker is first built on the resin, followed by the rest of the delivery agent backbone.

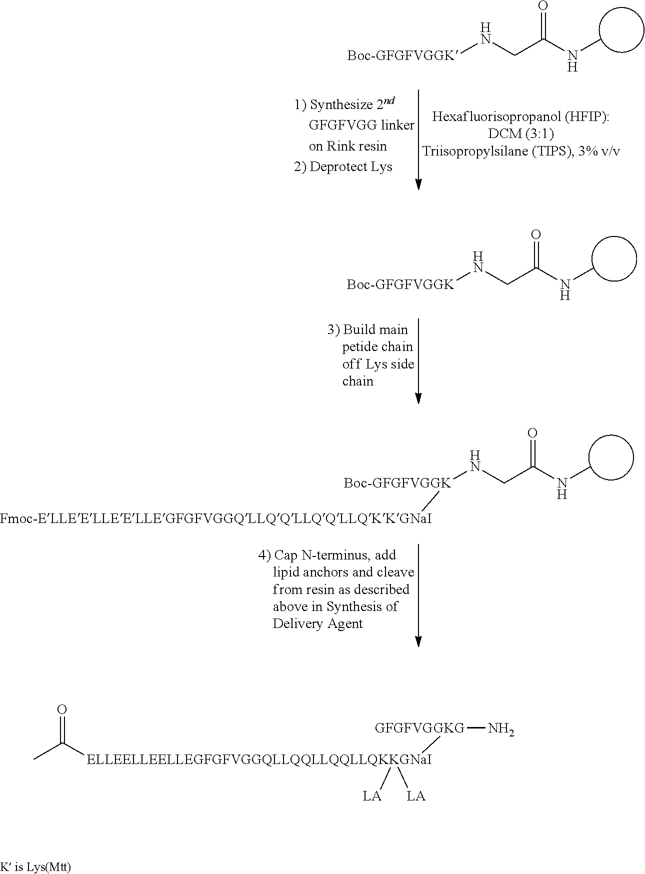

Morpholino Functionalization: Another functionalized Morpholino group is prepared by coupling a chemical linker group to the Morpholino to prepare it for coupling to the delivery agent illustrated above.

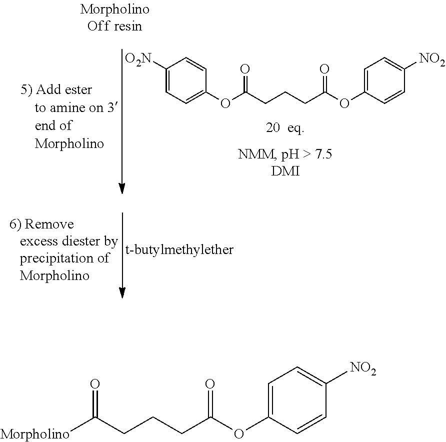

Morpholino-Delivery Agent Coupling: The functionalized Morpholino is then coupled to the delivery agent comprising the second cleavable linker such that the functionalized Morpholino is bound to an N-terminal glycine moiety of the second cleavable linker.

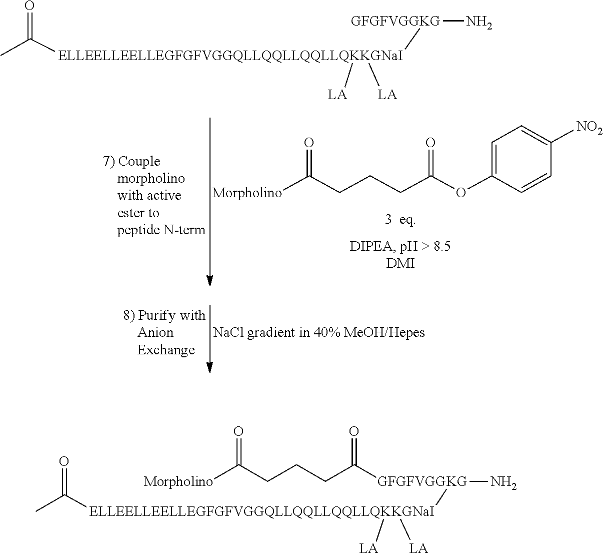

GalNac ligand Addition—Using the 217-2 GalNac embodiment as an example, a GalNac Ligand is added to the delivery agent at the lysine in position 35. The peptide is synthesized on resin as detailed previously including the addition of lipid anchors and an n-term acetyl group. During on-resin synthesis, this lysine is protected with a BOC group which is removed during peptide cleavage with TFA, leaving a free amine for coupling.

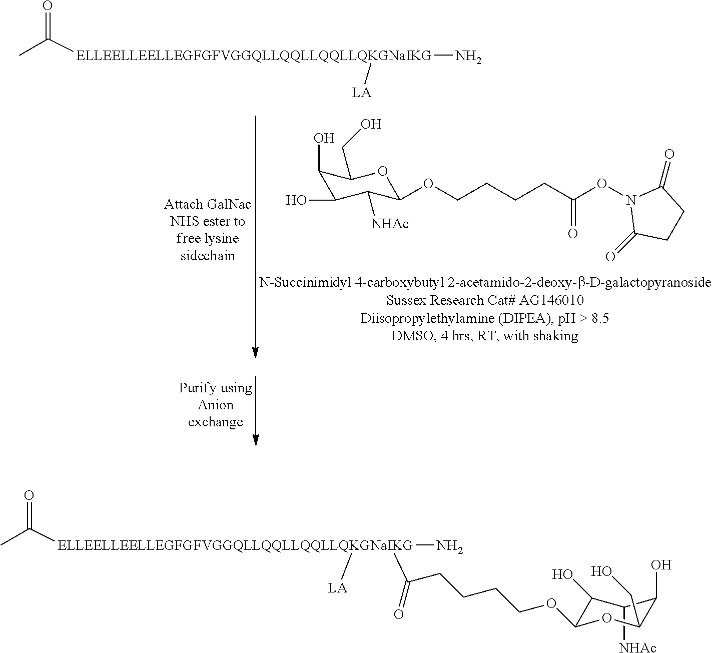

TrisGalNac ligand Addition—Using the 216-1 TrisGalNac embodiment as an example, the Tris-GalNac ligand is added to the lysine in position 32.

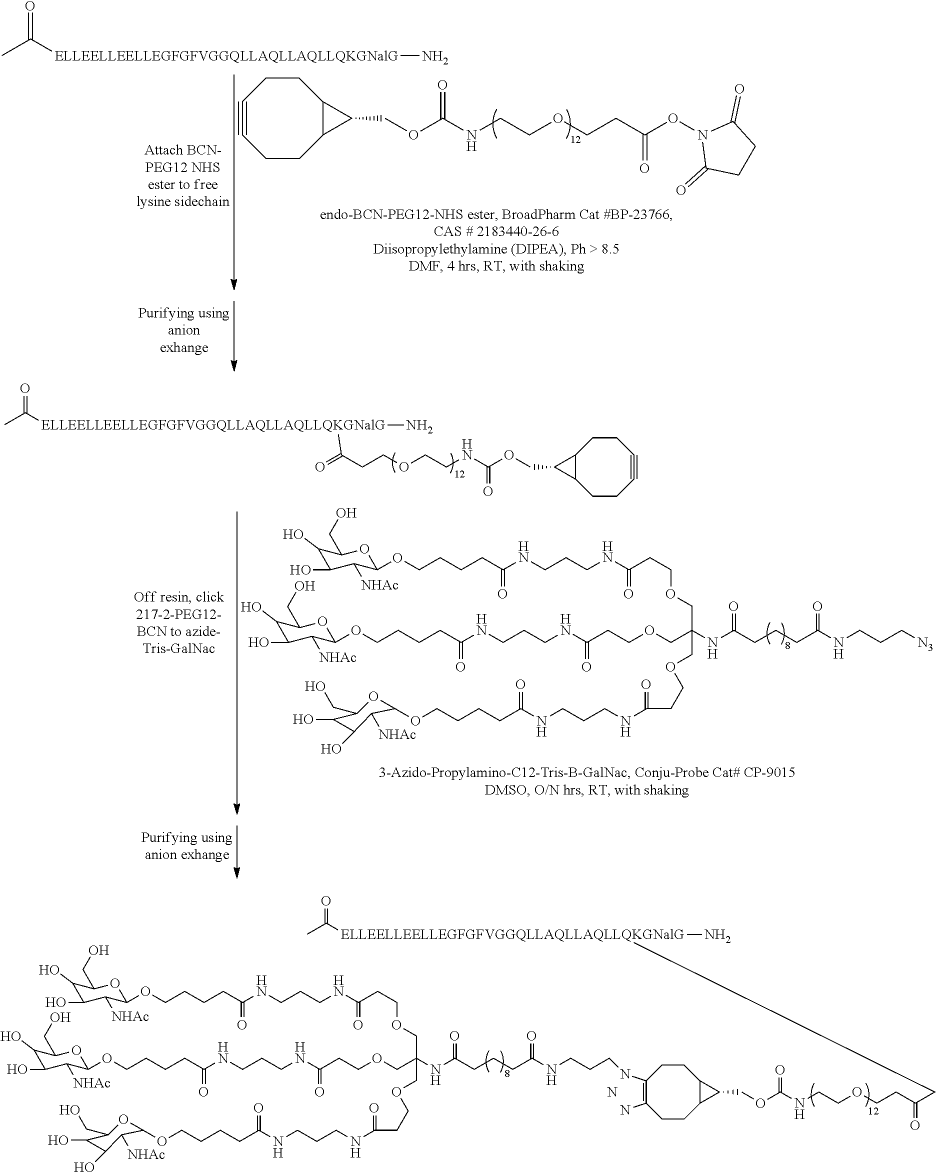

Evaluation of antibody-bound delivery agent activity in cells—To evaluate performance of delivery agent antibody conjugates, HeLa Luc/705 cells were either transfected or not transfected with a plasmid DNA vector that co-expresses Human transferrin receptor (hTFRC) and enhanced Green Fluorescent (EGFP) (pRP [Exp]-EGFP-EF1A>hTFRC [NM_003234.4], purchased from VectorBuilder Inc., Chicago IL, USA) using Lipofectamine 3000 transfection reagent (ThermoFisher Scientific, USA). After 24 hours, transfection reagent media or normal growth media was removed and replaced with treatment growth media containing 3 microMolar Morpholino (SEQ ID NO: 230) and either delivery agent antibody conjugates or control compounds (the antibody or delivery agent, unconjugated). After 20 hours of treatment (44 hours post-transfection), luciferase activity from Morpholino delivery was assessed using the Bright-Glo Luciferase Assay System; luminescence was measured using a Spectramax M3 microplate reader and reported as relative luminescence units (LUC). Treatment groups (transfected and untransfected) were blanked using untreated wells. Transfected cells in wells with 100 nanoMolar 216-1-PEG12BCN-antibody conjugate treatments were >50% dead and results were disregarded. Transfection performance was evaluated by measuring GFP fluorescence at 24 and 44 hours post-transfection in the microplate reader, and reported as relative fluorescence units (RFUs).
Synthesis of antibody-bound delivery agent—Using the 217-2 TGFR1 Ab embodiment as an example, a monoclonal antibody that binds Transferrin receptor was bound to the delivery agent. The Transferrin Receptor Monoclonal antibody (MEM-189, Catalog #MA1-21562) was purchased from ThermoFisher Scientific and activated with azides using the Invitrogen™ SiteClick™ Antibody Azido Modification Kit, also purchased from ThermoFisher (Catalog number: S20026). The protocol included with the SiteClick™ kit was used for the activation. The peptide is synthesized on resin as detailed previously including the addition of an n-term acetyl group. The lysine is deprotected in the cleavage of the delivery agent from the resin with TFA, leaving a free amine for coupling. An endo-BCN-PEG12-NHS ester purchased from BroadPharm (Product #BP-23766, CAS #2183440-26-6) was bound to the lysine side chain as detailed below. The azide-antibody and BCN-PEG12-peptide were then clicked to form the final 217-2 TGFR1 Ab.

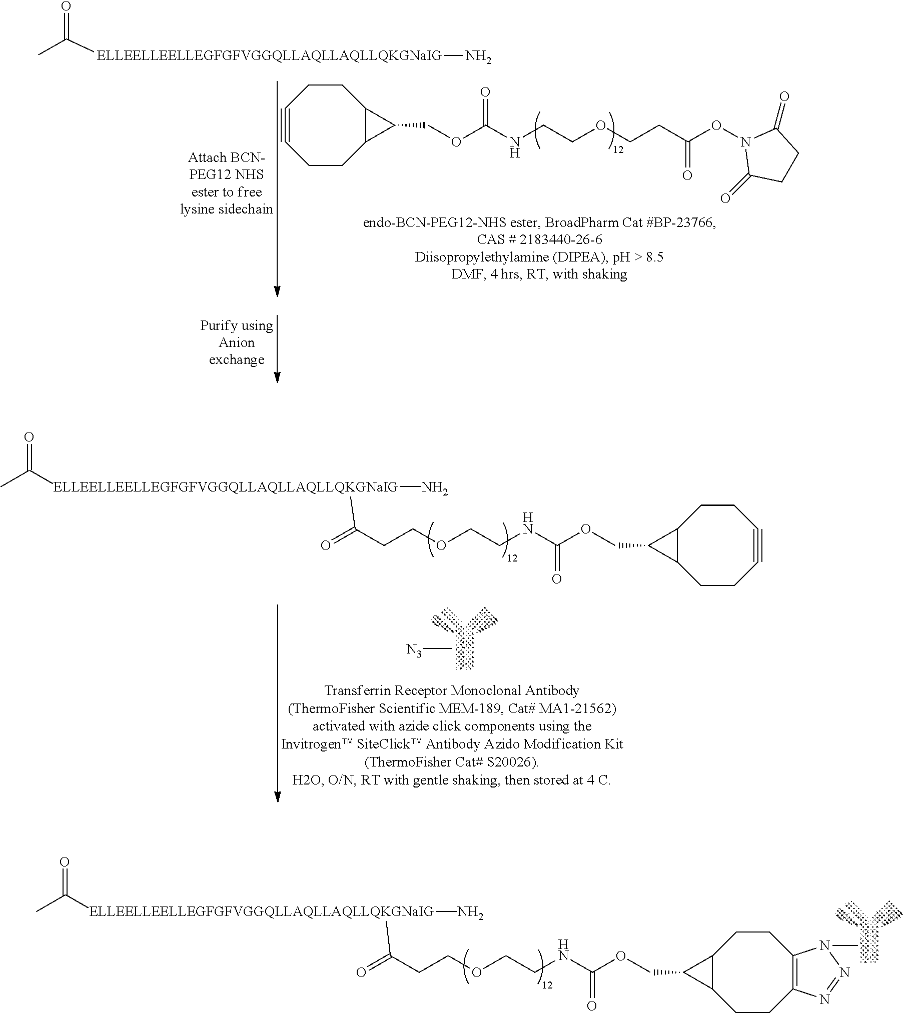

Delivery Agent-Antibody-Morpholino Synthesis—The delivery agent peptide is conjugated to a Morpholino through a disulfide bond cleavable linker at the Cys residue at position 35 on the peptide. The delivery agent is also conjugated to the transferrin receptor antibody via the Lys at position 32. A synthesis is described below.

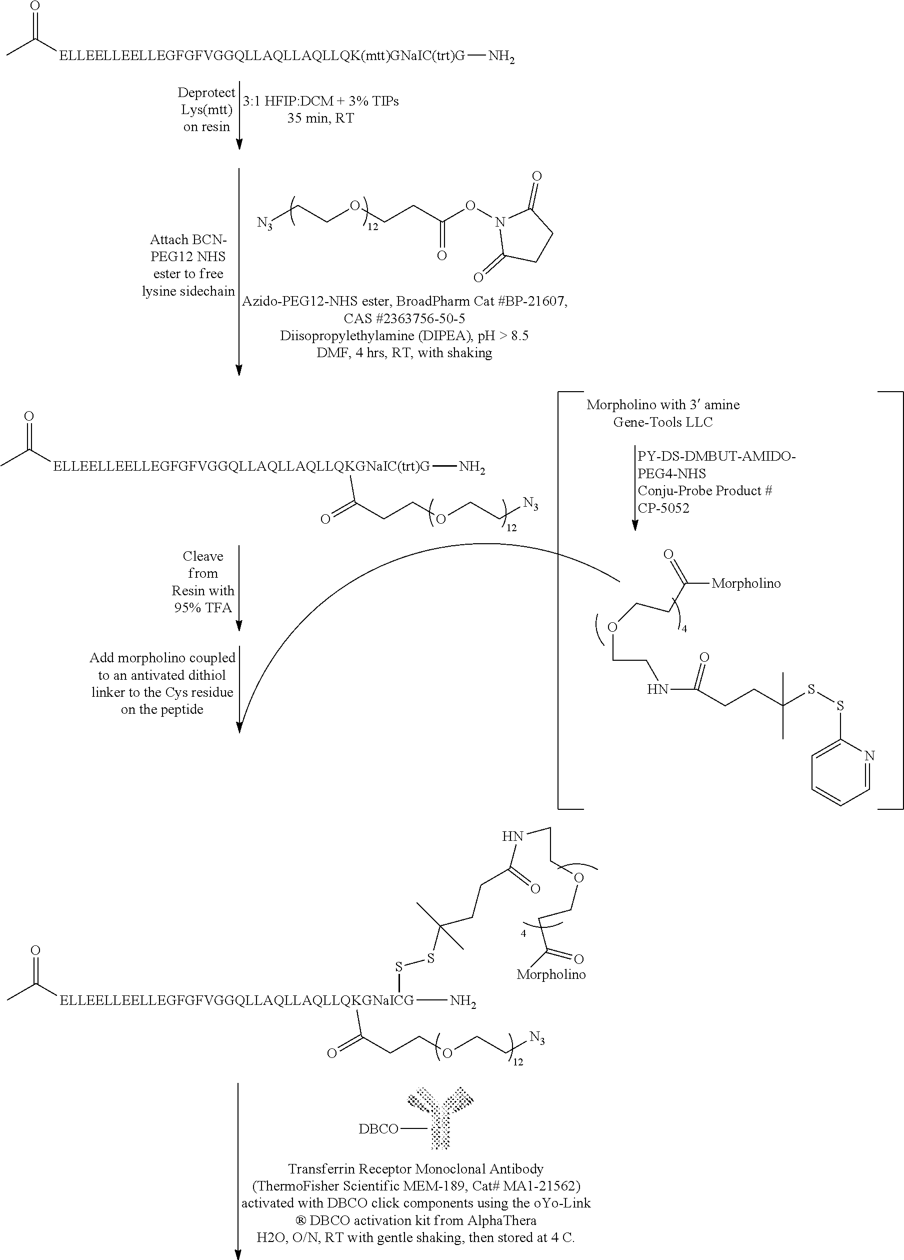

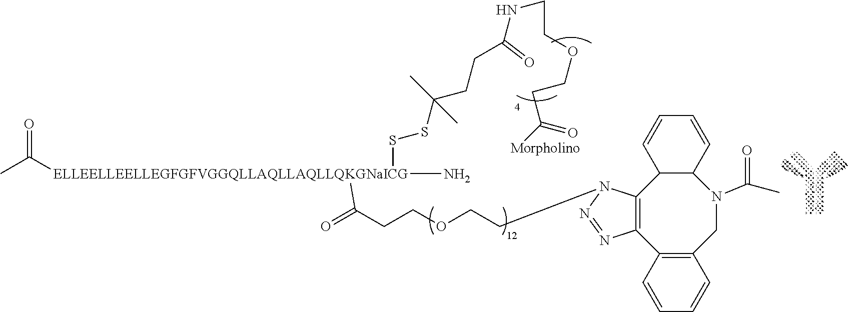

Biological Assay—To evaluate performance of delivery agent-antibody-Morpholino conjugates, HeLa Luc/705 cells are either transfected or not transfected with a plasmid DNA vector that co-expresses Human transferrin receptor (hTFRC) and enhanced Green Fluorescent (EGFP) (pRP [Exp]-EGFP-EF1A>hTFRC[NM_003234.4], to be from VectorBuilder Inc., Chicago IL, USA) using Lipofectamine 3000 transfection reagent (ThermoFisher Scientific, USA). After 24 hours, transfection reagent media or normal growth media are removed and replaced with treatment growth media containing delivery agent-antibody-Mopholino conjugates or control compounds (the antibody or delivery agent, unconjugated) with unconjugated Morpholino (SEQ ID NO: 230). After 20 hours of treatment (44 hours post-transfection), luciferase activity from Morpholino delivery is assessed using the Bright-Glo Luciferase Assay System; luminescence is measured using a Spectramax M3 microplate reader and reported as relative luminescence units (LUC). Treatment groups (transfected and untransfected) is blanked using untreated wells. Transfection performance is evaluated by measuring GFP fluorescence at 24 and 44 hours post-transfection in the microplate reader, and reported as relative fluorescence units (RFUs).

### Example 1

In this example, two different delivery agent examples were evaluated wherein the lytic peptide group comprised (i) one leucine at the end of the lytic peptide group sequence (the end closest to the C-terminus group); and (ii) two leucines at the end of the lytic peptide group sequence (the end closest to the C-terminus group). The specific lytic peptide group sequences are provided below:
23-6: fluor-[ELL EEL LEE LLE L]-[CL]-[Mask]-[2×K(LA)]-[G]
23-7: fluor-[ELL EEL LEE LLE LL]-[CL]-[Mask]-[2×K(LA)]-[G′]
SEQ ID NO: 16: ELLEELLEELLEL, wherein the N-terminal E at position 1 comprises the “fluor” group described above.
SEQ ID NO: 17: ELLEELLEELLELL, wherein the N-terminal E at position 1 comprises the “fluor” group described above.
SEQ ID NO: 18: ELLEELLEELLELGFGFVGGQLLQQLLQQLLOKKG, wherein the N-terminal E at position 1 comprises the “fluor” group described above, each K is functionalized with an LA group, and the C-terminal G at position 35 is G′ as described above.
SEQ ID NO: 19: ELLEELLEELLELLGFGFVGGQLLQQLLQQLLOKKG, wherein the N-terminal E at position 1 comprises the “fluor” group described above, each K is functionalized with an LA group, and the C-terminal G at position 36 is G′ as described above.

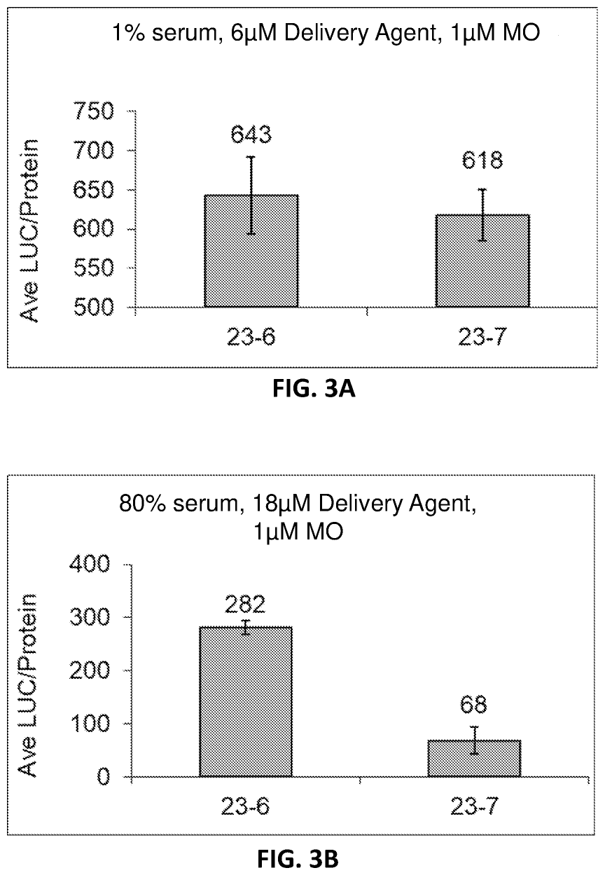

In this example, delivery agent examples were evaluated wherein either one or two leucines were added to the c-terminus of the lytic peptide. Both examples retained some activity under low and high serum conditions. Results are illustrated in FIGS. 3A and 3B.

### Example 2

In this example, delivery agent examples were evaluated wherein leucines of the lytic peptide group sequence were replaced with isoleucines. Endoporter (EP) was used as a positive control. Also evaluated was a delivery agent embodiment wherein two leucines were added to the C-terminus group of the lytic peptide group without a masking peptide sequence. The specific delivery agent structures evaluated in this example are provided below.
23-10: fluor-[EII EEI IEE IIE]-[CL]-[2×K(LA)]-[G′]
23-16: fluor-[Lytic]-[CL]-[2×K(LA)]-[G′]
23-20: fluor-[ELL EEL LEE LLE LL]-[CL]-[2×K(LA)]-[G′]
Positive control: Endoporter (EP)
SEQ ID NO: 20: EIIEEIIEEIIE, wherein the N-terminal E at position 1 comprises the “fluor” group described above,
SEQ ID NO: 21: EIIEEIIEEIIEGFGFVGGQLLOOLLQQLLOKKG, wherein the N-terminal E at position 1 comprises the “fluor” group described above, each K is functionalized with an LA group, and the C-terminal G at position 36 is G′ as described above.
SEQ ID NO: 22: ELLEELLEELLEGFGFVGGKKG, wherein the N-terminal E at position 1 comprises the “fluor” group described above, each K is functionalized with an LA group, and the C-terminal G at position 22 is G′ as described above.
SEQ ID NO: 23: ELLEELLEELLELLGFGFVGGKKG, wherein the N-terminal E at position 1 comprises the “fluor” group described above, each K is functionalized with an LA group, and the C-terminal G at position 24 is G′ as described above.

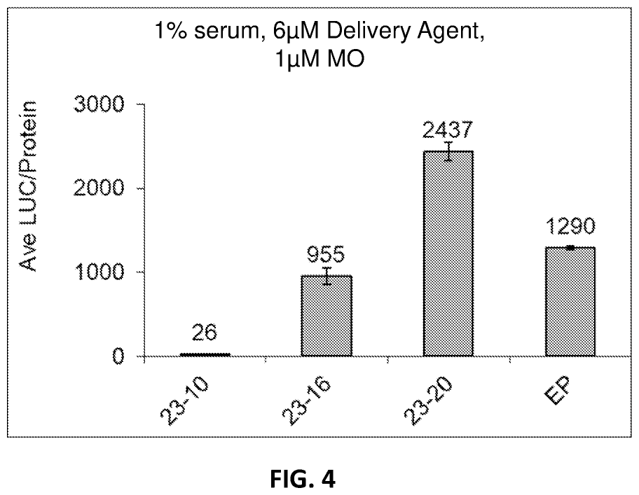

In this example, it was found that replacing leucines with isoleucines in the lytic peptide group attenuated activity, but whether or not this rendered the delivery agent completely inactive was not determined. Adding two leucines to the C-terminus of the lytic peptide group (delivery agent 23-20) increased activity relative to an exemplary delivery agent according to the present disclosure; however, in some tests, this lytic peptide group did not perform as well in an embodiment that also comprised a mask peptide group. Results are shown in FIG. 4.

### Example 3

In this example, the following delivery agent examples were evaluated for activity and/or toxicity.
30-2: fluor-[ELL QEL LQE LLE]-[CL]-[Mask]-[2×K(LA)]-[G′]
30-4: fluor-[ELL QEL LQE LLQ]-[CL]-[Mask]-[2×K(LA)]-[G′](poor water solubility)
30-6: fluor-[QLL EQL LEE LLE]-[CL]-[Mask]-[2×K(LA)]-[G′]0.30-10: fluor-[ELL EQL LQE LLE]-[CL]-[Mask]-[2×K(LA)]-[G′]
30-14: fluor-[QLL EEL LEE LLQ]-[CL]-[Mask]-[2×K(LA)]-[G′]
30-16: fluor-[ELL QEL LEQ LLE]-[CL]-[Mask]-[2×K(LA)]-[G′]
30-18: fluor-[(Lytic) D]-[CL]-[Mask]-[2×K(LA)]-[G′]
30-22: fluor-[Lytic]-[CL]-[Mask]-[2×K(LA)]-[G]
SEQ ID NO: 24: ELLQELLQELLE, wherein the N-terminal E at position 1 comprises the “fluor” group described above.
SEQ ID NO: 25: ELLQELLQELLEGFGFVGGQLLOOLLQQLLOKKG, wherein the N-terminal E at position 1 comprises the “fluor” group described above, each K is functionalized with an LA group, and the C-terminal G at position 34 is G′ as described above.
SEQ ID NO: 26: ELLQELLQELLQ, wherein the N-terminal E at position 1 comprises the “fluor” group described above.
SEQ ID NO: 27: ELLQELLQELLQGFGFVGGQLLQQLLQQLLOKKG, wherein the N-terminal E at position 1 comprises the “fluor” group described above, each K is functionalized with an LA group, and the C-terminal G at position 34 is G′ as described above.
SEQ ID NO: 28: QLLEQLLEELLE, wherein the N-terminal Q at position 1 comprises the “fluor” group described above.
SEQ ID NO: 29: QLLEQLLEELLEGFGFVGGQLLQQLLQQLLOKKG, wherein the N-terminal Q at position 1 comprises the “fluor” group described above, each K is functionalized with an LA group, and the C-terminal G at position 34 is G′ as described above.
SEQ ID NO: 30: ELLEQLLQELLE, wherein the N-terminal E at position 1 comprises the “fluor” group described above.
SEQ ID NO: 31: ELLEQLLQELLEGFGFVGGOLLQQLLQQLLOKKG, wherein the N-terminal E at position 1 comprises the “fluor” group described above, each K is functionalized with an LA group, and the C-terminal G at position 34 is G′ as described above.
SEQ ID NO: 32: QLLEELLEELLQ, wherein the N-terminal Q at position 1 comprises the “fluor” group described above.
SEQ ID NO: 33: QLLEELLEELLQGFGFVGGQLLQQLLQQLLOKKG, wherein the N-terminal Q at position 1 comprises the “fluor” group described above, each K is functionalized with an LA group, and the C-terminal G at position 34 is G′ as described above.
SEQ ID NO: 34: ELLQELLEQLLE, wherein the N-terminal E at position 1 comprises the “fluor” group described above.
SEQ ID NO: 35: ELLQELLEQLLEGFGFVGGQLLQQLLQQLLOKKG, wherein the N-terminal E at position 1 comprises the “fluor” group described above, each K is functionalized with an LA group, and the C-terminal G at position 34 is G′ as described above.
SEQ ID NO: 36: ELLEELLEELLE, comprising D amino acids and wherein the N-terminal E at position 1 comprises the “fluor” group described above.
SEQ ID NO: 37: ELLEELLEELLEGFGFVGGQLLQQLLQQLLOKKG, where the lytic peptide group comprises D amino acids and wherein the N-terminal E at position 1 comprises the “fluor” group described above, each K is functionalized with an LA group, and the C-terminal G at position 34 is G′ as described above.
SEQ ID NO: 246: ELLEELLEELLEGFGFVGGOLLQQLLQQLLOKKG, wherein the N-terminal E at position 1 comprises the “fluor” group described above, each K is functionalized with an LA group, and the C-terminal G at position 34 comprises a C-terminus modified with a t-butyl amine group)

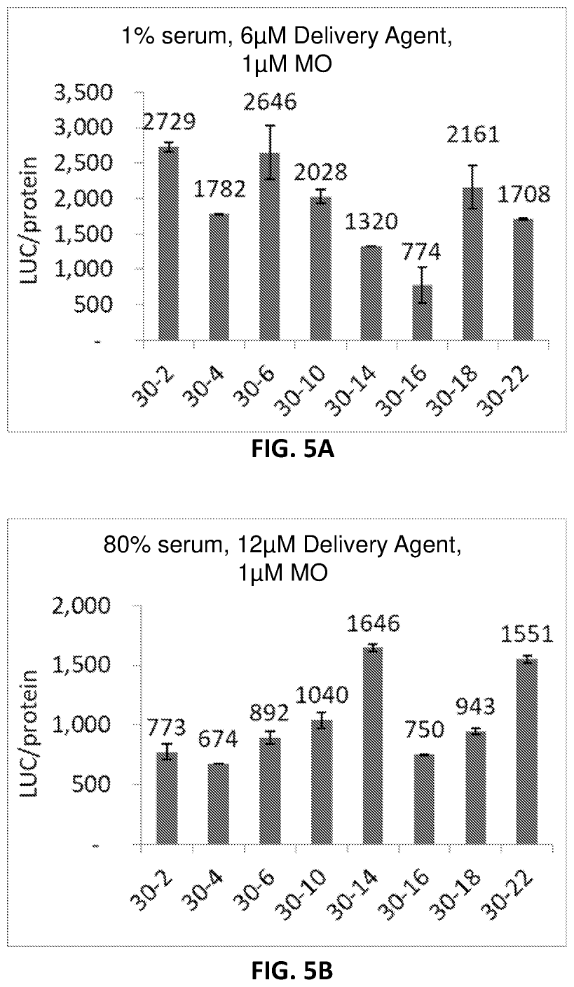

All of the above delivery agent examples showed activity; however, delivery agents 30-14 and 30-16 were toxic. The lytic peptide group of delivery agent 30-18 comprises D-amino acids. The activity for this compound was not much below the control (delivery agent 30-22) in either the 1% or 80% serum tests, which suggests that the chirality of amino acids used in the lytic peptide group may not have to match the Mask amino acid chirality in particular examples. It also was observed in some examples that three or fewer glutamates reduced water solubility. For example, delivery agent 30-4 had three glutamates in the lytic peptide group sequence and had poor water solubility; however, this delivery agent was not purified, and it currently is believed that purification may increase solubility. Results are shown in FIGS. 5A and 5B.

### Example 4

In this example, a delivery agent embodiment comprising a 16-mer lytic peptide group and a corresponding 16-mer masking peptide sequence were evaluated as compared to an embodiment comprising a 12-mer lytic peptide group and a corresponding 12-mer masking peptide sequence. The assessment was repeated, wherein the second assessment was conducted at lower concentrations. The specific delivery agent examples are described below.

### First Assessment:

40-1: fluor-[ELL EEL LEE LLE ELL E]-[CL]-[QLL QQL LQQ LLQ QLL Q]-[2×K(LA)]-[G′](16-mer)
40-46: fluor-[Lytic]-[CL]-[Mask]-[2×K(LA)]-[G] (12-mer)

### Second Assessment:

42-9: fluor-[ELL EEL LEE LLE ELL E]-[CL]-[QLL QQL LQQ LLQ QLL Q]-[2×K(LA)]-[G′] (16-mer)
42-39: fluor-[Lytic]-[CL]-[Mask]-[2×K(LA)]-[G′] (12-mer)
SEQ ID NO: 38: ELLEELLEELLEELLE, wherein the N-terminal E at position 1 comprises the “fluor” group described above.
SEQ ID NO: 39: ELLEELLEELLEELLEGFGFVGGQLLQQLLQQLLQQLLOKKG, wherein the N-terminal E at position 1 comprises the “fluor” group described above, each K is functionalized with an LA group, and the C-terminal G at position 42 is G′ as described above.

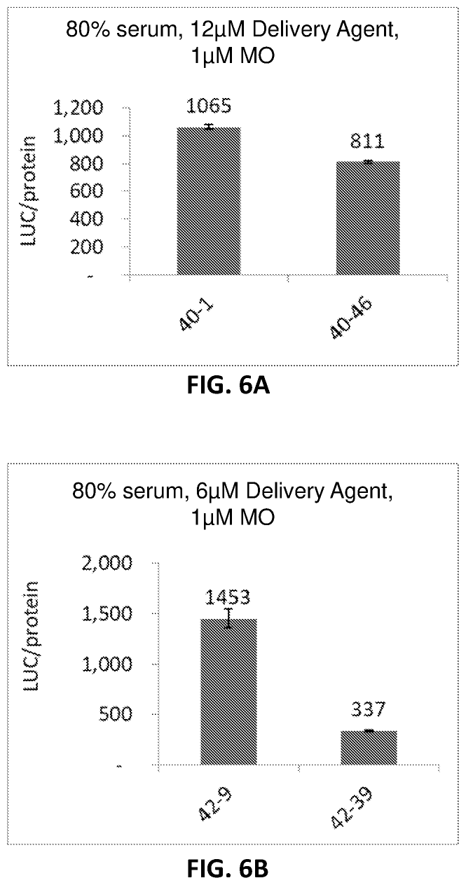

As shown in FIG. 6A, delivery agent 40-1, the 16-mer “lytic-cleavable linker-mask” sequence, had 1.3-fold greater activity than the normal 12-mer “lytic-cleavable linker-mask” sequence. The experiment was repeated with lower concentrations and showed a larger difference in activity (see FIG. 6B). Without being limited to a particular theory, it currently is believed that 12 μM delivery agent may have been close to saturating Morpholino activity.

### Example 5

In this example, a delivery agent embodiment comprising a 16-mer lytic peptide group and a corresponding 16-mer masking peptide sequence were evaluated as compared to an embodiment comprising a 12-mer lytic peptide group and a corresponding 12-mer masking peptide sequence. The assessment was conducted in 1% serum at 3 μM and was repeated. The specific delivery agent examples are described below.

### First Assessment:

40-1: fluor-[ELL EEL LEE LLE ELL E]-[CL]-[QLL QQL LQQ LLQ QLL Q]-[2×K(LA)]-[G′] (16-mer)
40-46: fluor-[Lytic]-[CL]-[Mask]-[2×K(LA)]-[G] (12-mer)

### Second Assessment:

42-9: fluor-[ELL EEL LEE LLE ELL E]-[CL]-[QLL QQL LQQ LLQ QLL Q]-[2×K(LA)]-[G′] (16-mer)
42-39: fluor-[Lytic]-[CL]-[Mask]-[2×K(LA)]-[G] (12-mer)

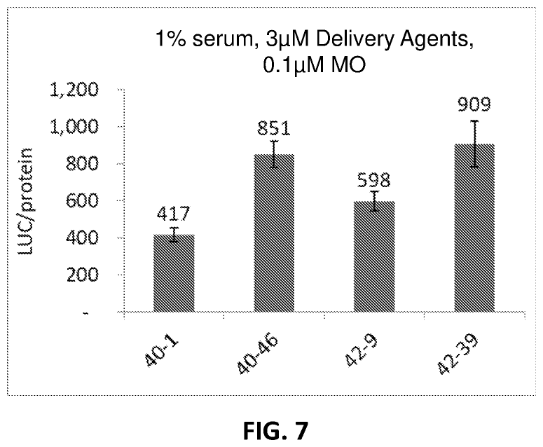

As shown in FIG. 7, the 16-mer delivery agent examples exhibited lower activity than the 12-mer “lytic-cleavable linker-mask” sequences, suggesting that the longer “lytic-cleavable linker-mask” sequence length can confer greater activity in high serum, but may exhibit lower activity in low serum in some aspects of the disclosure.

### Example 6

In this example, Helical Wheel diagrams were prepared for a delivery agent embodiment and a compound having lytic and mask peptide groups that did not comprise an “XYYX” pattern; these examples are described below.
23-26: fluor-[Lytic]-[CL]-[Mask]-[2×K(LA)]-[G]
50-22: fluor-[LEELLEE LEELLEE]-[CL]-[LQQLLQQ LQQLLQQ]-[2×K(LA)]-[G′]
SEQ ID NO: 40: LEELLEELEELLEE, wherein the N-terminal L at position 1 comprises the “fluor” group described above.
SEQ ID NO: 41: LQQLLQQLQQLLQQ
SEQ ID NO: 42: LEELLEELEELLEEGFGFVGGLQQLLQQLQQLLQQKKG, wherein the N-terminal L at position 1 comprises the “fluor” group described above, each K is functionalized with an LA group, and the C-terminal G at position 38 is G′ as described above.

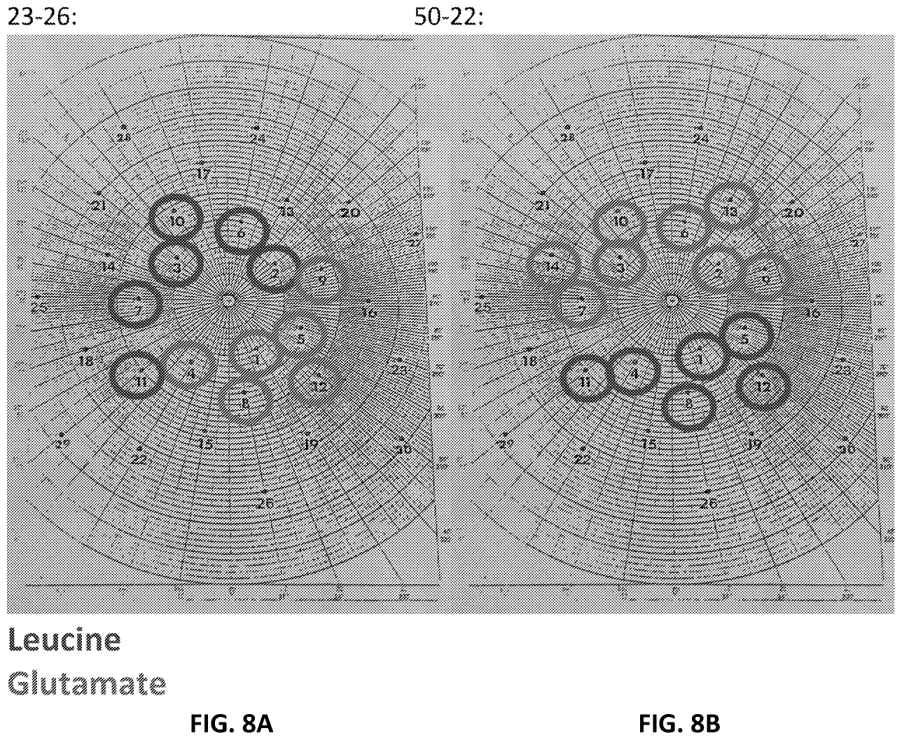

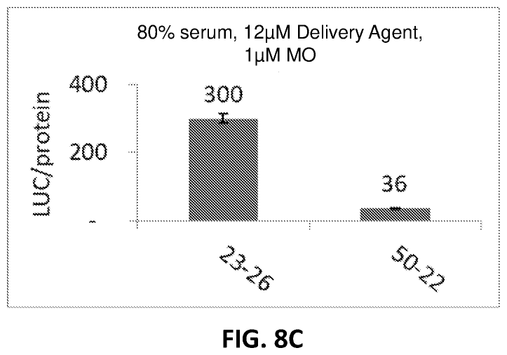

The Helical Wheel diagrams are shown in FIGS. 8A and 8B and further results are shown in FIG. 8C. As can be seen, each example retains glutamates on one side of the helix and leucines on the other; however, delivery agent 23-26 included an “XYYX” patterning (where X=E, D or Q and Y=L) which currently is believed to facilitate “lytic-cleavable linker-mask” sequence formation in particular examples.

### Example 7

In this example, modified delivery agents comprising glutamates at the N- or C-terminus of the lytic peptide group sequence were evaluated. A negative control of a morpholino alone was used, along with a positive control (exemplary delivery agent 23-26). The specific compounds evaluated in this example are described below.
Negative Control: Morpholino only
Positive Control: 23-26: fluor-[Lytic]-[CL]-[Mask]-[2×K(LA)]-[G′]
51-11: fluor-[EEE]-[ELL EEL LEE LLE ELL E]-[CL]-[QLL QQL LOQ LLQ QLL Q]-[2×K(LA)]-[G′]
51-12: fluor-[EE]-[ELL EEL LEE LLE ELL E]-[E]-[CL]-[QLL QQL LQQ LLQ QLL Q]-[2×K(LA)]-G′]
51-13: fluor-[E]-[ELL EEL LEE LLE ELL E]-[EE]-[CL]-[QLL QQL LQQ LLQ QLL Q]-[2×K(LA)]-[G]
51-14: fluor-[EE]-[ELL EEL LEE LLE ELL E]-[CL]-[QLL QQL LQQ LLQ QLL Q]-[2×K(LA)]-[G′]
51-15: fluor-[ELL EEL LEE LLE ELL E]-[EE]-[CL]-[QLL QQL LQQ LLQ QLL Q]-[2×K(LA)]-[G′]
51-16: fluor-[E]-[ELL EEL LEE LLE ELL E]-[E]-[CL]-[QLL QQL LQQ LLQ QLL Q]-[2×K(LA)]-[G]
51-17: fluor-[E]-[ELL EEL LEE LLE ELL E]-[CL]-[QLL QQL LQQ LLQ QLL Q]-[2×K(LA)]-[G′]
51-18: fluor-[ELL EEL LEE LLE ELL E]-[E]-[CL]-[QLL QQL LQQ LLQ QLL Q]-[2×K(LA)]-[G′]
SEQ ID NO: 43: EEEELLEELLEELLEELLE, wherein the N-terminal E at position 1 comprises the “fluor” group described above.
SEQ ID NO: 44: EEELLEELLEELLEELLEE, wherein the N-terminal E at position 1 comprises the “fluor” group described above.
SEQ ID NO: 45: EELLEELLEELLEELLEEE, wherein the N-terminal E at position 1 comprises the “fluor” group described above.
SEQ ID NO: 46: EEELLEELLEELLEELLE, wherein the N-terminal E at position 1 comprises the “fluor” group described above.
SEQ ID NO: 47: ELLEELLEELLEELLEEE, wherein the N-terminal E at position 1 comprises the “fluor” group described above.
SEQ ID NO: 48: EELLEELLEELLEELLEE, wherein the N-terminal E at position 1 comprises the “fluor” group described above.
SEQ ID NO: 49: EELLEELLEELLEELLE, wherein the N-terminal E at position 1 comprises the “fluor” group described above.
SEQ ID NO: 50: ELLEELLEELLEELLEE, wherein the N-terminal E at position 1 comprises the “fluor” group described above.
SEQ ID NO: 51: QLLQQLLQQLLQQLLQ
SEQ ID NO: 52: EEEELLEELLEELLEELLEGFGFVGGQLLQQLLQQLLQQLLQKKG, wherein the N-terminal E at position 1 comprises the “fluor” group described above, each K is functionalized with an LA group, and the C-terminal G at position 45 is G′ as described above.
SEQ ID NO: 53: EEELLEELLEELLEELLEEGFGFVGGQLLQQLLQQLLOOLLQKKG, wherein the N-terminal E at position 1 comprises the “fluor” group described above, each K is functionalized with an LA group, and the C-terminal G at position 45 is G′ as described above.
SEQ ID NO: 54: EELLEELLEELLEELLEEEGFGFVGGQLLQQLLQQLLQQLLOKKG, wherein the N-terminal E at position 1 comprises the “fluor” group described above, each K is functionalized with an LA group, and the C-terminal G at position 45 is G′ as described above.
SEQ ID NO: 55: EEELLEELLEELLEELLEGFGFVGGQLLQQLLQQLLQQLLOKKG, wherein the N-terminal E at position 1 comprises the “fluor” group described above, each K is functionalized with an LA group, and the C-terminal G at position 44 is G′ as described above.
SEQ ID NO: 56: ELLEELLEELLEELLEEEGFGFVGGQLLQQLLQQLLQQLLOKKG, wherein the N-terminal E at position 1 comprises the “fluor” group described above, each K is functionalized with an LA group, and the C-terminal G at position 44 is G′ as described above.
SEQ ID NO: 57: EELLEELLEELLEELLEEGFGFVGGQLLQQLLQQLLQQLLOKKG, wherein the N-terminal E at position 1 comprises the “fluor” group described above, each K is functionalized with an LA group, and the C-terminal G at position 44 is G′ as described above.
SEQ ID NO: 58: EELLEELLEELLEELLEGFGFVGGOLLQQLLQQLLQQLLOKKG, wherein the N-terminal E at position 1 comprises the “fluor” group described above, each K is functionalized with an LA group, and the C-terminal G at position 43 is G′ as described above.
SEQ ID NO: 59: ELLEELLEELLEELLEEGFGFVGGQLLQQLLQQLLQQLLOKKG, wherein the N-terminal E at position 1 comprises the “fluor” group described above, each K is functionalized with an LA group, and the C-terminal G at position 43 is G′ as described above.

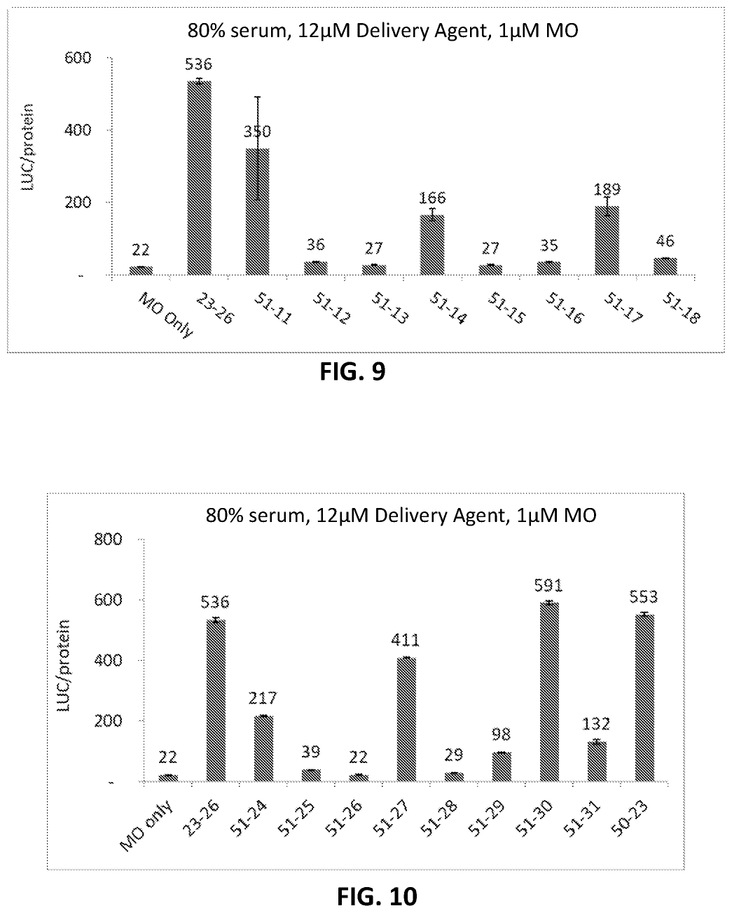

In this example, it was seen that adding glutamates to the C-terminus group in the above described modified delivery agents resulted in reduced activity relative to the positive control, but activity was still observed. Results are shown in FIG. 9.

### Example 8

In this example, similar evaluations as described in Example 7 were conducted with modified delivery agents having different lytic peptide group sequences than positive control delivery agent 23-26. The specific examples are described below.
Negative Control: Morpholino only
Positive Control: 23-26: fluor-[Lytic]-[CL]-[Mask]-[2×K(LA)]-[G]
51-24: fluor-[EEE]-[QLL EEL LEE LLQ]-[CL]-[Mask]-[2×K(LA]-[G]
51-25: fluor-[EE]-[QLL EEL LEE LLQ]-[E]-[CL]-[Mask]-[2×K(LA]-[G′]
51-26: fluor-[E]-[QLL EEL LEE LLQ]-[EE]-[CL]-[Mask]-[2×K(LA]-[G′]
51-27: fluor-[EE]-[QLL EEL LEE LLQ]-[CL]-[Mask]-[2×K(LA]-[G′]
51-28: fluor-[QLL EEL LEE LLQ]-[EE]-[CL]-[Mask]-[2×K(LA]-[G′]
51-29: fluor-[E]-[QLL EEL LEE LLQ]-[E]-[CL]-[Mask]-[2×K(LA]-[G′]
51-30: fluor-[E]-[QLL EEL LEE LLQ]-[CL]-[Mask]-[2×K(LA]-[G]
51-31: fluor-[QLL EEL LEE LLQ]-[E]-[CL]-[Mask]-[2×K(LA]-[G]
50-23: fluor-[E]-[QLL EEL LEE LLQ]-[CL]-[Mask]-[2×K(LA]-[G′]
SEQ ID NO: 60: EEEQLLEELLEELLQ, wherein the N-terminal E at position 1 comprises the “fluor” group described above.
SEQ ID NO: 61: EEQLLEELLEELLQE, wherein the N-terminal E at position 1 comprises the “fluor” group described above.
SEQ ID NO: 62: EQLLEELLEELLQEE, wherein the N-terminal E at position 1 comprises the “fluor” group described above.
SEQ ID NO: 63: EEQLLEELLEELLQ, wherein the N-terminal E at position 1 comprises the “fluor” group described above.
SEQ ID NO: 64: QLLEELLEELLQEE, wherein the N-terminal Q at position 1 comprises the “fluor” group described above.
SEQ ID NO: 65: EQLLEELLEELLQE, wherein the N-terminal E at position 1 comprises the “fluor” group described above.
SEQ ID NO: 66: EQLLEELLEELLQ, wherein the N-terminal E at position 1 comprises the “fluor” group described above.
SEQ ID NO: 67: QLLEELLEELLQE, wherein the N-terminal Q at position 1 comprises the “fluor” group described above.
SEQ ID NO: 68: EQLLEELLEELLQ, wherein the N-terminal E at position 1 comprises the “fluor” group described above.
SEQ ID NO: 69: EEEQLLEELLEELLQGFGFVGGQLLQQLLQQLLQKKG, wherein the N-terminal E at position 1 comprises the “fluor” group described above, each K is functionalized with an LA group, and the C-terminal G at position 37 is G′ as described above.
SEQ ID NO: 70: EEQLLEELLEELLQEGFGFVGGQLLQQLLQQLLQKKG, wherein the N-terminal E at position 1 comprises the “fluor” group described above, each K is functionalized with an LA group, and the C-terminal G at position 37 is G′ as described above.
SEQ ID NO: 71: EQLLEELLEELLQEEGFGFVGGQLLQQLLQQLLOKKG, wherein the N-terminal E at position 1 comprises the “fluor” group described above, each K is functionalized with an LA group, and the C-terminal G at position 37 is G′ as described above.
SEQ ID NO: 72: EEQLLEELLEELLQGFGFVGGQLLQQLLQQLLOKKG, wherein the N-terminal E at position 1 comprises the “fluor” group described above, each K is functionalized with an LA group, and the C-terminal G at position 36 is G′ as described above.
SEQ ID NO: 73: QLLEELLEELLQEEGFGFVGGQLLQQLLQQLLQKKG, wherein the N-terminal Q at position 1 comprises the “fluor” group described above, each K is functionalized with an LA group, and the C-terminal G at position 36 is G′ as described above.
SEQ ID NO: 74: EQLLEELLEELLQEGFGFVGGQLLQQLLQQLLOKKG, wherein the N-terminal E at position 1 comprises the “fluor” group described above, each K is functionalized with an LA group, and the C-terminal G at position 36 is G′ as described above.
SEQ ID NO: 75: EQLLEELLEELLQGFGFVGGQLLQQLLQQLLOKKG, wherein the N-terminal E at position 1 comprises the “fluor” group described above, each K is functionalized with an LA group, and the C-terminal G at position 35 is G′ as described above.
SEQ ID NO: 76: QLLEELLEELLQEGFGFVGGQLLQQLLQQLLOKKG, wherein the N-terminal Q at position 1 comprises the “fluor” group described above, each K is functionalized with an LA group, and the C-terminal G at position 35 is G′ as described above.
SEQ ID NO: 77: EQLLEELLEELLQGFGFVGGQLLQQLLQQLLQKKG, wherein the N-terminal E at position 1 comprises the “fluor” group described above, each K is functionalized with an LA group, and the C-terminal G at position 35 is G′ as described above.

This example establishes that an active “lytic-cleavable linker-mask” structure can be obtained for modified delivery agent examples comprising modifications to an “XYYX” motif (where in this example X=E or Q and Y=L). Results are shown in FIG. 10. As such, examples of the delivery agent can comprise multiple XYYX motif repeats wherein some of the “XYYX” motifs are different from others. This example also shows that in some examples of modified delivery agents, adding one or multiple amino acids to either the N- or C-terminus of the lytic peptide group can modify activity relative to the control delivery agent 23-26.

### Example 9

In this example, a delivery agent embodiment comprising an α-methyl leucine in the third amino acid position of the lytic peptide group was compared to examples comprising a leucine in the same position.

The specific delivery agent examples are described below.
Positive Control: Endoporter (EP)
56-3: fluor-[Lytic]-[CL]-[2×K(LA)]-[G′]
56-5: fluor-[ELa EEL LEE LLE]-[CL]-[2×K(LA)]-[G′]
23-26: fluor-[Lytic]-[CL]-[Mask]-[2×K(LA)]-[G]
SEQ ID NO: 78: ELXEELLEELLE, wherein the N-terminal E at position 1 comprises the “fluor” group described above and X is α-methyl leucine
SEQ ID NO: 79: ELXEELLEELLEGFGFVGGQLLQQLLQQLLOKKG, wherein the N-terminal E at position 1 comprises the “fluor” group described above, X is α-methyl leucine, each K is functionalized with an LA group, and the C-terminal G at position 34 is G′ as described above.

In the above examples, a is α-methyl leucine, which can have a strong propensity for alpha helix formation.

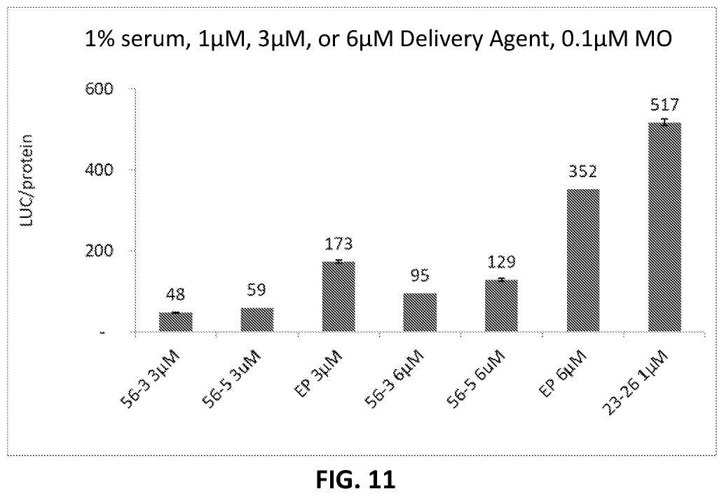

As shown in FIG. 11, this example establishes that, in some aspects of the disclosure, an α-methyl leucine can replace a leucine in the lytic peptide group with minimal change in activity for a lytic-CL-LA peptide component.

### Example 10

In this example, a delivery agent embodiment comprising an α-methyl leucine in the second amino acid position of the mask peptide group was compared to examples comprising a leucine in the same position. The specific delivery agent examples are described below.
Negative control: No treatment (This sample contained a morpholino but no delivery agent)
60-16: fluor-[EaLEEaLEELLE]-[CL]-[Mask]-[2×K((LA)]-[G′], n=5
60-17: fluor-[Lytic]-[CL]-[QαL QQL LQQ LLQ]-[2×K(LA)]-[G′], n=5
60-4: fluor-[Lytic]-[CL]-[Mask]-[2×K(LA)]-[G′], n=5
23-26: fluor-[Lytic]-[CL]-[Mask]-[2×K(LA)]-[G], n=7
62-9: fluor-[EαLEEαLEELLE]-[CL]-[Mask]-[2×K(LA)]-[G′], n=7
With reference to these compounds, K(LA) and a represent the structures shown below:

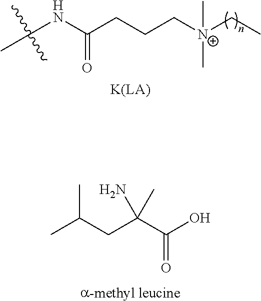

SEQ ID NO: 80: EXLEEXLEELLE, where the N-terminal E at position 1 comprises the “fluor” group described above, each X is α-methyl leucine
SEQ ID NO: 81: QXLQQLLOOLLQ, where X is α-methyl leucine
SEQ ID NO: 82: EXLEEXLEELLEGFGFVGGOLLQQLLQQLLOKKG, where the N-terminal E at position 1 comprises the “fluor” group described above, each X is α-methyl leucine, each K is functionalized with an LA group having n=5 per the formula “K(LA)” above, and the C-terminal G at position 34 is G′ as described above.
SEQ ID NO: 83: ELLEELLEELLEGFGFVGGQXLQQLLQQLLOKKG, where the N-terminal E at position 1 comprises the “fluor” group described above, X is α-methyl leucine, each K is functionalized with an LA group having n=5 per the formula “K(LA)” above, and the C-terminal G at position 34 is G′ as described above.
SEQ ID NO: 247: EXLEEXLEELLEGFGFVGGOLLQQLLQQLLOKKG, where the N-terminal E at position 1 comprises the “fluor” group described above, each X is α-methyl leucine, each K is functionalized with an LA group, and the C-terminal G at position 34 is G′ as described above.
SEQ ID NO: 248: ELLEELLEELLEGFGFVGGQLLQQLLQQLLOKKG, wherein the N-terminal E at position 1 comprises the “fluor” group described above, each K is functionalized with an LA group having n=5 per the formula “K(LA)” above, and the C-terminal G at position 34 is G′ as described above.

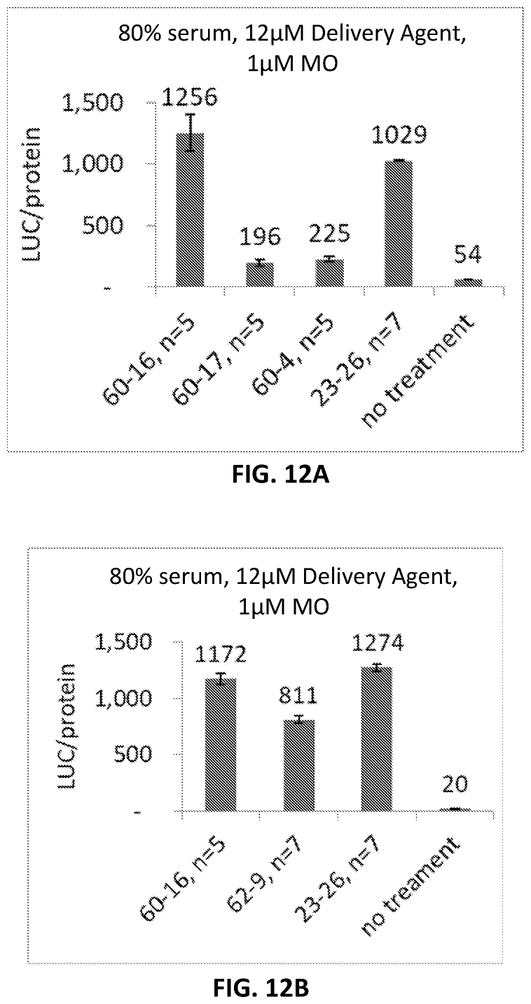

This example establishes that in some aspects of the disclosure, α-methyl leucines can replace leucines in either of the lytic and mask peptide groups (FIGS. 12A and 12B).

### Example 11

In this example, delivery agent examples comprising aspartic acid at various positions in the lytic peptide group were compared to an embodiment comprising glutamates in the same positions of the lytic peptide. The specific delivery agent examples are described below.
Positive Control: 23-26: fluor-[lytic]-[CL]-[Mask]-[2×K(LA)]-[G′]
Negative Control: Morpholino only
88-8: Cap-[ELL EEL LEE LLE]-[CL]-[Mask]-[2×K(LA)]-[GNalG′]
88-9: Cap-[DLL DDL LDD LLE]-[CL]-[Mask]-[2×K(LA)]-[GNalG′]
88-10: Cap-[DLL DEL LDE LLE]-[CL]-[Mask]-[2×K(LA)]-[GNalG′]
88-11: Cap-[ELL DEL LDE LLE]-[CL]-[Mask]-[2×K(LA)]-[GNalG′]
88-12: Cap-[ELL EDL LED LLE]-[CL]-[Mask]-[2×K(LA)]-[GNalG′]
SEQ ID NO: 2: DLLDDLLDDLLE, wherein the N-terminal D at position 1 comprises the “Cap” group described above.
SEQ ID NO: 84: DLLDELLDELLE, wherein the N-terminal D at position 1 comprises the “Cap” group described above.
SEQ ID NO: 85: ELLDELLDELLE, wherein the N-terminal E at position 1 comprises the “Cap” group described above.
SEQ ID NO: 86: ELLEDLLEDLLE, wherein the N-terminal E at position 1 comprises the “Cap” group described above.
SEQ ID NO: 87: ELLEELLEELLEGFGFVGGQLLQQLLQQLLOKKGXG, wherein the N-terminal E at position 1 comprises the “Cap” group described above, each K is functionalized with an LA group, X is Nal, and the C-terminal G at position 36 is G′ as described above.
SEQ ID NO: 88: DLLDDLLDDLLEGFGFVGGQLLQQLLQQLLQKKGXG, wherein the N-terminal D at position 1 comprises the “Cap” group described above, each K is functionalized with an LA group, X is Nal, and the C-terminal G at position 36 is G′ as described above.
SEQ ID NO: 89: DLLDELLDELLEGFGFVGGOLLQQLLQQLLOKKGXG, wherein the N-terminal D at position 1 comprises the “Cap” group described above, each K is functionalized with an LA group, X is Nal, and the C-terminal G at position 36 is G′ as described above.
SEQ ID NO: 90: ELLDELLDELLEGFGFVGGQLLQQLLQQLLQKKGXG, wherein the N-terminal E at position 1 comprises the “Cap” group described above, each K is functionalized with an LA group, X is Nal, and the C-terminal G at position 36 is G′ as described above.
SEQ ID NO: 91: ELLEDLLEDLLEGFGFVGGQLLQQLLQQLLOKKGXG, wherein the N-terminal E at position 1 comprises the “Cap” group described above, each K is functionalized with an LA group, X is Nal, and the C-terminal G at position 36 is G′ as described above.

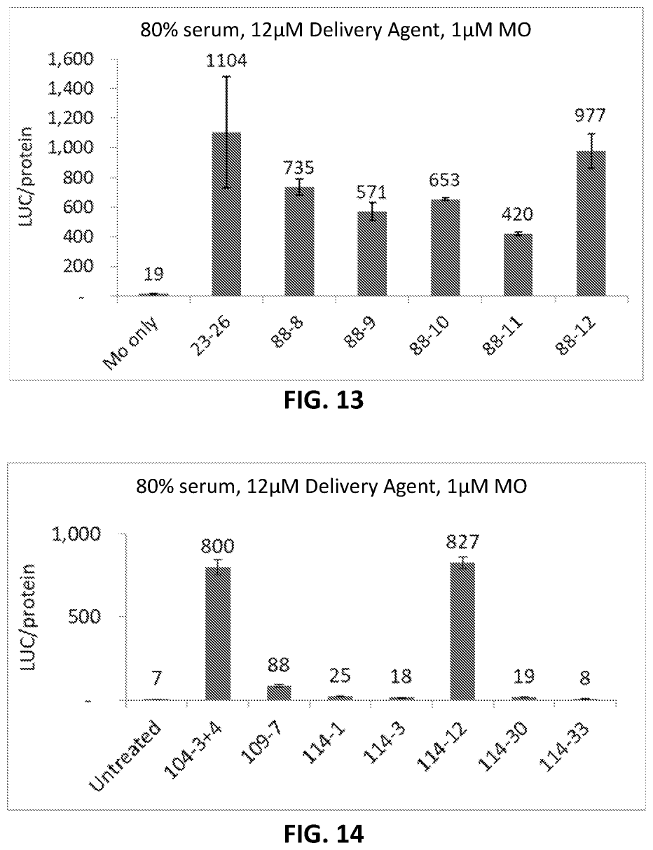

As shown in FIG. 13, replacing various glutamates in the lytic peptide group with aspartic acid can have a marginal impact on delivery agent activity level in some aspects of the disclosure.

### Example 12

In this example, delivery agent examples comprising lytic peptide groups of varying lengths having aspartic acid at various positions were compared. In addition, a delivery agent embodiment comprising a lytic peptide group with positively charged arginines replacing negatively charged amino acids was compared to an embodiment comprising negatively charged amino acids in the same positions of the lytic peptide. The specific delivery agent examples are described below.
Negative Control: Untreated (This sample contained a morpholino but no delivery agent)
Positive Control: 104-3+4: Cap-[DLL DDL LDD LLE]-[CL]-[Mask]-[2×K(LA)]-[GNalG′]
109-7: Cap-[RLL RRL LRR LLR]-[CL]-[Mask]-[2×K(LA)]-[GNalG′]
114-1: Asp-20-mer: Cap-[DLL DDL LDD LLD DLL DDL LE]-[CL]-[QLL QQL LQQ LLQ QLL QQL LQ]-[2×K(LA)]-[GNalG′]
114-3: Asp-16-mer: Cap-[DLL DDL LDD LLD DLL E]-[CL]-[QLL QQL LQQ LLQ QLL Q]-[2×K(LA)]-[GNalG′]
114-12: Asp-12-mer: Cap-[DLL DDL LDD LLE]-[CL]-[Mask]-[2×K(LA)]-[GNalG′]
114-30: Asp-8-mer: Cap-[DLL DDL LE]-[CL]-[QLL QQL LQ]-[2×K(LA)]-[GNalG′]
114-33: Asp-4-mer: Cap-[DLL E]-[CL]-[QLL Q]-[2×K(LA)]-[GNalG′]
SEQ ID NO: 92: RLLRRLLRRLLR, wherein the N-terminal R at position 1 comprises the “Cap” group described above.
SEQ ID NO: 93: DLLDDLLDDLLDDLLDDLLE, wherein the N-terminal D at position 1 comprises the “Cap” group described above.
SEQ ID NO: 94: DLLDDLLDDLLDDLLE, wherein the N-terminal D at position 1 comprises the “Cap” group described above.
SEQ ID NO: 95: DLLDDLLDDLLE, wherein the N-terminal D at position 1 comprises the “Cap” group described above.
SEQ ID NO: 96: DLLDDLLE, wherein the N-terminal D at position 1 comprises the “Cap” group described above.
SEQ ID NO: 97: DLLE, wherein the N-terminal D at position 1 comprises the “Cap” group described above.

SEQ ID NO: 98: QLLQQLLQQLLQQLLQQLLQ
SEQ ID NO: 99: QLLQQLLQQLLQQLLQ
SEQ ID NO: 100: QLLQQLLQ
SEQ ID NO: 101: QLLQ

SEQ ID NO: 102: RLLRRLLRRLLRGFGFVGGQLLQQLLQQLLOKKGXG, wherein the N-terminal R at position 1 comprises the “Cap” group described above, each K is functionalized with an LA group, X is Nal, and the C-terminal G at position 36 is G′ as described above.
SEQ ID NO: 103: DLLDDLLDDLLDDLLDDLLEGFGFVGGQLLQQLLQQLLQQLLQQLLQKKGXG, wherein the N-terminal D at position 1 comprises the “Cap” group described above, each K is functionalized with an LA group, X is Nal, and the C-terminal G at position 52 is G′ as described above. SEQ ID NO: 104: DLLDDLLDDLLDDLLEGFGFVGGQLLQQLLQQLLQQLLOKKGXG, wherein the N-terminal D at position 1 comprises the “Cap” group described above, each K is functionalized with an LA group, X is Nal, and the C-terminal G at position 44 is G′ as described above.
SEQ ID NO: 105: DLLDDLLDDLLEGFGFVGGQLLQQLLQQLLQKKGXG, wherein the N-terminal D at position 1 comprises the “Cap” group described above, each K is functionalized with an LA group, X is Nal, and the C-terminal G at position 36 is G′ as described above.
SEQ ID NO: 106: DLLDDLLEGFGFVGGQLLQQLLQKKGXG, wherein the N-terminal D at position 1 comprises the “Cap” group described above, each K is functionalized with an LA group, X is Nal, and the C-terminal G at position 28 is G′ as described above.
SEQ ID NO: 107: DLLEGFGFVGGQLLQKKGXG, wherein the N-terminal D at position 1 comprises the “Cap” group described above, each K is functionalized with an LA group, X is Nal, and the C-terminal G at position 20 is G′ as described above.

This example establishes that in some aspects of the disclosure, delivery agent activity is increased by use of a 12-mer lytic peptide group. However, although the 114-3 delivery agent showed low activity in the present example, the same design exhibited increased activity with use of glutamates rather than aspartic acid groups in the lytic peptide group (see Example 4). Thus, it is believed that, in some examples, a delivery agent embodiment comprising a lytic peptide group longer than 12 amino acids in length may exhibit similar or increased activity as compared to an embodiment having a 12-mer lytic peptide, when glutamates are used in the lytic peptide group rather than the aspartic acid groups. Further, the 109-7 delivery agent, with acidic amino acids in the lytic peptide group replaced with arginines in the same positions, is active. Results are shown in FIG. 14.

### Example 13

In this example, the below examples of a delivery agent were evaluated against a negative control. The delivery agent examples included structures as described below, wherein compound 114-9 has lytic and mask peptide group sequences that follow a XYYYX motif (wherein X=E or D, for the lytic peptide group; or Q, for the mask peptide group; and Y=L for both the lytic peptide and mask peptide groups).
Negative Control: morpholino without delivery agent
104-3+4: Cap-[DLL DDL LDD LLE]-[CL]-[Mask]-[2×K(LA)]-[GNalG′] (positive control)
114-12: Cap-[DLL DDL LDD LLE]-[CL]-[Mask]-[2×K(LA)]-[GNalG′] (internal control of synthesis run)
114-6: Cap-[DLD DLD LLD LDD LDL L]-[CL]-[QLQ QLQ LLQ LQQ LOL L]-[2×K(LA)]-[GNalG′]
114-9: Cap-[DLLLD DLLLD DLLLE]-[CL]-[QLLLQ QLLLQ QLLLQ]-[2×K(LA)]-[GNalG′]
SEQ ID NO: 109: DLDDLDLLDLDDLDLL, wherein the N-terminal D at position 1 comprises the “Cap” group described above.
SEQ ID NO: 110: DLLLDDLLLDDLLLE, wherein the N-terminal D at position 1 comprises the “Cap” group described above.

SEQ ID NO: 111: QLQQLQLLQLQQLQLL
SEQ ID NO: 112: QLLLQQLLLQQLLLQ

SEQ ID NO: 113: DLDDLDLLDLDDLDLLGFGFVGGQLQQLQLLQLQQLQLLKKGXG, wherein the N-terminal D at position 1 comprises the “Cap” group described above, each K is functionalized with an LA group, X is Nal, and the C-terminal G at position 44 is G′ as described above.
SEQ ID NO: 114: DLLLDDLLLDDLLLEGFGFVGGQLLLQQLLLQQLLLQKKGXG, wherein the N-terminal D at position 1 comprises the “Cap” group described above, each K is functionalized with an LA group, X is Nal, and the C-terminal G at position 42 is G′ as described above.

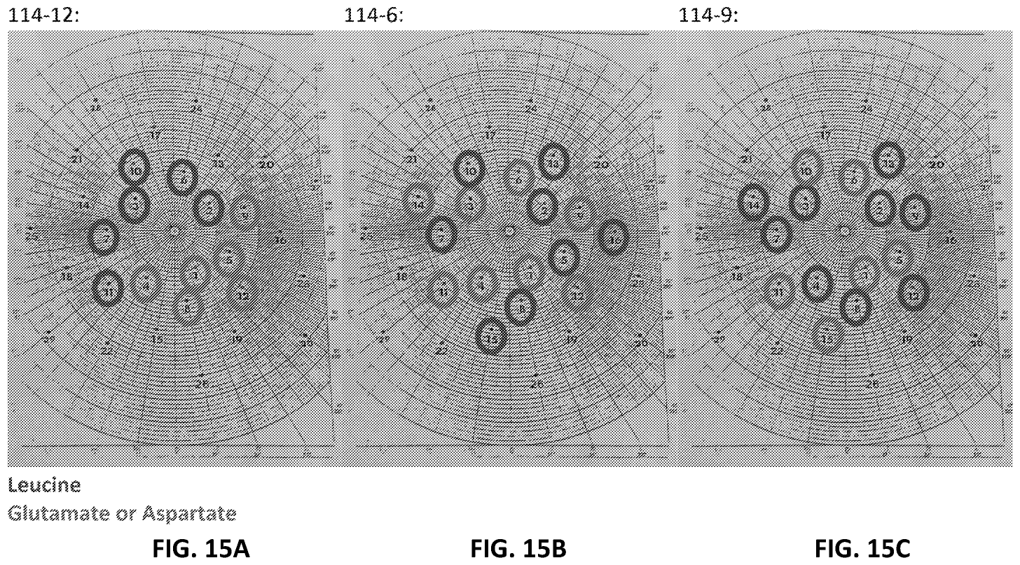

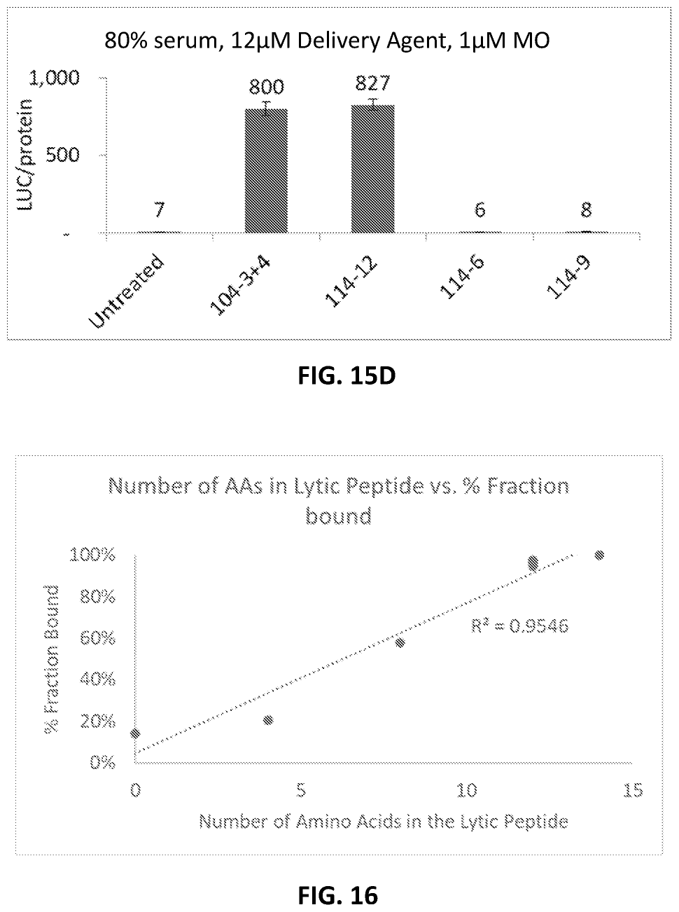

Neither of compounds 114-6 or 114-9 exhibited activity, which, without being limited to a single theory, is currently believed to indicate that lytic peptide and mask peptide groups following a XYYX sequence motif are desirable examples. Helical Wheel illustrations and graphical data are provided in FIGS. 15A-15C and FIG. 15D, respectively.

### Example 14

In this example, a TransilXL HSA (Human Serum Albumin) Binding Kit was used to evaluate delivery agent examples that are conjugated to a Morpholino compound. Agent examples are described below and the attachment site on the Morpholino is illustrated and is a covalent carbamate linkage to the 3′-secondary amine in the Morpholino backbone of the terminal subunit.
97-1: Cap-[ELL EEL LEE LLE LL]-[CL]-[1×K(LA)]-[GNal]-[K(N₃)]-[G]
97-2:12-mer: Cap-[Lytic]-[CL]-[2×K(LA)]-[GNal]-[K(N₃)]-[G′]
97-3:12-mer: Cap-[Lytic]-[CL]-[1K(LA)]-[GNal]-[K(N₃)]-[G′]
97-5:8-mer: Cap-[ELL EEL LE]-[CL]-[1×K(LA)]-[GNal]-[K(N₃)]-[G]
97-7:4-mer: Cap-[ELLE]-[CL]-[1×K(LA)]-[GNal]-[K(N₃)]-[G′]
91-6: Cap-[DLL DDL LDD LLE]-[CL]-[1×K(LA)]-[K(N₃)]-[GNalG′]
Positive control: Warfarin
MO:

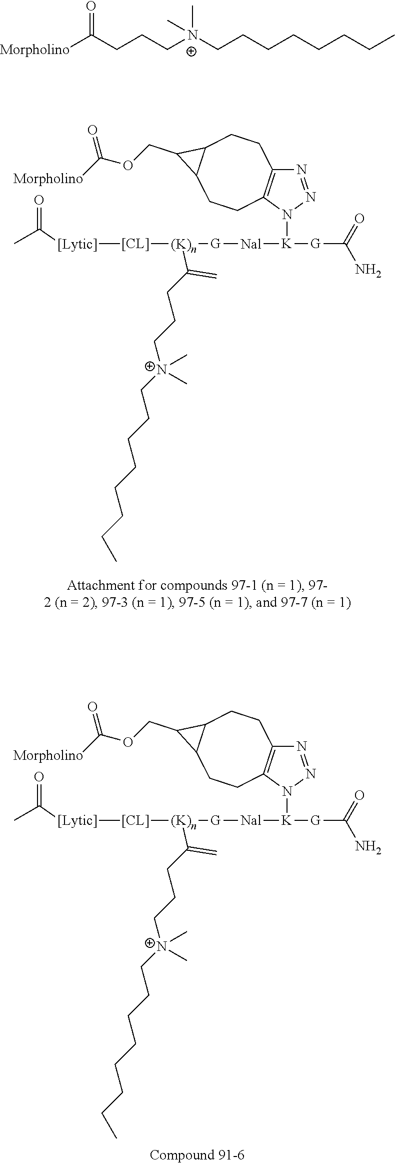

SEQ ID NO: 115: ELLEELLEELLELL, wherein the N-terminal E at position 1 comprises the “Cap” group described above.
SEQ ID NO: 116: ELLEELLE, wherein the N-terminal E at position 1 comprises the “Cap” group described above.
SEQ ID NO: 117: ELLE, wherein the N-terminal E at position 1 comprises the “Cap” group described above.
SEQ ID NO: 118: ELLEELLEELLELLGFGFVGGKGXXG, wherein the N-terminal E at position 1 comprises the “Cap” group described above, K is functionalized with an LA Group, X at position 24 is Nal, the X at position 25 is lysine having a side chain modified with an azide (“Lys(N₃)”), and the G at position 26 is G′ as described above.
SEQ ID NO: 119: ELLEELLEELLEGFGFVGGKKGXXG, wherein the N-terminal E at position 1 comprises the “Cap” group described above, each K is functionalized with an LA Group, X at position 23 is Nal, the X at position 24 is lysine having a side chain modified with an azide (“Lys(N₃)”), and the G at position 25 is G′ as described above.
SEQ ID NO: 120: ELLEELLEELLEGFGFVGGKGXXG, wherein the N-terminal E at position 1 comprises the “Cap” group described above, K is functionalized with an LA Group, X at position 22 is Nal, the X at position 23 is lysine having a side chain modified with an azide (“Lys(N₃)”), and the G at position 24 is G′ as described above.
SEQ ID NO: 121: ELLEELLEGFGFVGGKGXXG, wherein the N-terminal E at position 1 comprises the “Cap” group described above, K is functionalized with an LA Group, X at position 18 is Nal, the X at position 19 is lysine having a side chain modified with an azide (“Lys(N₃)”), and the G at position 20 is G′ as described above.
SEQ ID NO: 122: ELLEGFGFVGGKXGXG, wherein the N-terminal E at position 1 comprises the “Cap” group described above, K is functionalized with an LA Group, X at position 15 is Nal, the X at position 13 is lysine having a side chain modified with an azide (“Lys(N₃)”), and the G at position 16 is G′ as described above.
SEQ ID NO: 123: DLLDDLLDDLLEGFGFVGGKXGXG, wherein the N-terminal D at position 1 comprises the “Cap” group described above, K is functionalized with an LA Group, X at position 23 is Nal, the X at position 21 is lysine having a side chain modified with an azide (“Lys(N₃)”), and the G at position 24 is G′ as described above.

TABLE 1

| | | Fb (fraction MO | | | |
|---|---|---|---|---|---|
| Compound name | K_{d} HSA | bound to HSA) | | r² | TQI¹ |
| MO- 97-1 Conjugate | 1.19E−06 | 99.8% | ±0.00 | 0.8880 | 6.4 |
| MO- 97-2 Conjugate | 2.11E−05 | 96.5% | ±0.00 | 0.9990 | 9.7 |
| MO- 97-3 Conjugate | 1.63E−05 | 97.3% | ±0.00 | 0.9869 | 9.5 |
| MO- 97-5 Conjugate | 4.28E−04 | 57.9% | ±0.03 | 0.9850 | 9.4 |
| MO- 97-7 Conjugate | 2.29E−03 | 20.4% | ±0.08 | 0.9955 | 9.2 |
| MO-91-6 Conjugate | 3.27E−05 | 94.7% | ±0.00 | 0.9906 | 9.7 |
| MO | 3.57E−03 | 14.2% | ±0.13 | 0.4044 | 6.1 |
| Warfarin | 7.11E−05 | 89.2% | ±0.02 | 0.9917 | 9.6 |

¹TQI (Transil Quality Index):
between 7 and 10: good data quality |
between 5 and 7: compromised data quality |
below 5: poor data quality

The TransiIXL HSA Binding Kit from Sovicell tests the fraction of morpholino bound to serum albumin in the assay. In this example, it was determined that the length of the lytic peptide group conjugated to the morpholino positively correlates with fraction of morpholino bound to human serum albumin. Stated another way, the longer the peptide the greater the % peptide-Morpholino conjugate bound to serum. See FIG. 16 for graphical results. The examples described in this example did not comprise a mask peptide group sequence, thus it currently is believed that the mask peptide group sequence can help prevent delivery agent binding the serum.

### Example 15

In this example, delivery agent examples comprising different cleavable linkers were evaluated. The particular structures of the delivery agent examples are provided below.
Negative Control: Untreated (This sample contained a morpholino but no delivery agent)
104-3+4: Cap-[DLLDDLLDDLLE]-[CL]-[MASK]-[2×K(LA)]-[GNalG′]
114-12: Cap-[DLLDDLLDDLLE]-[CL]-[MASK]-[2×K(LA)]-[GNalG′]
114-18: Cap-[DLLDDLLDDLLE]-[PKPL]-[MASK]-[2×K(LA)]-[GNalG′]
114-21: Cap-[DLLDDLLDDLLE]-[VG]-[MASK]-[2×K(LA)]-[GNalG′]
114-24: Cap-[DLLDDLLDDLLE]-[V-Cit]-[MASK]-[2×K(LA)]-[GNalG′]
114-27: Cap-[DLLDDLLDDLLE]-[ ]-[MASK]-[2×K(LA)]-[GNalG′] (No Cleavable Link)
SEQ ID NO: 124: DLLDDLLDDLLEPKPLOLLQQLLQQLLOKKGXG, wherein the N-terminal D at position 1 comprises the “Cap” group described above, each K at positions 29 and 30 is functionalized with an LA group, X is Nal, and the C-terminal G at position 33 is G′ as described above.
SEQ ID NO: 125: DLLDDLLDDLLEVGQLLQQLLQQLLQKKGXG, wherein the N-terminal D at position 1 comprises the “Cap” group described above, each K is functionalized with an LA group, X is Nal, and the C-terminal G at position 31 is G′ as described above.
SEQ ID NO: 126: DLLDDLLDDLLEVXQLLQQLLQQLLOKKGXG, wherein the N-terminal D at position 1 comprises the “Cap” group described above, each K is functionalized with an LA group, X at position 14 is Cit, X at position 30 is Nal, and the C-terminal G at position 31 is G′ as described above.
SEQ ID NO: 127: DLLDDLLDDLLEQLLQQLLQQLLQKKGXG, wherein the N-terminal D at position 1 comprises the “Cap” group described above, each K is functionalized with an LA group, X is Nal, and the C-terminal G at position 29 is G′ as described above.

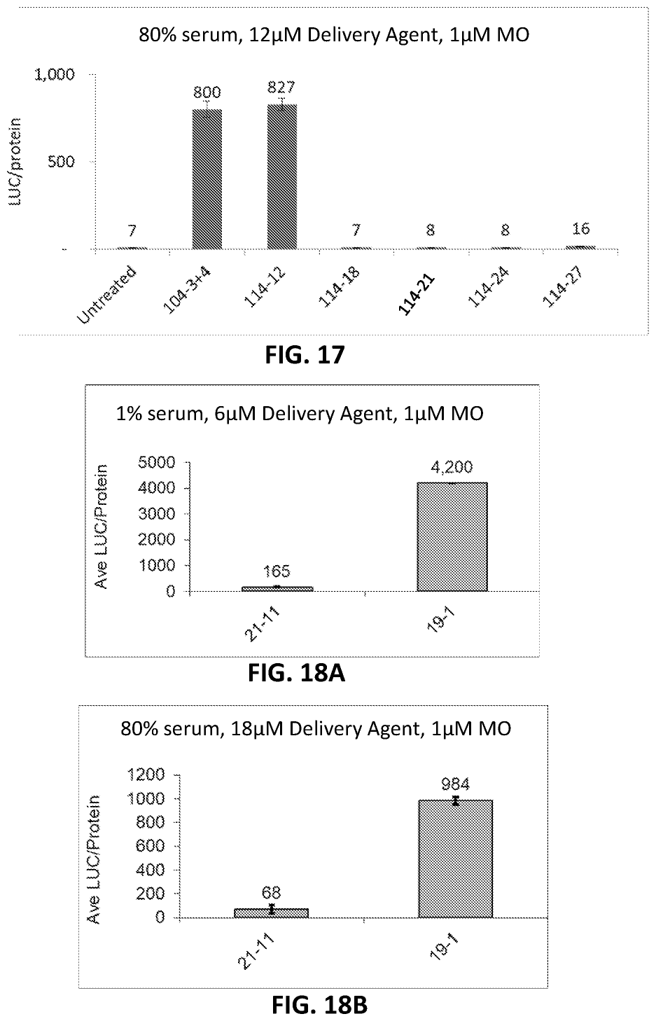

PKPL, VG, and V-Cit are commercially available linkers cleaved by Cathepsin B. The results illustrate that having a cleavable linker facilitates activity and further that cleavable linker examples having a sequence GFG FVG G can exhibit increased activity as compared with commercially available linkers and examples without a cleavable linker. See FIG. 17.

### Example 16

In this example, an exemplary delivery agent embodiment was compared with an embodiment comprising a string of seven glycines, which were not anticipated to be rapidly cleavable by the lysosomal enzyme, Cathepsin B. The examples are described below.
21-11: fluor-[Lytic]-[GGGGGGG]-[Mask]-[1×K(LA)]-[G′]
19-1: fluor-[Lytic]-[CL]-[Mask]-[1×K(LA)]-[G′]
SEQ ID NO: 128: ELLEELLEELLEGGGGGGGQLLQQLLOOLLQKG, wherein the N-terminal E at position 1 comprises the “Fluor” group described above, K is functionalized with an LA Group, and the C-terminal G at position 33 is G′ as described above.
SEQ ID NO: 129: ELLEELLEELLEGFGFVGGQLLQQLLQQLLQKG, wherein the N-terminal E at position 1 comprises the “Fluor” group described above, K is functionalized with an LA Group, and the C-terminal G at position 33 is G′ as described above.

The activity of the “lytic-cleavable linker-mask” sequence with the non-cleavable linker [GGGGGGG] is substantially lower than the delivery agent embodiment comprising the GFG FVG G sequence in both low

(FIG. 18A) and high (FIG. 18B) serum assays.

### Example 17

In this example, an exemplary delivery agent embodiment (36-47) was evaluated for activity as compared to an embodiment comprising a cleavable linker sequence of GFGFAhx (wherein Ahx=aminohexanoic acid), as described below.
36-47: fluor-[Lytic]-[CL]-[Mask]-[1×K(LA)]-[G′]
36-48: fluor-[Lytic]-[GFGFAhx]-[Mask]-[1×K(LA)]-[G′]
SEQ ID NO: 130: ELLEELLEELLEGFGFXQLLQQLLOOLLQKG, wherein the N-terminal E at position 1 comprises the “Fluor” group described above, K is functionalized with an LA Group, X is Ahx, and the C-terminal G at position 31 is G′ as described above.

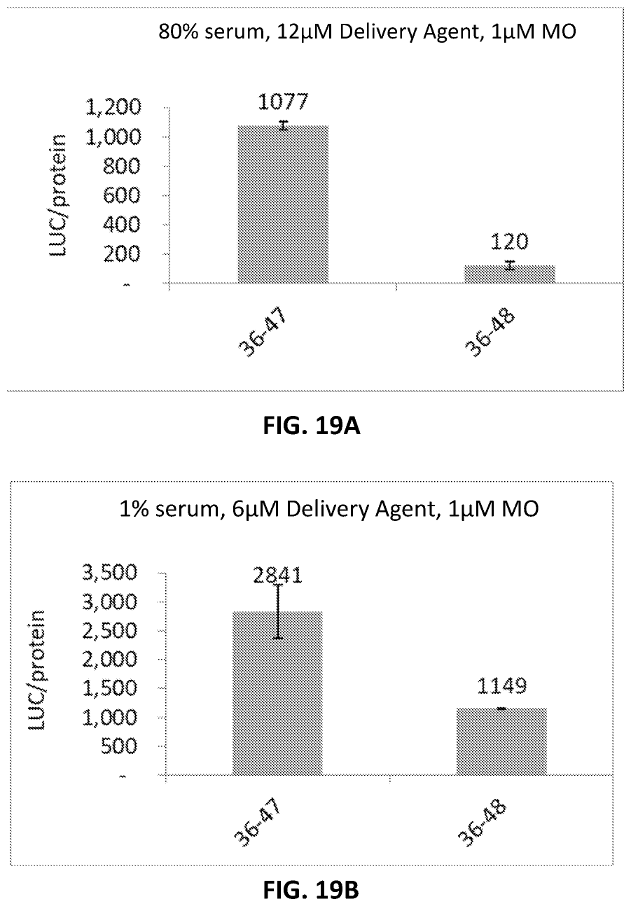

As shown in FIGS. FIGS. 19A and 19B, high serum tests with 36-47 produced 9-fold greater activity than 36-48 (FIG. 19A) and 2.5-fold greater activity in low serum (FIG. 19B), suggesting the cleavable link sequence can have an effect on activity.

### Example 18

In this example, modifications to the cleavable linker structure were evaluated, with the particular examples being described below.
96-1: Cap-[Lytic]-[GK(N₃)]-[GFGFVGG]-[Mask]-[2×K(LA)]-[GNalG′]
96-4: Cap-[Lytic]-[GFGFVGK(N₃)G]-[Mask]-[2×K(LA)]-[GNalG′]
96-5: Cap-[Lytic]-[GF(N₃)GFVGG]-[Mask]-[2×K(LA)]-[GNalG′]
96-6: Cap-[Lytic]-[GFGF(N₃)VGG]-[Mask]-[2×K(LA)]-[GNalG′]
96-7: Cap-[Lytic]-[CL]-[Mask]-[2×K(LA)]-[GNalG′]
wherein

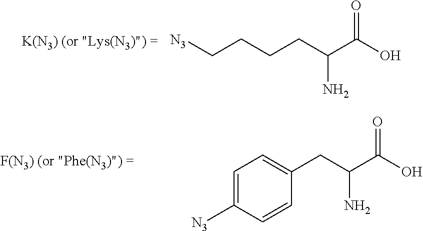 [SMILES: [N-]=[N+]=NCCCCC(N)C(=O)O ; [N-]=[N+]=Nc1ccc(CC(N)C(=O)O)cc1]

SEQ ID NO: 131: ELLEELLEELLEGXGFGFVGGQLLQQLLQQLLQKKGXG, wherein the N-terminal E at position 1 comprises the “Cap” group described above, X at position 14 is lysine having a side chain modified with an azide (“Lys(N₃)”), each K is functionalized with an LA Group, X at position 37 is Nal, and the C-terminal G at position 38 is G′ as described above.
SEQ ID NO: 132: ELLEELLEELLEGFGFVGXGQLLQQLLQQLLQKKGXG, wherein the N-terminal E at position 1 comprises the “Cap” group described above, X at position 19 is lysine having a side chain modified with an azide (“Lys(N₃)”), each K is functionalized with an LA Group, X at position 36 is Nal, and the C-terminal G at position 37 is G′ as described above.
SEQ ID NO: 133: ELLEELLEELLEGXGFVGGQLLQQLLQQLLOKKGXG, wherein the N-terminal E at position 1 comprises the “Cap” group described above, X at position 14 is a phenylalanine having a side chain modified with an azide (“Phe(N₃)”), each K is functionalized with an LA Group, X at position 35 is Nal, and the C-terminal G at position 36 is G′ as described above.
SEQ ID NO: 231: ELLEELLEELLEGFGXVGGQLLQQLLQQLLQKKGXG, wherein the N-terminal E at position 1 comprises the “Cap” group described above, X at position 16 is a phenylalanine having a side chain modified with an azide (“Phe(N₃)”), each K is functionalized with an LA Group, X at position 35 is Nal, and the C-terminal G at position 36 is G′ as described above.

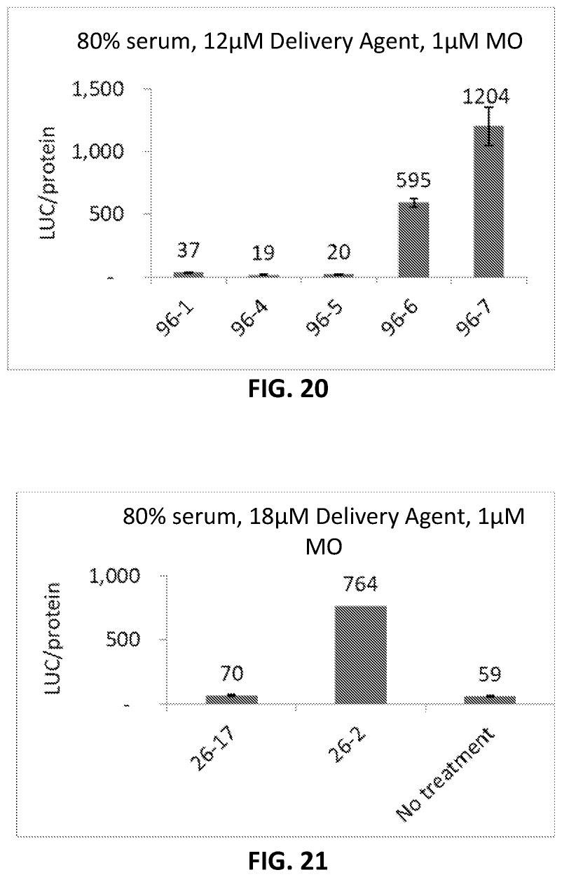

As can be seen in FIG. 20, modifying the cleavable link such that F is replaced with F(N₃) or adding a K(N₃) into the sequence reduced activity, except for compound 96-6, which showed roughly half the activity of delivery agent embodiment 96-7. Without being limited to a single theory, it currently is believed that the azide group (i.e., N₃) on either the lysine or phenylalanine may disrupt activity by reducing reactivity of the cleavable linker, sterically blocking interaction with a Cathepsin B active site or other methods unrelated to the cleavable link sequence. As such, delivery agent examples comprising cleavable linkers as provided by examples in this example may be improved (in terms of activity) by tuning the reactivity of the azide and/or other functional groups of the cleavable linker sequences.

### Example 19

In this example, different mask peptide group sequences were evaluated, namely delivery agent embodiment 26-2 and an embodiment comprising serines in the mask peptide group. The evaluated delivery agent examples are described below and results are shown in FIG. 21.
Negative Control: No treatment (This sample contained a morpholino but no delivery agent)
26-2: fluor-[Lytic]-[CL]-[MASK]-[1×K(LA)]-[G′]
26-17: fluor-[Lytic]-[CL]-[SLL SSL LSS LLS]-[1×K(LA)]-[G′]

SEQ ID NO: 134: SLLSSLLSSLLS

SEQ ID NO: 135: ELLEELLEELLEGFGFVGGSLLSSLLSSLLSKG, wherein the N-terminal E at position 1 comprises the “Fluor” group described above, K is functionalized with an LA Group, and the C-terminal G at position 33 is G′ as described above.

As illustrated in FIGS. 21, replacing 100% of glutamines with serines in the mask provides examples that are slightly active or inactive in high serum.

### Example 20

In this example, different mask peptide group sequences were evaluated, namely delivery agent embodiment 19-1 and an embodiment comprising alanines in the mask peptide group. The evaluated delivery agent examples are described below.
19-1: fluor-[Lytic]-[CL]-[MASK]-[1×K(LA)]-[G′]
19-3: fluor-[Lytic]-[CL]-[ALL AQL LAQ LLQ]-[1×K(LA)]-[G′]
19-5: fluor-[Lytic]-[CL]-[ALL AAL LAA LLA]-[1×K(LA)]-[G′] (Insoluble in cell Culture media: formed sheets of precipitant on cells)
Positive Control: Endoporter
Negative Control: Only Morpholino

SEQ ID NO: 136: ALLAQLLAQLLQ
SEQ ID NO: 137: ALLAALLAALLA

SEQ ID NO: 138: ELLEELLEELLEGFGFVGGALLAQLLAQLLOKG, wherein the N-terminal E at position 1 comprises the “Fluor” group described above, K is functionalized with an LA Group, and the C-terminal G at position 33 is G′ as described above.
SEQ ID NO: 139: ELLEELLEELLEGFGFVGGALLAALLAALLAKG, wherein the N-terminal E at position 1 comprises the “Fluor” group described above, K is functionalized with an LA Group, and the C-terminal G at position 33 is G′ as described above.

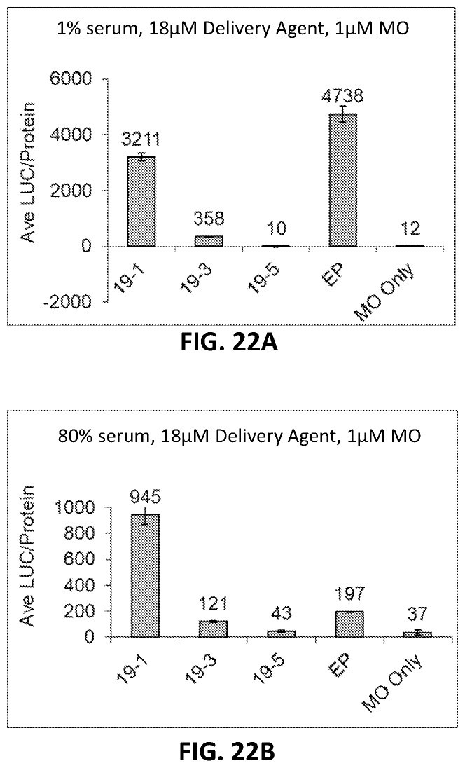

As illustrated in FIGS. 22A and 22B, replacing 50% to 100% of glutamines with alanines in the mask provides examples that are slightly active or inactive in high serum.

### Example 21

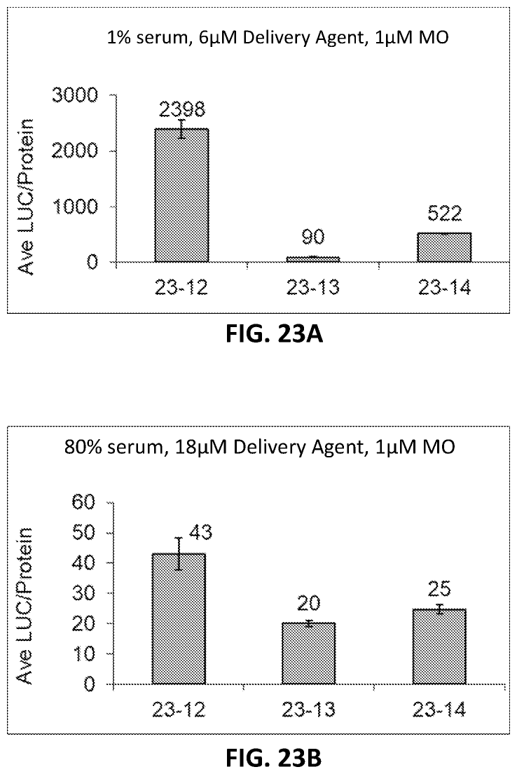

In this example, mask peptide group sequences wherein leucines were replaced with glutamines were evaluated in high and low serum environments. Data from this example (see FIGS. 23A and 23B) illustrate that, without being limited to a particular theory, in some examples, disruption of the XYYX (where in this example X=Q and Y=L) motif of a mask peptide group sequence can lower activity and also that using a second lytic peptide group sequence identical to the first lytic peptide group sequence may exhibit less masking.

### Compounds:

23-12: fluor-[Lytic]-[CL]-[QLL QQL LQQ LQQ]-[2×K(LA)]-[G]
23-13: fluor-[Lytic]-[CL]-[QQL QQL LQQ QQ]-[2×K(LA)]-[G]
23-14: fluor-[Lytic]-[CL]-[Lytic]-[2×K(LA)]-[G′]

SEQ ID NO: 140: QLLQQLLQQLQQ
SEQ ID NO: 141: QQL QQL LQQ LQQ

SEQ ID NO: 142: ELLEELLEELLEGFGFVGGQLLQQLLQQLQQKKG, wherein the N-terminal E at position 1 comprises the “Fluor” group described above, each K is functionalized with an LA Group, and the C-terminal G at position 34 is G′ as described above.
SEQ ID NO: 143: ELLEELLEELLEGFGFVGGQQLQQLLQQLQQKKG, wherein the N-terminal E at position 1 comprises the “Fluor” group described above, K is functionalized with an LA Group, and the C-terminal G at position 34 is G′ as described above.
SEQ ID NO: 144: ELLEELLEELLEGFGFVGGELLEELLEELLEKKG, wherein the N-terminal E at position 1 comprises the “Fluor” group described above, K is functionalized with an LA Group, and the C-terminal G at position 34 is G′ as described above.

### Example 22

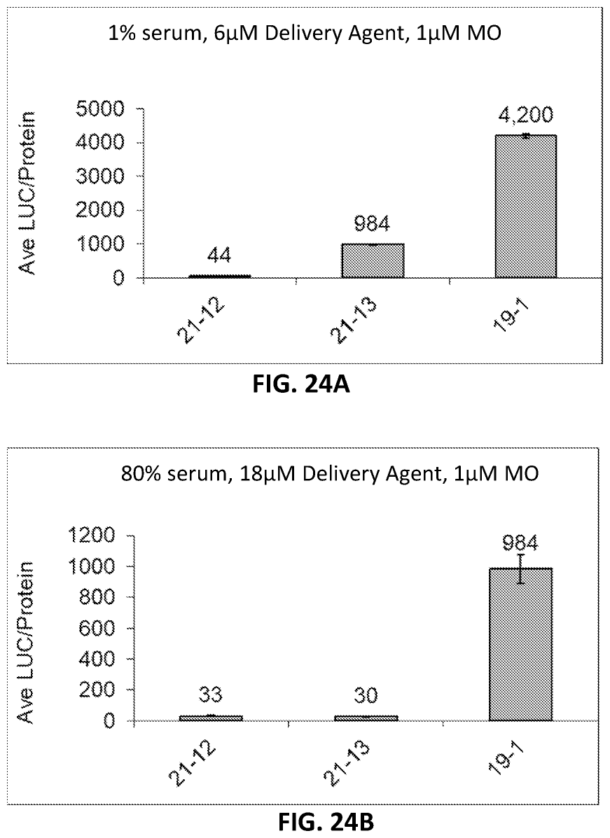

In this example, further delivery agent examples were evaluated, particularly an embodiment without a mask peptide group, an embodiment with a mask peptide group, and an embodiment wherein 100% of glutamines are replaced with aspartic acid in its negatively charged form of asparate. The particular delivery agents are described below and results are shown in FIGS. 24A and 24B.
21-12: fluor-[Lytic]-[CL]-[1×K(LA)]-[G]
21-13: fluor-[Lytic]-[CL]-[DLL DDL LDD LLD]-[1×K(LA)]-[G′]
19-1: fluor-[Lytic]-[CL]-[Mask]-[1×K(LA)]-[G′]

SEQ ID NO: 145: DLLDDLLDDLLD

SEQ ID NO: 146: ELLEELLEELLEGFGFVGGDLLDDLLDDLLDKG, wherein the N-terminal E at position 1 comprises the “Fluor” group described above, K is functionalized with an LA Group, and the C-terminal G at position 33 is G′ as described above.

### Example 23

In this example, further delivery agent examples were evaluated, particularly an embodiment without a mask peptide group and an embodiment with a mask peptide group. The particular delivery agents are described below.
23-15: fluor-[Lytic]-[CL]-[1×K(LA)]-[G′]
19-1: fluor-[Lytic]-[CL]-[Mask]-[1×K(LA)]-[G′]
SEQ ID NO: 147: ELLEELLEELLEGFGFVGGKG, wherein the N-terminal E at position 1 comprises the “Fluor” group described above, K is functionalized with an LA Group, and the C-terminal G at position 21 is G′ as described above.

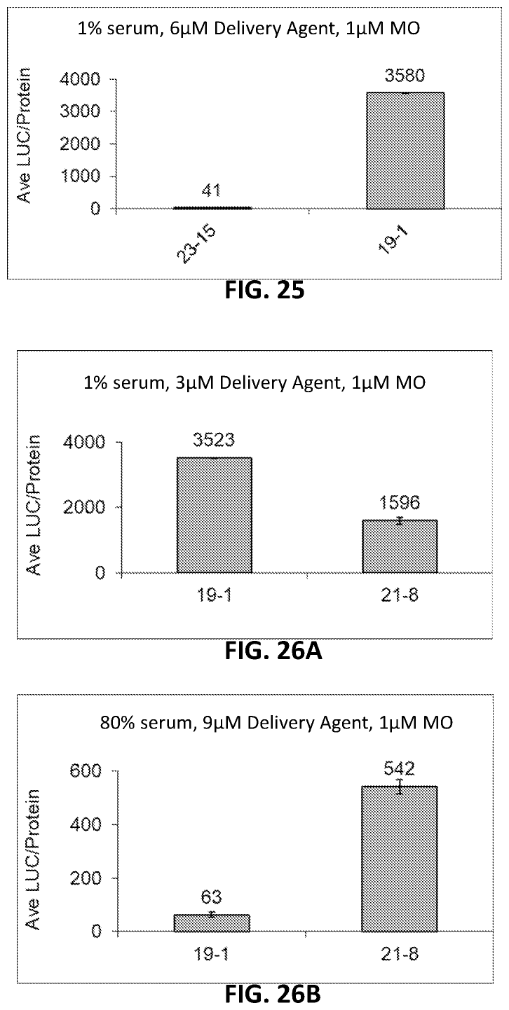

As illustrated in FIG. 25, in some aspects of the disclosure, presence of a mask peptide group improves activity of the delivery agent.

### Example 24

In this example, a mask peptide group sequence wherein two glutamines were replaced with two alanines were evaluated in high and low serum environments. The particular delivery agents are described below.
19-1: fluor-[Lytic]-[CL]-[MASK]-[1×K(LA)]-[G]
21-8: fluor-[Lytic]-[CL]-[QLL AQL LAQ LLQ]-[1×K(LA)]-[G′]

SEQ ID NO: 148: QLLAQLLAQLLQ

SEQ ID NO: 149: ELLEELLEELLEGFGFVGGOLLAQLLAQLLQKG, wherein the N-terminal E at position 1 comprises the “Fluor” group described above, K is functionalized with an LA Group, and the C-terminal G at position 33 is G′ as described above.

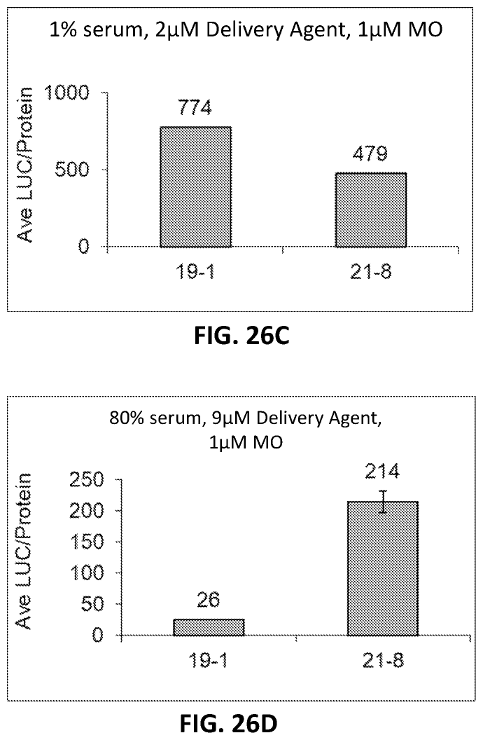

As illustrated in FIGS. 26A and 26B, replacing two glutamines with alanines in the mask peptide group of compound 21-8 improves activity of the delivery agent in 80% serum. This experiment was repeated with the same results (FIGS. 26C-26D).

### Example 25

In this experiment, delivery agent examples comprising (i) mask peptide groups having particular glutamines replaced by glutamates; and (ii) lytic peptide groups having certain glutamates replaced by glutamines were evaluated. The particular delivery agents are described below.
40-31: fluor-[QLL EEL LEE LLQ]-[CL]-[QLL QEL LEQ LLQ]-[K(Z)]-[G′]
40-32: fluor-[QLL EEL LEE LLQ]-[CL]-[QLL EQL LQQ LLE]-[K(Z)]-[G′]
40-33: fluor-[QLL EEL LEE LLQ]-[CL]-[QLL QQL LEQ LLE]-[K(Z)]-[G′]
40-34: fluor-[QLL EEL LEE LLQ]-[CL]-[ELL QEL LQE LLQ]-[K(Z)]-[G′]
40-35: fluor-[QLL EEL LEE LLQ]-[CL]-[ELL QQL LOQ LLE]-[K(Z)]-[G′]
40-36: fluor-[ELL EQL LQE LLE]-[CL]-[QLL EQL LQQ LLQ]-[K(Z)]-[G′]
40-37: fluor-[ELL EQL LQE LLE]-[CL]-[QLL EQL LQQ LLE]-[K(Z)]-[G′]
40-38: fluor-[ELL EQL LQE LLE]-[CL]-[QLL QQL LEQ LLE]-[K(Z)]-[G′]
40-39: fluor-[ELL EQL LQE LLE]-[CL]-[ELL QEL LQE LLQ]-[K(Z)]-[G′]
40-40: fluor-[ELL QEL LQE LLQ]-[CL]-[ELL QEL LOE LLQ]-[K(Z)]-[G′]
40-41: fluor-[ELL QEL LQE LLQ]-[CL]-[QLL EQL LEQ LLE]-[K(Z)]-[G′]
40-42: fluor-[ELL QQL LEQ LLE]-[CL]-[ELL QEL LQQ LLE]-[K(Z)]-[G′]
40-43: fluor-[QLL EQL LQQ LLE]-[CL]-[QLL EQL LQQ LLE]-[K(Z)]-[G′]
40-46: fluor-[Lytic]-[CL]-[MASK]-[2×K(LA)]-[G′]

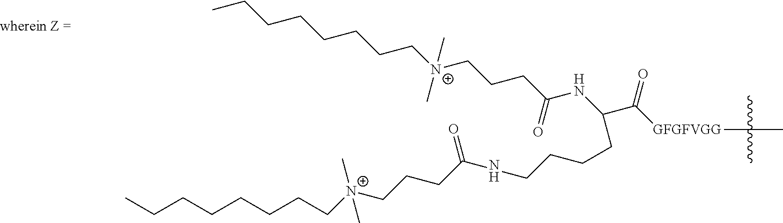

SEQ ID NO: 150: QLLEELLEELLQ, wherein the N-terminal Q at position 1 comprises the “Fluor” group described above.
SEQ ID NO: 151: ELLEQLLQELLE, wherein the N-terminal Q at position 1 comprises the “Fluor” group described above.
SEQ ID NO: 152: ELLOQLLEQLLE, wherein the N-terminal Q at position 1 comprises the “Fluor” group described above.
SEQ ID NO: 153: QLLEQLLOQLLE, wherein the N-terminal Q at position 1 comprises the “Fluor” group described above when this sequence is used as the lytic peptide group.

SEQ ID NO: 154: QLLQELLEQLLQ
SEQ ID NO: 155: ELLQQLLQQLLE
SEQ ID NO: 156: QLLEQLLQQLLQ
SEQ ID NO: 157: QLLEQLLEQLLE
SEQ ID NO: 158: ELLQELLQQLLE

SEQ ID NO: 159: QLLEELLEELLQGFGFVGGQLLQELLEQLLOKG, wherein the N-terminal Q at position 1 comprises the “Fluor” group described above, K is functionalized with a Z group as illustrated above and the C-terminal G at position 33 is G′ as described above.
SEQ ID NO: 160: QLLEELLEELLQGFGFVGGOLLEQLLQQLLEKG, wherein the N-terminal Q at position 1 comprises the “Fluor” group described above, K is functionalized with a Z group as illustrated above and the C-terminal G at position 33 is G′ as described above.
SEQ ID NO: 161: QLLEELLEELLQGFGFVGGQLLQQLLEQLLEKG, wherein the N-terminal Q at position 1 comprises the “Fluor” group described above, K is functionalized with a Z group as illustrated above and the C-terminal G at position 33 is G′ as described above.
SEQ ID NO: 162: QLLEELLEELLQGFGFVGGELLQELLQELLQKG, wherein the N-terminal Q at position 1 comprises the “Fluor” group described above. K is functionalized with a Z group as illustrated above and the C-terminal G at position 33 is G′ as described above.
SEQ ID NO: 163: QLLEELLEELLQGFGFVGGELLQQLLQQLLEKG, wherein the N-terminal Q at position 1 comprises the “Fluor” group described above, K is functionalized with a Z group as illustrated above and the C-terminal G at position 33 is G′ as described above.
SEQ ID NO: 164: ELLEQLLQELLEGFGFVGGOLLEQLLOQLLOKG, wherein the N-terminal E at position 1 comprises the “Fluor” group described above, K is functionalized with a Z group as illustrated above and the C-terminal G at position 33 is G′ as described above.
SEQ ID NO: 165: ELLEQLLQELLEGFGFVGGOLLEQLLQQLLEKG, wherein the N-terminal E at position 1 comprises the “Fluor” group described above, K is functionalized with a Z group as illustrated above and the C-terminal G at position 33 is G′ as described above.
SEQ ID NO: 166: ELLEQLLQELLEGFGFVGGQLLQQLLEQLLEKG, wherein the N-terminal E at position 1 comprises the “Fluor” group described above, K is functionalized with a Z group as illustrated above and the C-terminal G at position 33 is G′ as described above.
SEQ ID NO: 167: ELLEQLLQELLEGFGFVGGELLQELLQELLQKG, wherein the N-terminal E at position 1 comprises the “Fluor” group described above, K is functionalized with a Z group as illustrated above and the C-terminal G at position 33 is G′ as described above.
SEQ ID NO: 168: ELLQELLQELLQGFGFVGGELLQELLQELLOKG, wherein the N-terminal E at position 1 comprises the “Fluor” group described above, K is functionalized with a Z group as illustrated above and the C-terminal G at position 33 is G′ as described above.
SEQ ID NO: 169: ELLQELLQELLOGFGFVGGOLLEQLLEQLLEKG, wherein the N-terminal E at position 1 comprises the “Fluor” group described above, K is functionalized with a Z group as illustrated above and the C-terminal G at position 33 is G′ as described above.
SEQ ID NO: 170: ELLQQLLEQLLEGFGFVGGELLQELLQQLLEKG, wherein the N-terminal E at position 1 comprises the “Fluor” group described above, K is functionalized with a Z group as illustrated above and the C-terminal G at position 33 is G′ as described above.
SEQ ID NO: 171: QLLEQLLQQLLEGFGFVGGOLLEQLLQQLLEKG, wherein the N-terminal Q at position 1 comprises the “Fluor” group described above, K is functionalized with a Z group as illustrated above and the C-terminal G at position 33 is G′ as described above.

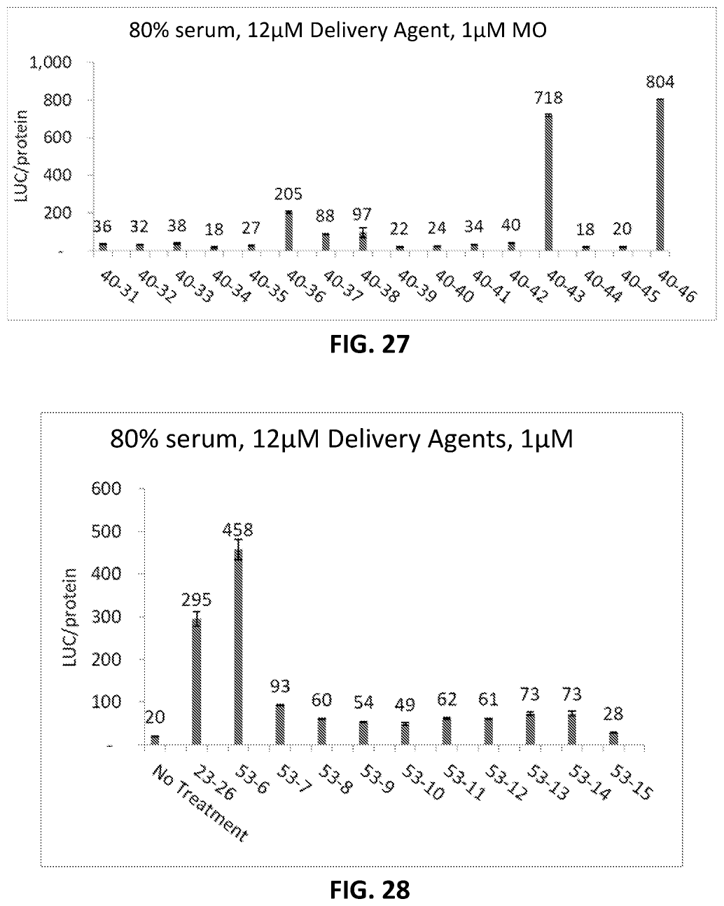

As illustrated in FIG. 27, in some aspects of the disclosure, delivery agents retain activity when the bolded positions are modified relative to the peptide sequence 40-46 with glutamines replacing glutamates in the lytic peptide group and glutamates replacing glutamines in the mask peptide group.

### Example 26

In this experiment, delivery agent examples comprising mask peptide groups having a glutamine replaced by a glutamate and lytic peptide groups having an additional N-terminal glutamate were evaluated. In the examples of this example, the additional N-terminal glutamate of the lytic peptide group can improve solubility. The particular delivery agents are described below and results are shown in FIG. 28.
Negative control: No treatment (This sample contained a morpholino but no delivery agent)
Positive control: 23-26: fluor-[Lytic]-[CL]-[Mask]-[2×K(LA)]-[G′]
53-6: fluor-[E]-[ELL EEL LEE LLE ELL E]-[CL]-[QLL QQL LOQ LLQ QLL Q]-[2×K(LA)]-[G′]
53-7: fluor-[E]-[ELL EEL LEE LLE ELL E]-[CL]-[ELL QQL LQQ LLQ QLL Q]-[2×K(LA)]-[G′]
53-8: fluor-[E]-[ELL EEL LEE LLE ELL E]-[CL]-[QLL EQL LOQ LLQ QLL Q]-[2×K(LA)]-[G′]
53-9: fluor-[E]-[ELL EEL LEE LLE ELL E]-[CL]-[QLL QEL LQQ LLQ QLL Q]-[2×K(LA)]-[G′]
53-10: fluor-[E]-[ELL EEL LEE LLE ELL E]-[CL]-[QLL QQL LEQ LLQ QLL Q]-[2×K(LA)]-[G′]
53-11: fluor-[E]-[ELL EEL LEE LLE ELL E]-[CL]-[QLL QQL LQE LLQ QLL Q]-[2×K(LA)]-[G′]
53-12: fluor-[E]-[ELL EEL LEE LLE ELL E]-[CL]-[QLL QQL LQQ LLE QLL Q]-[2×K(LA)]-[G′]
53-13: fluor-[E]-[ELL EEL LEE LLE ELL E]-[CL]-[QLL QQL LQQ LLQ ELL Q]-[2×K(LA)]-[G′]
53-14: fluor-[E]-[ELL EEL LEE LLE ELL E]-[CL]-[QLL QQL LQQ LLQ QLL E]-[2×K(LA)]-[G′]
53-15: fluor-[E]-[ELL EEL LEE LLE ELL E]-[CL]-[ELL QQL LQQ LLQ QLL E]-[2×K(LA)]-[G′]

SEQ ID NO: 172: QLLQQLLQQLLQQLLQ
SEQ ID NO: 173: ELLQQLLQQLLQQLLQ
SEQ ID NO: 174: QLLEQLLQQLLQQLLQ
SEQ ID NO: 175: QLLQELLQQLLQQLLQ
SEQ ID NO: 176: QLLQQLLEQLLQQLLQ
SEQ ID NO: 177: QLLQQLLQELLQQLLQ
SEQ ID NO: 178: QLLQQLLQQLLEQLLQ
SEQ ID NO: 179: QLLQQLLQQLLQELLQ
SEQ ID NO: 180: QLLQQLLQQLLQQLLE
SEQ ID NO: 181: ELLQQLLQQLLQQLLE

SEQ ID NO: 182: EELLEELLEELLEELLEGFGFVGGQLLQQLLQQLLQQLLOKKG, wherein the N-terminal E at position 1 comprises the “Fluor” group described above, each K is functionalized with an LA group, and the C-terminal G at position 43 is G′ as described above.
SEQ ID NO: 183: EELLEELLEELLEELLEGFGFVGGELLQQLLQQLLQQLLOKKG, wherein the N-terminal E at position 1 comprises the “Fluor” group described above, each K is functionalized with an LA group, and the C-terminal G at position 43 is G′ as described above.
SEQ ID NO: 184: EELLEELLEELLEELLEGFGFVGGOLLEQLLOQLLQQLLOKKG, wherein the N-terminal E at position 1 comprises the “Fluor” group described above, each K is functionalized with an LA group, and the C-terminal G at position 43 is G′ as described above.
SEQ ID NO: 185: EELLEELLEELLEELLEGFGFVGGQLLQELLQQLLQQLLOKKG, wherein the N-terminal E at position 1 comprises the “Fluor” group described above, each K is functionalized with an LA group, and the C-terminal G at position 43 is G′ as described above.
SEQ ID NO: 186: EELLEELLEELLEELLEGFGFVGGQLLQQLLEQLLQQLLOKKG, wherein the N-terminal E at position 1 comprises the “Fluor” group described above, each K is functionalized with an LA group, and the C-terminal G at position 43 is G′ as described above.
SEQ ID NO: 187: EELLEELLEELLEELLEGFGFVGGQLLQQLLQELLQQLLQKKG, wherein the N-terminal E at position 1 comprises the “Fluor” group described above, each K is functionalized with an LA group, and the C-terminal G at position 43 is G′ as described above.
SEQ ID NO: 188: EELLEELLEELLEELLEGFGFVGGQLLQQLLQQLLEQLLQKKG, wherein the N-terminal E at position 1 comprises the “Fluor” group described above, each K is functionalized with an LA group, and the C-terminal G at position 43 is G′ as described above.
SEQ ID NO: 189: EELLEELLEELLEELLEGFGFVGGQLLQQLLQQLLQELLQKKG, wherein the N-terminal E at position 1 comprises the “Fluor” group described above, each K is functionalized with an LA group, and the C-terminal G at position 43 is G′ as described above.
SEQ ID NO: 190: EELLEELLEELLEELLEGFGFVGGOLLQQLLOOLLQQLLEKKG, wherein the N-terminal E at position 1 comprises the “Fluor” group described above, each K is functionalized with an LA group, and the C-terminal G at position 43 is G′ as described above.
SEQ ID NO: 191: EELLEELLEELLEELLEGFGFVGGELLQQLLQQLLQQLLEKKG, wherein the N-terminal E at position 1 comprises the “Fluor” group described above, each K is functionalized with an LA group, and the C-terminal G at position 43 is G′ as described above.

Without being limited to a particular theory, replacing a glutamine with a negatively charged amino acid in the mask can reduce activity if the change is not compensated in the lytic peptide group by substituting one or more glutamines in place of the one/multiple glutamates.

### Example 27

In this example, a delivery agent embodiment without a lytic peptide group was evaluated. The particular delivery agents are described below.
96-10: Cap-[Mask]-[2×K(LA)-[GNalG′], Insoluble in Water so dissolved in DMSO.
23-26: fluor-[Lytic]-[CL]-[Mask]-[2×K(LA)]-[G]
26-13A: fluor-[Lytic]-[CL]-[Mask]-[2×K(LA)]-[G′]-[CH₂CH(CH₃)₂] (C-terminus group is isobutyl-amide)
Negative control: Morpholino only (MO) in DMSO
SEQ ID NO: 192: QLLQQLLQQLLOKKGXG, wherein the N-terminal Q at position 1 comprises the “Cap” group described above, each K is functionalized with an LA group, X is Nal, and the C-terminal G at position 17 is G′ as described above.
SEQ ID NO: 250: ELLEELLEELLEGFGFVGGQLLQQLLQQLLOKKG, wherein the N-terminal E at position 1 comprises the “Fluor” group described above, each K is functionalized with an LA group, and the C-terminal G at position 34 comprises a C-terminus modified with a i-butyl amine group

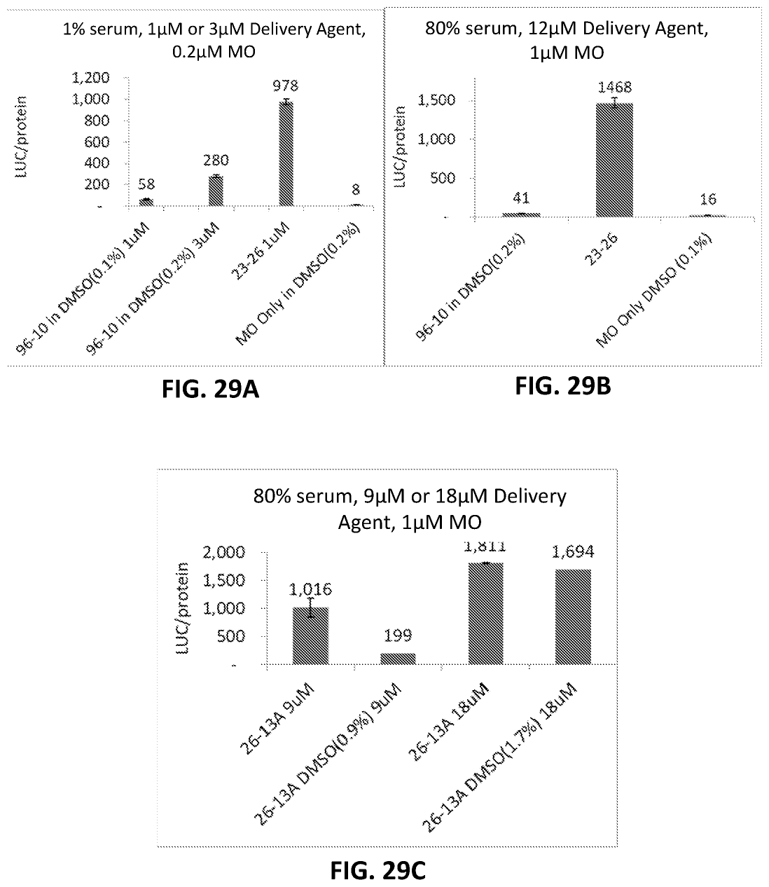

Results are provided by FIGS. 29A-29C. In this example, the embodiment designated “96-10,” which comprises a mask peptide group but no lytic peptide group, shows little activity compared to the control, embodiment 23-26. Embodiment 96-10 is poorly soluble in water. DMSO is commonly used as a solubilizing agent for poorly water-soluble molecules. In FIG. 29C the embodiment designated “23-13A” has activity when combined with DMSO, suggesting that DMSO does not terminate the activity of some aspects of the disclosure. Without being limited to a particular theory, the poor activity of embodiment 96-10 is likely not due to the addition of DMSO but rather because the sequence is not conducive to substantial activity leading to endosomal escape.

### Example 28

In this example, delivery agent examples comprising one, two, or three anchor groups were evaluated and compared in 80% serum. The particular delivery agents are described below.
21-1: fluor-[Lytic]-[CL]-[Mask]-[1×K(LA)]-[G′]
21-4: fluor-[Lytic]-[CL]-[Mask]-[2×K(LA)]-[G′]
21-6: fluor-[Lytic]-[CL]-[Mask]-[3×K(LA)]-[G′]
Negative control: Morpholino only (MO only)
SEQ ID NO: 193: ELLEELLEELLEGFGFVGGOLLQQLLQQLLOKKKG, wherein the N-terminal E at position 1 comprises the “Fluor” group described above, each K is functionalized with an LA group, and the C-terminal G at position 35 is G′ as described above.

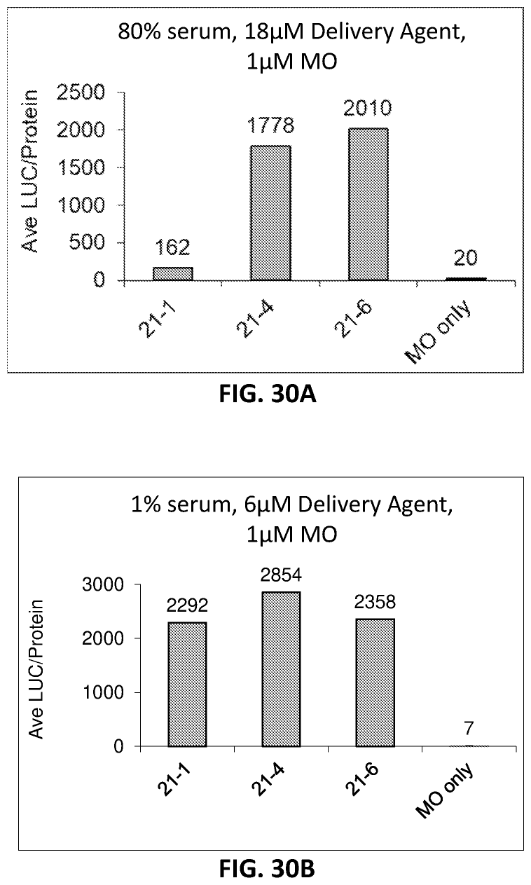

As illustrated in FIGS. 30A and 30B, in some aspects of the disclosure, delivery agent (18 μM) activity in 80% serum is lowest when only one anchor group was present. Additionally, delivery agent activity in this experiment was higher for three anchor groups than for two anchor groups.

### Example 29

In this example, delivery agent examples comprising one, two, or three anchor groups were evaluated and compared at various concentrations in 80% serum. The particular delivery agents are described below:
19-1: fluor-[Lytic]-[CL]-[Mask]-[1×K(LA)]-[G′]
21-4: fluor-[Lytic]-[CL]-[Mask]-[2×K(LA)]-[G′]
21-6: fluor-[Lytic]-[CL]-[Mask]-[3×K(LA)]-[G′]
Negative control: Morpholino only (MO only)

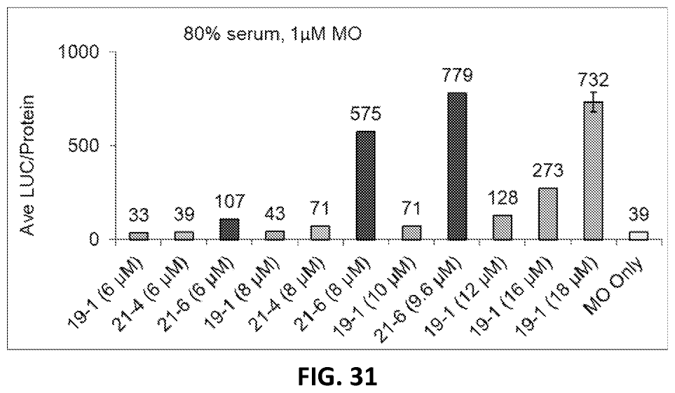

As illustrated in FIG. 31, in this embodiment, at each concentration of delivery agent, delivery agent activity in 80% serum increased as the number of anchor groups increased.

### Example 30

In this example, delivery agent examples comprising one or two anchor groups were evaluated and compared in 80% serum. The particular delivery agents are described below:
23-27: fluor-[Lytic]-[CL]-[Mask]-[1×K(LA)]-[G]
23-4: fluor-[Lytic]-[CL]-[Mask]-[2×K(LA)]-[G′]

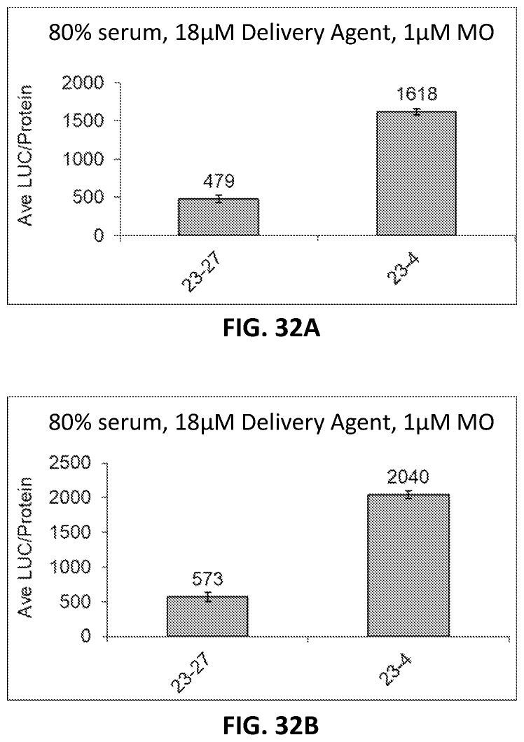

As shown in FIG. 32A, delivery agent activity in this experiment was higher for two anchor groups than for one anchor groups. FIG. 32B shows the second repetition of this experiment.

### Example 31

In this example, delivery agent examples comprising one, two, or three anchor groups were evaluated and compared in 80% serum. The particular delivery agents are described below:
19-1: fluor-[Lytic]-[CL]-[Mask]-[1×K(LA)]-[G]
23-4: fluor-[Lytic]-[CL]-[Mask]-[2×K(LA)]-[G′]
23-5: fluor-[Lytic]-[CL]-[Mask]-[3×K(LA)]-[G′]

As illustrated in FIG. 33, in this embodiment, delivery agent activity in 80% serum increased as the number of anchor groups increased.

### Example 32

In this example, delivery agents comprising anchor groups having either quaternary or secondary amines were evaluated and compared. The particular delivery agents are described below. In the chemical structures below “a” represents an anchor group containing a quaternary amine, “b” represents an anchor group having a secondary amine, and “n” denotes the number of methylene units in the carbon chain of the anchor group(s).
26-7: fluor-[Lytic]-[CL]-[Mask]-[1×K(LA)], where:
26-7a: K(LA)=a, n=7
26-7a′: K(LA)=a, n=9
26-7b: K(LA)=b, n=7
26-7b′: K(LA)=b, n=9
26-14: fluor-[Lytic]-[CL]-[Mask]-[2×K(LA)], where:
26-14A: K(LA)=a, n=7
26-14B: K(LA)=b, n=7
The C-terminus group in delivery agents 26-7a-b′ and 26-14A/B is N—CH₂—CH₃—(CH₂)₂. In some aspects of the disclosure, this modification does not impact activity.

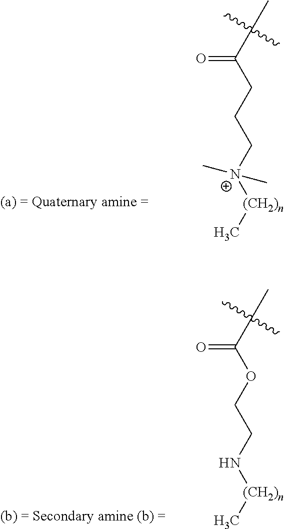

SEQ ID NO: 252: ELLEELLEELLEGFGFVGGOLLQQLLOOLLOK, wherein the N-terminal E at position 1 comprises the “Fluor” group described above, K is functionalized with quaternary amine (a) illustrated above, wherein n is 9 and further comprises a C-terminus functionalized with an isobutyl group (CH₂—CH₃—(CH₂)₂)
SEQ ID NO: 253: ELLEELLEELLEGFGFVGGQLLQQLLQQLLQK, wherein the N-terminal E at position 1 comprises the “Fluor” group described above, K is functionalized with secondary amine (b) illustrated above, wherein n is 7 and further comprises a C-terminus functionalized with an isobutyl group (CH₂—CH₃—(CH₂)₂)
SEQ ID NO: 254: ELLEELLEELLEGFGFVGGOLLQQLLOOLLQK, wherein the N-terminal E at position 1 comprises the “Fluor” group described above, K is functionalized with secondary amine (b) illustrated above, wherein n is 9 and further comprises a C-terminus functionalized with an isobutyl group (CH₂—CH₃—(CH₂)₂)
SEQ ID NO: 255: ELLEELLEELLEGFGFVGGOLLQQLLQQLLOKK, wherein the N-terminal E at position 1 comprises the “Fluor” group described above, each K is functionalized with secondary amine (b) illustrated above, wherein n is 7 and further comprises a C-terminus functionalized with an isobutyl group (CH₂—CH₃—(CH₂)₂)

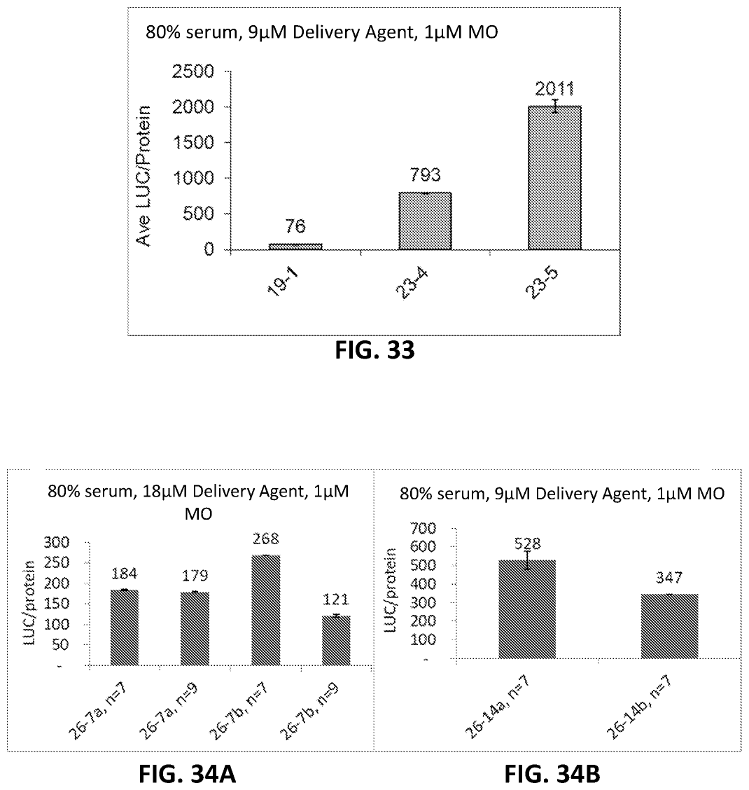

As illustrated in FIGS. 34A and 34B, both quaternary and secondary amines in the anchor groups produce delivery agents that have activity.

### Example 33

In this example, delivery agent examples comprising five, seven, nine, or eleven methylenes in the carbon chain of the anchor groups were evaluated and compared at various concentrations in 80% serum. The particular delivery agents are described below:
26-14: fluor-[Lytic]-[CL]-[Mask]-[2×K(LA)], wherein:
26-14A: n=5 carbon
26-14B: n=7 carbon
26-14C: n=9 carbon
26-14D: n=11 carbon
The C-terminus group in delivery agent 26-14 is N—CH₂—CH₃—(CH₂)₂. In some aspects of the disclosure, this modification does not impact activity.

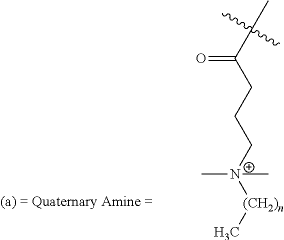

SEQ ID NO: 256: ELLEELLEELLEGFGFVGGQLLOOLLQQLLOKK, wherein the N-terminal E at position 1 comprises the “Fluor” group described above, each K is functionalized with quaternary amine (a) illustrated above, wherein n is 11 and further comprises a C-terminus functionalized with an isobutyl group (CH₂—CH₃—(CH₂)₂)

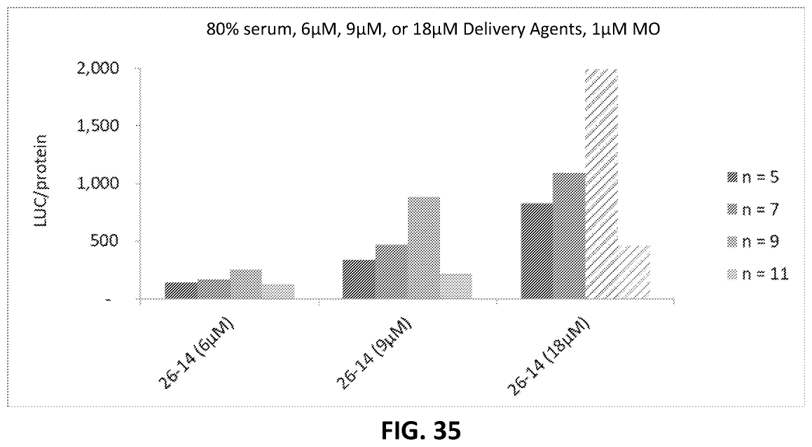

As illustrated in FIG. 35, in this embodiment, delivery agent 26-14 exhibits highest activity with a nine-carbon anchor group. Dashed lines in FIG. 35 denote observed toxicity.

### Example 34

In this example, delivery agent examples comprising anchor groups of varying lengths were compared at two concentrations in 80% serum. The particular delivery agents are described below:
26-15: fluor-[Lytic]-[CL]-[Mask]-[3×K(LA)]-[G′]
with:
26-15A: n=5 carbon
26-15B: n=7 carbon
The C-terminus group of delivery agent 26-15 is N—CH₂—CH₃—(CH₂)₂. In some aspects of the disclosure, this modification does not impact activity.
Negative control: Morpholino Only (MO only)

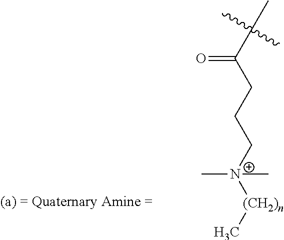

SEQ ID NO: 251: ELLEELLEELLEGFGFVGGOLLQQLLOOLLQKKKG, wherein the N-terminal E at position 1 comprises the “Fluor” group described above, each K is functionalized with quaternary amine (a) illustrated above, wherein n is 5 and the C-terminal G at position 34 comprises a C-terminus modified with a i-butyl amine group

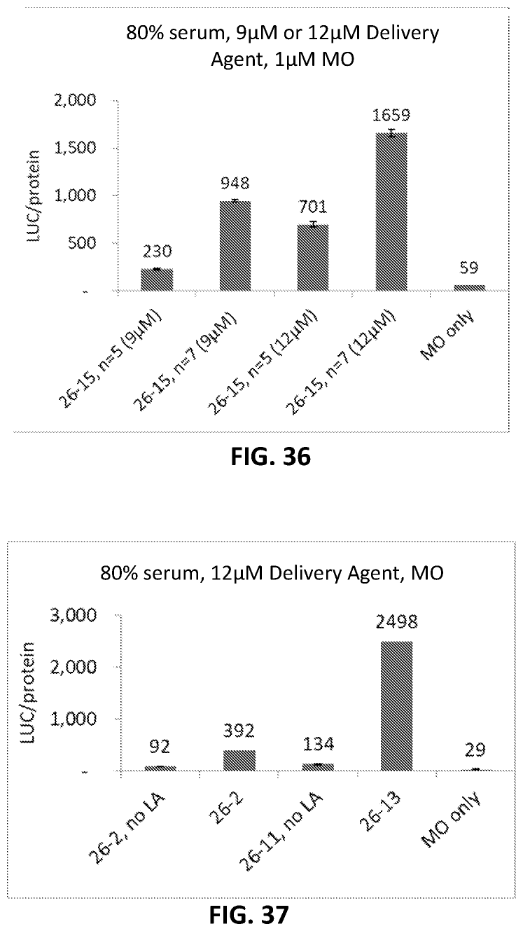

As illustrated in FIG. 36, in this embodiment, delivery agent 26-15 exhibits highest activity with a carbon chain seven carbons in length as indicated above.

### Example 35

In this example, delivery agent examples comprising anchor groups with or without aliphatic tails were evaluated in 80% serum. The particular delivery agents are described below:
26-2A, no LA: fluor-[Lytic]-[CL]-[Mask]-[1×K]-[G]
26-2B: fluor-[Lytic]-[CL]-[Mask]-[1×K(LA)]-[G′]
26-11, no LA: fluor-[Lytic]-[CL]-[Mask]-[2×K]
26-13: fluor-[Lytic]-[CL]-[Mask]-[2×K(LA)]
Negative control: Morpholino Only (MO only)
SEQ ID NO: 257: ELLEELLEELLEGFGFVGGOLLQQLLQQLLOKKG, wherein the N-terminal E at position 1 comprises the “Fluor” group described above, the C-terminal G at position 34 is G′ as described above.
SEQ ID NO: 258: ELLEELLEELLEGFGFVGGQLLQQLLOOLLQKK, wherein the N-terminal E at position 1 comprises the “Fluor” group described above.
SEQ ID NO: 194: ELLEELLEELLEGFGFVGGQLLOOLLQQLLOKK, wherein the N-terminal E at position 1 comprises the “Fluor” group described above, each K is functionalized with an LA Group.

As shown in FIG. 37, in this embodiment, delivery agents comprising anchor groups without aliphatic tails exhibited decreased activity as compared to examples of delivery agents having anchor groups with aliphatic tails.

### Example 36

In this example, delivery agent examples with and without anchor groups were evaluated. The particular delivery agents are described below:
33-24: fluor-[Lytic]-[CL]-[Mask]-[G′]
23-26: fluor-[Lytic]-[CL]-[Mask]-[2×K(LA)]-[G]
Negative controls: Morpholino only (MO Only) or No treatment

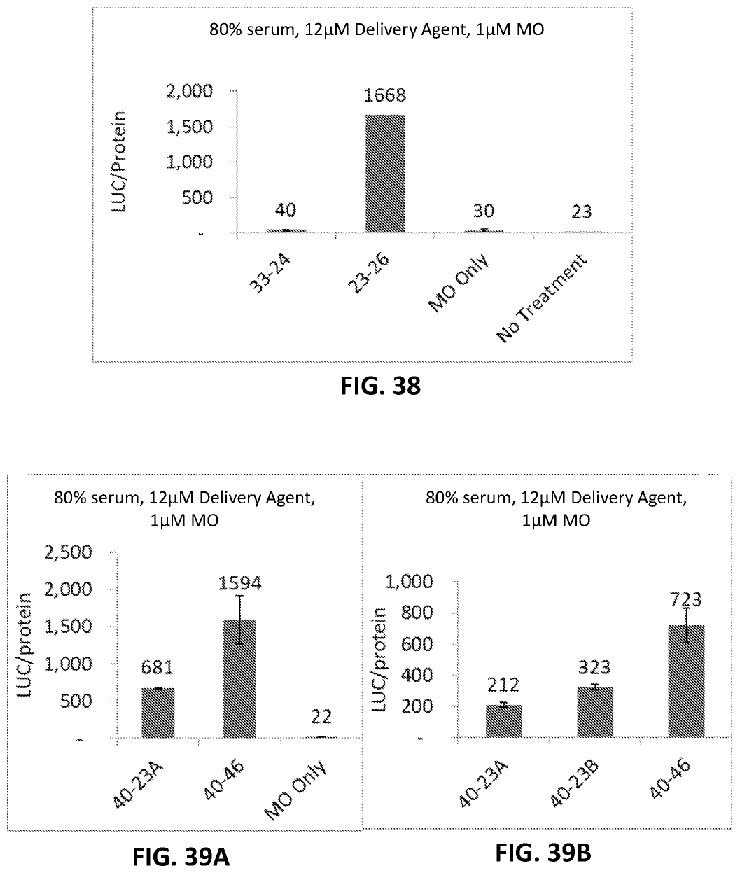

As shown in FIG. 38, in some aspects of the disclosure, delivery agents with no anchor groups exhibit no activity in high serum conditions.

### Example 37

In this example, delivery agents having different configurations of the anchor groups were evaluated. The particular delivery agents are described below:
Negative control: Morpholino Only (MO only)
40-23A: fluor-[Lytic]-[CL]-[MASK]-[K(Z)]-[G′]
40-46: fluor-[Lytic]-[CL]-[MASK]-[2×K(LA)]-[G′]
40-23B: fluor-[Lytic]-[CL]-[Mask]-[K(Y)]-[G]

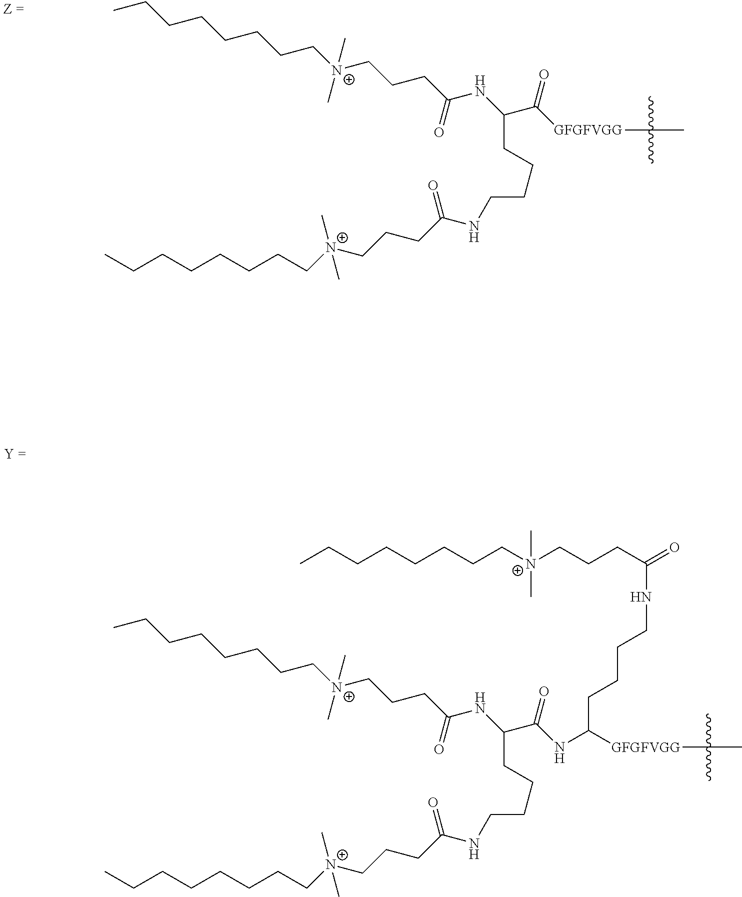

SEQ ID NO: 259: ELLEELLEELLEGFGFVGGOLLOQLLOQLLOKG, wherein the N-terminal E at position 1 comprises the “Fluor” group described above, K is functionalized with a Y group as illustrated above and the C-terminal G at position 33 is G′ as described above.
SEQ ID NO: 271: ELLEELLEELLEGFGFVGGOLLQQLLQQLLQKG, wherein the N-terminal E at position 1 comprises the “Fluor” group described above, K is functionalized with a Z group as illustrated above and the C-terminal G at position 33 is G′ as described above.

As illustrated in FIGS. 39A and 39B, in particular examples, the positioning of the anchor groups directly C-terminal to the mask peptide group results in delivery agents having high activity.

### Example 38

In this example, delivery agent examples comprising two, three, or four anchor groups having various structures shown below were evaluated. The particular delivery agents are described below:
21-4: fluor-[Lytic]-[CL]-[Mask]-[2×K(LA)]-[G′]
21-4a: LA=a, n=7
21-4b: LA=b, n=6
21-4b′: LA=b, n=8
21-6: fluor-[Lytic]-[CL]-[Mask]-[3×K(LA)]-[G′]
21-6a: LA=a, n=7
21-6b: LA=b, n=6
21-7: fluor-[Lytic]-[CL]-[Mask]-[4×K(LA)]-[G]
21-7b: LA=b, n=6

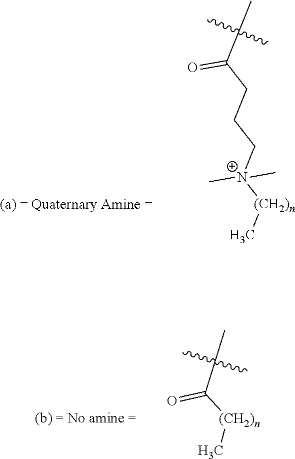

SEQ ID NO: 260: ELLEELLEELLEGFGFVGGQLLQQLLQQLLOKKG, wherein the N-terminal E at position 1 comprises the “Fluor” group described above, each K is functionalized with group (b) illustrated above, wherein n is 6, and the C-terminal G at position 34 is G′ as described above.
SEQ ID NO: 261: ELLEELLEELLEGFGFVGGQLLQQLLQQLLOKKG, wherein the N-terminal E at position 1 comprises the “Fluor” group described above, each K is functionalized with group (b) illustrated above, wherein n is 8, and the C-terminal G at position 34 is G′ as described above.
SEQ ID NO: 262: ELLEELLEELLEGFGFVGGOLLQQLLQQLLOKKKG, wherein the N-terminal E at position 1 comprises the “Fluor” group described above, each K is functionalized with group (b) illustrated above, wherein n is 6, and the C-terminal G at position 35 is G′ as described above.
SEQ ID NO: 195: ELLEELLEELLEGFGFVGGQLLQQLLOOLLQKKKKG, wherein the N-terminal E at position 1 comprises the “Fluor” group described above, each K is functionalized with group (b) illustrated above, wherein n is 6, and the C-terminal G at position 36 is G′ as described above.

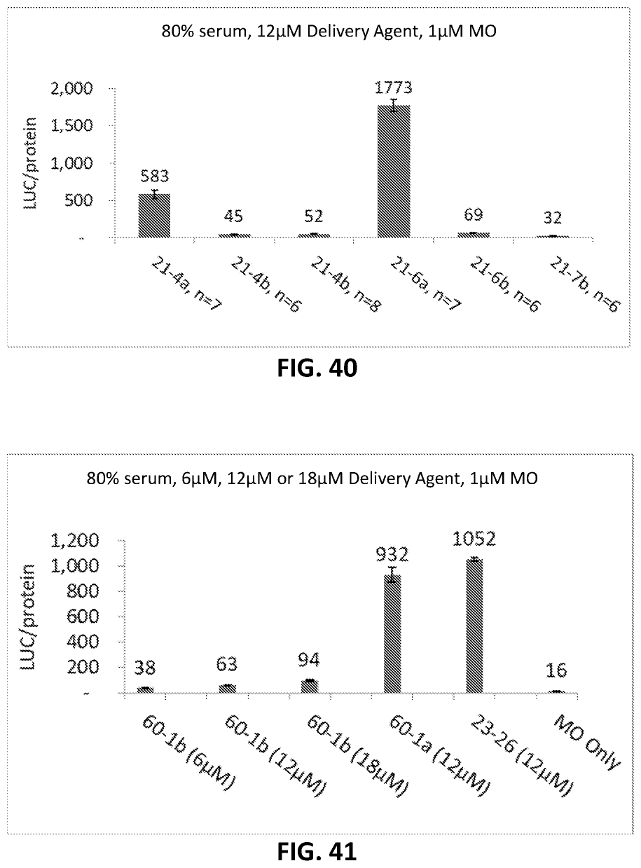

As shown in FIG. 40, delivery agents comprising simple alkyl chain anchor groups were inactive. This example suggests that, in some aspects of the disclosure, an amine group that is positively charged at physiological pH contributes to delivery agent activity.

### Example 39

In this example, delivery agent examples comprising a perfluoro chain (comprising eight fluorinated carbons, and having a total length of eleven carbons) rather than an alkyl chain (having a length of eight carbons) on the anchor groups were evaluated. The particular delivery agents are described below:
60-1: fluor-[Lytic]-[CL]-[Mask]-[2×K(LA)]-[G′]
60-1a: LA=a
60-1b: LA=b
23-26: fluor-[Lytic]-[CL]-[Mask]-[2×K(LA)]-[G′]
Negative control: Morpholino Only (MO only)

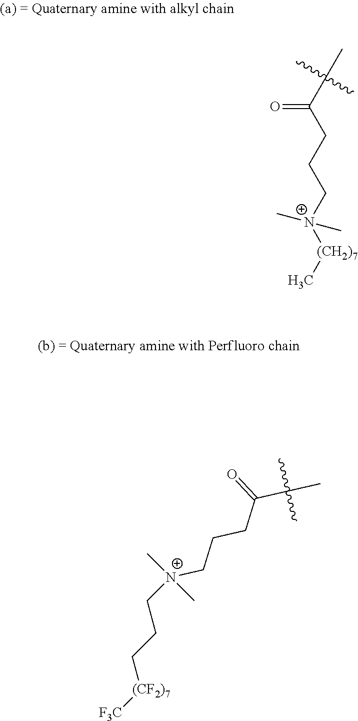

SEQ ID NO: 263: ELLEELLEELLEGFGFVGGOLLQQLLOOLLQKKG, wherein the N-terminal E at position 1 comprises the “Fluor” group described above, each K is functionalized with perfluoro-containing group (b) illustrated above and the C-terminal G at position 34 is G′ as described above.

As shown in FIG. 41, in this embodiment, delivery agents having anchor groups with perfluoro chains exhibited reduced activity compared to delivery agents having anchor groups with alkyl chains.

### Example 40

In this example, delivery agent examples having two, eight-carbon anchor groups or two arginines substituted for the anchor groups, and comprising lytic peptide groups wherein all glutamates except the C-terminal glutamate are replaced with aspartic acid, were evaluated. The particular delivery agents are described below:
109-1: Cap-[DLL DDL LDD LLE]-[CL]-[Mask]-[2×K(LA)]-[GNalG′]
109-8: Cap-[DLL DDL LDD LLE]-[CL]-[Mask]-[RR]-[GNalG′]
104-3+4: Cap-[DLL DDL LDD LLE]-[CL]-[Mask]-[2×K(LA)]-[GNalG′]
Negative control: Morpholino only (MO only)
SEQ ID NO: 196: DLLDDLLDDLLEGFGFVGGQLLQQLLQQLLORRGXG, wherein the N-terminal D at position 1 comprises the “Cap” group described above, X is Nal and the C-terminal G at position 36 is G′ as described above.

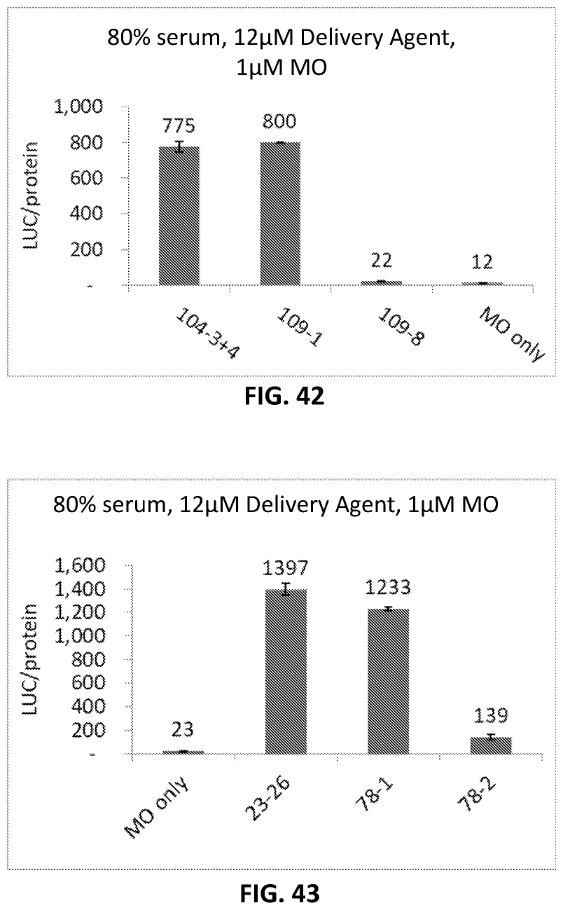

As shown in FIG. 42, delivery agent 109-1 exhibited activity comparable to that of a delivery agent having glutamates rather than aspartic acid groups in the lytic peptide group. However, delivery agent 109-8, with arginines substituted for the two, eight-carbon anchor groups, was inactive. These data suggest that, in some aspects of the disclosure, anchor groups having an alkyl chain and an amine group that is positively charged at physiological pH contribute to delivery agent activity.

In some aspects of the disclosure, synthesis of a delivery agent comprising a lytic peptide group wherein all glutamates except the C-terminal glutamate are replaced with aspartic acid groups is comparable in activity to that of a delivery agent comprising a lytic peptide group bearing glutamates rather than aspartic acid groups. Further, in this example, it was found that a C-terminal glutamate in the lytic peptide group improved delivery agent synthesis quality as compared to delivery agents having an aspartic acid at the same position. During solid phase peptide synthesis, an aspartic acid N-terminal to a glycine can undergo aspartimide formation, resulting in chain termination and other unwanted side-products (Neumann et al., Nat Commun. 11:982, 2020). It is believed retaining a glutamate in the C-terminal position of the lytic peptide group alleviates this problem.

### Example 41

In this example, delivery agent examples having a tryptophan at the N-terminus of the lytic peptide group or between two glycines at the C-terminus group of the delivery agent were evaluated. All samples in this example were crude (unpurified). The particular delivery agents are described below:
Negative Control: Morpholino only
Positive Control: 23-26: Fluor-[Lytic]-[CL]-[Mask]-[2×K(LA)]-[G′]
78-1: Cap-[Lytic]-[CL]-[Mask]-[2×K(LA)]-[GWG′]
78-2: Cap-W-[Lytic]-[CL]-[Mask]-[2×K(LA]]-[G′]
SEQ ID NO: 264: ELLEELLEELLEGFGFVGGQLLQQLLQQLLQKKGWG, wherein the N-terminal E at position 1 comprises the “Cap” group described above, each K is functionalized with an LA group, the C-terminal G at position 36 is G′ as described above, and the N-terminal E comprises a C-terminus modified with an acetyl group.
SEQ ID NO: 197: WELLEELLEELLEGFGFVGGQLLQQLLQQLLOKKG, wherein the N-terminal W at position 1 comprises the “Cap” group described above, each K is functionalized with an LA group, the C-terminal G at position 35 is G′ as described above, and the N-terminal W comprises a C-terminus modified with an acetyl group.

As illustrated in FIG. 43, in this embodiment, the presence of a tryptophan at the N-terminus of the lytic peptide group attenuated delivery agent activity.

### Example 42

In this example, delivery agent examples having varied N- and C-terminus group structures were evaluated. All samples except for the positive control were tested as both crude (C) and purified (P) preparations. Samples were purified using Q-Sepharose High performance resin and a NaCl gradient. The particular delivery agents are described below:
Negative Control: Morpholino only
Positive Control: 23-26: Fluor-[Lytic]-[CL]-[Mask]-[2×K(LA)]-[G′]
85-2: Fluor-[Lytic]-[CL]-[Mask]-[2×K(LA)]-[G′]
86-1: Cap-[Lytic]-[CL]-[Mask]-[2×K(LA)]-[GWG′]
86-4: Cap-[Lytic]-[CL]-[Mask]-[2×K(LA)]-[GNalG′]
SEQ ID NO: 249: ELLEELLEELLEGFGFVGGOLLQQLLQQLLOKKGXG, wherein the N-terminal E at position 1 comprises the “Cap” group described above, each K is functionalized with an LA group, X is Nal, the C-terminal G at position 36 is G′ as described above.

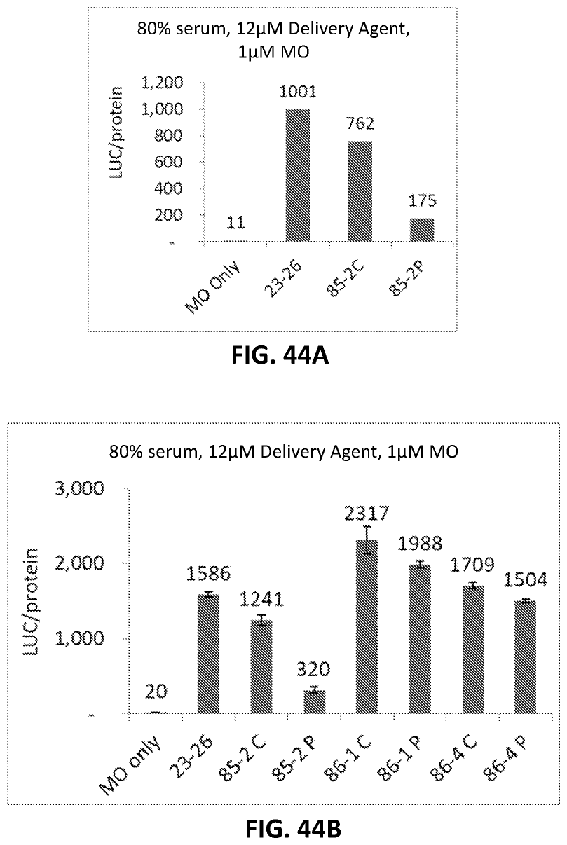

As illustrated in FIGS. 44A and 44B, delivery agents 86-1 and 86-4 did not lose activity after purification, while activity of delivery agent 85-2 was reduced by almost 4-fold. These data suggest that, in some aspects of the disclosure, the presence of a fluorophore on the N-terminus of the lytic peptide group leads to a reduction in activity in purified delivery agents as compared to the same delivery agents in a crude form.

### Example 43

In this example, delivery agent examples comprising a C-terminal glycine or a C-terminal G-Nal-Nal-G were evaluated. Delivery agent 86-5 was tested as both a crude (C) and purified (P) preparation. Samples were purified using Q-Sepharose High performance resin and a NaCl gradient. The particular delivery agents are described below:
Negative Control: Morpholino only
Positive Control: 23-26: Fluor-[Lytic]-[CL]-[Mask]-[2×K(LA)]-[G′]
86-5: Cap-[Lytic]-[CL]-[Mask]-[2×K(LA)]-[GNalNal]-G′
SEQ ID NO: 198: ELLEELLEELLEGFGFVGGQLLQQLLQQLLQKKGXXG, wherein the N-terminal E at position 1 comprises the “Cap” group described above, each K is functionalized with an LA group, each X is Nal, and the C-terminal G at position 37 is G′ as described above.

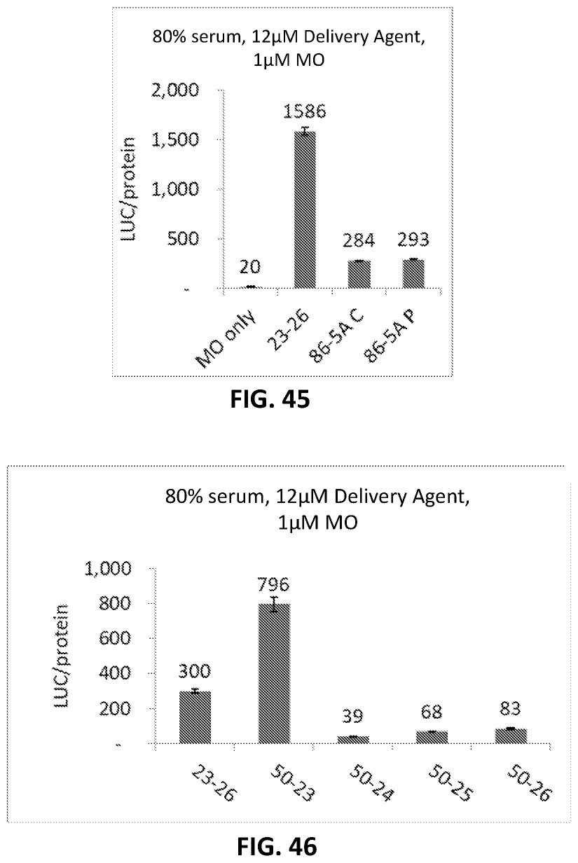

As seen in FIG. 45, the delivery agent having an N-terminal glycine exhibited increased activity than the delivery agent having an N-terminal G-Nal-Nal-G (where Nal is naphthylalanine), regardless of delivery agent purity.

### Example 44

In this example, delivery agent examples bearing additional glutamate residues in various locations were evaluated. The particular delivery agents are described below.
23-26: fluor-[Lytic]-[CL]-[Mask]-[2×K(LA)]-[G]
50-23: fluor-[E]-[QLL EEL LEE LLQ]-[CL]-[QLL QQL LQQ LLQ]-[2×K(LA)]-[G′]
50-24: fluor-[QLL EEL LEE LLQ]-[CL]-[QLL QQL LQQ LLQ]-[E]-[2×K(LA)]-[E]-[G′]
50-25: fluor-[QLL EEL LEE LLQ]-[CL]-[QLL QQL LQQ LLQ]-[2×K(LA)]-[E]-[G]
50-26: fluor-[QLL EEL LEE LLQ]-[CL]-[QLL QQL LQQ LLQ]-[E]-[2×K(LA)]-[G′]
SEQ ID NO: 199: EQLLEELLEELLQGFGFVGGQLLQQLLOOLLQKKG, wherein the N-terminal E at position 1 comprises the “Fluor” group described above, each K is functionalized with an LA group and the C-terminal G at position 35 is G′ as described above.
SEQ ID NO: 200: QLLEELLEELLQGFGFVGGQLLQQLLQQLLQEKKEG, wherein the N-terminal Q at position 1 comprises the “Fluor” group described above, each K is functionalized with an LA group and the C-terminal G at position 36 is G′ as described above.
SEQ ID NO: 201: QLLEELLEELLQGFGFVGGQLLQQLLQQLLOKKEG, wherein the N-terminal Q at position 1 comprises the “Fluor” group described above, each K is functionalized with an LA group and the C-terminal G at position 35 is G′ as described above.
SEQ ID NO: 202: QLLEELLEELLQGFGFVGGQLLQQLLOOLLQEKKG, wherein the N-terminal Q at position 1 comprises the “Fluor” group described above, each K is functionalized with an LA group and the C-terminal G at position 35 is G′ as described above.

Glutamates were added to the delivery agent examples shown, to increase delivery agent solubility. As shown in FIG. 46. In these examples, adding glutamates C-terminal to the mask peptide or anchor groups attenuated delivery agent activity. Further, this example suggests that, in some aspects of the disclosure, the lytic peptide group is functional even with incomplete hydrogen-bonding pairs (the lytic peptide group of delivery agent 50-23 comprises an odd number of glutamates and glutamines).

### Example 45

In this example, delivery agent examples comprising two lytic peptide groups with different combinations of glutamates and glutamines, and a second cleavable linker at the C-terminus of the mask peptide group, were evaluated. The particular delivery agents are described below:
30-3: fluor-[ELL QEL LQE LLE]-[CL]-[ELL QEL LQE LLE]-[CL]-[2×K(LA)]-[G]
30-5: fluor-[ELL QEL LQE LLQ]-[CL]-[ELL QEL LQE LLQ]-[CL]-[2×K(LA)]-[G′]
30-7: fluor-[QLL EQL LEE LLE]-[CL]-[QLL EQL LEE LLE]-[CL]-[2×K(LA)]-[G′]
30-9: fluor-[QLL EQL LEQ LLE]-[CL]-[QLL EQL LEQ LLE]-[CL]-[2×K(LA)]-[G′]
30-11: fluor-[QLL EQL LEQ LLE]-[CL]-[QLL EQL LEQ LLE]-[CL]-[2×K(LA)]-[G′]
30-15: fluor-[QLL EEL LEE LLQ]-[CL]-[QLL EEL LEE LLQ]-[CL]-[2×K(LA)]-[G′]
30-17: fluor-[ELL QEL LEQ LLE]-[CL]-[ELL QEL LEQ LLE]-[CL]-[2×K(LA)]-[G′]
30-22: fluor-[Lytic]-[CL]-[Mask]-[2×K(LA)]-[G′] (internal control of synthesis run)
23-26: fluor-[Lytic]-[CL]-[Mask]-[2×K(LA)]-[G] (positive control)

SEQ ID NO: 203: ELLQELLQELLE
SEQ ID NO: 204: ELLQELLQELLQ
SEQ ID NO: 205: QLLEQLLEELLE
SEQ ID NO: 206: QLLEELLEELLQ
SEQ ID NO: 207: ELLQELLEQLLE

SEQ ID NO: 208: ELLQELLQELLEGFGFVGGELLQELLQELLEGFGFVGGKKG, wherein the N-terminal E at position 1 comprises the “Fluor” group described above, each K is functionalized with an LA group and the C-terminal G at position 41 is G′ as described above.
SEQ ID NO: 209: ELLQELLQELLQGFGFVGGELLQELLQELLQGFGFVGGKKG, wherein the N-terminal E at position 1 comprises the “Fluor” group described above, each K is functionalized with an LA group and the C-terminal G at position 41 is G′ as described above.
SEQ ID NO: 210: QLLEQLLEELLEGFGFVGGOLLEQLLEELLEGFGFVGGKKG, wherein the N-terminal Q at position 1 comprises the “Fluor” group described above, each K is functionalized with an LA group and the C-terminal G at position 41 is G′ as described above.
SEQ ID NO: 211: QLLEQLLEQLLEGFGFVGGQLLEQLLEQLLEGFGFVGGKKG, wherein the N-terminal Q at position 1 comprises the “Fluor” group described above, each K is functionalized with an LA group and the C-terminal G at position 41 is G′ as described above.
SEQ ID NO: 212: QLLEQLLEQLLEGFGFVGGOLLEQLLEQLLEGFGFVGGKKG, wherein the N-terminal Q at position 1 comprises the “Fluor” group described above, each K is functionalized with an LA group and the C-terminal G at position 41 is G′ as described above.

SEQ ID NO: 213: QLLEELLEELLQGFGFVGGOLLEELLEELLQGFGFVGGKKG, wherein the N-terminal Q at position 1 comprises the “Fluor” group described above, each K is functionalized with an LA group and the C-terminal G at position 41 is G′ as described above.
SEQ ID NO: 214: ELLQELLEQLLEGFGFVGGELLQELLEQLLEGFGFVGGKKG, wherein the N-terminal E at position 1 comprises the “Fluor” group described above, each K is functionalized with an LA group and the C-terminal G at position 41 is G′ as described above.

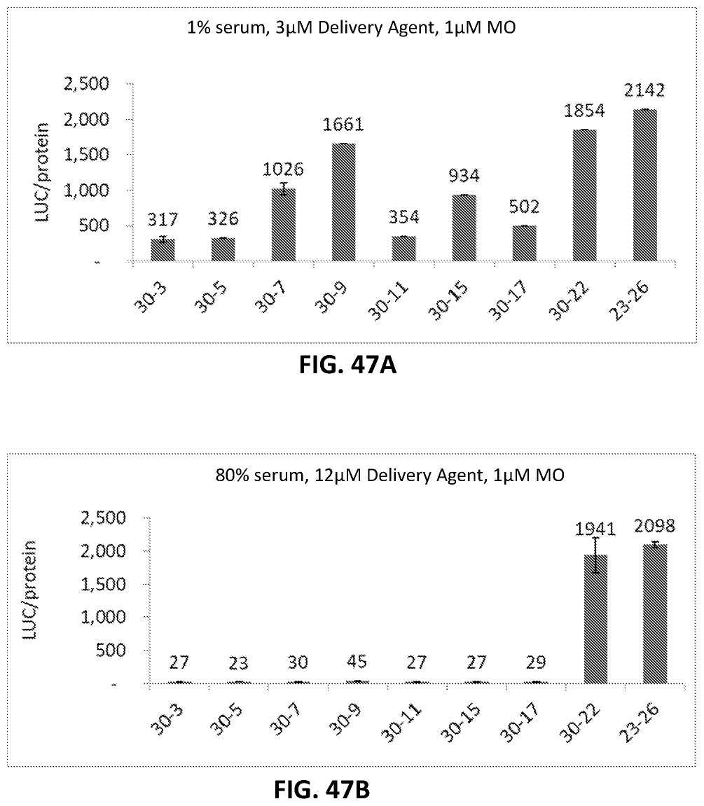

As illustrated in FIGS. 47A and 47B, delivery agent examples comprising two lytic peptide groups with different combinations of glutamates and glutamines, and a second cleavable linker at the C-terminus of the mask peptide group exhibited reduced activity in 80% serum compared to the positive control.

### Example 46

In this example, delivery agent examples wherein a single anchor group is located in different sections of the delivery agent were evaluated. In embodiment 19-17A the fluor is on the N-terminus whereas in embodiment 19-17C the anchor is on the N-terminus of the peptide. The particular delivery agents are described below:
Negative Control: Morpholino (MO) only
Positive Control: Endoporter (EP)·
19-1: fluor-[Lytic]-[CL]-[Mask]-[1×K(LA)]-[G′]
19-17A: fluor-[1×K(LA)]-[Mask]-[CL]-[Lytic]-[G′]
19-17C: LA-[1×K(fluor)]-[Mask]-[CL]-[Lytic]-[G′]
SEQ ID NO: 265: KQLLQQLLQQLLQGFGFVGGELLEELLEELLEG, wherein K is functionalized with an LA group on its side chain group and its N-terminus is functionalized with the fluorophore group described above, and the C-terminal G at position 33 is G′ as described above.
SEQ ID NO: 215: KELLEELLEELLEGFGFVGGOLLQQLLQQLLQG, wherein K is functionalized with an LA group at its N-terminus and its side chain is functionalized with the fluorophore group described above, and the C-terminal G at position 33 is G′ as described above.

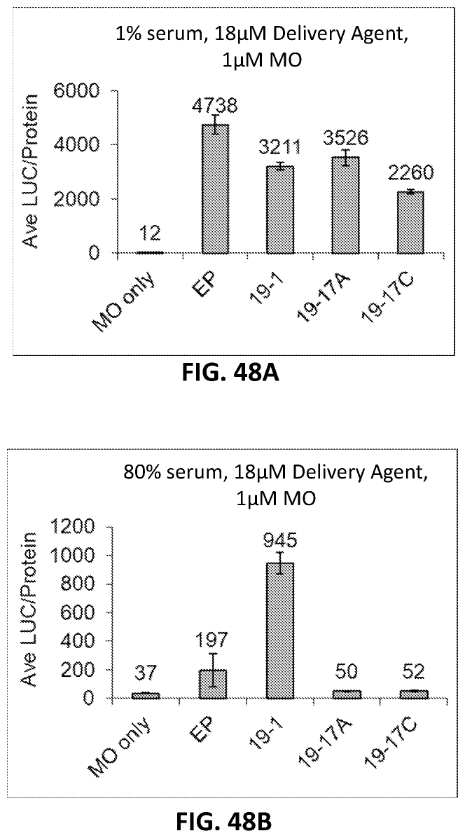

As can be seen in FIGS. 48A and 48B, the delivery agent examples of this example all showed similar activity levels in a low (1%) serum cell culture experiment; however, in high (80%) serum, delivery agents 19-17A and 19-17C exhibited reduced activity. As demonstrated in Example 48, a delivery agent of the form fluor-[Mask]-[1×K(LA)]-[CL]-[Lytic]-[G′], wherein the anchor group is placed after the mask peptide group exhibits activity in high serum.

### Example 47

In this example, delivery agent examples comprising anchor groups in different locations within the delivery agent structure were evaluated. The particular delivery agents are described below:
Negative Control: Morpholino (MO) only
21-4: fluor-[Lytic]-[CL]-[Mask]-[2×K(LA)]-[G′]
21-5: fluor-[Lytic]-[CL]-[1×K(LA)]-[Mask]-[1×K(LA)]-[G′]
SEQ ID NO: 216: ELLEELLEELLEGFGFVGGKQLLQQLLQQLLQKG, wherein the N-terminal E at position 1 comprises the “Fluor” group described above, each K is functionalized with an LA group and the C-terminal G at position 34 is G′ as described above.

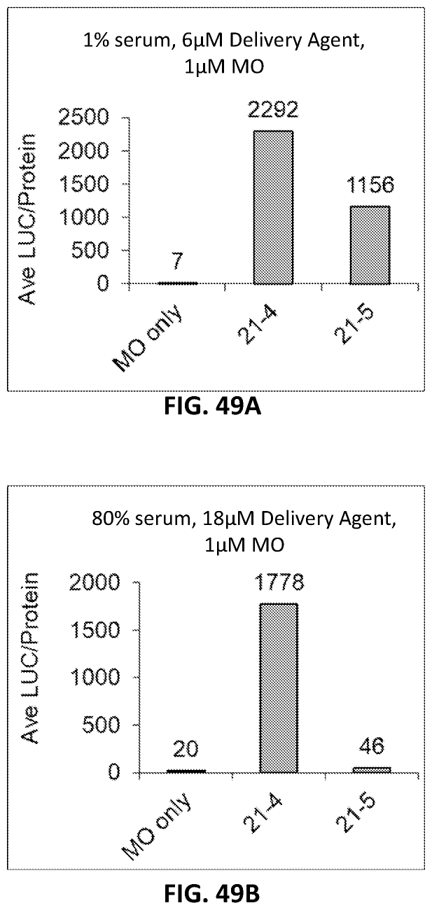

As shown in FIGS. 49A and 49B, delivery agent examples comprising an anchor group at the N-terminus of the mask peptide group showed reduced activity in 1% and 80% serum.

### Example 48

In this example, delivery agent examples wherein segments of the delivery agent are rearranged compared to one another, were evaluated. The particular delivery agents are described below:
Negative Control: Morpholino (MO) only
19-1: fluor-[Lytic]-[CL]-[Mask]-[1×K(LA)]-[G′]
22-11: fluor-[Mask]-[1×K(LA)]-[CL]-[Lytic]-[G′]
SEQ ID NO: 217: QLLQQLLQQLLQKGFGFVGGELLEELLEELLEG, wherein the N-terminal Q at position 1 comprises the “Fluor” group described above, K is functionalized with an LA group and the C-terminal G at position 33 is G′ as described above.

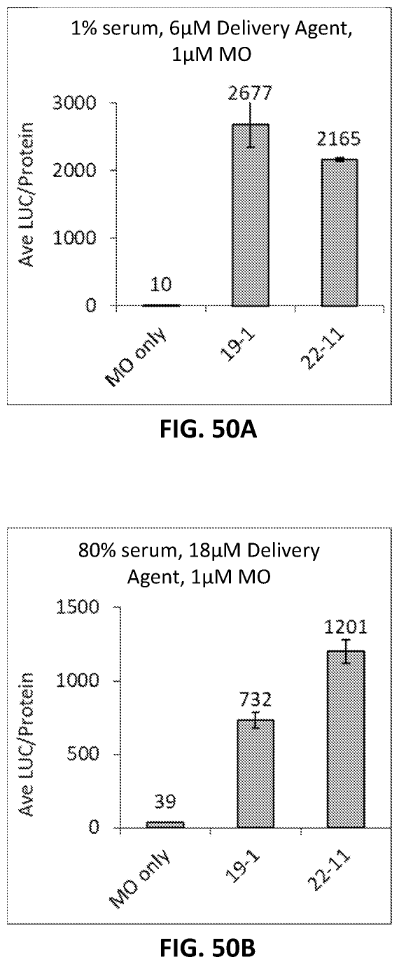

As illustrated in FIGS. 50A and 50B, in some aspects of the disclosure, a delivery agent wherein the mask peptide group is N-terminal to both the cleavable linker and the lytic peptide group, and the anchor groups are C-terminal to the mask peptide group, can have activity. That placing the anchor groups C-terminal to the mask peptide group is beneficial in some aspects of the disclosure and is evident from a comparison of delivery agents 19-17A (Example 46) and 21-5 (Example 47) with delivery agent 22-11. Delivery agents 19-17A and 21-5 both have anchor groups N-terminal to the mask peptide group, and both exhibit reduced activity in high serum. However, delivery agent 22-11, with the anchor group at the C-terminus of the mask peptide group, retains substantial activity in high serum experiments.

### Example 49

In this example, delivery agent examples comprising anchor groups at different locations in the structure of the delivery agent were evaluated. The particular delivery agents are described below:
24-1: fluor-[Mask]-[1×K(LA)]-[CL]-[Lytic]-[G]
23-27: fluor-[Lytic]-[CL]-[Mask]-[1×K(LA)]-[G′]
23-1: fluor-[Mask]-[2×K(LA)]-[CL]-[Lytic]-[G′]
23-4: fluor-[Lytic]-[CL]-[Mask]-[2×K(LA)]-[G′]
SEQ ID NO: 218: QLLQQLLQQLLQKKGFGFVGGELLEELLEELLEG, wherein the N-terminal Q at position 1 comprises the “Fluor” group described above, each K is functionalized with an LA group and the C-terminal G at position 34 is G′ as described above.

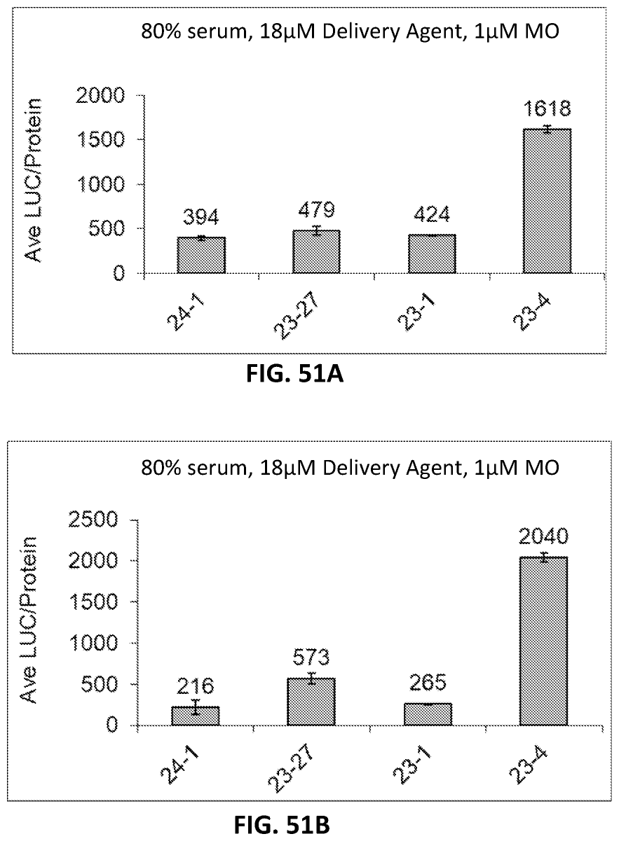

As seen in FIG. 51A, in some aspects of the disclosure, an increase in delivery agent activity resulting from increased numbers of anchor groups is observed primarily when the anchor groups are at or close to the C-terminal end of the delivery agent. FIG. 51B shows a repetition of the same experiment.

### Example 50

In this example, delivery agent examples comprising different numbers of anchor groups located in different regions of the delivery agents were evaluated. The particular delivery agents are described below:
23-1: fluor-[Mask]-[2×K(LA)]-[CL]-[Lytic]-[G′]
23-2: fluor-[Mask]-[3×K(LA)]-[CL]-[Lytic]-[G′]
23-3: fluor-[Mask]-[4×K(LA)]-[CL]-[Lytic]-[G′]
23-4: fluor-[Lytic]-[CL]-[Mask]-[2×K(LA)]-[G]
23-5: fluor-[Lytic]-[CL]-[Mask]-[3×K(LA)]-[G′]

SEQ ID NO: 219: QLLQQLLQQLLQKKKGFGFVGGELLEELLEELLEG, wherein the N-terminal Q at position 1 comprises the “Fluor” group described above, each K is functionalized with an LA group and the C-terminal G at position 35 is G′ as described above.
SEQ ID NO: 220: QLLQQLLQQLLQKKKKGFGFVGGELLEELLEELLEG, wherein the N-terminal Q at position 1 comprises the “Fluor” group described above, each K is functionalized with an LA group and the C-terminal G at position 36 is G′ as described above.

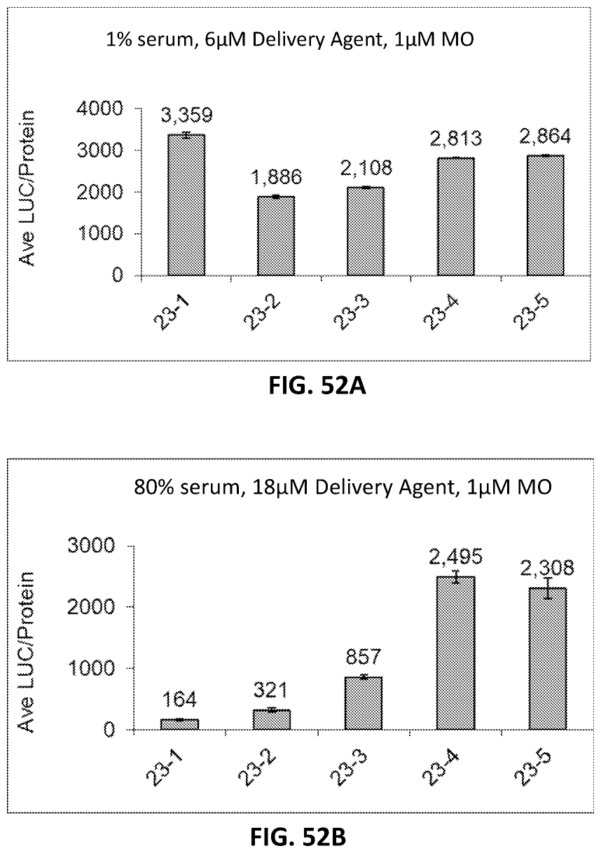

As illustrated in FIGS. 52A and 52B, these data suggest that in some aspects of the disclosure, adding anchor groups C-terminal to the Lytic-CL-Mask portion of the delivery agents of this example improved activity of the delivery agents. More modest improvements in activity were seen when anchor groups were added to the delivery agent toward the center of the delivery agent.

### Example 51

In this example, delivery agent examples having different structures were evaluated. The particular delivery agents are described below:
Neg Control: Morpholino (MO) only
21-4: fluor-[Lytic]-[CL]-[Mask]-[2×K(LA)]-[G′]
22-1: shown below
22-12: shown below

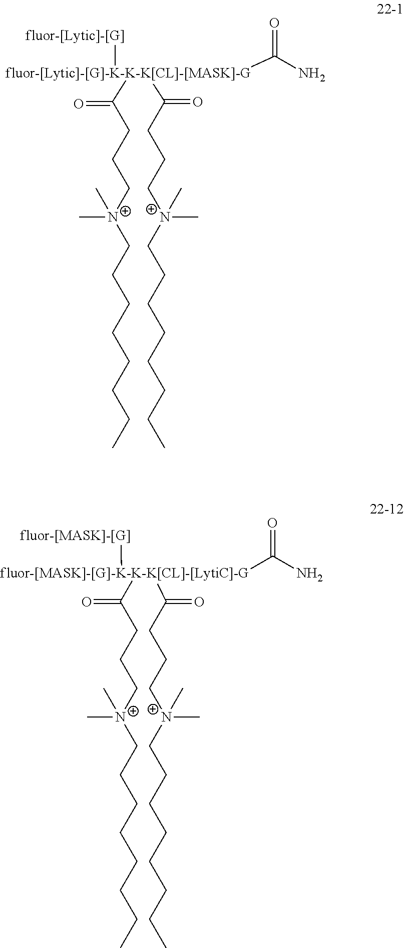

SEQ ID NO: 221: ELLEELLEELLEGKKKGFGFVGGQLLQQLLOOLLQG, wherein the N-terminal E at position 1 comprises the “Fluor” group described above, K at position 14 is a modified amino acid wherein the side chain of the lysine is linked via an amide bond to SEQ ID NO: 245, each K at position 15 and 16 is functionalized with an LA group and the C-terminal G at position 36 is G′ as described above.
SEQ ID NO: 245: GELLEELLEELLE, wherein the glycine at position 1 is linked via an amide bond to the side chain of the lysine at position number 14 of SEQ ID NO: 221 and the E at position 13 comprises the “Fluor” group described above.
SEQ ID NO: 222: QLLQQLLQQLLQGKKKGFGFVGGELLEELLEELLEG, wherein the N-terminal Q at position 1 comprises the “Fluor” group described above, K at position 14 is a modified amino acid wherein the side chain of the lysine is linked via an amide bond to SEQ ID NO: 245, each K at position 15 and 16 is functionalized with an LA group and the C-terminal G at position 36 is G′ as described above.
SEQ ID NO: 266: GOLLQQLLOOLLQ, wherein the glycine at position 1 is linked via an amide bond to the side chain of the lysine at position number 14 of SEQ ID NO: 222 and the Q at position 13 comprises the “Fluor” group described above.

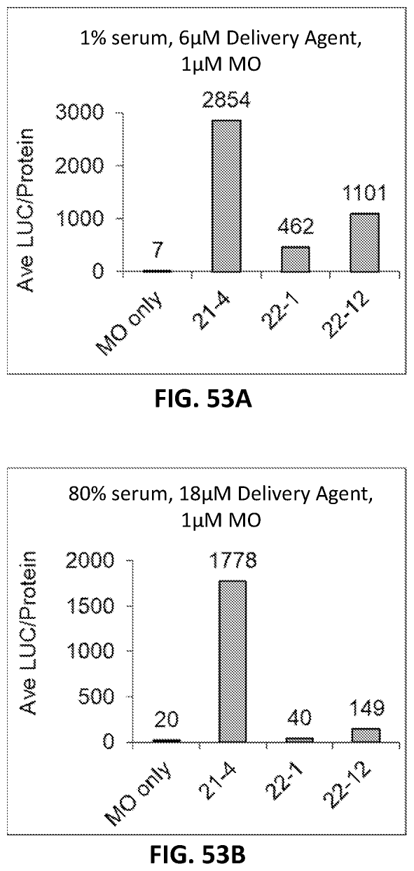

As shown in FIGS. 53A and 53B, each of the delivery agent examples of this example exhibited activity in high and low serum conditions. Delivery agent 21-4 exhibited higher activity than delivery agents 22-1 and 22-12.

### Example 52

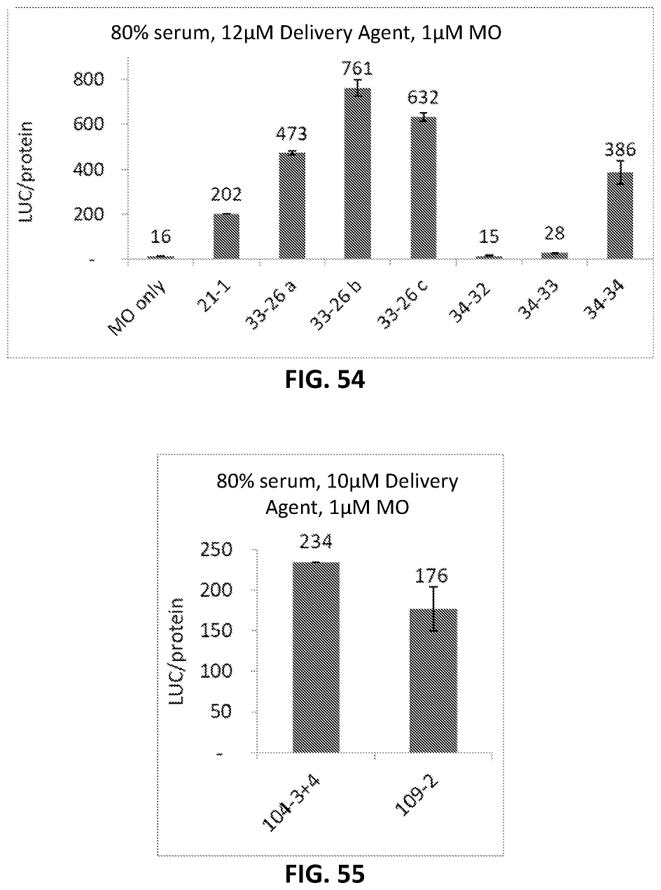

In this example, delivery agent examples comprising anchor groups with different structures, or comprising more than one cleavable linker, were evaluated. Results are shown in FIG. 54. The particular delivery agents are described below:
Negative Control: Morpholino (MO) only
Positive Control: 21-1: fluor-[Lytic]-[CL]-[Mask]-[1×K(LA)]-[G]
33-26a: fluor-[Lytic]-[CL]-[Mask]-[K(Y¹)]-[G]
33-26b: fluor-[Lytic]-[CL]-[Mask]-[K(Y²)]-[G]
33-26c: fluor-[Lytic]-[CL]-[Mask]-[K(Y³)]-[G]
34-32: fluor-[Lytic]-[CL]-[1×K(LA)]-[CL]-[Mask]-[G′]
34-33: fluor-[Lytic]-[CL2]-[1×K(LA)]-[CL2]-[Mask]-[G′]
34-34: fluor-[ELL EQL LQE LLE]-[CL2]-[1×K(LA)]-[CL2]-[QLL QEL LEQ LLQ]-[G′]

### Where:

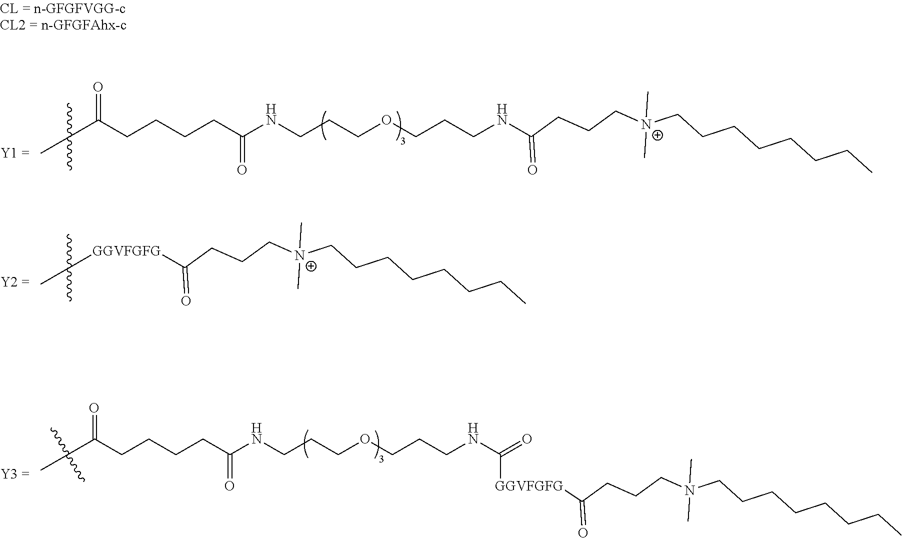

SEQ ID NO: 267: ELLEELLEELLEGFGFVGGQLLOOLLQQLLOKG, wherein the N-terminal E at position 1 comprises the “Fluor” group described above, K is functionalized with Y¹ illustrated above and the C-terminal G at position 33 is G′ as described above.
SEQ ID NO: 268: ELLEELLEELLEGFGFVGGOLLQQLLOOLLQKG, wherein the N-terminal E at position 1 comprises the “Fluor” group described above, K is functionalized with Y² illustrated above and the C-terminal G at position 33 is G′ as described above.
SEQ ID NO: 269: ELLEELLEELLEGFGFVGGQLLQQLLOQLLQKG, wherein the N-terminal E at position 1 comprises the “Fluor” group described above, K is functionalized with Y³ illustrated above and the C-terminal G at position 33 is G′ as described above.
SEQ ID NO: 223: ELLEELLEELLEGFGFVGGKGFGFVGGQLLQQLLQQLLQG, wherein the N-terminal E at position 1 comprises the “Fluor” group described above, K is functionalized with an LA group and the C-terminal G at position 40 is G′ as described above.

SEQ ID NO: 224: ELLEELLEELLEGFGFXKGFGFXQLLQQLLQQLLQG, wherein the N-terminal E at position 1 comprises the “Fluor” group described above, each X is Ahx, each K is functionalized with an LA group, and the C-terminal G at position 36 is G′ as described above.
SEQ ID NO: 225: ELLEQLLQELLEGFGFXKGFGFXQLLQELLEQLLQG, wherein the N-terminal E at position 1 comprises the “Fluor” group described above, each X is Ahx, each K is functionalized with an LA group, and the C-terminal G at position 36 is G′ as described above.

As can be seen, delivery agents 33-26a, 33-26b, and 33-26c, wherein an anchor group is extended away from the main delivery agent structure and includes its own cleavable linker, exhibit activity in high serum conditions.

Delivery agents 34-32, 34-33, and 34-34 each included two cleavable linkers as described above, with delivery agent 34-34 comprising two lytic peptide groups (and no mask peptide group) rather than one lytic peptide group and one mask peptide group. Of these three examples, delivery agent 34-34 exhibited the most activity in high serum conditions.

### Example 53

In this example, a delivery agent embodiment comprising an additional cleavable linker group at the C-terminus group of the delivery agent was evaluated. The particular delivery agents are described below:
104-3+4: Cap-[DLL DDL LDD LLE]-[CL]-[Mask]-[2×K(LA)]-[GNalG′]
109-2: Cap-[DLL DDL LDD LLE]-[CL]-[Mask]-[2×K(LA)]-[GNal]-[K(Z)]-[G′]

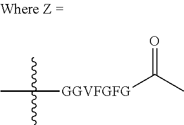

SEQ ID NO: 226: DLLDDLLDDLLEGFGFVGGQLLQQLLQQLLQKKGXXG, wherein the N-terminal D at position 1 comprises the “Cap” group described above, X at position 35 is Nal, X at position 36 is lysine modified with a Z group as illustrated above, each K is functionalized with an LA group, and the C-terminal G at position 37 is G′ as described above.

As shown in FIG. 55, in some aspects of the disclosure, a second cleavable linker can be added onto the side chain amine of a lysine on the C-terminus group of a delivery agent. In this example, only minor loss of activity was observed as a result of the additional cleavable linker. In particular examples, this modification can serve as a site of conjugation for therapeutics and other molecules (see Examples 55-63). In other examples, the lysine can also serve as an attachment site in the absence of the additional cleavable linker.

### Example 54

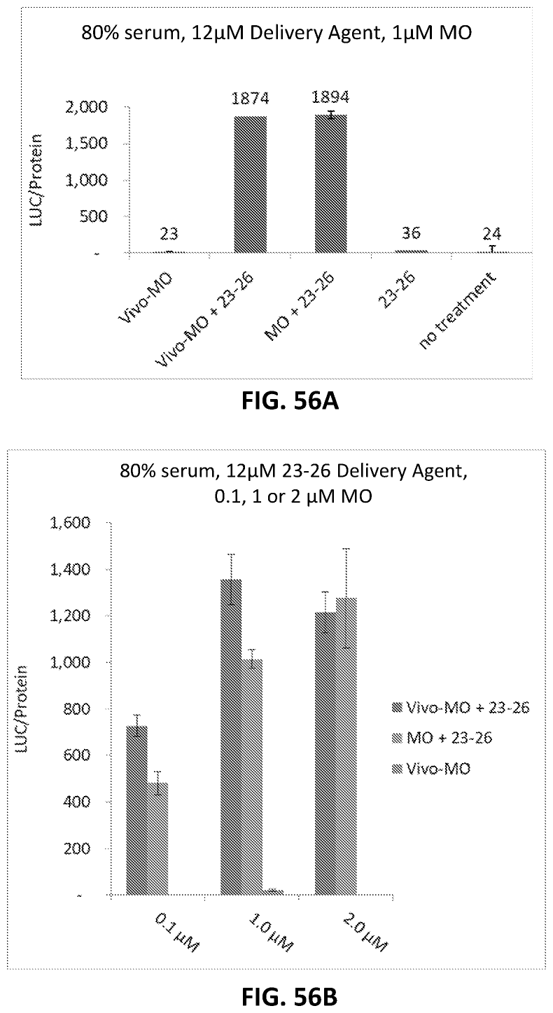

In this example, delivery agent examples were administered to cells in culture in combination with a morpholino, and morpholino activity was evaluated. Results are shown in FIGS. 56A-56B. The particular delivery agents are described below:
Negative control: No treatment
Vivo Morpholinos (Vivo-MO) only (www.gene-tools.com/vivomorpholinos)
23-26: fluor-[Lytic]-[CL]-[Mask]-[2×K(LA)]-[G]
Vivo Morpholino+23-26
23-26+Morpholino

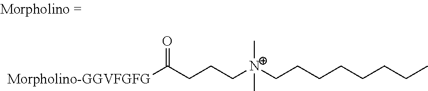

In this example, the Vivo-MO and delivery agent 23-26 were not pre-mixed; rather, they were added to the cell culture wells sequentially. Additionally, the morpholinos of this experiment were positively charged. As can be seen, administration of a delivery agent with a morpholino substantially increases morpholino activity in cell cultures.

### Example 55

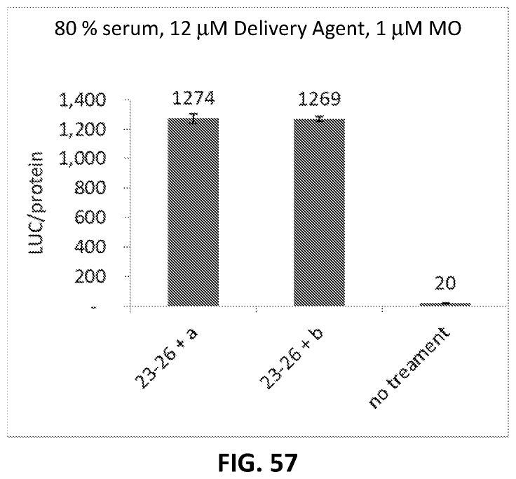

In this example, delivery agent examples were administered to cells in culture in combination with a morpholino, and morpholino activity was evaluated. Results are shown in FIG. 57. As can be seen, in this example, morpholino activity is enhanced by administering a morpholino to a cell with a delivery agent. The morpholinos in this case are uncharged (neutral) molecules. The particular delivery agents are described below:
No treatment
23-26: fluor-[Lytic]-[CL]-[Mask]-[2×K(LA)]-[G]
   23-26+a
   23-26+b

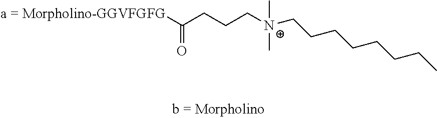

In some aspects of the disclosure activity is observed for a neutral and a positively charged morpholino.

### Example 56

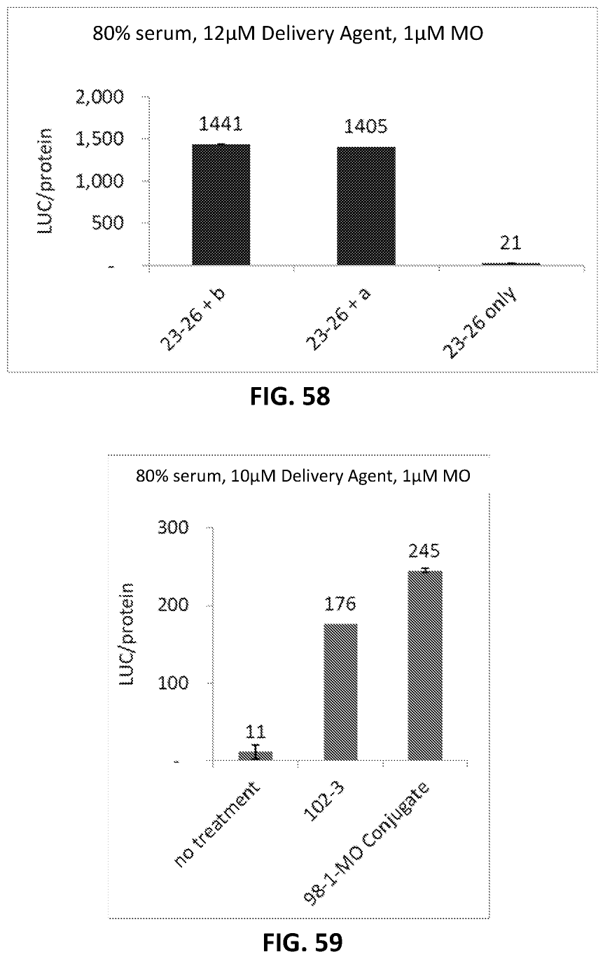

In this example, delivery agent examples were administered to cells in culture in combination with a morpholino, and morpholino activity was evaluated. Delivery agent 23-26 was not structurally modified with components “a” or “b,” illustrated below, but was administered in a mixture with these components. Results are shown in FIG. 58. As can be seen, in this example, morpholino activity is enhanced by administering a morpholino to a cell with a delivery agent. The morpholinos in this case are negatively charged molecules (the peptide-based moiety conjugated to the morpholino has a net negative charge). The particular delivery agents are described below:
23-26: fluor-[Lytic]-[CL]-[Mask]-[2×K(LA)]-[G′]
   23-26+a
   23-26+b

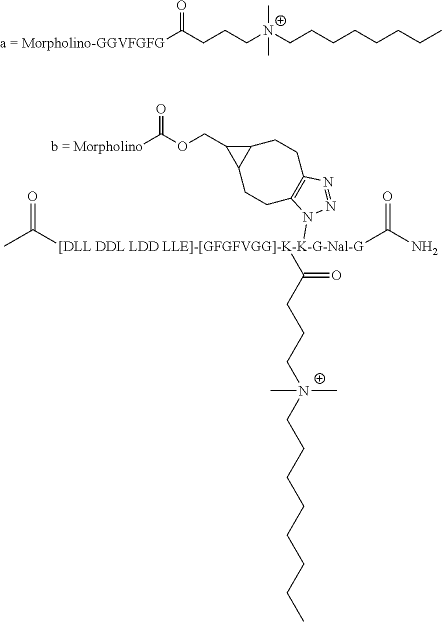

### Example 57

In this example, delivery agent examples were administered to cells in culture in combination with a morpholino, and morpholino activity was evaluated. In this embodiment, a morpholino was conjugated to a delivery agent. Results are shown in FIG. 59. As can be seen, in this example, morpholino activity is enhanced by administering a morpholino to a cell conjugated to a delivery agent. This example further shows that in some aspects of the disclosure, a morpholino can be conjugated to a delivery agent using a disulfide linker that can be cleaved in the lysosome. The particular delivery agents are described below:
Negative control: No treatment (This sample contained a morpholino but no delivery agent)
102-3: Cap-[Lytic]-[CL]-[Mask]-[2×K(LA)]-[GNalG′]
98-1-MO Conjugate:

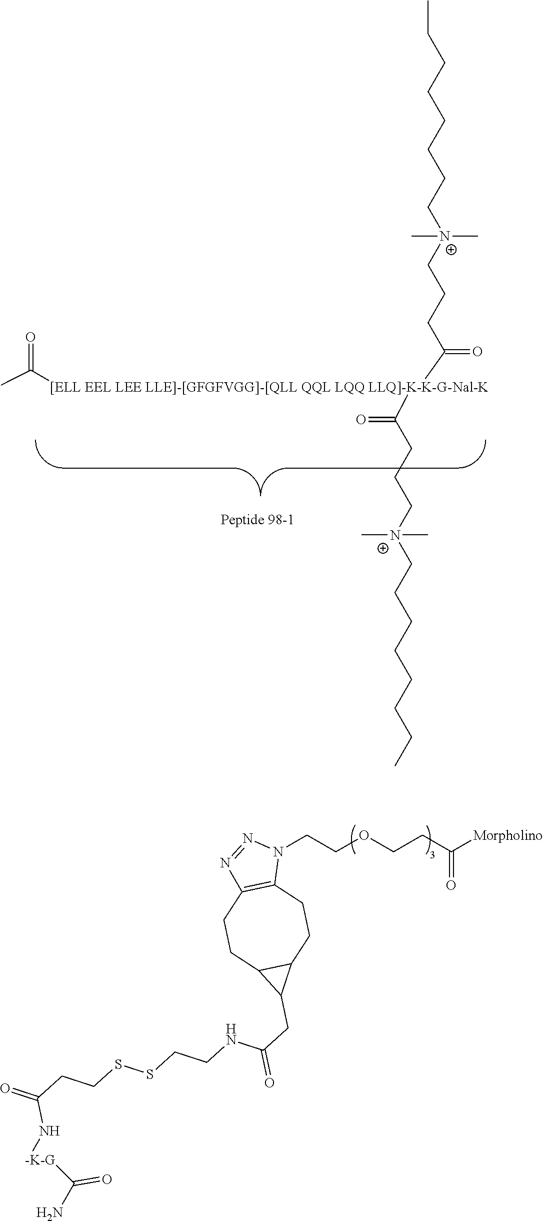

### Example 58

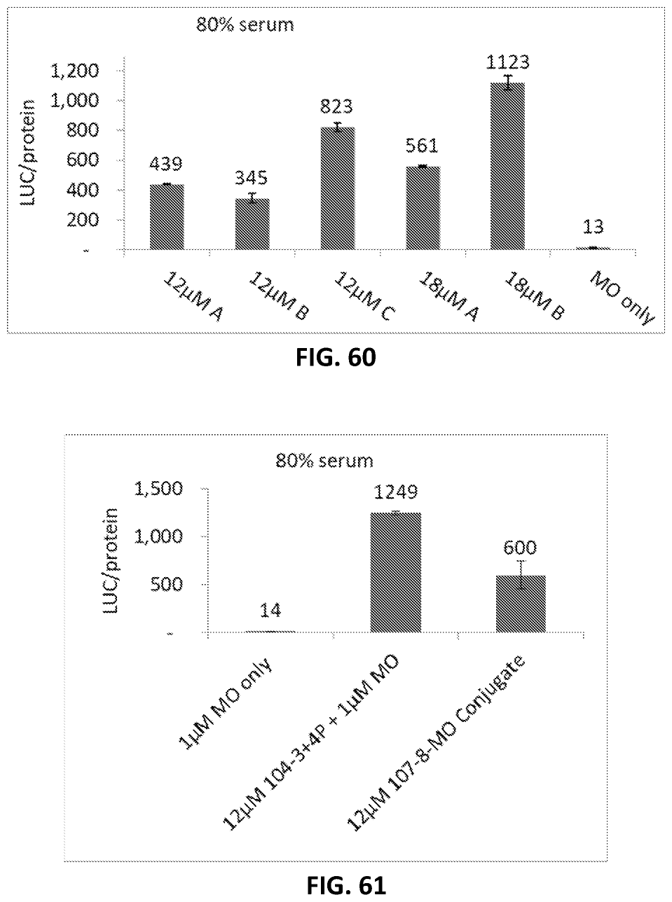

In this example, delivery agent examples were administered to cells in culture in combination with a morpholino, and morpholino activity was evaluated. In this embodiment, a morpholino was administered to cells either unconjugated in the presence of a delivery agent or conjugated to a delivery agent. Results are shown in FIG. 60. As can be seen, in this example, morpholino activity is enhanced by administering a morpholino to a cell either unconjugated in the presence of the delivery agent or conjugated to the delivery agent. In some examples, a morpholino-delivery agent conjugate may exhibit decreased solubility as compared to the morpholino and delivery agent separately. Without being limited to any single theory, in this experiment, this solubility difference could account for the decreased activity of the morpholino-delivery agent conjugate compared to the morpholino and delivery agent administered unconjugated. The particular delivery agents are described below:
104-3+4: Cap-[DLLDDLLDDLLE]-[CL]-[Mask]-[2×K(LA)]-[GNalG′]
104-6-Morpholino conjugate:

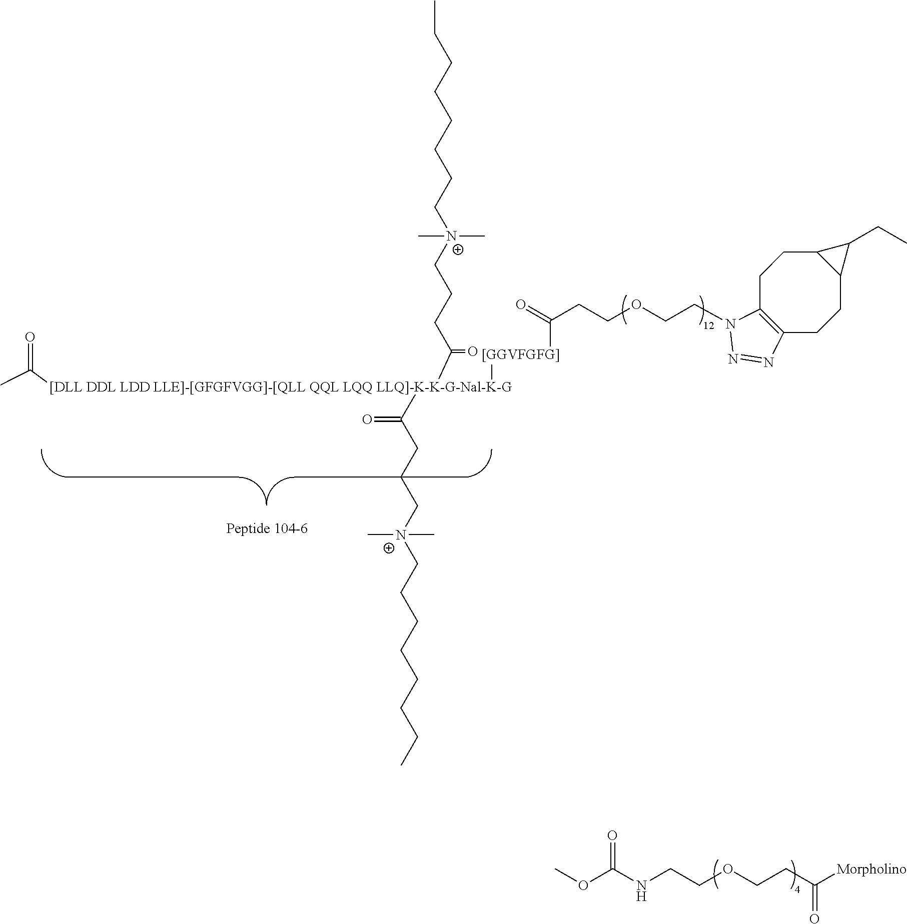

A=100% of 12 μM 104-6-Morpholino conjugate
B=50% of 12 μM 104-6-Morpholino conjugate+50% of 12 μM 104-3+4=12 μM overall delivery agent
C=100% of 12 μM 104-3+4+1 μM morpholino (not conjugated)

### Example 59

In this example, delivery agent examples were administered to cells in culture in combination with a morpholino, and morpholino activity was evaluated. In this embodiment, a morpholino was conjugated to a delivery agent. The particular delivery agents are described below:
Negative Control: Morpholino only
Positive Control: 104-3+4: Cap-[DLLDDLLDDLLE]-[CL]-[Mask]-[2×K(LA)]-[GNalG′]
MO-107-8 Conjugate in deoxycholic acid:

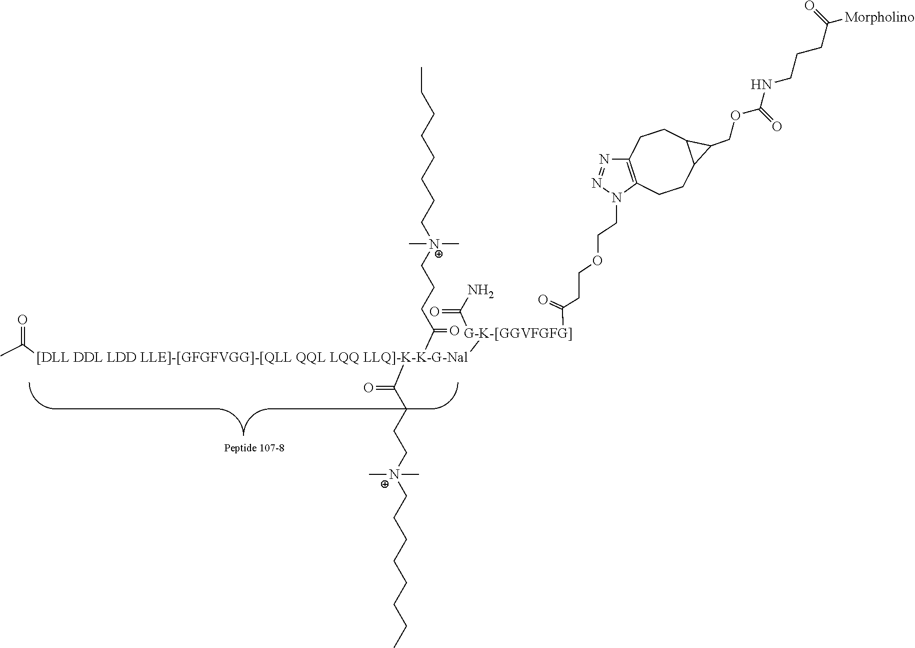

As can be seen in FIG. 61, in this example, morpholino activity is enhanced by administering a morpholino to a cell conjugated to a delivery agent, as compared to delivery of the morpholino alone. In this example, the 107-8-morpholino conjugate was combined with deoxycholic acid. Without being limited to any particular theory, combining the conjugate with deoxycholic acid may increase solubility of the conjugate in water.

### Example 60

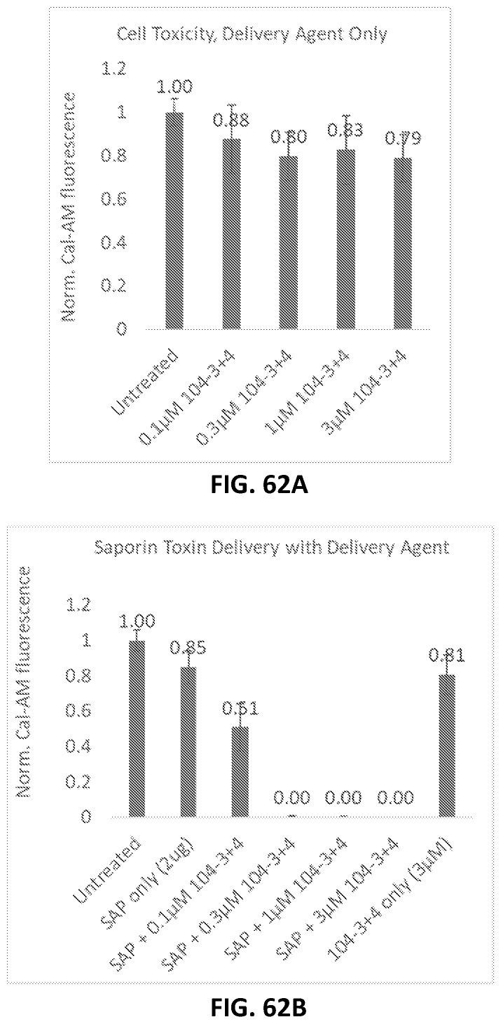

In this example, delivery agent examples were administered to cells in cell culture in combination with saporin, a 30 kDa protein toxin. The particular delivery agents are described below:
104-3+4: Cap-[DLLDDLLDDLLE]-[CL]-[Mask]-[2×K(LA)]-[GNalG′]
Saporin (SAP): Ribosome inactivating protein, plant-derived toxin Results of a calcein AM toxicity assay are shown in FIGS. 62A and 62B. As can be seen, in this example, saporin alone has only minimal effects on cell survival. However, the number of live cells decreased when saporin was administered with 0.1 μM of the delivery agent embodiment.

### Example 61

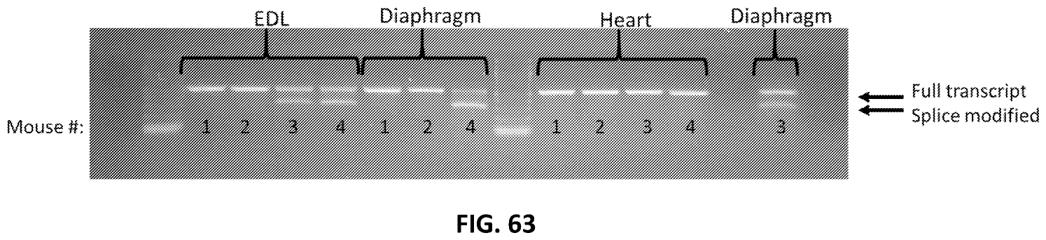

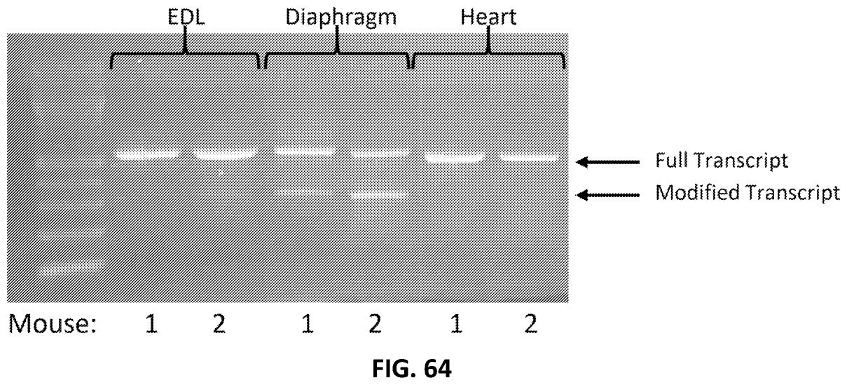

In this example, mouse tail-vein injections were performed using a mixture of a delivery agent embodiment and a morpholino that splice-modifies Dystrophin pre-mRNA. In vivo delivery was through tail vein injections on consecutive days. Activity of the morpholino in the muscle tissues lead to conversion of the full mRNA transcript to a modified transcript. Animal were sacrificed, muscle tissue was dissected, mRNA was isolated, and RT-PCR was used to amplify DNA, which was then analyzed using gel electrophoresis (FIG. 63).

As can be seen, administration of the morpholino in combination with a delivery agent successfully delivered the morpholino into the cytosol of cells in two different muscle tissues: (1) the extensor digitorum longus (EDL) and (2) the diaphragm.

The particular delivery agents are described below:
21-4: fluor-[Lytic]-[CL]-[Mask]-[2×K(LA)]-[G′]
Morpholino (MO): The standard morpholino structure, as described above, where the morpholino sequence targets exon 23 of the Dystrophin pre-mRNA in mice. The DMD Morpholino sequence was 5′-GGCCAAACCTCGGCTTACCTGAAAT-3′ (SEQ ID NO: 229).

### Mice:

Mouse 1:3 injections of 9 mg/Kg 21-4 and 12 mg/Kg MO
Mouse 2:3 injections of 8 mg/Kg 21-4 and 21 mg/Kg MO
Mouse 3:3 injections of 16 mg/Kg 21-4 and 11 mg/Kg MO
Mouse 4:3 injections of 16 mg/Kg 21-4 and 23 mg/Kg MO

Mice were 6 weeks old±3 days. For the first two injections, all mice were healthy. For the third injection, mice 3 and 4 were less active, but were otherwise healthy. Results showed that mice 1 and 2 had no conversion in any tissue. Mouse 3 had roughly 30-40% conversion in the EDL and roughly 40% in the diaphragm, and no conversion in the heart. Mouse 4 had roughly 50-60% conversion in the EDL and roughly 80 to 90% in the diaphragm, and no conversion in the heart.

### Example 62

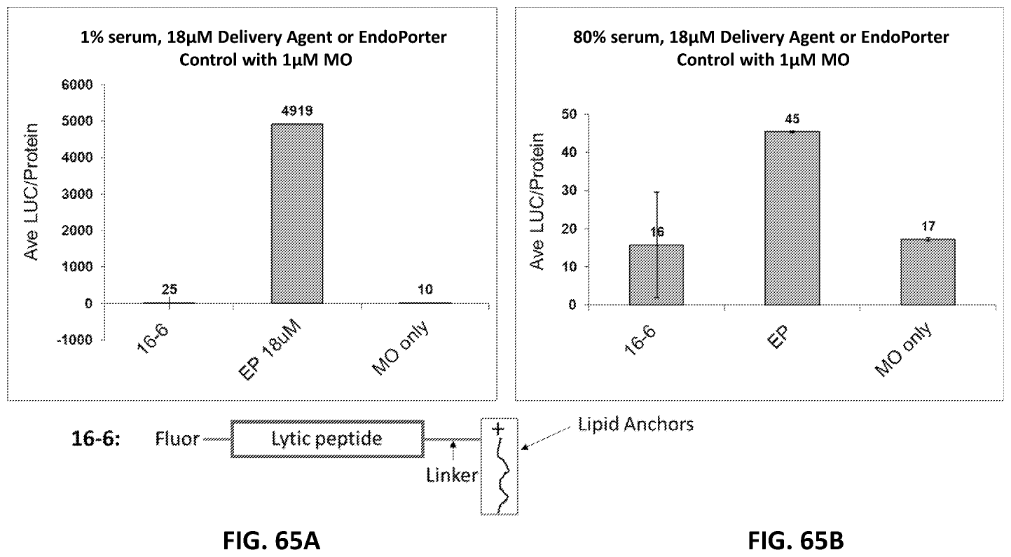

In this example, mouse tail-vein injections were performed using a mixture of a delivery agent embodiment and a morpholino that splice-modifies Dystrophin pre-mRNA. In vivo delivery was through tail vein injections on consecutive days. Activity of the morpholino in the muscle tissues lead to conversion of the full mRNA transcript to a modified transcript. Animal were sacrificed, muscle tissue was dissected, mRNA was isolated, and RT-PCR was used to amplify DNA, which was then analyzed using gel electrophoresis (FIG. 65).

As can be seen, administration of the morpholino in combination with a delivery agent successfully delivered the morpholino into the cytosol of cells in two different muscle tissues: (1) the extensor digitorum longus (EDL) and (2) the diaphragm.

The particular delivery agents are described below:
33-3: fluor-[Lytic]-[CL]-[Mask]-[2×K(LA)]
Morpholino (MO): Our standard morpholino structure, as described above, where the morpholino targets the Dystrophin pre-RNA in mice.
SEQ ID NO: 228: ELLEELLEELLEGFGFVGGQLLQQLLQQLLQKK, wherein the N-terminal E at position 1 comprises the “Fluor” group described above, each K is functionalized with an LA group.

### Mice:

Mouse 1:3 injections of 11 mg/Kg 33-3 and 16 mg/kg MO
Mouse 2:2 injections of 11 mg/Kg 33-3 and 16 mg/kg MO

Mice were 20 weeks old±3 days. Mice 1 and 2 were healthy after the first injection, but exhibited signs of illness after the 2^{nd} injection, with partial recovery overnight. Mouse 1 exhibited signs of illness after the third injection but partially recovered overnight, although some weight was lost. The morpholino exhibited strong activity in the diaphragm of mouse 2 with only two injections.

### Mouse 1: 0% EDL, 30% Diaphragm, 0% Heart

### Mouse 2: 5% EDL, 50% Diaphragm, 0% Heart

### Example 63

In this example, a delivery agent embodiment lacking a mask peptide group was administered to cells in culture in combination with a morpholino, and morpholino activity was evaluated. Endoporter, a commercially available delivery peptide, is used as a positive control in this experiment. Results are shown in FIGS. 65A-65B. As can be seen, in this example, morpholino activity is not enhanced by administering a morpholino to a cell with this embodiment in either high or low serum conditions. The particular delivery agent is described below:
16-6: fluor-[G]-[Lytic]-[CL]-[1×K(LA)]-[G′]
SEQ ID NO: 232: GELLEELLEELLEGFGFVGGKG, wherein the N-terminal G at position 1 comprises the “Fluor” group described above, K is functionalized with an LA group and the C-terminal G at position 22 is G′ as described above.

### Example 64

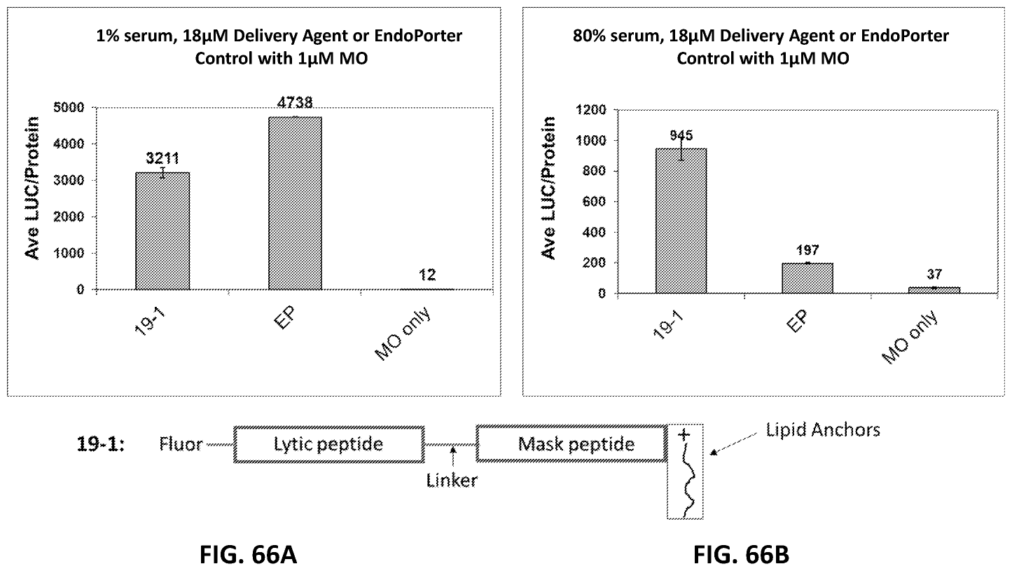

In this example, a delivery agent embodiment was administered to cells in culture in combination with a morpholino, and morpholino activity was evaluated. Results are shown in FIGS. 66A-66B. As can be seen, in this example, morpholino activity is enhanced by administering a morpholino to a cell with a delivery agent. The particular delivery agent is described below:
19-1: fluor-[Lytic]-[CL]-[Mask]-[1×K(LA)]-[G]

Without being limited to any single theory, the increased morpholino activity when morpholino is combined with the delivery agent 19-1 as compared to 16-6 (from Example 64) is believed to result from the inclusion of a mask peptide group in 19-1.

### Example 65

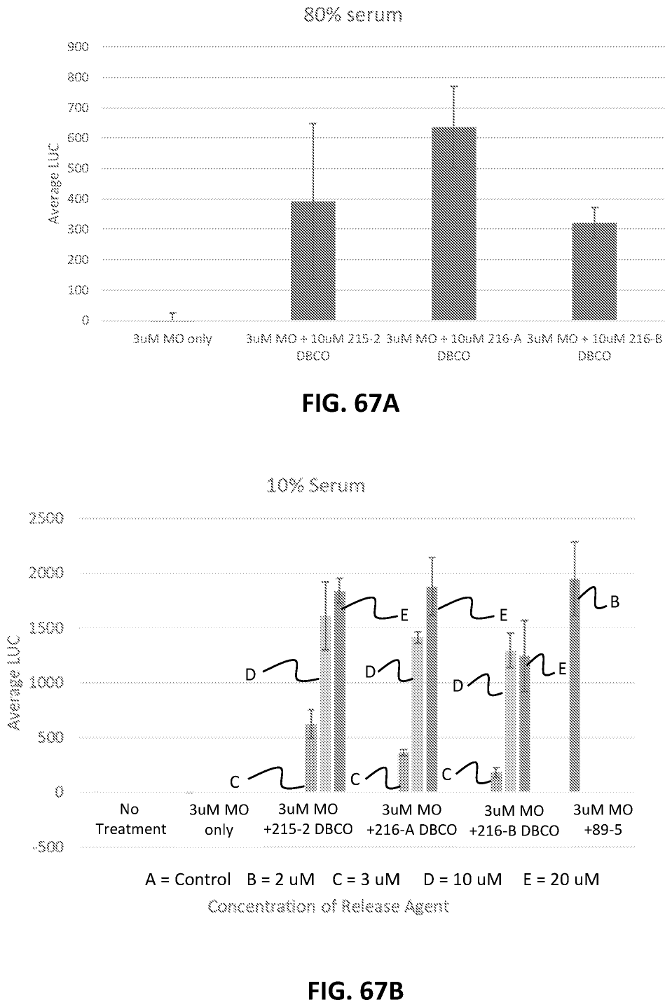

In this example, delivery agent examples comprising a PEG12-DBCO moiety were evaluated in 10% and 80% serum. DBCO-PEG12-NHS ester (CAS #2093934-94-0) was purchased from BroadPharm (Cat #BP-24149). As shown in FIG. 67A and FIG. 67B, in this embodiment, delivery agents comprising a PEG12-DBCO moiety exhibited activity in high serum. These data suggest that, in some aspects of the disclosure, delivery agents comprising uncharged DBCO groups can be used in place of a positively charge LA group.

The particular delivery agents are described below:
Negative control: No treatment
Negative control: MO only (Morpholino only)
Positive control: 89-5: cap-[ELL EEL LEE LLE]-[GFG FVG G]-[QL LQQ LLQ QLL Q]-[2×K(LA)]-[GNalG′]
215-2 DBCO: cap-[QLL EQL LQQ LLE]-[GFG FVG G]-[QL LEQ LLQ QLL E]-[1×K(Y)]-[GNalG′]
216-A DBCO: cap-[ELL EEL LEE LLE]-[GFG FVG G]-[QL LAQ LLA QLL Q]-[1×K(Y)]-[G NalG′]
216-B DBCO: cap-[ELL EEL LEE LLE]-[GFG FVG G]-[QL LQQ LLQ QLL Q]-[1×K(Y)]-[GNalG′]

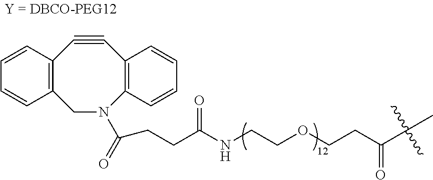

SEQ ID NO: 272: QLLEQLLQQLLEGFGFVGGQLLEQLLQQLLEKGXG, wherein X is naphthylalanine (Nal), the C-terminal G at position 35 is a modified glycine comprising a —C(O)—NH₂ group, and in some aspects of the disclosure, the N-terminal Q comprises an N-terminus modified with an acetyl group. In example 215-2, K comprises the Y group shown above.
SEQ ID NO: 273: ELLEELLEELLEGFGFVGGQLLAQLLAQLLQKGXG, wherein X is naphthylalanine (Nal), the C-terminal G at position 35 is a modified glycine comprising a —C(O)—NH₂ group, and in some aspects of the disclosure, the N-terminal E comprises an N-terminus modified with an acetyl group. In example 216-A, K comprises the Y group shown above.
SEQ ID NO: 274: ELLEELLEELLEGFGFVGGOLLQQLLQQLLOKGXG, X is naphthylalanine (Nal), the C-terminal G at position 35 is a modified glycine comprising a —C(O)—NH₂ group, and in some aspects of the disclosure, the N-terminal Q comprises an N-terminus modified with an acetyl group. In example 216-B, K comprises the Y shown above.

### Example 66

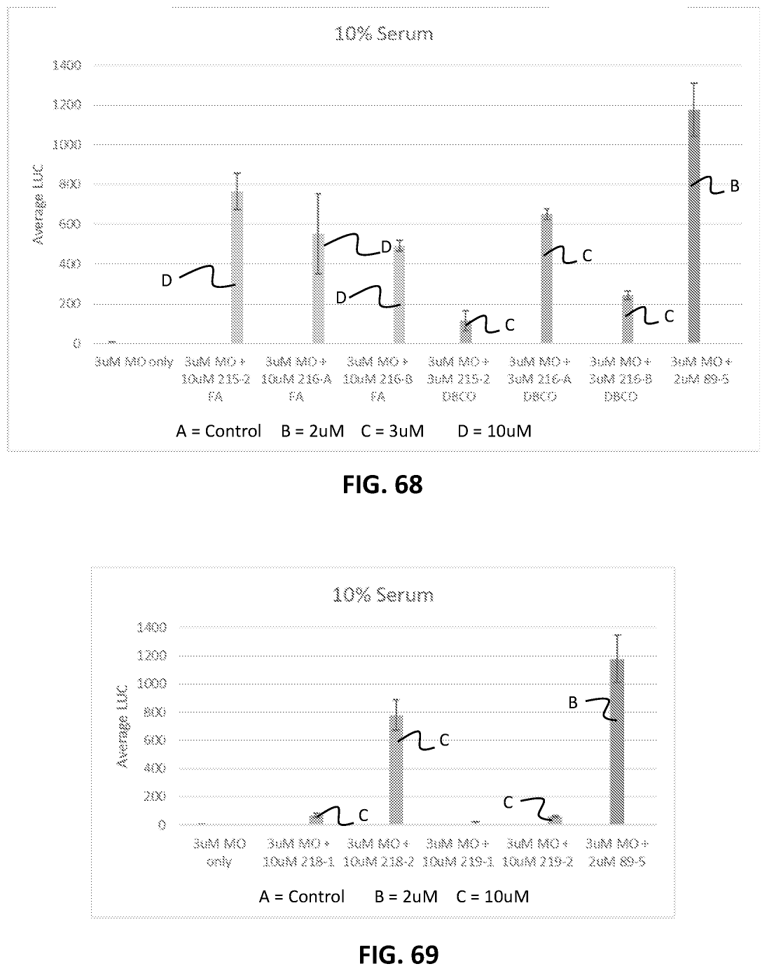

In this example, delivery agent examples comprising an unbound lysine sidechain or a PEG12-DBCO moiety bound to a lysine sidechain were evaluated in 10% serum. As illustrated in FIG. 68, in this embodiment, delivery agents comprising the PEG12-DBCO moiety exhibited greater activity than similar delivery agents comprising a lysine sidechain. The particular delivery agents are described below:
Negative control: MO only (Morpholino only)
Positive control: 89-5: cap-[ELL EEL LEE LLE]-[GFG FVG G]-[QL LQQ LLQ QLL Q]-[2×K(LA)]-[GNalG′]
215-2 DBCO: cap-[QLL EQL LQQ LLE]-[GFG FVG G]-[QL LEQ LLQ QLL E]-[1×K(Y)]-[GNalG′]
216-A DBCO: cap-[ELL EEL LEE LLE]-[GFG FVG G]-[QL LAQ LLA QLL Q]-[1×K(Y)]-[G NalG′]
216-B DBCO: cap-[ELL EEL LEE LLE]-[GFG FVG G]-[QL LQQ LLQ QLL Q]-[1×K(Y)]-[GNalG′]
215-2 FA: cap-[QLL EQL LQQ LLE]-[GFG FVG G]-[QL LEQ LLQ QLL E]-[1×K]-[GNalG′]
216-A FA: cap-[ELL EEL LEE LLE]-[GFG FVG G]-[QL LAQ LLA QLL Q]-[1×K]-[GNalG′]
216-B FA: cap-[ELL EEL LEE LLE]-[GFG FVG G]-[QL LQQ LLQ QLL Q]-[1×K]-[GNalG]′
   In the above examples comprising a functionalized K residue at position 32, Y=DBCO-PEG12. Examples 215-2 FA, 216-A FA, and 216-B comprise SEQ ID NOs: 272, 273, and 274, respectively. In Examples 215-2 FA, 216-A FA, and 216-B, the K at position 32 is not functionalized and thus comprises the free amine.

### Example 67

In this example, delivery agent examples comprising a lytic peptide group with positively charged arginines replacing negatively charged amino acids were compared to an embodiment comprising negatively charged amino acids in the same positions of the lytic peptide. Results are shown in FIG. 69. This example shows that delivery agents remain active even when acidic amino acids in the lytic peptide group are replaced with arginines in the same positions. The specific delivery agent examples are described below.
Negative control: MO only (Morpholino only)
Positive control: 89-5: cap-[ELL EEL LEE LLE]-[GFG FVG G]-[QL LQQ LLQ QLL Q]-[2×K(LA)]-[GNalG′]
218-1: cap-[RLL RRL LRR LLR]-[GFG FVG G]-[QL LQQ LLQ QLL Q]-[2×K(LA)]-[GNalG]
218-2: cap-[RLL RRL LRR LLR]-[GFG FVG G]-[QL LQQ LLQ QLL Q]-[GNalG′]
219-1: cap-[RLL RRL LRR LLR]-[2×K(LA)]-[GNalG′]
219-2: cap-[RLL RRL LRR LLR]-[GNalG′]
   SEQ ID NO: 275: RLLRRLLRRLLRGFGFVGGQLLQQLLQQLLQGXG, wherein X is naphthylalanine (Nal), the C-terminal G at position 34 is a modified glycine comprising a —C(O)—NH₂ group, and in some aspects of the disclosure, and the N-terminal R comprises an N-terminus modified with an acetyl group.
   SEQ ID NO: 276: RLLRRLLRRLLRKKGXG, wherein the N-terminal R at position 1 comprises the “Cap” group described above, each K is functionalized with an LA group, X is Nal, and the C-terminal G at position 17 is G′ as described above.
   SEQ ID NO: 277: RLLRRLLRRLLRGXG, wherein the N-terminal R at position 1 comprises the “Cap” group described above, X is Nal, and the C-terminal G at position 15 is G′ as described above.

### Example 68

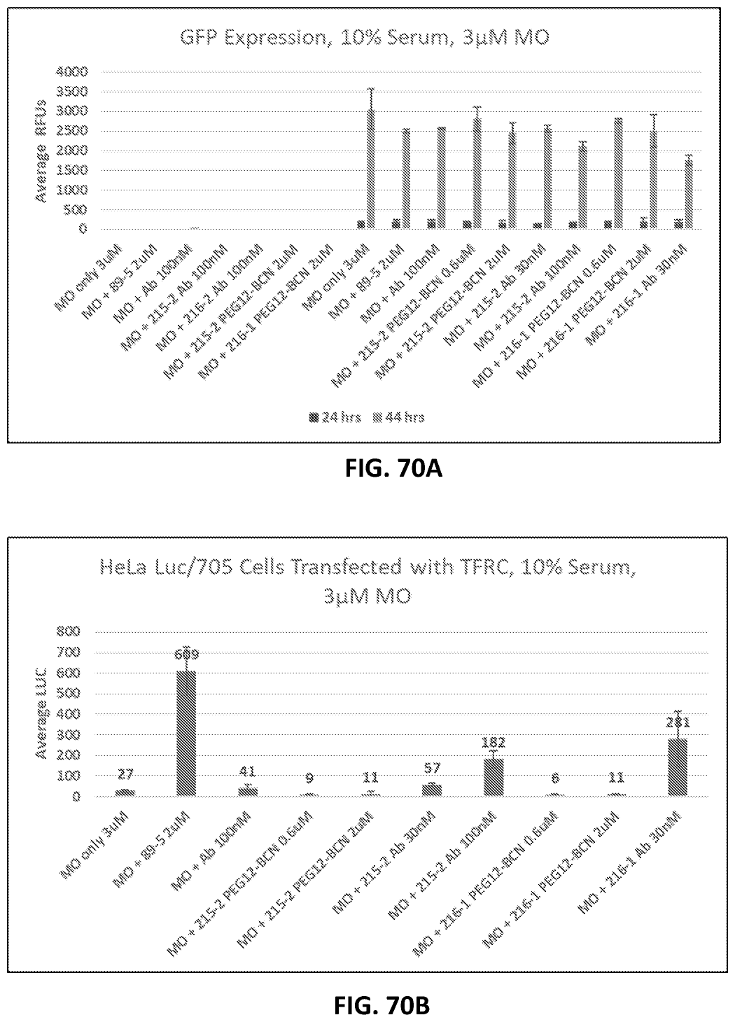

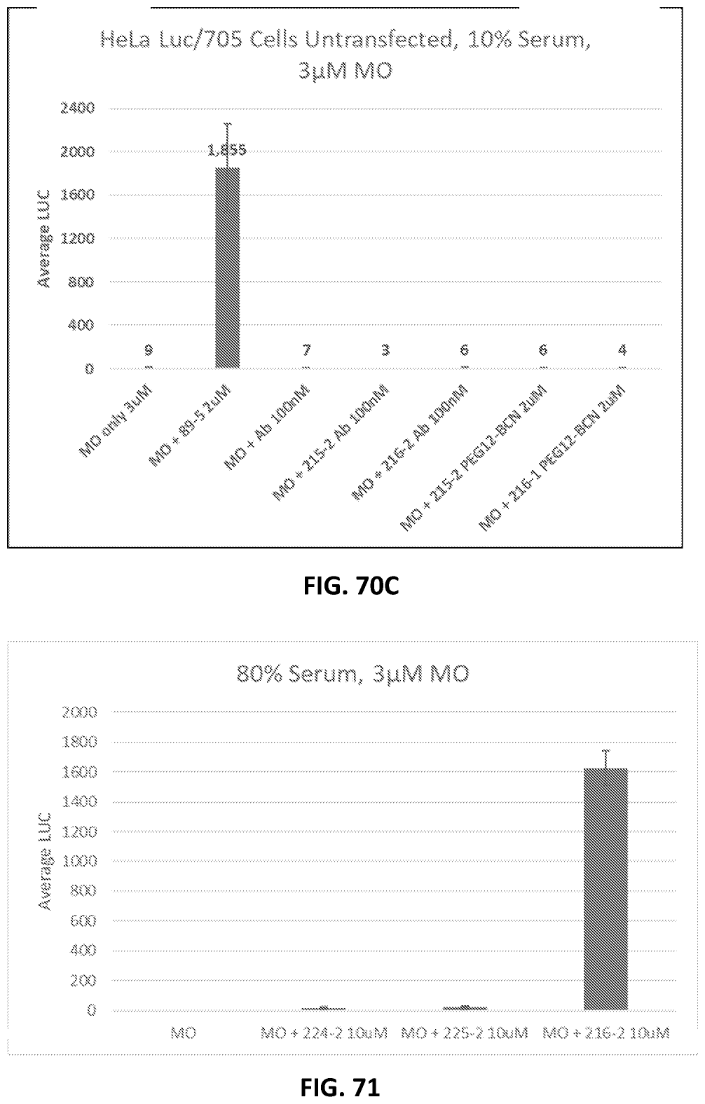

In this example, delivery agent embodiments comprising a monoclonal antibody for targeting transferrin receptors were evaluated in 10% serum in Hela cells either with or without transient transfection of TRFC plasmid. The TRFC plasmid is used to overexpress human transferrin receptor in the Hela cells. FIG. 70A shows fluorescence measured from Hela Luc/705 cells transfected with a plasmid DNA vector containing both the human transferrin receptor gene and green fluorescent protein gene. Fluorescence is relatively proportional to expression levels of enhanced green fluorescent protein (EGFP) and indicative of successful transfection and cell number. In this example, the delivery agents embodiments were tested in 10% serum in cells that were transfected with a plasmid containing the Transferrin receptor gene and in cells that were not transfected. Excess peptide delivery agent was not removed from the final Antibody-bound delivery agent after the click conjugation reaction. Controls are provided to show that the excess delivery agent is inactive. Concentrations of peptide in the control samples (215-2 PEG12-BCN and 216-1 PEG12-BCN) are equivalent to concentrations of peptide in the antibody-conjugated samples (215-2 Ab and 216-1 Ab) in the biological assay. The embodiment 215-2 Ab tested at 30 nM of antibody contains a total of 0.6 μM peptide delivery agent. The embodiment 215-2 Ab tested at 100 nM antibody contains 2 μM delivery agent peptide. The embodiment 216-1 Ab tested at 30 nM antibody contains 0.6 μM delivery agent peptide. FIG. 70B shows that both antibody-conjugated embodiments are active relative to the unconjugated controls. FIG. 70C shows that there is no activity of antibody-conjugated embodiments in untransfected cells. The particular delivery agents are described below:
Negative control: MO only (Morpholino only)
Positive control: 89-5: cap-[ELL EEL LEE LLE]-[GFG FVG G]-[QL LQQ LLQ QLL Q]-[2×K(LA)]-[GNalG′]
215-2 PEG12-BCN: cap-[QLL EQL LQQ LLE]-[GFG FVG G]-[QL LEQ LLQ QLL E]-[1×K(Y)]-[G NalG′]
216-1 PEG12-BCN: cap-[ELL EEL LEE LLE]-[GFG FVG G]-[QL LAQ LLA QLL Q]-[1×K(Y)]-[G NalG′]
215-2 Ab: cap-[QLL EQL LQQ LLE]-[GFG FVG G]-[QL LEQ LLQ QLL E]-[1×K(Z)]-[G NalG′]
216-1 Ab: cap-[ELL EEL LEE LLE]-[GFG FVG G]-[QL LAQ LLA QLL Q]-[1×K(Z)]-[G NalG′]
Ab: Transferrin Receptor Monoclonal antibody (MEM-189)
   Where Y is a PEG12-BCN linker (see below)

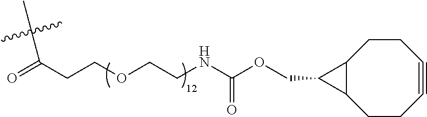

and Z is a PEG12 linker attached to a Transferrin Receptor Monoclonal antibody (see below)

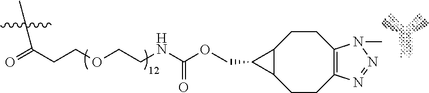

### Example 69

In this example, the following delivery agent embodiments were evaluated for activity in 80% serum. Results are shown in FIG. 71. Neither of compounds 225-2 or 224-2 exhibited activity, which, without being limited to a single theory, is currently believed to indicate that the mask peptide group is necessary for activity in high serum.
Negative control: MO only (Morpholino only)
224-2: cap-[ELL EEL LEE LLE]-[GNalG′]
225-2: cap-[ELL EEL LEE LLE]-[2×K(LA)]-[GNalG′]
217-2: cap-[ELL EEL LEE LLE]-[GF GFV GG]-[Q LLQ QLL QQL LQ]-[1×K(LA)]-[GNalG′]
   SEQ ID NO: 278: ELLEELLEELLEGXG, wherein X is naphthylalanine (Nal), the C-terminal G at position 15 is a modified glycine comprising a —C(O)—NH₂ group, and in some embodiments, the N-terminal E comprises an N-terminus modified with an acetyl group.
   SEQ ID NO: 279: ELLEELLEELLEKGXG, wherein the N terminal E at position 1 comprises the “Cap” group described above, each K is functionalized with an LA group, X is Nal, and the C-terminal G at position 16 is G′ as described above.
   Example 217-2 comprises SEQ ID NO: 274, wherein the N terminal E at position 1 comprises the “Cap” group described above, each K is functionalized with an LA group. X is Nal, and the C-terminal G at position 35 is G′ as described above.

### Example 70

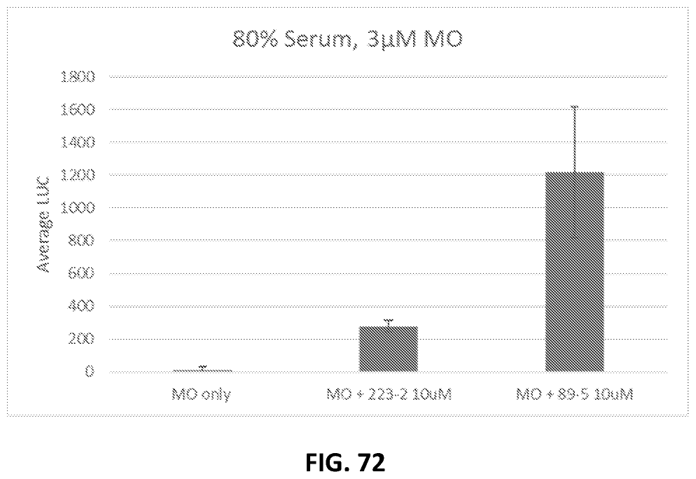

In this example, a delivery agent embodiment where two leucines in the mask peptide were replaced with two alanines in the same position was evaluated for activity in 80% serum. Results are shown in FIG. 72.
Negative control: MO only (Morpholino only)
223-2: cap-[ELL EEL LEE LLE]-[GF GFV GG]-[Q LLQ QAA QQL LQ]-[K(LA)]-[GNalG′]
Positive control: 89-5: cap-[ELL EEL LEE LLE]-[GFG FVG G]-[QL LQQ LLQ QLL Q]-[2×K(LA)]-[GNalG′]
   SEQ ID NO: 280: ELLEELLEELLEGFGFVGGOLLQQAAQQLLQKGXG, wherein K provides an anchor group, X is naphthylalanine (Nal), the C-terminal G at position 35 is a modified glycine comprising a —C(O)—NH₂ group, and in some embodiments, the N-terminal E comprises an N-terminus modified with an acetyl group.

### Example 71

In this example, a GalNac ligand and TriGalNac ligands are used in conjunction with exemplary delivery agent embodiments, such as is described below:
Cell lines: Hepatic cell line; and Hepatic cell line where the Asialoglycoprotein receptor (ASGR1) is overexpressed
Negative control: No treatment
Negative control: MO only (Morpholino only)
Positive control: 89-5: cap-[ELL EEL LEE LLE]-[GFG FVG G]-[QL LQQ LLQ QLL Q]-[2×K(LA)]-[GNalG′]
216-1 GalNac: cap-[ELL EEL LEE LLE]-[GFG FVG G]-[QL LAQ LLA QLL Q]-[1×K(Y)]-[G NalG′]
216-1 TrisGalNac: cap-[ELL EEL LEE LLE]-[GFG FVG G]-QL LAQ LLA QLL Q]-[1×K(Z)]-IG NalG′]

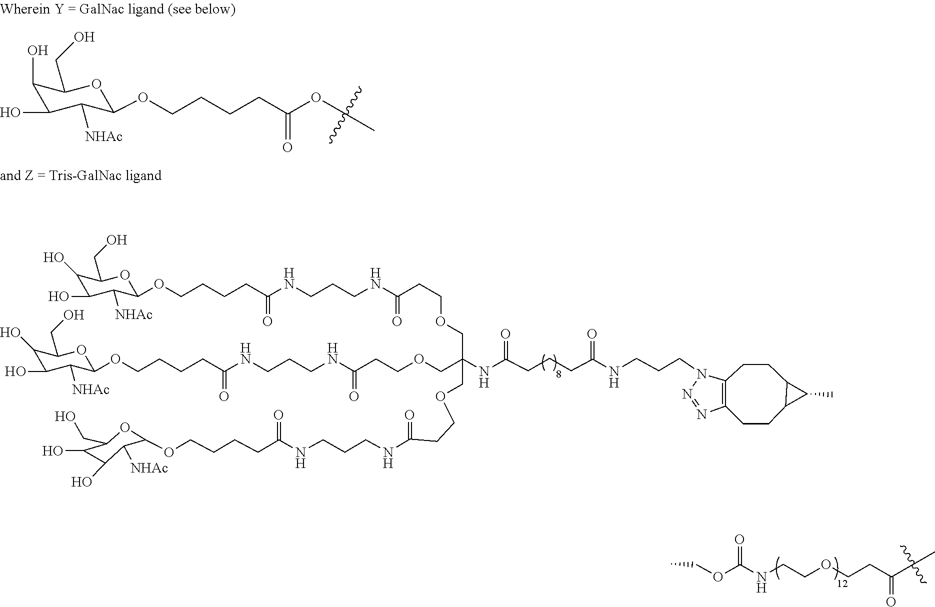

Both the GalNac ligand and the Tris-GalNac ligand should enable cell surface binding of the delivery agent vial receptor-ligand interactions with the ASGR1 receptor on hepatic cells. GalNac and Tris-GalNac have been shown to traffic to the lysosome after binding cell surface ASGR1 receptors (see Ahn G, Banik S M, Miller C L, Riley N M, Cochran J R, Bertozzi C R. LYTACs that engage the asialoglycoprotein receptor for targeted protein degradation. Nat Chem Biol. 2021 September; 17(9):937-946.). Enhanced delivery of a morpholino+GalNac or Tris-GalNac ligated delivery agent should be observed relative to morpholino with the delivery agent absent GalNac or Tris-GalNac ligands. A Mopholino sequence targeting RNA present in hepatocytes can be used. Changes in protein levels resulting from RNA binding can be assayed using assay methods recognized in the art with the benefit of the present disclosure.

### Example 72

In this example, a delivery agent comprising a monoclonal antibody for targeting transferrin receptors is evaluated in 10% serum in Hela cells either with or without transient transfection of TRFC plasmid. The TRFC plasmid is used to overexpress human transferrin receptor in the Hela cells. The delivery agent-morpholino conjugate that is bound to the antibody should have activity in the cells where the transferrin receptor is expressed and not in cells where the receptor is not expressed. The delivery agent-morpholino without an antibody bound should not have activity regardless of transferrin receptor expression. Conjugating the morpholino to the antibody-bound delivery agent should improve activity in vivo by ensuring the morpholino, antibody and delivery agent contact the cell simultaneously. The delivery agents for use in this example can include:
Negative control: No treatment.
Negative control: MO only (Morpholino only)
Positive control: 89-5: cap-[ELL EEL LEE LLE]-[GFG FVG G]-[QL LQQ LLQ QLL Q]-[2×K(LA)]-[GNalG′]
Delivery agent-Morpholino-Ab conjugate: cap-[ELL EEL LEE LLE]-[GFG FVG G]-[QL LAQ LLA QLL Q]-[1×K(Z)]-[G NalC(Y)G′]
Delivery agent-Morpholino conjugate: cap-[ELL EEL LEE LLE]-[GFG FVG G]-[QL LAQ LLA QLL Q]-[1×K(Z′)]-[G NalC(Y)G′]

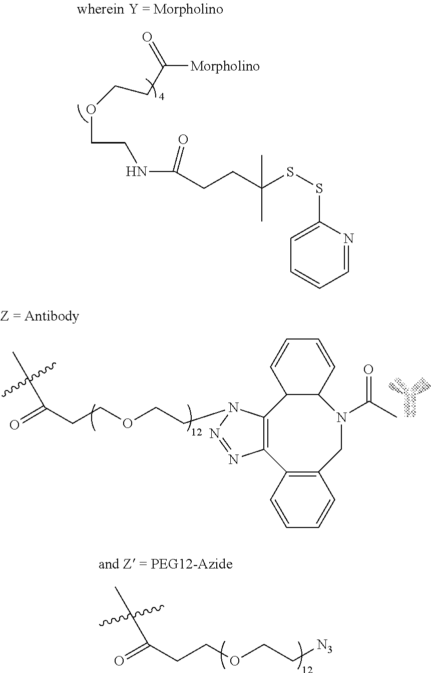

In view of the many possible embodiments to which the principles of the present disclosure may be applied, it should be recognized that the illustrated embodiments are only preferred examples of the present disclosure and should not be taken as limiting the scope of the present disclosure. Rather, the scope is defined by the following claims. We therefore claim as our invention all that comes within the scope and spirit of these claims.

1. A peptide-based delivery agent comprising:
a lytic peptide group having a structure according to a formula [X¹Y¹Y¹X¹]ₘ, wherein
each X¹ independently for each occurrence is an amino acid, or a derivative thereof, provided that at least one X¹ of the lytic peptide group is a basic amino acid or an acidic amino acid;
each Y¹ independently for each occurrence is a non-polar amino acid or a derivative thereof; and
m is an integer selected from 2 to 8;
a cleavable linker group;
a mask peptide group having a structure according to a formula [X²Y²Y²X²]_{m′}, wherein
each X² independently for each occurrence is an amino acid, or a derivative thereof, provided that at least one X² of the mask peptide group is glutamine;
each Y² independently for each occurrence is a non-polar amino acid or a derivative thereof; and
m′ is an integer selected from 2 to 8.

2. The peptide-based delivery agent of claim 1, further comprising:
(i) an anchor group selected from a heteroaliphatic group, a dibenzocyclooctyne compound, an antibody or antibody fragment, a biotin group, an avidin group, a streptavidin group, or a neutravidin group; a targeting group selected from a cell, an antibody or antibody fragment, a peptide, a biomimetic peptide, an aptamer, a sugar, or a small targeting molecule; or a combination thereof;
(ii) an N-terminus group; and
(iii) a C-terminus group;
wherein the peptide-based delivery agent has a structure according to Formula IA or Formula IB 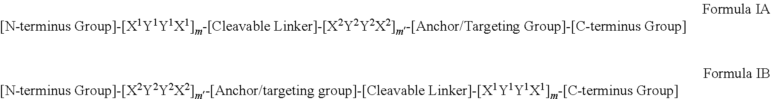
wherein
the cleavable linker is an amino acid sequence that is two to ten amino acids in length;
the anchor group, if present, is selected from a heteroaliphatic group, a dibenzocyclooctyne compound, an antibody or antibody fragment, a biotin group, an avidin group, a streptavidin group, or a neutravidin group;
the targeting group, if present, is selected from a cell, an antibody or antibody fragment, a peptide, a biomimetic peptide, an aptamer, a sugar, or a small targeting molecule; or a combination thereof;
the C-terminus group comprises an amine-terminated glycine moiety; and
the N-terminus group comprises a capping group or a fluorophore.

3. The peptide-based delivery agent of claim 1, wherein each X¹ independently for each occurrence is glutamic acid, glutamine, arginine, alanine, aspartic acid, or a derivative thereof, provided that if an X¹ is alanine, then at least one further X¹ is glutamine, glutamic acid, aspartic acid, arginine, or a derivative thereof.

4. The peptide-based delivery agent of claim 1, wherein each Y¹ independently for each occurrence and each Y² independently for each occurrence is leucine, α-methyl leucine, alanine, or a derivative thereof.

5. The peptide-based delivery agent of claim 1, wherein each X², other than the at least one X² of the mask peptide group that is glutamine as recited by claim 1, independently for each occurrence is glutamine, glutamic acid, aspartic acid, alanine, or a derivative thereof.

6. The peptide-based delivery agent of claim 1, wherein m is 3 and m′ is 3; or m is 4 and m′ is 4.

7. The peptide-based delivery agent of claim 2, wherein each X¹ is glutamic acid; each Y¹ and each Y² is leucine; each X², other than the at least one X² of the mask peptide group that is glutamine, is glutamine or alanine; m is 3; and m′ is 3; or each X¹, other than the X¹ that is directly attached to the cleavable linker, is aspartic acid; each Y¹ and each Y² is leucine; each X², other than the at least one X² of the mask peptide group that is glutamine, is glutamine or alanine; m is 3; and m′ is 3.

8. The peptide-based delivery agent of claim 1, wherein the cleavable linker is cleavable by cathepsin B and comprises an amino acid sequence two to ten amino acids in length.

9. The peptide-based delivery agent of claim 1, wherein the cleavable linker comprises an amino acid sequence at least 70% identical or 85% identical to SEQ ID NO: 3.

10. The peptide-based delivery agent of claim 2, wherein the anchor group comprises:
(i) an amino acid portion that is a lysine moiety; and
(ii) a tail group that comprises (a) a functional group provided by a 2-aminoethyl hydrogen carbonate group, a 4-aminobutanoic acid group, a —CH(NH₂)C(O)—group, or a —CH(N⁺Me₃)C(O)— group; and (b) an aliphatic tail bound to the functional group comprising 6 to 12 carbon atoms.

11. The peptide-based delivery agent of claim 10, wherein the anchor group is selected from

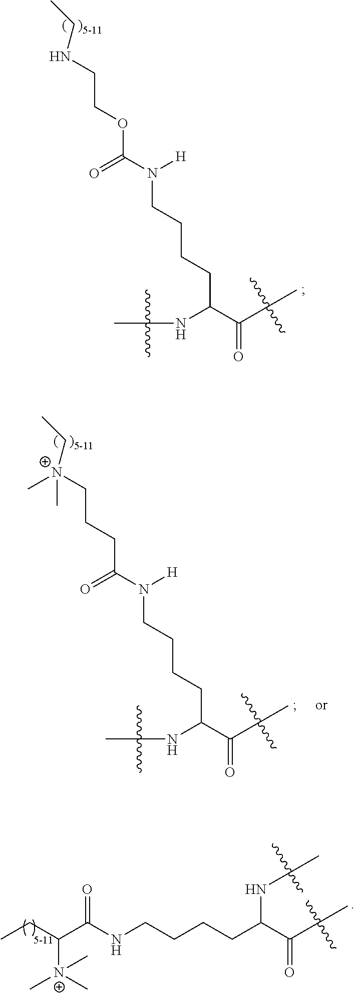

12. The peptide-based delivery agent of claim 2, wherein a plurality of anchor groups is present.

13. The peptide-based delivery agent of claim 2, wherein the lytic peptide group provides an N-terminus group of the peptide-based delivery agent and the anchor group provides a C-terminus group of the peptide-based delivery agent, wherein (i) the N-terminus group is bound to a carbonyl-containing group; and (ii) the C-terminus group is bound to an amine-terminated glycine moiety, a fluorophore, or a combination thereof.

14. The peptide-based delivery agent of claim 2, wherein:
(i) the lytic peptide group has a structure [ELLE]₃; the cleavable linker has an amino acid sequence of SEQ ID NO: 3; the mask peptide group has a structure [QLLQ]₃; and the peptide-based delivery agent comprises the anchor group; and wherein (a) the lytic peptide group provides an N-terminus that is bound to an acetyl group, and (b) the anchor group is bound to an amine-terminated glycine moiety having a structure -G-Nal-G′, wherein G′ is a modified glycine comprising a —C(O)—N(R^{a})₂ group, wherein each R^{a} independently is hydrogen or aliphatic; or
(ii) the lytic peptide group has a structure [ELLE]₄; the cleavable linker has an amino acid sequence of SEQ ID NO: 3; the mask peptide group has a structure [QLLQ]₄; and peptide-based delivery agent comprises two anchor groups and are bound directly to one another; and wherein (a) the lytic peptide group provides an N-terminus that is bound to an acetyl group, and (b) the anchor group is bound to a modified glycine comprising a —C(O)—N(R^{a})₂ group, wherein each R^{a} independently is hydrogen or aliphatic; or
(iii) the lytic peptide group has a structure [QLLE]-[QLLQ]-[QLLE]; the cleavable linker has an amino acid sequence of SEQ ID NO: 3; the mask peptide group has a structure [QLLE]-[QLLQ]-[QLLE]; and the peptide-based delivery agent comprises the anchor group; and wherein (a) the lytic peptide group provides an N-terminus that is bound to an acetyl group, and (b) the anchor group is bound to an amine-terminated glycine moiety having a structure-G-K-G′, wherein G is glycine, K is lysine, and G′ is a modified glycine comprising a —C(O)—N(R^{a})₂ group, wherein each R^{a} independently is hydrogen or aliphatic;
(iv) the lytic peptide group has a structure [ELLE]₃; the cleavable linker has an amino acid sequence of SEQ ID NO: 3; the mask peptide group has a structure [QLLA]-[QLLA]-[QLLQ]; and the peptide-based delivery agent comprises the anchor group; and wherein (a) the lytic peptide group provides an N-terminus that is bound to an acetyl group, and (b) the anchor group is bound to an amine-terminated glycine moiety having a structure -G-K-G′, wherein G is glycine, K is lysine, and G′ is a modified glycine comprising a —C(O)—N(R^{a})₂ group, wherein each R^{a} independently is hydrogen or aliphatic; or
(v) the lytic peptide group has a structure [DLLD]-[DLLD]-[DLLE]; the cleavable linker has an amino acid sequence of SEQ ID NO: 3; the mask peptide group has a structure [QLLQ]₃; and the peptide-based delivery agent comprises the anchor group; and wherein (a) the lytic peptide group provides an N-terminus that is bound to an acetyl group, and (b) the anchor group is bound to an amine-terminated glycine moiety having a structure-G-K-G′, wherein G is glycine, K is lysine, and G′ is a modified glycine comprising a —C(O)—N(R^{a})₂ group, wherein each R^{a} independently is hydrogen or aliphatic.

15. The peptide based delivery agent of claim 2, having an amino acid sequence at least 80%, at least 85%, at least 90%, at least 95%, at least 96%, at least 97%, at least 98%, at least 99%, or 100% identical to any one of SEQ ID NOs: 12, 272, 273, or 274 and wherein K at position 32 comprises a linking group selected from Formula A, B, or C, and wherein the linking group is attached to one or more DBCO groups.

16. A composition, comprising:
the peptide-based delivery agent of claim 1; and
a therapeutic agent selected from a chemotherapeutic, an antisense molecule, a therapeutic antibody, an immune therapeutic, an antibiotic, an antidepressant, or a combination thereof.

17. The composition of claim 16, wherein the therapeutic agent is a morpholino comprising a nucleic acid sequence at least 80%, at least 85%, at least 90%, at least 95%, at least 96%, at least 97%, at least 98%, at least 99%, or 100% identical to any one of SEQ ID NOs: 229, 240, 241, 242, 243, and 244.

18. The composition of claim 16, wherein (i) the peptide-based delivery agent comprises an antibody as the anchor group or the targeting group (ii) the therapeutic agent is a morpholino and wherein the morpholino is indirectly covalently bound to the peptide-based delivery agent through a linking group.

19. The composition of claim 16, formulated for administration by injection, aerosol delivery, intranasal administration, oral administration, topical administration, or a combination thereof.

20. A method, comprising contacting a cell in vitro or in vivo with the composition of claim 16, wherein the peptide-based delivery agent of the composition delivers the therapeutic agent to the cell's cytosol and contacting the cell with the composition induces:
lysis of a membrane following internalization of the therapeutic agent and the peptide-based delivery agent into the cell; or
pore formation in a membrane following internalization of the therapeutic agent and the peptide-based delivery agent into the cell; or
local disruption/destabilization of a membrane following internalization of the therapeutic agent and the peptide-based delivery agent into the cell.

21-23. (canceled)

24. A method of identifying a therapeutic compound, comprising:
contacting a cell with the peptide-based delivery agent of claim 1 and one or more compounds;
determining an effect of the one or more compounds on the contacted cell; and
comparing the effect of the one or more compounds on the contacted cell to a control; wherein a differential effect of the one or more compounds on the contacted cell relative to the control indicates that the one or more compounds is a therapeutic compound.

25. The method of claim 24, wherein the method is a quantitative high-throughput screening method and wherein the effect of the one or more compounds on the contacted cell comprises:
reduced survival of the contacted cell compared to the control;
increased survival of the contacted cell compared to the control;
induction of a phenotype of interest in the contacted cell compared to the control;
increased expression of one or more genes in the contacted cell compared to the control; and/or
decreased expression of one or more genes in the contacted cell compared to the control.

26-27. (canceled)
